(19) United States
(12) Patent Application Publication
(10) Pub. No.: US 2022/0133768 A1
(43) Pub. Date: May 5, 2022

(54) CRISPR/RNA-GUIDED NUCLEASE-RELATED METHODS AND COMPOSITIONS FOR TREATING RHO-ASSOCIATED AUTOSOMAL-DOMINANT RETINITIS PIGMENTOSA (ADRP)

(71) Applicant: EDITAS MEDICINE, INC., Cambridge, MA (US)

(72) Inventors: Benjamin Aryeh DINER, Somerville, MA (US); Deepak REYON, Somerville, MA (US)

(73) Assignee: EDITAS MEDICINE, INC., Cambridge, MA (US)

(21) Appl. No.: 17/433,975

(22) PCT Filed: Feb. 25, 2020
(86) PCT No.: PCT/US20/19766

§ 371 Date: Aug. 25, 2021

Related U.S. Application Data
(60) Provisional application No. 62/810,320, filed on Feb. 25, 2019.

(51) Int. Cl. A61K 31/7088; A61P 27/02; C12N 15/113; C12N 15/90; C12N 15/11; C12N 9/22

(52) U.S. Cl. CPC A61K 31/7088; A61P 27/02; C12N 9/22; C12N 15/90; C12N 15/111; C12N 15/113

(57) ABSTRACT

CRISPR/RNA-guided nuclease-related compositions and methods for treatment of RHO-associated retinitis pigmentosa, e.g., autosomal-dominant retinitis pigmentosa (adRP).

### PRIORITY CLAIM

The present application claims the benefit of United States Provisional Patent Application No. 62/810,320, filed Feb. 25, 2019, the subject matter of which is hereby incorporated by reference in its entirety, as if fully set forth herein.

### FIELD

The disclosure relates to CRISPR/RNA-guided nuclease-related methods and components for editing a target nucleic acid sequence, and applications thereof in connection with autosomal dominant retinitis pigmentosa (ADRP).

### BACKGROUND

Retinitis pigmentosa (RP), an inherited retinal dystrophy that affects photoreceptors and retinal pigment epithelium cells, is characterized by progressive retinal deterioration and atrophy, resulting in a gradual loss of vision and ultimately leading to blindness in affected patients. RP can be caused by both homozygous and heterozygous mutations and can present in various forms, for example, as autosomal-dominant RP (adRP), autosomal recessive RP (arRP) or X-linked RP (X-LRP). Treatment options for RP are limited, and no approved treatment that can arrest or reverse RP progression is currently available.

### SUMMARY

Some aspects of the strategies, methods, compositions, and treatment modalities provided herein address a key unmet need in the field by providing new and effective means of delivering genome editing systems to the affected cells and tissues of subjects suffering from autosomal-dominant retinitis pigmentosa (adRP). Some aspects of this disclosure provide strategies, methods, and compositions for the introduction of genome editing systems targeted to the adRP associated gene rhodopsin into retinal cells. Such strategies, methods, and compositions are useful, in some embodiments, for editing adRP associated variants of the rhodopsin gene, e.g., for inducing gene editing events that result in loss-of-function of such rhodopsin variants. In some embodiments, such strategies, methods, and compositions are useful as treatment modalities for administration to a subject in need thereof, e.g., to a subject having an autosomal-dominant form of RP. The strategies, methods, compositions, and treatment modalities provided herein thus represent an important step forward in the development of clinical interventions for the treatment of RP, e.g., for the treatment of adRP.

The RHO gene encodes the rhodopsin protein and is expressed in retinal photoreceptor (PR) rod cells. Rhodopsin is a G protein-coupled receptor expressed in the outer segment of rod cells and is a critical element of the phototransduction cascade. Defects in the RHO gene are typically characterized by decreased production of wild-type rhodopsin and/or expression of mutant rhodopsin which lead to interruptions in photoreceptor function and corresponding vision loss. Mutations in RHO typically result in degeneration of PR rod cells first, followed by degeneration of PR cone cells as the disease progresses. Subjects with RHO mutations experience progressive loss of night vision, as well as loss of peripheral visual fields followed by loss of central visual fields. Exemplary RHO mutations are provided in Table A.

Some aspects of the present disclosure provide strategies, methods, compositions, and treatment modalities for altering a RHO gene sequence, e.g., altering the sequence of a wild type and/or of a mutant RHO gene, e.g., in a cell or in a patient having adRP, by insertion or deletion of one or more nucleotides mediated by an RNA-guided nuclease (e.g., Cas9 or Cpfl molecule) and one or more guide RNAs (gRNAs), resulting in loss of function of the RHO gene sequence. This type of alteration is also referred to as “knocking out” the RHO gene. Some aspects of the present disclosure provide strategies, methods, compositions, and treatment modalities for expressing exogenous RHO, e.g., in a cell subjected to an RNA-guided nuclease-mediated knock-out of RHO, e.g., by delivering an exogenous RHO complementary DNA (cDNA) sequence encoding a functional rhodopsin protein (e.g., a wild-type rhodopsin protein).

In certain embodiments, a 5′ region of the RHO gene (e.g., 5′ untranslated region (UTR), exon 1, exon 2, intron 1, the exon 1/intron 1 border or the exon 2/intron 1 border) is targeted by an RNA-guided nuclease to alter the gene. In certain embodiments, any region of the RHO gene (e.g., a promoter region, a 5′untranslated region, a 3′ untranslated region, an exon, an intron, or an exon/intron border) is targeted by an RNA-guided nuclease to alter the gene. In certain embodiments, a non-coding region of the RHO gene (e.g., an enhancer region, a promoter region, an intron, 5′ UTR, 3′UTR, polyadenylation signal) is targeted to alter the gene. In certain embodiments, a coding region of the RHO gene (e.g., early coding region, an exon) is targeted to alter the gene. In certain embodiments, a region spanning an exon/intron border of the RHO gene (e.g., exon 1/intron 1, exon 2/intron 1) is targeted to alter the gene. In certain embodiments, a region of the RHO gene is targeted which, when altered, results in a stop codon and knocking out the RHO gene. In certain embodiments, alteration of the mutant RHO gene occurs in a mutation-independent manner, which provides the benefit of circumventing the need to develop therapeutic strategies for each RHO mutation set forth in Table A.

In an embodiment, after treatment, one or more symptoms associated with adRP (e.g., nyctalopia, abnormal electroretinogram, cataract, visual field defect, rod-cone dystrophy, or other symptom(s) known to be associated with adRP) is ameliorated, e.g., progression of adRP is delayed, inhibited, prevented or halted, PR cell degeneration is delayed, inhibited, prevented and/or halted, and/or visual loss is ameliorated, e.g., progression of visual loss is delayed, inhibited, prevented, or halted. In an embodiment, after treatment, progression of adRP is delayed, e.g., PR cell degeneration is delayed. In an embodiment, after treatment, progression of adRP is reversed, e.g., function of existing PR rod cells and cone cells and/or birth of new PR rod cells and cone cells is increased/enhanced and/or visual loss e.g., progression of visual loss is delayed, inhibited, prevented, or halted.

In an embodiment, CRISPR/RNA-guided nuclease-related methods and components and compositions of the disclosure provide for the alteration (e.g., knocking out) of a mutant RHO gene associated with adRP, by altering the sequence at a RHO target position, e.g., by creating an indel resulting in loss-of-function of the affected RHO gene or allele, e.g., a nucleotide substitution resulting in a truncation, nonsense mutation, or other type of loss-of-function of an encoded RHO gene product, e.g., of the encoded RHO mRNA or RHO protein; a deletion of one or more nucleotides resulting in a truncation, nonsense mutation, or other type of loss-of-function of an encoded RHO gene product, e.g., of the encoded RHO mRNA or RHO protein, e.g., a single nucleotide, double nucleotide, or other frame-shifting deletion, or a deletion resulting in a premature stop codon; or an insertion resulting in a truncation, nonsense mutation, or other type of loss-of-function of an encoded RHO gene product, e.g., of the encoded RHO mRNA or RHO protein e.g., a single nucleotide, double nucleotide, or other frame-shifting insertion, or an insertion resulting in a premature stop codon. In some embodiments, CRISPR/RNA-guided nuclease-related methods and components and compositions of the disclosure provide for the alteration (e.g., knocking out) of a mutant RHO gene associated with adRP, by altering the sequence at a RHO target position, e.g., creating an indel that results in nonsense-mediated decay of an encoded gene product, e.g., an encoded RHO transcript.

In one aspect, disclosed herein is a gRNA molecule, e.g., an isolated or non-naturally occurring gRNA molecule, comprising a targeting domain which is complementary with a target domain from the RHO gene.

In an embodiment, the targeting domain of the gRNA molecule is configured to provide a cleavage event, e.g., a double strand break or a single strand break, sufficiently close to an RHO target position, in the RHO gene to allow alteration in the RHO gene, resulting in disruption (e.g., knocking out) of the RHO gene activity, e.g., a loss-of-function of the RHO gene, for example, characterized by reduced or abolished expression of a RHO gene product (e.g., a RHO transcript or a RHO protein), or by expression of a dysfunctional or non-functional RHO gene product (e.g., a truncated RHO protein or transcript). In an embodiment, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150 or 200 nucleotides of an RHO target position. The break, e.g., a double strand or single strand break, can be positioned upstream or downstream of an RHO target position, in the RHO gene.

In an embodiment, a second gRNA molecule comprising a second targeting domain is configured to provide a cleavage event, e.g., a double strand break or a single strand break, sufficiently close to the RHO target position, in the RHO gene, to allow alteration in the RHO gene, either alone or in combination with the break positioned by said first gRNA molecule. In an embodiment, the targeting domains of the first and second gRNA molecules are configured such that a cleavage event, e.g., a double strand or single strand break, is positioned, independently for each of the gRNA molecules, within 1, 2, 3, 4, 5, 10, 15, 20, 25, 30, 35, 40, 45, 50, 60, 70, 80, 90, 100, 150 or 200 nucleotides of the target position. In an embodiment, the breaks, e.g., double strand or single strand breaks, are positioned on both sides of a nucleotide of a RHO target position, in the RHO gene. In an embodiment, the breaks, e.g., double strand or single strand breaks, are positioned on one side, e.g., upstream or downstream, of a nucleotide of a RHO target position, in the RHO gene.

In an embodiment, a single strand break is accompanied by an additional single strand break, positioned by a second gRNA molecule, as discussed below. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150 or 200 nucleotides of a RHO target position. In an embodiment, the first and second gRNA molecules are configured such, that when guiding a Cas9 nickase, a single strand break will be accompanied by an additional single strand break, positioned by a second gRNA, sufficiently close to one another to result in alteration of a RHO target position, in the RHO gene. In an embodiment, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In an embodiment, the two gRNA molecules are configured to position cuts at the same position, or within a few nucleotides of one another, on different strands, e.g., essentially mimicking a double strand break.

In an embodiment, a double strand break can be accompanied by an additional double strand break, positioned by a second gRNA molecule, as is discussed below. For example, the targeting domain of a first gRNA molecule is configured such that a double strand break is positioned upstream of a RHO target position, in the RHO gene, e.g., within 1, 2, 3, 4, 5, 10, 15, 20, 25, 30, 35, 40, 45, 50, 60, 70, 80, 90, 100, 150 or 200 nucleotides of the target position; and the targeting domain of a second gRNA molecule is configured such that a double strand break is positioned downstream of a RHO target position, in the RHO gene, e.g., within 1, 2, 3, 4, 5, 10, 15, 20, 25, 30, 35, 40, 45, 50, 60, 70, 80, 90, 100, 150 or 200 nucleotides of the target position.

In an embodiment, a double strand break can be accompanied by two additional single strand breaks, positioned by a second gRNA molecule and a third gRNA molecule. For example, the targeting domain of a first gRNA molecule is configured such that a double strand break is positioned upstream of a RHO target position, in the RHO gene, e.g., within 1, 2, 3, 4, 5, 10, 15, 20, 25, 30, 35, 40, 45, 50, 60, 70, 80, 90, 100, 150 or 200 nucleotides of the target position; and the targeting domains of a second and third gRNA molecule are configured such that two single strand breaks are positioned downstream of a RHO target position, in the RHO gene, e.g., within 1, 2, 3, 4, 5, 10, 15, 20, 25, 30, 35, 40, 45, 50, 60, 70, 80, 90, 100, 150 or 200 nucleotides of the target position. In an embodiment, the targeting domain of the first, second and third gRNA molecules are configured such that a cleavage event, e.g., a double strand or single strand break, is positioned, independently for each of the gRNA molecules.

In an embodiment, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of a RHO target position, in the RHO gene, e.g., within 1, 2, 3, 4, 5, 10, 15, 20, 25, 30, 35, 40, 45, 50, 60, 70, 80, 90, 100, 150 or 200 nucleotides of the target position; and the targeting domains of a third and fourth gRNA molecule are configured such that two single strand breaks are positioned downstream of a RHO target position, in the RHO gene, e.g., within 1, 2, 3, 4, 5, 10, 15, 20, 25, 30, 35, 40, 45, 50, 60, 70, 80, 90, 100, 150 or 200 nucleotides of the target position.

It is contemplated herein that when multiple gRNAs are used to generate (1) two single stranded breaks in close proximity (2) one double stranded break and two paired nicks flanking a RHO target position (e.g., to remove a piece of DNA) or (3) four single stranded breaks, two on each side of a RHO target position, that they are targeting the same RHO target position. It is further contemplated herein that multiple gRNAs may be used to target more than one RHO target position in the same gene.

In some embodiments, the targeting domain of the first gRNA molecule and the targeting domain of the second gRNA molecules are complementary to opposite strands of the target nucleic acid molecule. In some embodiments, the gRNA molecule and the second gRNA molecule are configured such that the PAMs are oriented outward.

In an embodiment, the targeting domain of a gRNA molecule is configured to avoid unwanted target chromosome elements, such as repeat elements, e.g., Alu repeats, in the target domain. The gRNA molecule may be a first, second, third and/or fourth gRNA molecule.

In an embodiment, the RHO target position is a target position located in exon 1 or exon 2 of the RHO gene and the targeting domain of a gRNA molecule comprises a sequence that is the same as, or differs by no more than 1, 2, 3, 4, or 5 nucleotides from, a targeting domain sequence from Table 1. In some embodiments, the targeting domain is selected from those in Table 1. In an embodiment, the RHO target position is a target position located in the 5′ UTR region of the RHO gene and the targeting domain of a gRNA molecule comprises a sequence that is the same as, or differs by no more than 1, 2, 3, 4, or 5 nucleotides from, a targeting domain sequence from any one of Table 2. In some embodiments, the targeting domain is selected from those in Table 2. In an embodiment, the target position is a target position located in intron 1 of the RHO gene and the targeting domain of a gRNA molecule comprises a sequence that is the same as, or differs by no more than 1, 2, 3, 4, or 5 nucleotides from, a targeting domain sequence from any one of Table 3. In some embodiments, the targeting domain is selected from those in Table 3. In an embodiment, the target position is a target position located in the RHO gene and the targeting domain of a gRNA molecule comprises a sequence that is the same as, or differs by no more than 1, 2, 3, 4, or 5 nucleotides from, a targeting domain sequence from any one of Table 18. In some embodiments, the targeting domain is selected from those in Table 18. In an embodiment, the gRNA, e.g., a gRNA comprising a targeting domain, which is complementary with the RHO gene, is a modular gRNA. In other embodiments, the gRNA is a unimolecular or chimeric gRNA.

In an embodiment, the targeting domain which is complementary with the RHO gene is 17 nucleotides or more in length. In an embodiment, the targeting domain is 17 nucleotides in length. In other embodiments, the targeting domain is 18 nucleotides in length. In still other embodiments, the targeting domain is 19 nucleotides in length. In still other embodiments, the targeting domain is 20 nucleotides in length. In still other embodiments, the targeting domain is 21 nucleotides in length. In still other embodiments, the targeting domain is 22 nucleotides in length. In still other embodiments, the targeting domain is 23 nucleotides in length. In still other embodiments, the targeting domain is 24 nucleotides in length. In still other embodiments, the targeting domain is 25 nucleotides in length. In still other embodiments, the targeting domain is 26 nucleotides in length.

A gRNA as described herein may comprise from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a gRNA comprises a linking domain of no more than 25 nucleotides in length; a proximal and tail domain, that taken together, are at least 20 nucleotides in length; and a targeting domain of 17, 18, 19 or 20 nucleotides in length.

In another embodiment, a gRNA comprises a linking domain of no more than 25 nucleotides in length; a proximal and tail domain, that taken together, are at least 30 nucleotides in length; and a targeting domain of 17, 18, 19 or 20 nucleotides in length. In another embodiment, a gRNA comprises a linking domain of no more than 25 nucleotides in length; a proximal and tail domain, that taken together, are at least 30 nucleotides in length; and a targeting domain of 17, 18, 19 or 20 nucleotides in length.

In another embodiment, a gRNA comprises a linking domain of no more than 25 nucleotides in length; a proximal and tail domain, that taken together, are at least 40 nucleotides in length; and a targeting domain of 17, 18, 19 or 20 nucleotides in length.

A cleavage event, e.g., a double strand or single strand break, is generated by an RNA-guided nuclease (e.g., a Cas9 or Cpfl molecule). The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule). In certain embodiments, the RNA-guided nuclease may be a Cpfl molecule.

In some embodiments, the RNA-guided nuclease (e.g., eaCas9 molecule or Cpfl molecule) catalyzes a double strand break.

In some embodiments, the eaCas9 molecule comprises HNH-like domain cleavage activity but has no, or no significant, N-terminal RuvC-like domain cleavage activity. In this case, the eaCas9 molecule is an HNH-like domain nickase, e.g., the eaCas9 molecule comprises a mutation at D10, e.g., D10A. In other embodiments, the eaCas9 molecule comprises N-terminal RuvC-like domain cleavage activity but has no, or no significant, HNH-like domain cleavage activity. In this instance, the eaCas9 molecule is an N-terminal RuvC-like domain nickase, e.g., the eaCas9 molecule comprises a mutation at H840, e.g., H840A.

In certain embodiments, the Cas9 molecule may be a self-inactivating Cas9 molecule designed for transient expression of the Cas9 protein.

In an embodiment, a single strand break is formed in the strand of the target nucleic acid to which the targeting domain of said gRNA is complementary. In another embodiment, a single strand break is formed in the strand of the target nucleic acid other than the strand to which the targeting domain of said gRNA is complementary.

In another aspect, disclosed herein is a nucleic acid, e.g., an isolated or non-naturally occurring nucleic acid, e.g., DNA, that comprises (a) a sequence that encodes a gRNA molecule comprising a targeting domain, as disclosed herein.

In an embodiment, the nucleic acid encodes a gRNA molecule, e.g., a first gRNA molecule, comprising a targeting domain configured to provide a cleavage event, e.g., a double strand break or a single strand break, sufficiently close to a RHO target position, in the RHO gene to allow alteration in the RHO gene. In an embodiment, the nucleic acid encodes a gRNA molecule, e.g., the first gRNA molecule, comprising a targeting domain comprising a sequence that is the same as, or differs by no more than 1, 2, 3, 4, or 5 nucleotides from, a targeting domain sequence selected from those set forth in Tables 1-3 and 18. In an embodiment, the nucleic acid encodes a gRNA molecule comprising a targeting domain sequence selected from those set forth in Tables 1-3 and 18.

In an embodiment, the nucleic acid encodes a modular gRNA, e.g., one or more nucleic acids encode a modular gRNA. In other embodiments, the nucleic acid encodes a chimeric gRNA. The nucleic acid may encode a gRNA, e.g., the first gRNA molecule, comprising a targeting domain comprising 17 nucleotides or more in length. In one embodiment, the nucleic acid encodes a gRNA, e.g., the first gRNA molecule, comprising a targeting domain that is 17 nucleotides in length. In other embodiments, the nucleic acid encodes a gRNA, e.g., the first gRNA molecule, comprising a targeting domain that is 18 nucleotides in length. In still other embodiments, the nucleic acid encodes a gRNA, e.g., the first gRNA molecule, comprising a targeting domain that is 19 nucleotides in length. In still other embodiments, the nucleic acid encodes a gRNA, e.g., the first gRNA molecule, comprising a targeting domain that is 20 nucleotides in length.

In an embodiment, a nucleic acid encodes a gRNA comprising from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a nucleic acid encodes a gRNA e.g., the first gRNA molecule, comprising a linking domain of no more than 25 nucleotides in length; a proximal and tail domain, that taken together, are at least 20 nucleotides in length; and a targeting domain of 17, 18, 19 or 20 nucleotides in length.

In an embodiment, a nucleic acid encodes a gRNA e.g., the first gRNA molecule, comprising a linking domain of no more than 25 nucleotides in length; a proximal and tail domain, that taken together, are at least 30 nucleotides in length; and a targeting domain of 17, 18, 19 or 20 nucleotides in length.

In an embodiment, a nucleic acid encodes a gRNA e.g., the first gRNA molecule, comprising a linking domain of no more than 25 nucleotides in length; a proximal and tail domain, that taken together, are at least 30 nucleotides in length; and a targeting domain of 17, 18, 19 or 20 nucleotides in length.

In an embodiment, a nucleic acid encodes a gRNA comprising e.g., the first gRNA molecule, a linking domain of no more than 25 nucleotides in length; a proximal and tail domain, that taken together, are at least 40 nucleotides in length; and a targeting domain of 17, 18, 19 or 20 nucleotides in length.

In an embodiment, a nucleic acid comprises (a) a sequence that encodes a gRNA molecule e.g., the first gRNA molecule, comprising a targeting domain that is complementary with a RHO target domain in the RHO gene as disclosed herein, and further comprising (b) a sequence that encodes an RNA-guided nuclease (e.g., Cas9 or Cpfl molecule).

The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule).

A nucleic acid disclosed herein may comprise (a) a sequence that encodes a gRNA molecule comprising a targeting domain that is complementary with a RHO target domain in the RHO gene as disclosed herein; (b) a sequence that encodes an RNA-guided nuclease (e.g., Cas9 or Cpfl molecule); (c) a RHO cDNA molecule; and further comprises (d)(i) a sequence that encodes a second gRNA molecule described herein having a targeting domain that is complementary to a second target domain of the RHO gene, and optionally, (ii) a sequence that encodes a third gRNA molecule described herein having a targeting domain that is complementary to a third target domain of the RHO gene; and optionally, (iii) a sequence that encodes a fourth gRNA molecule described herein having a targeting domain that is complementary to a fourth target domain of the RHO gene.

In an embodiment, the RHO cDNA molecule is a double stranded nucleic acid. In some embodiments, the RHO cDNA molecule comprises a nucleotide sequence, e.g., of one or more nucleotides, encoding rhodopsin protein. In certain embodiments, the RHO cDNA molecule is not codon modified. In certain embodiments, the RHO cDNA molecule is codon modified to provide resistance to hybridization with a gRNA molecule. In certain embodiments, the RHO cDNA molecule is codon modified to provide improved expression of the encoded RHO protein (e.g., SEQ ID NOs:13-18). In certain embodiments, the RHO cDNA molecule may include a nucleotide sequence comprising exon 1, exon 2, exon 3, exon 4, and exon 5 of the RHO gene. In certain embodiments, the RHO cDNA may include an intron (e.g., SEQ ID NOs:4-7). In certain embodiments, the RHO cDNA molecule may include a nucleotide sequence comprising exon 1, intron 1, exon 2, exon 3, exon 4, and exon 5 of the RHO gene. In certain embodiments, the RHO cDNA molecule may include one or more of a nucleotide sequence comprising or consisting of the sequences selected from exon 1, intron 1, exon 2, intron 2, exon 3, intron 3, exon 4, intron 4, and exon 5 of the RHO gene. In certain embodiments, the intron comprises one or more truncations at a 5′ end of intron 1, a 3′ end of intron 1, or both.

In an embodiment, a nucleic acid encodes a second gRNA molecule comprising a targeting domain configured to provide a cleavage event, e.g., a double strand break or a single strand break, sufficiently close to a RHO target position, in the RHO gene, to allow alteration in the RHO gene, either alone or in combination with the break positioned by said first gRNA molecule.

In an embodiment, a nucleic acid encodes a third gRNA molecule comprising a targeting domain configured to provide a cleavage event, e.g., a double strand break or a single strand break, sufficiently close to a RHO target position, in the RHO gene to allow alteration in the RHO gene, either alone or in combination with the break positioned by the first and/or second gRNA molecule.

In an embodiment, a nucleic acid encodes a fourth gRNA molecule comprising a targeting domain configured to provide a cleavage event, e.g., a double strand break or a single strand break, sufficiently close to a RHO target position, in the RHO gene to allow alteration either alone or in combination with the break positioned by the first gRNA molecule, the second gRNA molecule and the third gRNA molecule.

In an embodiment, the nucleic acid encodes a second gRNA molecule. The second gRNA is selected to target the same RHO target position, as the first gRNA molecule. Optionally, the nucleic acid may encode a third gRNA, and further optionally, the nucleic acid may encode a fourth gRNA molecule. The third gRNA molecule and the fourth gRNA molecule are selected to target the same RHO target position, as the first and second gRNA molecules.

In an embodiment, the nucleic acid encodes a second gRNA molecule comprising a targeting domain comprising a sequence that is the same as, or differs by no more than 1, 2, 3, 4, or 5 nucleotides from, a targeting domain sequence selected from those set forth in Tables 1-3 and 18. In an embodiment, the nucleic acid encodes a second gRNA molecule comprising a targeting domain selected from those set forth in Tables 1-3 and 18. In an embodiment, when a third or fourth gRNA molecule are present, the third and fourth gRNA molecules may independently comprise a targeting domain comprising a sequence that is the same as, or differs by no more than 1, 2, 3, 4, or 5 nucleotides from, a targeting domain sequence selected from those set forth in Tables 1-3 and 18. In a further embodiment, when a third or fourth gRNA molecule are present, the third and fourth gRNA molecules may independently comprise a targeting domain selected from those set forth in Tables 1-3 and 18.

In an embodiment, the nucleic acid encodes a second gRNA which is a modular gRNA, e.g., wherein one or more nucleic acid molecules encode a modular gRNA. In other embodiments, the nucleic acid encoding a second gRNA is a chimeric gRNA. In other embodiments, when a nucleic acid encodes a third or fourth gRNA, the third and fourth gRNA may be a modular gRNA or a chimeric gRNA. When multiple gRNAs are used, any combination of modular or chimeric gRNAs may be used.

A nucleic acid may encode a second, a third, and/or a fourth gRNA comprising a targeting domain comprising 17 nucleotides or more in length. In an embodiment, the nucleic acid encodes a second gRNA comprising a targeting domain that is 17 nucleotides in length. In other embodiments, the nucleic acid encodes a second gRNA comprising a targeting domain that is 18 nucleotides in length. In still other embodiments, the nucleic acid encodes a second gRNA comprising a targeting domain that is 19 nucleotides in length. In still other embodiments, the nucleic acid encodes a second gRNA comprising a targeting domain that is 20 nucleotides in length.

In an embodiment, a nucleic acid encodes a second, a third, and/or a fourth gRNA comprising from 5′ to 3′: a targeting domain; a first complementarity domain; a linking domain; a second complementarity domain; a proximal domain; and a tail domain. In some embodiments, the proximal domain and tail domain are taken together as a single domain.

In an embodiment, a nucleic acid encodes a second, a third, and/or a fourth gRNA comprising a linking domain of no more than 25 nucleotides in length; a proximal and tail domain, that taken together, are at least 20 nucleotides in length; and a targeting domain of 17, 18, 19 or 20 nucleotides in length.

In an embodiment, a nucleic acid encodes a second, a third, and/or a fourth gRNA comprising a linking domain of no more than 25 nucleotides in length; a proximal and tail domain, that taken together, are at least 30 nucleotides in length; and a targeting domain of 17, 18, 19 or 20 nucleotides in length.

In an embodiment, a nucleic acid encodes a second, a third, and/or a fourth gRNA comprising a linking domain of no more than 25 nucleotides in length; a proximal and tail domain, that taken together, are at least 30 nucleotides in length; and a targeting domain of 17, 18, 19 or 20 nucleotides in length.

In an embodiment, a nucleic acid encodes a second, a third, and/or a fourth gRNA comprising a linking domain of no more than 25 nucleotides in length; a proximal and tail domain, that taken together, are at least 40 nucleotides in length; and a targeting domain of 17, 18, 19 or 20 nucleotides in length.

As described above, a nucleic acid may comprise (a) a sequence encoding a gRNA molecule comprising a targeting domain that is complementary with a target domain in the RHO gene, (b) a sequence encoding an RNA-guided nuclease (e.g., Cas9 or Cpfl molecule), and (c) a RHO cDNA molecule sequence. In some embodiments, (a), (b), and (c) are present on the same nucleic acid molecule, e.g., the same vector, e.g., the same viral vector, e.g., the same adeno-associated virus (AAV) vector. In an embodiment, the nucleic acid molecule is an AAV vector. Exemplary AAV vectors that may be used in any of the described compositions and methods include an AAV5 vector, a modified AAV5 vector, AAV2 vector, a modified AAV2 vector, an AAV3 vector, a modified AAV3 vector, an AAV6 vector, a modified AAV6 vector, an AAV8 vector and an AAV5 vector.

In other embodiments, (a) is present on a first nucleic acid molecule, e.g. a first vector, e.g., a first viral vector, e.g., a first AAV vector; and (b) and (c) are present on a second nucleic acid molecule, e.g., a second vector, e.g., a second vector, e.g., a second AAV vector. The first and second nucleic acid molecules may be AAV vectors.

In other embodiments, (a) and (b) are present on a first nucleic acid molecule, e.g. a first vector, e.g., a first viral vector, e.g., a first AAV vector; and (c) is present on a second nucleic acid molecule, e.g., a second vector, e.g., a second vector, e.g., a second AAV vector. The first and second nucleic acid molecules may be AAV vectors.

In other embodiments, (a) and (c) are present on a first nucleic acid molecule, e.g. a first vector, e.g., a first viral vector, e.g., a first AAV vector; and (b) is present on a second nucleic acid molecule, e.g., a second vector, e.g., a second vector, e.g., a second AAV vector. The first and second nucleic acid molecules may be AAV vectors.

In other embodiments, (a) is present on a first nucleic acid molecule, e.g. a first vector, e.g., a first viral vector, e.g., a first AAV vector; (b) is present on a second nucleic acid molecule, e.g., a second vector, e.g., a second vector, e.g., a second AAV vector; and (c) is present on a third nucleic acid molecule, e.g., a third vector, e.g., a third vector, e.g., a third AAV vector. The first, second, and third nucleic acid molecules may be AAV vectors.

In other embodiments, the nucleic acid may further comprise (d)(i) a sequence that encodes a second gRNA molecule as described herein. In some embodiments, the nucleic acid comprises (a), (b), (c), and (d)(i). Each of (a), (b), (c), and (d)(i) may be present on the same nucleic acid molecule, e.g., the same vector, e.g., the same viral vector, e.g., the same adeno-associated virus (AAV) vector. In an embodiment, the nucleic acid molecule is an AAV vector.

In other embodiments, (a) and (d)(i) are on different vectors. For example, (a) may be present on a first nucleic acid molecule, e.g. a first vector, e.g., a first viral vector, e.g., a first AAV vector; and (d)(i) may be present on a second nucleic acid molecule, e.g., a second vector, e.g., a second vector, e.g., a second AAV vector. In an embodiment, the first and second nucleic acid molecules are AAV vectors.

In other embodiments, (b) and (d)(i) are on different vectors. For example, (b) may be present on a first nucleic acid molecule, e.g. a first vector, e.g., a first viral vector, e.g., a first AAV vector; and (d)(i) may be present on a second nucleic acid molecule, e.g., a second vector, e.g., a second vector, e.g., a second AAV vector. In an embodiment, the first and second nucleic acid molecules are AAV vectors.

In other embodiments, (c) and (d)(i) are on different vectors. For example, (c) may be present on a first nucleic acid molecule, e.g. a first vector, e.g., a first viral vector, e.g., a first AAV vector; and (d)(i) may be present on a second nucleic acid molecule, e.g., a second vector, e.g., a second vector, e.g., a second AAV vector. In an embodiment, the first and second nucleic acid molecules are AAV vectors.

In another embodiment, (a) and (d)(i) are present on the same nucleic acid molecule, e.g., the same vector, e.g., the same viral vector, e.g., an AAV vector. In an embodiment, the nucleic acid molecule is an AAV vector. In an alternate embodiment, (a) and (d)(i) are encoded on a first nucleic acid molecule, e.g., a first vector, e.g., a first viral vector, e.g., a first AAV vector; and a second and third of (a) and (d)(i) are encoded on a second nucleic acid molecule, e.g., a second vector, e.g., a second vector, e.g., a second AAV vector. The first and second nucleic acid molecule may be AAV vectors.

In another embodiment, (b) and (d)(i) are present on the same nucleic acid molecule, e.g., the same vector, e.g., the same viral vector, e.g., an AAV vector. In an embodiment, the nucleic acid molecule is an AAV vector. In an alternate embodiment, (b) and (d)(i) are encoded on a first nucleic acid molecule, e.g., a first vector, e.g., a first viral vector, e.g., a first AAV vector; and a second and third of (b) and (d)(i) are encoded on a second nucleic acid molecule, e.g., a second vector, e.g., a second vector, e.g., a second AAV vector. The first and second nucleic acid molecule may be AAV vectors.

In another embodiment, (c) and (d)(i) are present on the same nucleic acid molecule, e.g., the same vector, e.g., the same viral vector, e.g., an AAV vector. In an embodiment, the nucleic acid molecule is an AAV vector. In an alternate embodiment, (c) and (d)(i) are encoded on a first nucleic acid molecule, e.g., a first vector, e.g., a first viral vector, e.g., a first AAV vector; and a second and third of (c) and (d)(i) are encoded on a second nucleic acid molecule, e.g., a second vector, e.g., a second vector, e.g., a second AAV vector. The first and second nucleic acid molecule may be AAV vectors.

In another embodiment, each of (a), (b), and (d)(i) are present on the same nucleic acid molecule, e.g., the same vector, e.g., the same viral vector, e.g., an AAV vector. In an embodiment, the nucleic acid molecule is an AAV vector. In an alternate embodiment, one of (a), (b), and (d)(i) is encoded on a first nucleic acid molecule, e.g., a first vector, e.g., a first viral vector, e.g., a first AAV vector; and a second and third of (a), (b), and (d)(i) is encoded on a second nucleic acid molecule, e.g., a second vector, e.g., a second vector, e.g., a second AAV vector. The first and second nucleic acid molecule may be AAV vectors.

In another embodiment, each of (b), (c), and (d)(i) are present on the same nucleic acid molecule, e.g., the same vector, e.g., the same viral vector, e.g., an AAV vector. In an embodiment, the nucleic acid molecule is an AAV vector. In an alternate embodiment, one of (b), (c), and (d)(i) is encoded on a first nucleic acid molecule, e.g., a first vector, e.g., a first viral vector, e.g., a first AAV vector; and a second and third of (b), (c), and (d)(i) is encoded on a second nucleic acid molecule, e.g., a second vector, e.g., a second vector, e.g., a second AAV vector. The first and second nucleic acid molecule may be AAV vectors.

In another embodiment, each of (a), (c), and (d)(i) are present on the same nucleic acid molecule, e.g., the same vector, e.g., the same viral vector, e.g., an AAV vector. In an embodiment, the nucleic acid molecule is an AAV vector. In an alternate embodiment, one of (a), (c), and (d)(i) is encoded on a first nucleic acid molecule, e.g., a first vector, e.g., a first viral vector, e.g., a first AAV vector; and a second and third of (a), (c), and (d)(i) is encoded on a second nucleic acid molecule, e.g., a second vector, e.g., a second vector, e.g., a second AAV vector. The first and second nucleic acid molecule may be AAV vectors.

In an embodiment, (a) is present on a first nucleic acid molecule, e.g., a first vector, e.g., a first viral vector, a first AAV vector; and (b), (c), and (d)(i) are present on a second nucleic acid molecule, e.g., a second vector, e.g., a second vector, e.g., a second AAV vector. The first and second nucleic acid molecule may be AAV vectors.

In other embodiments, (b) is present on a first nucleic acid molecule, e.g., a first vector, e.g., a first viral vector, e.g., a first AAV vector; and (a), (c), and (d)(i) are present on a second nucleic acid molecule, e.g., a second vector, e.g., a second vector, e.g., a second AAV vector. The first and second nucleic acid molecule may be AAV vectors.

In other embodiments, (c) is present on a first nucleic acid molecule, e.g., a first vector, e.g., a first viral vector, e.g., a first AAV vector; and (a), (b), and (d)(i) are present on a second nucleic acid molecule, e.g., a second vector, e.g., a second vector, e.g., a second AAV vector. The first and second nucleic acid molecule may be AAV vectors.

In other embodiments, (d)(i) is present on a first nucleic acid molecule, e.g., a first vector, e.g., a first viral vector, e.g., a first AAV vector; and (a), (b), and (c) are present on a second nucleic acid molecule, e.g., a second vector, e.g., a second vector, e.g., a second AAV vector. The first and second nucleic acid molecule may be AAV vectors.

In another embodiment, each of (a), (b), (c), and (d)(i) are present on different nucleic acid molecules, e.g., different vectors, e.g., different viral vectors, e.g., different AAV vector. For example, (a) may be on a first nucleic acid molecule, (b) on a second nucleic acid molecule, (c) on a third nucleic acid molecule, and (d)(i) on a fourth nucleic acid molecule. The first, second, third, and fourth nucleic acid molecule may be AAV vectors.

In another embodiment, when a third and/or fourth gRNA molecule are present, each of (a), (b), (c), (d)(i), (d)(ii) and (d)(iii) may be present on the same nucleic acid molecule, e.g., the same vector, e.g., the same viral vector, e.g., an AAV vector. In an embodiment, the nucleic acid molecule is an AAV vector. In an alternate embodiment, each of (a), (b), (c), (d)(i), (d)(ii) and (d)(iii) may be present on the different nucleic acid molecules, e.g., different vectors, e.g., the different viral vectors, e.g., different AAV vectors. In further embodiments, each of (a), (b), (c), (d)(i), (d)(ii) and (d)(iii) may be present on more than one nucleic acid molecule, but fewer than six nucleic acid molecules, e.g., AAV vectors.

The nucleic acids described herein may comprise a promoter operably linked to the sequence that encodes the gRNA molecule of (a), e.g., a promoter described herein. The nucleic acid may further comprise a second promoter operably linked to the sequence that encodes the second, third and/or fourth gRNA molecule of (d), e.g., a promoter described herein. The promoter and second promoter differ from one another. In some embodiments, the promoter and second promoter are the same.

The nucleic acids described herein may further comprise a promoter operably linked to the sequence that encodes the RNA-guided nuclease (e.g., Cas9 or Cpfl molecule) of (b), e.g., a promoter described herein. In certain embodiments, the promoter operably linked to the sequence that encodes the RNA-guided nuclease of (b) comprises a rod-specific promoter. In certain embodiments, the rod-specific promoter may be a human RHO promoter. In certain embodiments, the human RHO promoter may be a minimal RHO promoter (e.g., SEQ ID NO:44).

The nucleic acids described herein may further comprise a promoter operably linked to the RHO cDNA molecule of (c), e.g., a promoter described herein. In certain embodiments, the promoter operably linked to the RHO cDNA molecule of (c) comprises a rod-specific promoter. In certain embodiments, the rod-specific promoter may be a human RHO promoter. In certain embodiments, the human RHO promoter may be a minimal RHO promoter (e.g., SEQ ID NO:44). In certain embodiments, the nucleic acids may further comprise a 3′ UTR nucleotide sequence downstream of the RHO cDNA molecule. In certain embodiments, the 3′ UTR nucleotide sequence downstream of the RHO cDNA molecule may comprise a RHO gene 3′ UTR nucleotide sequence. In certain embodiments, the 3′ UTR nucleotide sequence downstream of the RHO cDNA molecule may comprise a 3′ UTR nucleotide sequence of an mRNA encoding a highly expressed protein. For example, in certain embodiments, the 3′ UTR nucleotide sequence downstream of the RHO cDNA molecule may comprise an α-globin 3′ UTR nucleotide sequence. In certain embodiments, the 3′ UTR nucleotide sequence downstream of the RHO cDNA molecule may comprise a (3-globin 3′ UTR nucleotide sequence. In certain embodiments, the 3′ UTR nucleotide sequence comprises one or more truncations at a 5′ end of said 3′ UTR nucleotide sequence, a 3′ end of said 3′ UTR nucleotide sequence, or both.

In another aspect, disclosed herein is a composition comprising (a) a gRNA molecule comprising a targeting domain that is complementary with a target domain in the RHO gene, as described herein. The composition of (a) may further comprise (b) an RNA-guided nuclease (e.g., Cas9 or Cpfl molecule as described herein). Cpfl is also sometimes referred to as Cas12a. A composition of (a) and (b) may further comprise (c) a RHO cDNA molecule. A composition of (a), (b), and (c) may further comprise (d) a second, third and/or fourth gRNA molecule, e.g., a second, third and/or fourth gRNA molecule described herein.

In another aspect, disclosed herein is a method of altering a cell, e.g., altering the structure, e.g., altering the sequence, of a target nucleic acid of a cell, comprising contacting said cell with: (a) a gRNA that targets the RHO gene, e.g., a gRNA as described herein; (b) an RNA-guided nuclease (e.g., Cas9 or Cpfl molecule as described herein); and (c) a RHO cDNA molecule; and optionally, (d) a second, third and/or fourth gRNA that targets RHO gene, e.g., a gRNA.

In some embodiments, the method comprises contacting said cell with (a) and (b).

In some embodiments, the method comprises contacting said cell with (a), (b), and (c).

In some embodiments, the method comprises contacting said cell with (a), (b), (c) and (d).

The gRNA of (a) and optionally (d) may comprise a targeting domain sequence selected from those set forth in Tables 1-3 and 18, or may comprise a targeting domain sequence that differs by no more than 1, 2, 3, 4, or 5 nucleotides from a targeting domain sequence set forth in any of Tables 1-3 and 18.

In some embodiments, the method comprises contacting a cell from a subject suffering from or likely to develop adRP. The cell may be from a subject having a mutation at a RHO target position.

In some embodiments, the cell being contacted in the disclosed method is a cell from the eye of the subject, e.g., a retinal cell, e.g., a photoreceptor cell. The contacting may be performed ex vivo and the contacted cell may be returned to the subject's body after the contacting step. In other embodiments, the contacting step may be performed in vivo.

In some embodiments, the method of altering a cell as described herein comprises acquiring knowledge of the presence of a mutation in the RHO gene, in said cell, prior to the contacting step. Acquiring knowledge of a mutation in the RHO gene, in the cell may be by sequencing the RHO gene, or a portion of the RHO gene.

In some embodiments, the contacting step of the method comprises contacting the cell with a nucleic acid, e.g., a vector, e.g., an AAV vector, that expresses at least one of (a), (b), and (c). In some embodiments, the contacting step of the method comprises contacting the cell with a nucleic acid, e.g., a vector, e.g., an AAV vector, that expresses each of (a), (b), and (c). In another embodiment, the contacting step of the method comprises delivering to the cell an RNA-guided nuclease (e.g., Cas9 or Cpfl molecule) of (b) and a nucleic acid which encodes a gRNA (a), a RHO cDNA (c), and optionally, a second gRNA (d)(i), and further optionally, a third gRNA (d)(iv) and/or fourth gRNA (d)(iii).

In some embodiments, the contacting step of the method comprises contacting the cell with a nucleic acid, e.g., a vector, e.g., an AAV vector, that expresses at least one of (a), (b), (c) and (d). In some embodiments, the contacting step of the method comprises contacting the cell with a nucleic acid, e.g., a vector, e.g., an AAV vector, that expresses each of (a), (b), and (c). In another embodiment, the contacting step of the method comprises delivering to the cell an RNA-guided nuclease (e.g., Cas9 or Cpfl molecule) of (b), a nucleic acid which encodes a gRNA (a) and a RHO cDNA molecule (c), and optionally, a second gRNA (d)(i), and further optionally, a third gRNA (d)(iv) and/or fourth gRNA (d)(iii).

In an embodiment, contacting comprises contacting the cell with a nucleic acid, e.g., a vector, e.g., an AAV vector, e.g., an AAV5 vector, a modified AAV5 vector, an AAV2 vector, a modified AAV2 vector, an AAV3 vector, a modified AAV3 vector, an AAV6 vector, a modified AAV6 vector, an AAV8 vector or an AAV5 vector.

In an embodiment, contacting comprises delivering to the cell an RNA-guided nuclease (e.g., Cas9 or Cpfl molecule) of (b), as a protein or an mRNA, and a nucleic acid which encodes (a) and (c) and optionally (d).

In an embodiment, contacting comprises delivering to the cell an RNA-guided nuclease (e.g., Cas9 or Cpfl molecule) of (b), as a protein or an mRNA, said gRNA of (a), as an RNA, and optionally said second gRNA of (d), as an RNA, and the RHO cDNA molecule (c) as a DNA.

In an embodiment, contacting comprises delivering to the cell a gRNA of (a) as an RNA, optionally said second gRNA of (d) as an RNA, and a nucleic acid that encodes the RNA-guided nuclease (e.g., Cas9 or Cpfl molecule) of (b), and the RHO cDNA molecule (c) as a DNA.

In another aspect, disclosed herein is a method of treating a subject suffering from or likely to develop adRP, e.g., altering the structure, e.g., sequence, of a target nucleic acid of the subject, comprising contacting the subject (or a cell from the subject) with:

(a) a gRNA that targets the RHO gene, e.g., a gRNA disclosed herein;

(b) an RNA-guided nuclease, e.g., a Cas9 or Cpfl molecule disclosed herein; and

(c) a RHO cDNA molecule; and

optionally, (d)(i) a second gRNA that targets the RHO gene, e.g., a second gRNA disclosed herein, and

further optionally, (d)(ii) a third gRNA, and still further optionally, (d)(iii) a fourth gRNA that target the RHO gene, e.g., a third and fourth gRNA disclosed herein.

In some embodiments, contacting comprises contacting with (a) and (b).

In some embodiments, contacting comprises contacting with (a), (b), and (c).

In some embodiments, contacting comprises contacting with (a), (b), (c), and (d)(i).

In some embodiments, contacting comprises contacting with (a), (b), (c), (d)(i) and (d)(ii).

In some embodiments, contacting comprises contacting with (a), (b), (c), (d)(i), (d)(ii) and (d)(iii).

The gRNA of (a) or (d) (e.g., (d)(i), (d)(ii), or (d)(iii) may comprise a targeting domain sequence selected from any of those set forth in Tables 1-3 and 18, or may comprise a targeting domain sequence that differs by no more than 1, 2, 3, 4, or 5 nucleotides from a targeting domain sequence set forth in any of Tables 1-3 and 18.

In an embodiment, the method comprises acquiring knowledge of the presence of a mutation in the RHO gene, in said subject.

In an embodiment, the method comprises acquiring knowledge of the presence of a mutation in the RHO gene, in said subject by sequencing the RHO gene or a portion of the RHO gene.

In an embodiment, the method comprises altering a RHO target position in a RHO gene resulting in knocking out the RHO gene and providing exogenous RHO cDNA.

When the method comprises altering a RHO target position and providing exogenous RHO cDNA, an RNA-guided nuclease (e.g., Cas9 or Cpfl molecule) of (b), at least one guide RNA (e.g., a guide RNA of (a) and a RHO cDNA molecule (c) are included in the contacting step.

In an embodiment, a cell of the subject is contacted ex vivo with (a), (b), (c) and optionally (d). In an embodiment, said cell is returned to the subject's body.

In an embodiment, a cell of the subject is contacted is in vivo with (a), (b), (c) and optionally (d).

In an embodiment, the cell of the subject is contacted in vivo by intravenous delivery of (a), (b), (c) and optionally (d).

In an embodiment, contacting comprises contacting the subject with a nucleic acid, e.g., a vector, e.g., an AAV vector, described herein, e.g., a nucleic acid that encodes at least one of (a), (b), (c) and optionally (d).

In an embodiment, contacting comprises delivering to said subject said RNA-guided nuclease (e.g., Cas9 or Cpfl molecule) of (b), as a protein or mRNA, and a nucleic acid which encodes (a), a RHO cDNA molecule of (c) and optionally (d).

In an embodiment, contacting comprises delivering to the subject the RNA-guided nuclease (e.g., Cas9 or Cpfl molecule) of (b), as a protein or mRNA, the gRNA of (a), as an RNA, a RHO cDNA molecule of (c) and optionally the second gRNA of (d), as an RNA.

In an embodiment, contacting comprises delivering to the subject the gRNA of (a), as an RNA, optionally said second gRNA of (d), as an RNA, a nucleic acid that encodes the RNA-guided nuclease (e.g., Cas9 or Cpfl molecule) of (b), and a RHO cDNA molecule of (c).

In an embodiment, a cell of the subject is contacted ex vivo with (a), (b), (c), and optionally (d). In an embodiment, said cell is returned to the subject's body.

In an embodiment, a cell of the subject is contacted is in vivo with (a), (b), (c) and optionally (d). In an embodiment, the cell of the subject is contacted in vivo by intravenous delivery of (a), (b), (c) and optionally (d).

In an embodiment, contacting comprises contacting the subject with a nucleic acid, e.g., a vector, e.g., an AAV vector, described herein, e.g., a nucleic acid that encodes at least one of (a), (b), (c) and optionally (d).

In an embodiment, contacting comprises delivering to said subject said RNA-guided nuclease (e.g., Cas9 or Cpfl molecule) of (b), as a protein or mRNA, and a nucleic acid which encodes (a), (c) and optionally (d).

In an embodiment, contacting comprises delivering to the subject the RNA-guided nuclease (e.g., Cas9 or Cpfl molecule) of (b), as a protein or mRNA, the gRNA of (a), as an RNA, and optionally the second gRNA of (d), as an RNA, and further optionally the RHO cDNA molecule of (c) as a DNA.

In an embodiment, contacting comprises delivering to the subject the gRNA of (a), as an RNA, optionally said second gRNA of (d), as an RNA, and a nucleic acid that encodes the RNA-guided nuclease (e.g., Cas9 or Cpfl molecule) of (b), and the RHO cDNA molecule of (c) as a DNA.

In another aspect, disclosed herein is a reaction mixture comprising a, gRNA, a nucleic acid, or a composition described herein, and a cell, e.g., a cell from a subject having, or likely to develop adRP, or a subject having a mutation in the RHO gene.

In another aspect, disclosed herein is a kit comprising, (a) gRNA molecule described herein, or nucleic acid that encodes the gRNA, and one or more of the following:

(b) an RNA-guided nuclease molecule, e.g., a Cas9 or Cpfl molecule described herein, or a nucleic acid or mRNA that encodes the RNA-guided nuclease;

(c) a RHO cDNA molecule;

(d)(i) a second gRNA molecule, e.g., a second gRNA molecule described herein or a nucleic acid that encodes (d)(i);

(d)(ii) a third gRNA molecule, e.g., a second gRNA molecule described herein or a nucleic acid that encodes (d)(ii);

(d)(iii) a fourth gRNA molecule, e.g., a second gRNA molecule described herein or a nucleic acid that encodes (d)(iii).

In an embodiment, the kit comprises nucleic acid, e.g., an AAV vector, that encodes one or more of (a), (b), (c), (d)(i), (d)(ii), and (d)(iii).

In certain embodiments, the vector or nucleic acid may include a sequence set forth in one or more of SEQ ID NOs:8-11.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

Headings, including numeric and alphabetical headings and subheadings, are for organization and presentation and are not intended to be limiting.

Other features and advantages of the disclosure will be apparent from the detailed description, drawings, and from the claims.

### DESCRIPTION OF THE DRAWINGS

The accompanying drawings exemplify certain aspects and embodiments of the present disclosure. The depictions in the drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic, two- or three dimensional structures; these depictions are intended to be illustrative, rather than limiting or binding to any particular model or theory regarding their structure.

FIG. 1 illustrates the genome editing strategy implemented in certain embodiments of the disclosure. Step 1 includes knocking out (“KO”) or alteration of the RHO gene, for example, in the RHO target position of exon 1. Knocking out the RHO gene results in loss of function of the endogenous RHO gene (e.g., a mutant RHO gene). Step 2 includes replacing the RHO gene with an exogenous RHO cDNA including a minimal RHO promoter and a RHO cDNA.

FIG. 2 is a schematic of an exemplary dual AAV delivery system that may be used for a variety of applications, including without limitation, the alteration of the RHO target position, according to certain embodiments of the disclosure. Vector 1 shows an AAV5 genome, which encodes ITRs, a GRK1 promoter, and a Cas9 molecule flanked by NLS sequences. Vector 2 shows an AAV5 genome, which encodes ITRs, a minimal RHO promoter, a RHO cDNA molecule, a U6 promoter, and a gRNA. In certain embodiments, the AAV vectors may be delivered via subretinal injection.

FIG. 3 is a schematic of an exemplary dual AAV delivery system that may be used for a variety of applications, including without limitation, the alteration of the RHO target position, according to certain embodiments of the disclosure. Vector 1 shows an AAV5 genome, which encodes a minimal RHO promoter and a Cas9 molecule. Vector 2 shows an AAV5 genome, which encodes a minimal RHO promoter, a RHO cDNA molecule, a U6 promoter, and a gRNA. In certain embodiments, the AAV vectors may be delivered via subretinal injection.

FIG. 4 depicts indels of the RHO gene in HEK293 cells formed by dose-dependent gene editing using ribonucleoproteins (RNPs) comprising RHO-3, RHO-7, or RHO-10 gRNAs (Table 17) and Cas9. Increasing concentrations of RNP were delivered to HEK293 cells. Indels of the RHO gene were assessed using next generation sequencing (NGS). Data from RNP comprising RHO-3 gRNA, RHO-10 gRNA, or RHO-7 gRNA are represented by circles, squares, and triangles, respectively. Data from control plasmid (expressing Cas9 with scrambled gRNA that does not target a sequence within the human genome) are represented by X.

FIG. 5 shows details characterizing the predicted gRNA RHO alleles generated by RHO-3, RHO-7, or RHO-10 gRNAs (Table 17). As shown in the schematic of the human RHO cDNA and corresponding exons at the bottom of FIG. 5, RHO-3, RHO-10, and RHO-7 gRNAs are predicted to cut the RHO cDNA at Exon 1, the Exon 2/Intron 2 border, and the Exon 1/Intron 1 border, respectively. The target site positions for RHO-3, RHO-10, and RHO-7 gRNAs are located at bases encoding amino acids (AA) 96, 174, and 120 of the RHO protein, respectively. The protein lengths for each resulting construct for the predicted −1, −2, and −3 frame shifts are set forth. For RHO-3, a 1 base deletion at position 96 results in a truncated protein that is 95 amino acids long, a 2 base deletion at position 96 results in a truncated protein that is 120 amino acids long, a 3 base deletion at position 96 results in a truncated protein that is 347 amino acids long. For RHO-10, a 1 base deletion at position 174 results in a truncated protein that is 215 amino acids long, a 2 base deletion at position 174 results in a truncated protein that is 328 amino acids long, a 3 base deletion at position 174 results in a truncated protein that is 347 amino acids long. For RHO-7, a 1 base deletion at position 120 results in a truncated protein that is 142 amino acids long, a 2 base deletion at position 120 results in a truncated protein that is 142 amino acids long, a 3 base deletion at position 120 results in a truncated protein that is 347 amino acids long. FIG. 6. provides schematics of the predicted truncated proteins.

FIG. 6 shows schematics of the predicted RHO alleles generated by RHO-3, RHO-7, or RHO-10 gRNAs (Table 17). RHO alleles were predicted based on deletions of 1, 2, or 3 base pairs at the RHO-3, RHO-7, or RHO-10 cut sites. RHO Exons are represented by dark grey, stop codons are represented by black, missense protein is represented by stripes, deletions are represented by light grey.

FIGS. 7A and 7B show the viability of HEK293 cells expressing wild-type or mock-edited RHO alleles. Schematics of RHO alleles predicted to be generated by RHO-3, RHO-7, and RHO-10 gRNAs (Table 17) having 1 base pair (bp), 2 bp or 3 bp deletions are illustrated in FIG. 6. RHO mutations predicted to be generated from RHO-3, RHO-7, and RHO-10 gRNAs (i.e., mock-edited RHO alleles) were generated using either WT-RHO cDNA or

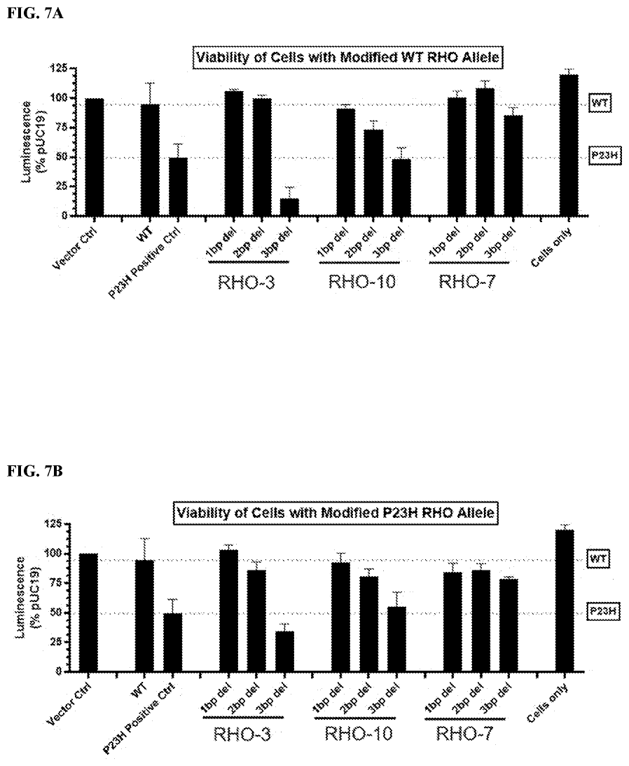

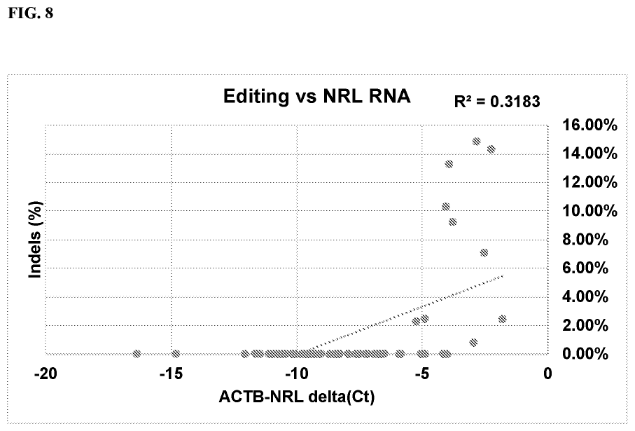

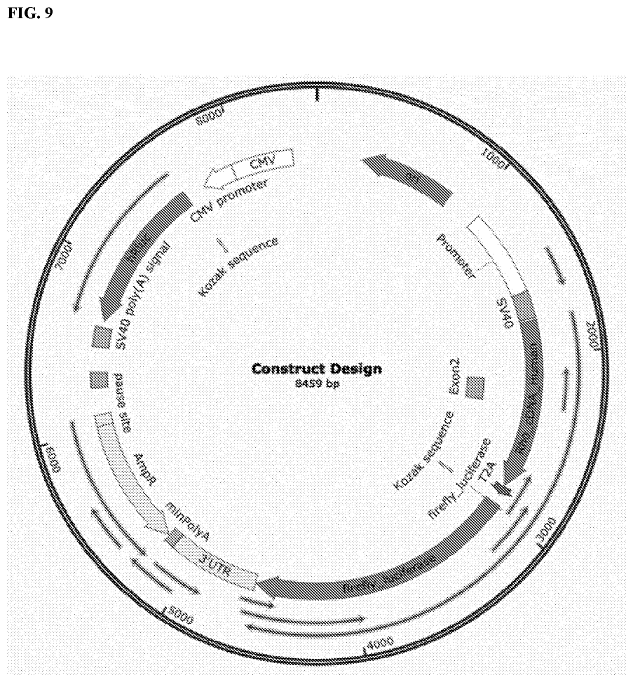

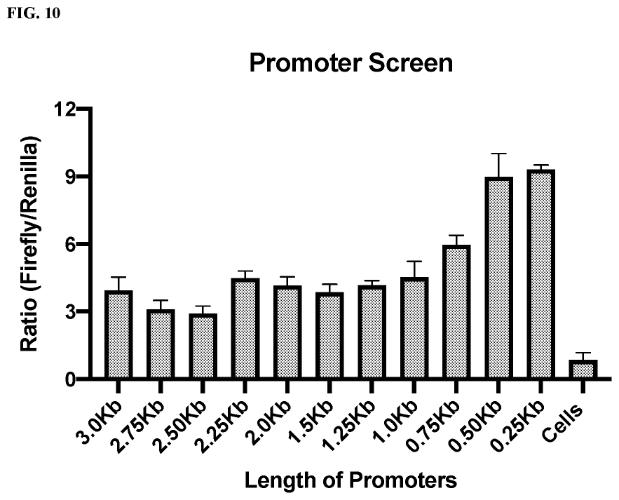

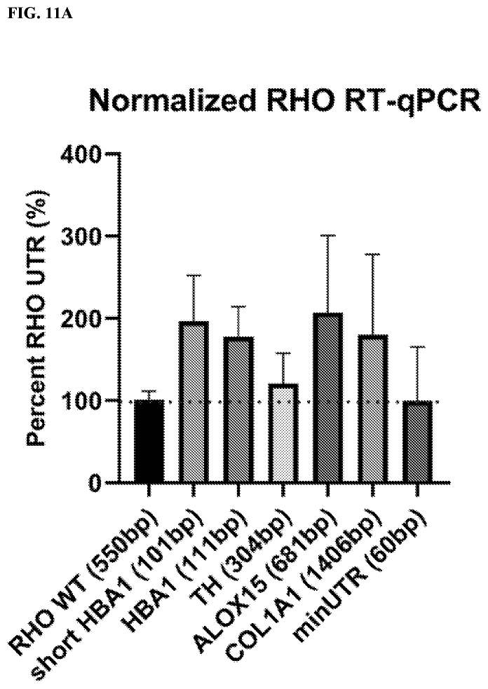

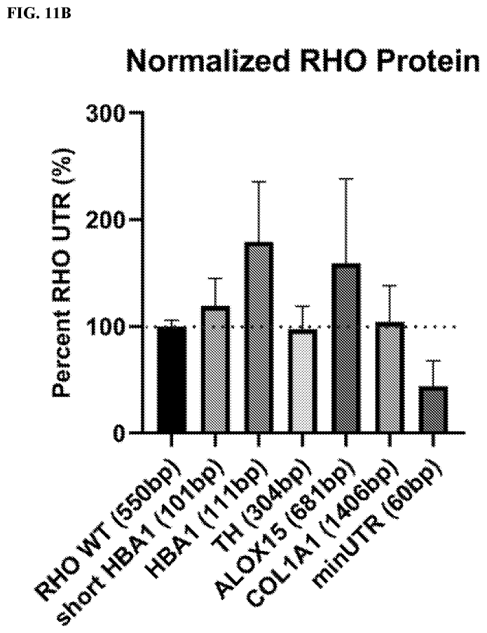

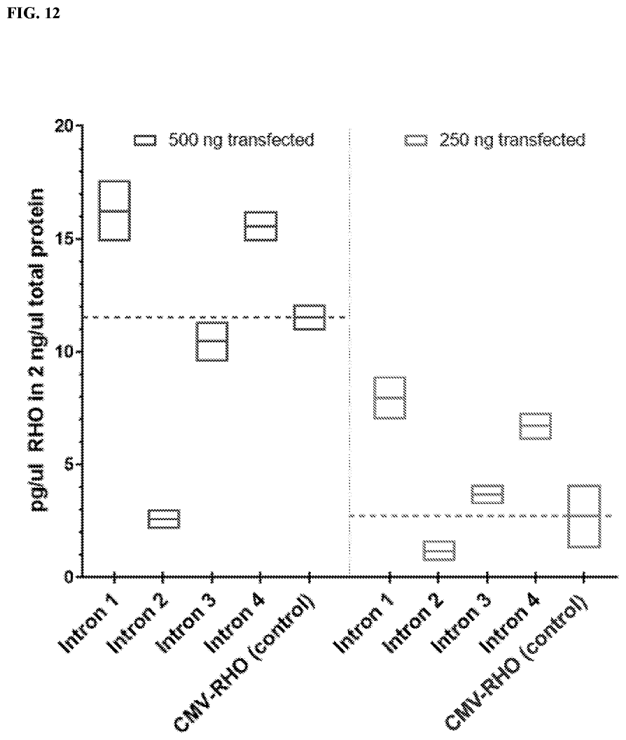

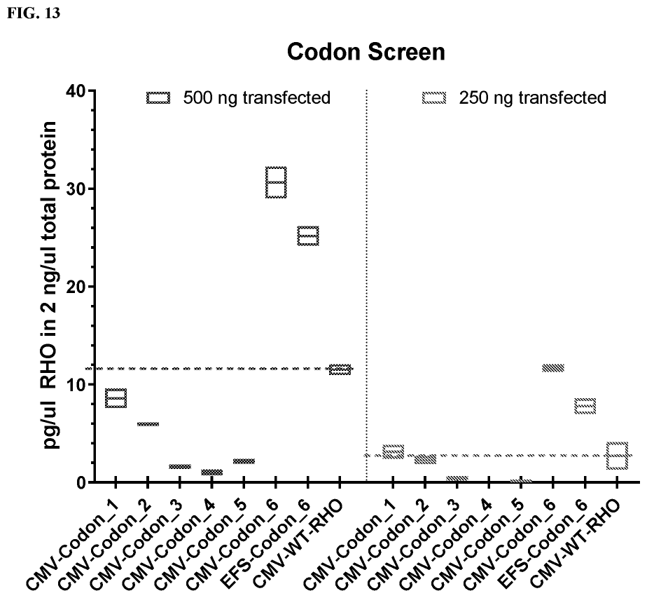

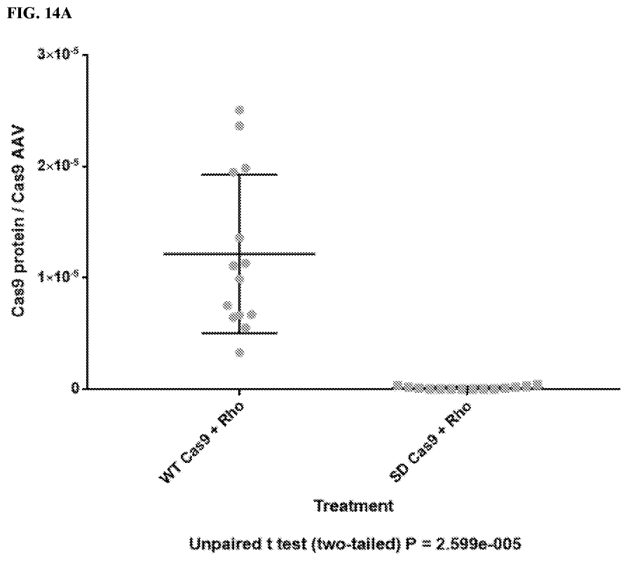

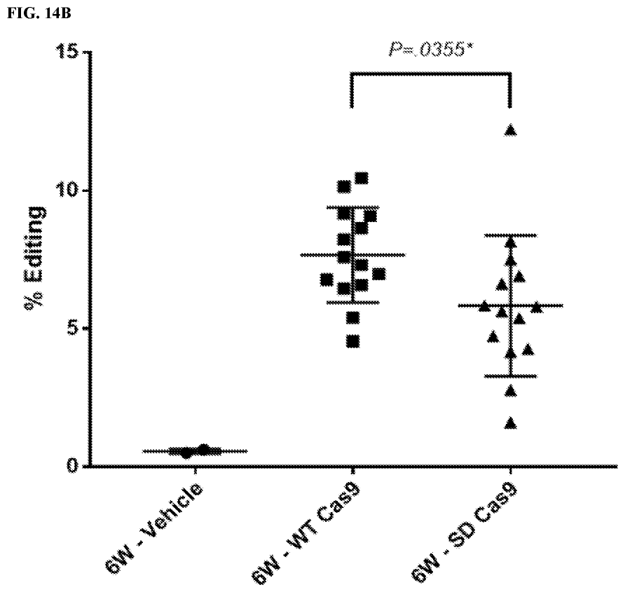

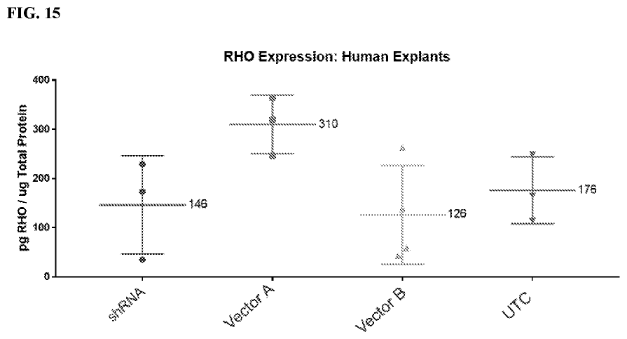

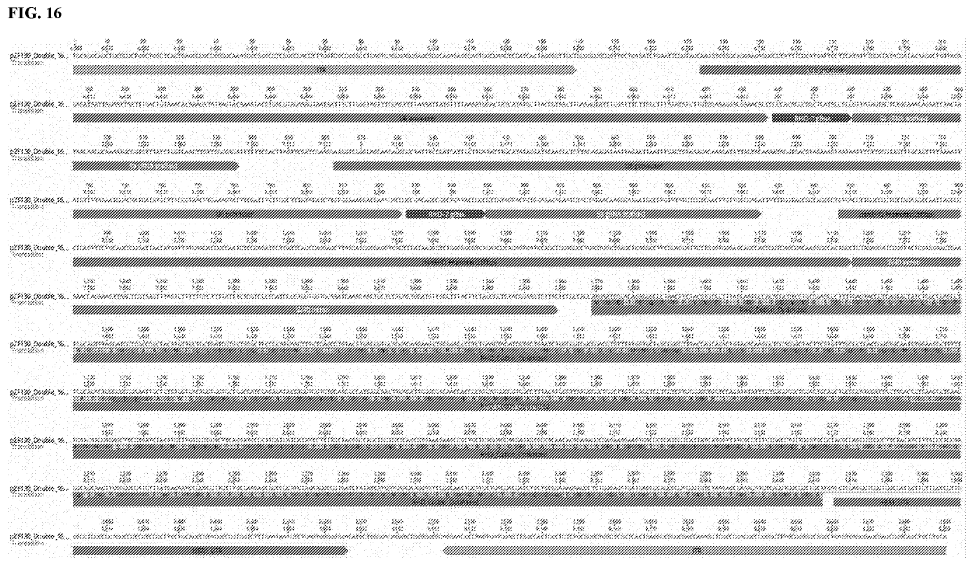

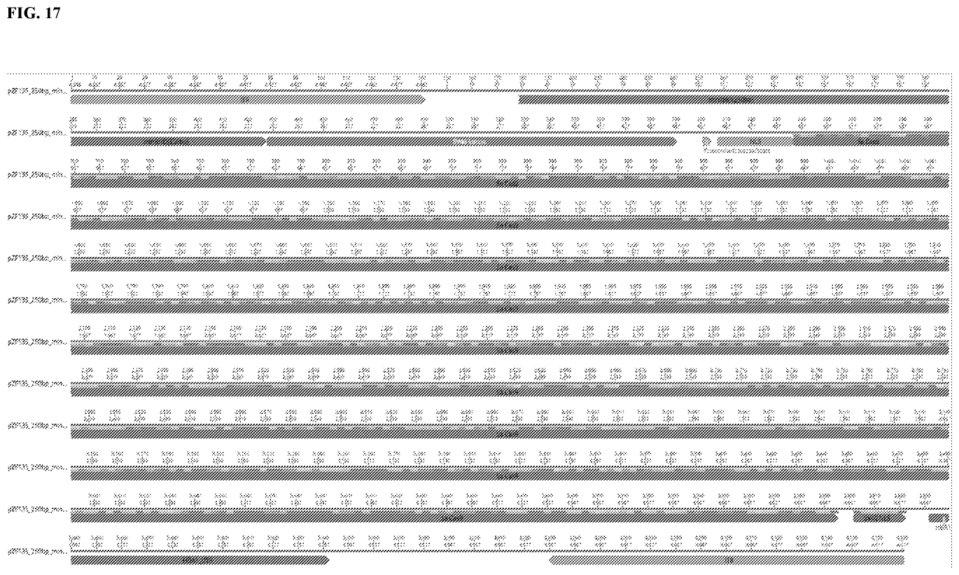

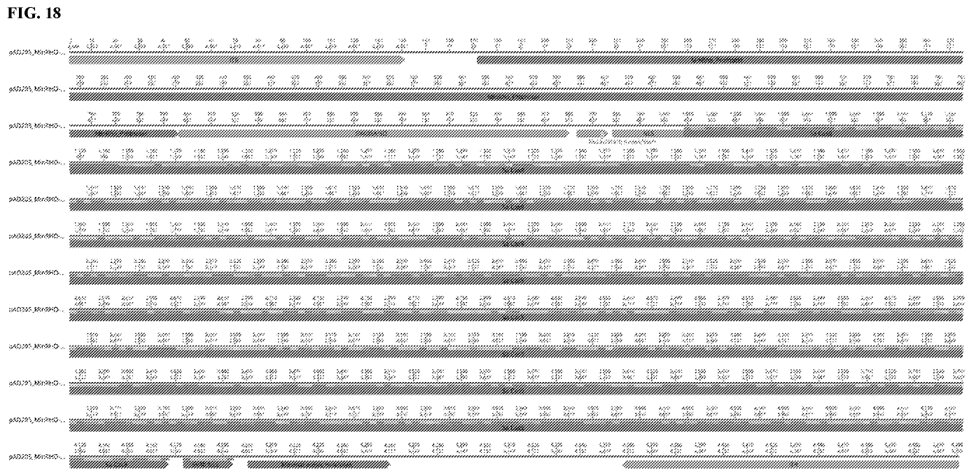

RHO cDNA expressing the P23H RHO variant. Wild-type RHO, mock-edited RHO alleles, or RHO alleles expressing the P23H RHO variant were cloned into mammalian expression plasmids, lipofected into HEK293 cells and assessed for cell viability after 48 hours using the ATPLite Luminescence Assay by Perkin Elmer. FIG. 7A shows viability depicted by luminescence of cells with modified WT RHO alleles. FIG. 7B shows viability depicted by luminescence of cells with modified P23H RHO alleles. The upper dotted line represents the level of luminescence from WT RHO alleles and the lower dotted line represents the level of luminescence from the P23H RHO alleles.

FIG. 8 shows editing of rod photoreceptors in non-human primate (NHP) explants using RHO-9 gRNA (Table 1). RNA from a rod-specific mRNA (neural retina leucine zipper (NRL)) was extracted from the explants and measured to determine the percentage of rods present in the explants. RNA from beta actin (ACTB) was also measured to determine the total number of cells. The x-axis shows the delta between ACTB and NRL RNA levels as measured by RT-PCR, which is a measure for the percentage of rods in the explant at the time of lysing the explants. Indels of the RHO gene were assessed using next generation sequencing (NGS). Each circle represents data from a different explant.

FIG. 9 shows a schematic of the plasmid for the dual luciferase system used for optimizing the RHO replacement vector.

FIG. 10 depicts the ratio of firefly/renilla luciferase luminescence using the dual luciferase system to test the effects of different lengths of the RHO promoter on RHO expression. The lengths of the RHO promoter that were tested ranged from 3.0 Kb to 250 bp.

FIGS. 11A and 11B depict the effects on RHO mRNA and RHO protein expression of adding various 3′ UTRs to the RHO replacement vector. The HBA1 3′ UTR (SEQ ID NO:38), short HBA1 3′ UTR (SEQ ID NO:39), TH 3′ UTR (SEQ ID NO:40), COL1A1 3′UTR (SEQ ID NO:41), ALOX15 3′UTR (SEQ ID NO:42), and minUTR (SEQ ID NO:56) were tested. FIG. 11A shows results using RT-qPCR to measure RHO mRNA expression. FIG. 11B shows results using a RHO ELISA assay to measure RHO protein expression.

FIG. 12 depicts the effects on RHO protein expression of inserting different RHO introns into RHO cDNA in the RHO replacement vector. The various RHO cDNA sequences with inserted introns (i.e, Introns 1-4) are set forth in SEQ ID NOs: 4-7, respectively.

FIG. 13 depicts the effects on RHO protein expression of using wild-type or different codon optimized RHO constructs in the RHO replacement vector. The various codon optimized RHO cDNA sequences (i.e., Codon 1-6) are set forth in SEQ ID NOs: 13-18, respectively. The RHO cDNAs were under the control of a CMV or EFS promoter.

FIGS. 14A and 14B depict in vivo editing of the RHO gene and knock down of Cas9 using a self-limiting Cas9 vector system (“SD”). FIG. 14A shows successful knockdown of Cas9 levels using the self-limiting Cas9 vector system (i.e., “SD Cas9+Rho”). FIG. 14B shows successful editing using the self-limiting Cas9 vector system (i.e., “SD Cas9”).

FIG. 15 depicts RHO expression in human explants. Explants were transduced with “shRNA”: transduction of retinal explants with shRNA targeting the RHO gene and a replacement vector providing a RHO cDNA (as published in Cideciyan 2018); “Vector A”: a two-vector system (Vector 1 comprising saCas9 driven by the minimal RHO promoter (250 bp), and Vector 2 comprising a codon-optimized RHO cDNA (codon-6) and comprising a HBA1 3′ UTR under the control of the minimal 250 bp RHO promoter, as well as as the RHO-9 gRNA (Table 1) under the control of a U6 promoter); “Vector B”: a two-vector system identical to “Vector A” except for Vector 2 comprising a wt RHO cDNA; and “UTC”: untransduced control.

FIG. 16 is a schematic of an exemplary AAV vector (SEQ ID NO:11) according to certain embodiments of the disclosure. The schematic shows an AAV5 genome comprising and encoding an ITR (SEQ ID NO:92), a first U6 promoter (SEQ ID NO:78), a first RHO-7 gRNA (comprising a RHO-7 gRNA targeting domain (SEQ ID NO:606) (DNA) and SEQ ID NO:12), a second U6 promoter (SEQ ID NO:78), a second RHO-7 gRNA (comprising a RHO-7 gRNA targeting domain (SEQ ID NO:606) (DNA) and SEQ ID NO:12), a minimum RHO Promoter (250 bp) (SEQ ID NO:44), an SV40 Intron (SEQ ID NO:94), a codon optimized RHO cDNA (SEQ ID NO:18), HBA1 3′ UTR (SEQ ID NO:38), a minipolyA (SEQ ID NO:56), and a right ITR (SEQ ID NO:93). In certain embodiments, the AAV vector may be delivered via subretinal injection.

FIG. 17 is a schematic of an exemplary AAV vector (SEQ ID NO:10) according to certain embodiments of the disclosure. The schematic shows an AAV5 genome comprising and encoding an ITR (SEQ ID NO:92), a minimum RHO Promoter (250 bp) (SEQ ID NO:44), an SV40 Intron (SEQ ID NO:94), an NLS sequence, an S. aureus Cas9 sequence, an SV40 NLS, an HBA1 3′ UTR (SEQ ID NO:38), and a right ITR (SEQ ID NO:93). In certain embodiments, the AAV vector may be delivered via subretinal injection.

FIG. 18 is a schematic of an exemplary AAV vector (SEQ ID NO:9) according to certain embodiments of the disclosure. The schematic shows an AAV5 genome comprising and encoding an ITR (SEQ ID NO:92), a minimum RHO Promoter, an SV40 SA/SD, an NLS, an S. aureus Cas9 sequence, an SV40 NLS, a minipolyA (SEQ ID NO:56), and a right ITR (SEQ ID NO:93). In certain embodiments, the AAV vector may be delivered via subretinal injection.

### DETAILED DESCRIPTION

### Definitions

“Domain”, as used herein, is used to describe segments of a protein or nucleic acid. Unless otherwise indicated, a domain is not required to have any specific functional property.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Modulator”, as used herein, refers to an entity, e.g., a drug, that can alter the activity (e.g., enzymatic activity, transcriptional activity, or translational activity), amount, distribution, or structure of a subject molecule or genetic sequence. In an embodiment, modulation comprises cleavage, e.g., breaking of a covalent or non-covalent bond, or the forming of a covalent or non-covalent bond, e.g., the attachment of a moiety, to the subject molecule. In an embodiment, a modulator alters the, three dimensional, secondary, tertiary, or quaternary structure, of a subject molecule. A modulator can increase, decrease, initiate, or eliminate a subject activity.

“Polypeptide”, as used herein, refers to a polymer of amino acids having less than 100 amino acid residues. In an embodiment, it has less than 50, 20, or 10 amino acid residues.

“Replacement”, or “replaced”, as used herein with reference to a modification of a molecule does not require a process limitation but merely indicates that the replacement entity is present.

“RHO target position,” as that term is used herein, refers to a target position, e.g., one or more nucleotides, in or near the RHO gene, that are targeted for alteration using the methods described herein. In certain embodiments, alteration of the RHO target position, e.g., by substitution, deletion, or insertion, may result in disruption (e.g., “knocking out”) of the RHO gene. In certain embodiments, the RHO target position may be located in a 5′ region of the RHO gene (e.g., 5′ UTR, exon 1, exon 2, intron 1, the exon 1/intron 1 border, or the exon 2/intron 1 border), a non-coding region of the RHO gene (e.g., an enhancer region, a promoter region, an intron, 5′ UTR, 3′UTR, polyadenylation signal), or a coding region of the RHO gene (e.g., early coding region, an exon (e.g., exon 1, exon 2, exon 3, exon 4, exon 5), or an exon/intron border (e.g., exon 1/intron1, exon 2/intron 1) of the RHO gene.

“Small molecule”, as used herein, refers to a compound having a molecular weight less than about 2 kD, e.g., less than about 2 kD, less than about 1.5 kD, less than about 1 kD, or less than about 0.75 kD.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human. In other embodiments, the subject is poultry.

“Treat”, “treating” and “treatment”, as used herein, mean the treatment of a disease in a mammal, e.g., in a human, including (a) inhibiting the disease, i.e., arresting or preventing its development; (b) relieving the disease, i.e., causing regression of the disease state; and (c) curing the disease.

“X” as used herein in the context of an amino acid sequence, refers to any amino acid (e.g., any of the twenty natural amino acids) unless otherwise specified.

Autosomal-Dominant Retinitis Pigmentosa (adRP)

Retinitis pigmentosa (RP) affects between 50,000 and 100,000 people in the United States. RP is a group of inherited retinal dystrophies that affect photoreceptors and retinal pigment epithelium cells. The disease causes retinal deterioration and atrophy, and is characterized by progressive deterioration of vision, ultimately resulting in blindness.

Typical disease onset is during the teenage years, although some subjects may present in early adulthood. Subjects initially present with poor night vision and declining peripheral vision. In general, visual loss proceeds from the peripheral visual field inwards. The majority of subjects are legally blind by the age of 40. The central visual field may be spared through the late stages of the disease, so that some subjects may have normal visual acuity within a small visual field into their 70's. However, the majority of subjects lose their central vision as well between the age of 50 and 80 (Berson 1990). Upon examination, a subject may have one or more of bone spicule pigmentation, narrowing of the visual fields and retinal atrophy.

There are over 60 genes and hundreds of mutations that cause RP. Autosomal dominant RP (adRP), accounts for 15-25% of RP. Autosomal recessive RP (arRP) accounts for 5-20% of RP. X-linked RP (X-LRP) accounts for 5-15% of RP (Daiger 2007). In general, adRP often has the latest presentation, arRP has a moderate presentation and X-LRP has the earliest presentation.

Autosomal-dominant retinitis pigmentosa (adRP) is caused by heterozygous mutations in the rhodopsin (RHO) gene. Mutations in the RHO gene account for 25-30% of cases of adRP.

The RHO gene encodes the rhodopsin protein. Rhodopsin is a G protein-coupled receptor expressed in the outer segment of retinal photoreceptor (PR) rod cells and is a critical element of the phototransduction cascade. Light absorbed by rhodopsin causes 11-cis retinal to isomerize into all-trans retinal. This conformational change allows rhodopsin to couple with transducin, which is the first step in the visual signaling cascade. Heterozygous mutations in the RHO gene cause a decreased production of wild-type rhodopsin and/or expression of mutant rhodopsin. This leads to poor function of the phototransduction cascade and declining function in rod PR cells. Over time, there is atrophy of rod PR cells and eventually atrophy of cone PR cells as well. This causes the typical phenotypic progression of cumulative vision loss experienced by RP subjects. Subjects with RHO mutations experience progressive loss of peripheral visual fields followed by loss of central visual fields (the latter measured by decreases in visual acuity).

Exemplary RHO mutations are provided in Table A.

TABLE A
RHO Mutations (Group A Mutations)

| Number | Mutation |
|---|---|
| 1 | Pro23His |
| 2 | Pro23Leu |
| 3 | Thr58Arg |
| 4 | Pro347Thr |
| 5 | Pro347Ala |
| 6 | Pro347Ser |
| 7 | Pro347Gly |
| 8 | Pro347Leu |
| 9 | Pro347Arg |
| 10 | Thr 4 Lys |
| 11 | Asn 15 Ser |
| 12 | Thr 17 Met |
| 13 | Gln 28 His |
| 14 | Leu 40 Arg |
| 15 | Met 44 Thr |
| 16 | Phe 45 Leu |
| 17 | Leu 46 Arg |
| 18 | Gly 51 Arg |
| 19 | Gly 51 Val |
| 20 | Gly 51 Ala |
| 21 | Pro 53 Arg |
| 22 | Thr 58 Arg |
| 23 | Gln 64 stop |
| 24 | Val 87 Asp |
| 25 | Gly 89 Asp |
| 26 | Gly 106 Arg |
| 27 | Gly 106 Trp |
| 28 | Gly 109 Arg |
| 29 | Cys 110 Tyr |
| 30 | Cys 110 Phe |
| 31 | Gly 114 Asp |
| 32 | Gly 114 Val |
| 33 | Leu 125 Arg |
| 34 | Ser 127 Phe |
| 35 | Leu 131 Pro |
| 36 | Arg 135 Gly |
| 37 | Arg 135 Trp |
| 38 | Arg 135 Leu |
| 39 | Arg 135 Pro |
| 40 | Tyr 136 stop |
| 41 | Val 137 Met |
| 42 | Cys 140 Ser |
| 43 | Ala 164 Val |
| 44 | Ala 164 Glu |
| 45 | Cys 167 Arg |
| 46 | Cys 167 Trp |
| 47 | Pro 171 Glu |
| 48 | Pro 171 Ser |
| 49 | Pro 171 Leu |
| 50 | Pro 171 Gln |
| 51 | Tyr 178 Asn |
| 52 | Tyr 178 Cys |
| 53 | Pro 180 Ala |
| 54 | Glu 181 Lys |
| 55 | Gly 182 Ser |
| 56 | Gln 184 Pro |
| 57 | Ser 186 Pro |
| 58 | Ser 186 Trp |
| 59 | Cys 187 Tyr |
| 60 | Gly 188 Arg |
| 61 | Gly 188 Glu |
| 62 | Asp 190 Asn |
| 63 | Asp 190 Tyr |
| 64 | Asp 190 Gly |
| 65 | Thr 193 Met |
| 66 | Met 207 Arg |
| 67 | Val 209 Met |
| 68 | His 211 Arg |
| 69 | His 211 Pro |
| 70 | Pro 215 Thr |
| 71 | Met 216 Arg |
| 72 | Met 216 Lys |
| 73 | Phe 220 Cys |
| 74 | Cys 222 Arg |
| 75 | Pro 267 Leu |
| 76 | Pro 267 Arg |
| 77 | Ser 270 Arg |
| 78 | Thr 289 Pro |
| 79 | Lys 296 Glu |
| 80 | Lys 296 Met |
| 81 | Ser 297 Arg |
| 82 | Gln 312 stop |
| 83 | Leu 328 Pro |
| 84 | Thr 342 Met |
| 85 | Gln 344 stop |
| 86 | Val 345 Leu |
| 87 | Val 345 Met |
| 88 | Ala 346 Pro |
| 89 | stop 349 Glu |
| 90 | Glu 150 Lys |
| 91 | Gly 174 Ser |
| 92 | Glu 249 ter |
| 93 | Gly 284 Ser |

Treatment for RP is limited and there is currently no approved treatment that substantially reverses or halts the progression of disease in adRP. In an embodiment, Vitamin A supplementation may delay onset of disease and slow progression. The Argus II retinal implant was approved for use in the United States in 2013. The Argus II retinal implant is an electrical implant that offers minimal improvement in vision in subjects with RP. For example, the best visual acuity achieved in trials by the device was 20/1260. However, legal blindness is defined as 20/200 vision.

### Overview

As provided herein, the inventors have designed a therapeutic strategy that provides an alteration that comprises disrupting the mutant RHO gene by the insertion or deletion of one or more nucleotides mediated by an RNA-guided nuclease (e.g., Cas9 or Cpfl) as described below and providing a functional RHO cDNA. This type of alteration is also referred to as “knocking out” the mutant RHO gene and results in a loss of function of the mutant RHO gene. While not wishing to be bound by theory, knocking out the mutant RHO gene and providing a functional exogenous RHO cDNA maintains appropriate levels of rhodopsin protein in PR rod cells. This therapeutic strategy has the benefit of disrupting all known mutant alleles related to adRP, for example, the RHO mutations in Table A.

In certain embodiments, the 5′ UTR region (e.g., 5′ UTR, exon 1, exon 2, intron 1, exon 1/intron 1, or exon 2/intron 1 border) of a mutant RHO gene, is targeted to alter (i.e., knockout (e.g., eliminate expression of)) the mutant RHO gene.

In certain embodiments, the coding region (e.g., an exon, e.g., an early coding region) of the mutant RHO gene, is targeted to alter (i.e., knockout (e.g., eliminate expression of)) the mutant RHO gene. For example, the early coding region of the mutant RHO gene includes the sequence immediately following a start codon, within a first exon of the coding sequence, or within 500 bp of the start codon (e.g., less than 500, 450, 400, 350, 300, 250, 200, 150, 100 or 50 bp).

In certain embodiments, a non-coding region of the mutant RHO gene (e.g., an enhancer region, a promoter region, an intron, 5′ UTR, 3′UTR, polyadenylation signal) is targeted to alter (i.e., knockout (e.g., eliminate expression of)) the mutant RHO gene.

In certain embodiments, an exon/intron border of the mutant RHO gene (e.g., exon 1/intron 1, exon 2/intron 1) is targeted to alter (i.e., knockout (e.g., eliminate expression of)) the mutant RHO gene. In certain embodiments, targeting an exon/intron border provides the benefit of being able to use an exogenous RHO cDNA molecule that is not codon-modified to be resistant to cutting by a gRNA.

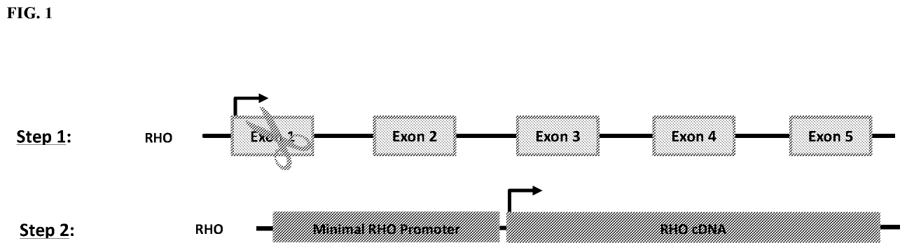

FIG. 1 shows a schematic of one embodiment of a therapeutic strategy to knockout an endogenous RHO gene and provide an exogenous RHO cDNA. In one embodiment, CRISPR/RNA-guided nuclease genome editing systems may be used to alter (i.e., knockout (e.g., eliminate expression of)) exon 1 or exon 2 of the RHO gene. In certain embodiments, the RHO gene may be mutated RHO gene. In certain embodiments, the mutated RHO gene may comprise one or more RHO mutations in Table A. Alteration of exon 1 or exon 2 of the RHO gene results in disruption of the endogenous mutated RHO gene.

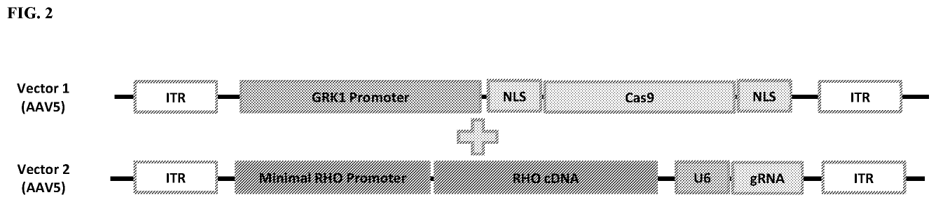

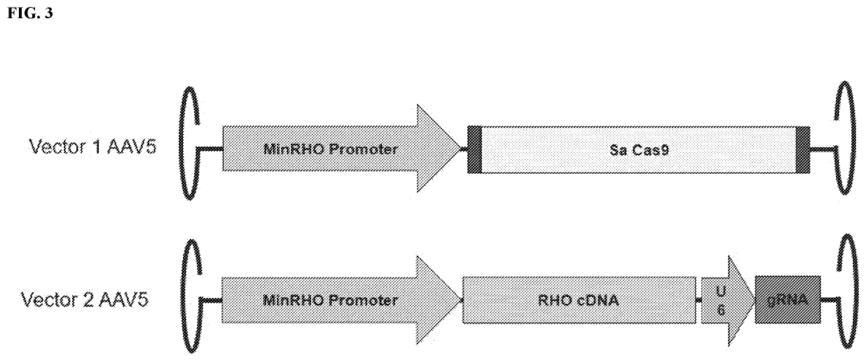

In certain embodiments, the therapeutic strategy may be accomplished using a dual-vector system. In certain aspects, the disclosure focuses on AAV vectors encoding CRISPR/RNA-guided nuclease genome editing systems and a replacement RHO cDNA, and on the use of such vectors to treat adRP disease. Exemplary vector genomes are schematized in FIG. 2, which illustrates certain fixed and variable elements of these vectors: inverted terminal repeats (ITRs), at least one gRNA sequence and a promoter sequences to drive its expression, an RNA-guided nuclease (e.g., Cas9) coding sequence and another promoter to drive its expression, nuclear localization signal (NLS) sequences, and a RHO cDNA sequence and another promoter to drive its expression. Each of these elements is discussed in detail herein. Additional exemplary vector genomes are schematized in FIG. 3, which illustrates certain fixed and variable elements of these vectors: at least one gRNA sequence and a promoter sequence to drive its expression (e.g., U6 promoter), an RNA-guided nuclease (e.g., S. aureus Cas9) coding sequence and another promoter to drive its expression (e.g., minimal RHO promoter), and a RHO cDNA sequence and another promoter to drive its expression (e.g., minimal RHO promoter). Additional exemplary vectors and sequences for use with the strategies described herein are set forth in FIGS. 16-18 and SEQ ID NOs:8-11.

In certain embodiments, the AAV vector used herein may be a self-limiting vector system as described in WO2018/106693, published on Jun. 14, 2018, and entitled Systems and Methods for One-Shot guide RNA (ogRNA) Targeting of Endogenous and Source DNA, the entire contents of which are incorporated herein by reference.

As shown in FIG. 1, in certain embodiments, a dual vector system may be used to knockout expression of mutant RHO gene and deliver an exogenous RHO cDNA to restore expression of wild-type rhodopsin protein. In certain embodiments, one AAV vector genome may comprise ITRs and an RNA-guided nuclease coding sequence and promoter sequence to drive its expression and one or more NLS sequences. In certain embodiments, a second AAV vector genome may comprise ITRs, a RHO cDNA sequence and a promoter to drive its expression, one gRNA sequence and promoter sequence to drive its expression.

While not wishing to be bound by theory, knocking out the RHO gene and replacing it with functional exogenous RHO cDNA maintains appropriate levels of rhodopsin protein in PR rod cells. Restoring appropriate levels of functional rhodopsin protein in rod PR cells maintains the phototransduction cascade and may delay or prevent PR cell death in subjects with adRP.

In some embodiments, a method disclosed herein is characterized by knocking out a variant of the RHO gene that is associated with adRP, e.g., a RHO mutant gene or allele described herein, and restoring wild-type RHO protein expression in a subject in need thereof, e.g., in a subject suffering from or predisposed to adRP. For example, in some embodiments, the methods provided herein are characterized by knocking out a mutant RHO allele in a subject having a mutant and a wild-type RHO allele, and restoring expression of wild-type rhodopsin protein in rod PR cells. In some embodiments, such methods feature knocking out the mutant allele while leaving the wild-type allele intact. In other embodiments, such methods feature knocking out both the mutant and the wild-type allele. In some embodiments, the methods are characterized by knocking out a mutant allele of the RHO gene and providing an exogenous wild-type protein, e.g., via expression of a cDNA encoding wild-type RHO protein. In some embodiments, knocking out expression of a mutant allele (and, optionally, a wild-type allele), and restoring wild-type RHO protein expression, e.g., via expression of an exogenous RHO cDNA, in a subject in need thereof, e.g., a subject suffering from or predisposed to adRP, ameliorates at least one symptom associated with adRP. In some embodiments, such an amelioration includes, for example, improving the subject's vision. In some embodiments, such an amelioration includes, for example, delaying adRP disease progression, e.g., as compared to an expected progression without clinical intervention. In some embodiments, such an amelioration includes, for example, arresting adRP disease progression. In some embodiments, such an amelioration includes, for example, preventing or delaying the onset of adRP disease in a subject.

In an embodiment, a method described herein comprises treating allogenic or autologous retinal cells ex vivo. In an embodiment, ex vivo treated allogenic or autologous retinal cells are introduced into the subject.

In an embodiment, a method described herein comprises treating an embryonic stem cell, an induced pluripotent stem cell or a cell derived from an iPS cell, a hematopoietic stem cell, a neuronal stem cell or a mesenchymal stem cell ex vivo. In an embodiment, ex vivo treated embryonic stem cells, induced pluripotent stem cells, hematopoietic stem cells, neuronal stem cells or a mesenchymal stem cells are introduced into the subject. In an embodiment, the cell is an induced pluripotent stem cells (iPS) cell or a cell derived from an iPS cell, e.g., an iPS cell generated from the subject, modified to knock out one or more mutated RHO genes and express functional exogenous RHO DNA and differentiated into a retinal progenitor cell or a retinal cell, e.g., retinal photoreceptor cell, and injected into the eye of the subject, e.g., subretinally, e.g., in the submacular region of the retina.

In an embodiment, a method described herein comprises treating autologous stem cells ex vivo. In an embodiment, ex vivo treated autologous stem cells are returned to the subject.

In an embodiment, the subject is treated in vivo, e.g., by a viral (or other mechanism) that targets cells from the eye (e.g., a retinal cell, e.g., a photoreceptor cell, e.g., a cone photoreceptor cell, e.g., a rod photoreceptor cell, e.g., a macular cone photoreceptor cell).

In an embodiment, the subject is treated in vivo, e.g., by a viral (or other mechanism) that targets a stem cell (e.g., an embryonic stem cell, an induced pluripotent stem cell or a cell derived from an iPS cell, a hematopoietic stem cell, a neuronal stem cell or a mesenchymal stem cell).

In an embodiment, treatment is initiated in a subject prior to disease onset. In a particular embodiment, treatment is initiated in a subject who has tested positive for one or more mutations in the RHO gene.

In an embodiment, treatment is initiated in a subject after disease onset.

In an embodiment, treatment is initiated in an early stage of adRP disease. In an embodiment, treatment is initiated after a subject presents with gradually declining vision. In an embodiment, repair of the RHO gene after adRP onset but early in the disease course will prevent progression of the disease.

In an embodiment, treatment is initiated in a subject in an advanced stage of disease. While not wishing to be bound by theory, it is held that advanced stage treatment will likely preserve a subject's visual acuity (in the central visual field), which is important for subject function and performance of activities of daily living.

In an embodiment, treatment of a subject prevents disease progression. While not wishing to be bound by theory, it is held that initiation of treatment for subjects at all stages of disease (e.g., prophylactic treatment, early stage adRP, and advanced stage adRP) will prevent RP disease progression and be of benefit to subjects.

In an embodiment, treatment is initiated after determination that the subject, e.g., an infant or newborn, teenager, or adult, is positive for a mutation in the RHO gene, e.g., a mutation described herein.

In an embodiment, treatment is initiated after determination that the subject is positive for a mutation in the RHO gene, e.g., a mutation described herein, but prior to manifestation of a symptom of the disease.

In an embodiment, treatment is initiated after determination that the subject is positive for a mutation in the RHO gene, e.g., a mutation described herein, and after manifestation of a symptom of the disease.

In an embodiment, treatment is initiated in a subject at the appearance of a decline in visual fields.

In an embodiment, treatment is initiated in a subject at the appearance of declining peripheral vision.

In an embodiment, treatment is initiated in a subject at the appearance of poor night vision and/or night blindness.

In an embodiment, treatment is initiated in a subject at the appearance of progressive visual loss.

In an embodiment, treatment is initiated in a subject at the appearance of progressive constriction of the visual field.

In an embodiment, treatment is initiated in a subject at the appearance of one or more indications consistent with adRP upon examination of a subject. Exemplary indications include, but are not limited to, bone spicule pigmentation, narrowing of the visual fields, retinal atrophy, attenuated retinal vasculature, loss of retinal pigment epithelium, pallor of the optic nerve, and/or combinations thereof.

In an embodiment, a method described herein comprises subretinal injection, submacular injection, suprachoroidal injection, or intravitreal injection, of gRNA or other components described herein, e.g., an RNA-guided nuclease (e.g., Cas9 or Cpfl molecule) and a RHO cDNA molecule.

In an embodiment, a gRNA or other components described herein, e.g., an RNA-guided nuclease (e.g., Cas9 or Cpfl molecule) and a RHO cDNA molecule are delivered, e.g., to a subject, by AAV, lentivirus, nanoparticle, or parvovirus, e.g., a modified parvovirus designed to target cells from the eye (e.g., a retinal cell, e.g., a photoreceptor cell, e.g., a cone photoreceptor cell, e.g., a rod photoreceptor cell, e.g., a macular cone photoreceptor cell).

In an embodiment, a gRNA or other components described herein, e.g., an RNA-guided nuclease (e.g., Cas9 or Cpfl molecule) and a RHO cDNA molecule are delivered, e.g., to a subject, by AAV, lentivirus, nanoparticle, or parvovirus, e.g., a modified parvovirus designed to target stem cells (e.g., an embryonic stem cell, an induced pluripotent stem cell or a cell derived from an iPS cell, a hematopoietic stem cell, a neuronal stem cell or a mesenchymal stem cell).

In an embodiment, a gRNA or other components described herein, e.g., an RNA-guided nuclease (e.g., Cas9 or Cpfl molecule) and a RHO cDNA molecule are delivered, ex vivo, by electroporation.

In an embodiment, CRISPR/RNA-guided nuclease components are used to knock out the mutant RHO gene which gives rise to the disease.

I. gRNA Molecules

The terms guide RNA and gRNA refer to any nucleic acid that promotes the specific association (or “targeting”) of an RNA-guided nuclease such as a Cas9 or a Cpfl to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for example by duplexing). gRNAs and their component parts are described throughout the literature (see, e.g., Briner 2014, which is incorporated by reference; see also Cotta-Ramusino).

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the RNA-guided nuclease/gRNA complex. As type II CRISPR systems were adapted for use in gene editing, it was discovered that the crRNA and tracrRNA could be joined into a single unimolecular or chimeric gRNA, for example by means of a four nucleotide (e.g., GAAA) “tetraloop” or “linker” sequence bridging complementary regions of the crRNA (at its 3′ end) and the tracrRNA (at its 5′ end) (Mali 2013; Jiang 2013; Jinek 2012; all incorporated by reference herein).

Guide RNAs, whether unimolecular or modular, include a targeting domain that is fully or partially complementary to the target domain within a target sequence (e.g., a double-stranded DNA sequence in the genome of a cell where editing is desired). In certain embodiments, a RHO target sequence encompasses, comprises, or is proximal to a RHO target position. Targeting domains are referred to by various names in the literature, including without limitation “guide sequences” (Hsu 2013, incorporated by reference herein), “complementarity regions” (Cotta-Ramusino), “spacers” (Briner 2014), and generically as “crRNAs” (Jiang 2013). Irrespective of the names they are given, targeting domains are typically 10-30 nucleotides in length, preferably 16-24 nucleotides in length (for example, 16, 17, 18, 19, 20, 21, 22, 23 or 24 nucleotides in length), and are at or near the 5′ terminus of in the case of a Cas9 gRNA, and at or near the 3′ terminus in the case of a Cpfl gRNA. The nucleic acid sequence complementary to the target domain, i.e., the nucleic acid sequence on the complementary DNA strand of the double-stranded DNA that comprises the target domain, is referred to herein as the “protospacer.”

The “protospacer-adjacent motif” (PAM) sequence takes its name from its sequential relationship to the “protospacer” sequence. Together with protospacer sequences, PAM sequences define target sequences and/or target positions for specific RNA-guided nuclease/gRNA combinations. Various RNA-guided nucleases may require different sequential relationships between PAMs and protospacers.

For example, in general, Cas9 nucleases recognize PAM sequences that are 3′ of the protospacer:

5′ - - - [protospacer] [PAM] - - - 3′
3′ - - - [target domain] - - - 5′

For another example, in general, Cpfl recognizes PAM sequences that are 5′ of the protospacer:

5′ - - - [PAM] [protospacer] - - - 3′
3′ - - - [target domain] - - - 5′

In some embodiments described herein, RHO protospacers and exemplary suitable targeting domains are described. Those of ordinary skill in the art will be aware of additional suitable guide RNA targeting domains that can be used to target an RNA-guided nuclease to a given protospacer, e.g., targeting domains that comprise additional or less nucleotides, or that comprise one or more nucleotide mismatches when hybridized to a target domain.

In addition to the targeting domains, gRNAs typically (but not necessarily, as discussed below) include a plurality of domains that influence the formation or activity of gRNA/Cas9 complexes. For example, as mentioned above, the duplexed structure formed by first and secondary complementarity domains of a gRNA (also referred to as a repeat:anti-repeat duplex) interacts with the recognition (REC) lobe of Cas9 and may mediate the formation of Cas9/gRNA complexes (Nishimasu 2014; Nishimasu 2015; both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015). A first stem-loop near the 3′ portion of the second complementarity domain is referred to variously as the “proximal domain,” (Cotta-Ramusino) “stem loop 1” (Nishimasu 2014; Nishimasu 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three). A description of conserved stem loop structures (and gRNA structures more generally) organized by species is provided in Briner 2014.

Skilled artisans will appreciate that gRNAs can be modified in a number of ways, some of which are described below, and these modifications are within the scope of disclosure. For economy of presentation in this disclosure, gRNAs may be presented by reference solely to their targeting domain sequences.

gRNA Modifications

The activity, stability, or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into a population of cells, particularly the cells of the present invention. As noted above, the term “innate immune response” includes a cellular response to exogenous nucleic acids, including single stranded nucleic acids, generally of viral or bacterial origin, which involves the induction of cytokine expression and release, particularly the interferons, and cell death.

One common 3′ end modification is the addition of a poly A tract comprising one or more (and typically 5-200) adenine (A) residues. The poly A tract can be contained in the nucleic acid sequence encoding the gRNA, or can be added to the gRNA during chemical synthesis, or following in vitro transcription using a polyadenosine polymerase (e.g., E. coli Poly(A)Polymerase). In vivo, poly-A tracts can be added to sequences transcribed from DNA vectors through the use of polyadenylation signals. Examples of such signals are provided in Maeder.

Some exemplary gRNA modifications useful in the context of the present RNA-guided nuclease technology are provided herein, and the skilled artisan will be able to ascertain additional suitable modifications that can be used in conjunction with the gRNAs and treatment modalities disclosed herein based on the present disclosure. Suitable gRNA modifications include, without limitations, those described in U.S. Patent Application No. US 2017/0073674 A1 and International Publication No. WO 2017/165862 A1, the entire contents of each of which are incorporated by reference herein.

II. Methods for Designing gRNAs

Methods for designing gRNAs are described herein, including methods for selecting, designing and validating target domains. Exemplary targeting domains are also provided herein. Targeting domains discussed herein can be incorporated into the gRNAs described herein.

Methods for selection and validation of target sites as well as off-target analyses are described, e.g., in Mali 2013; Hsu 2013; Fu 2014; Heigwer 2014; Bae 2014; Xiao 2014. For example, a software tool can be used to optimize the choice of gRNA within a user's target site, e.g., to minimize total off-target activity across the genome. Off target activity may be other than cleavage. For each possible gRNA choice using S. pyogenes Cas9, the tool can identify all off-target sites (preceding either NAG or NGG PAMs) across the genome that contain up to certain number (e.g., 1, 2, 3, 4, 5, 6, 7, 8, 9, or 10) of mismatched base-pairs. The cleavage efficiency at each off-target site can be predicted, e.g., using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool.

The targeting domains discussed herein can be incorporated into the gRNAs described herein.

### Exemplary Protospacers and Targeting Domains

Guide RNAs targeting various positions within the RHO gene for use with S. aureus Cas9 were identified. Following identification, the gRNAs were ranked into three tiers. The gRNAs in tier 1 were selected based on cutting in exon 1 and exon 2 of the RHO gene. Tier 1 guides exhibited >9% editing in T-cells. For selection of tier 2 gRNAs, selection was based on cutting in the 5′ UTR of the RHO gene. Tier 2 gRNAs exhibited >10% editing in T-cells. Tier 3 gRNAs were selected based cutting in intron 1 of the RHO gene. Tier 3 gRNAs exhibit >10% editing in T-cells.

Table 1 provides targeting domains for an exon 1 or exon 2 RHO target position in the RHO gene selected according to the first-tier parameters. The targeting domains were selected based on cutting in exon 1 or exon 2 of the RHO gene and exhibiting >9% editing in T-cells. It is contemplated herein that the targeting domain hybridizes to the strand complementary to the target domain sequence provided through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases).

TABLE 1
Tier 1

| | | | | Targeting |
|---|---|---|---|---|
| Location | | Indel | Targeting | Domain |
| in RHO | gRNA | Fraction | Domain | (DNA)/ |
| gene | ID | Window | (RNA) | Protospacer |
| utr5_0; | RHO-1 | 0.2284375 | GUCA | GTCA |
| cds 0 | | | GCCA | GCCA |
| | | | CAAG | CAAG |
| | | | GGCC | GGCC |
| | | | ACAG | ACAG |
| | | | CC | CC |
| | | | (SEQ | (SEQ |
| | | | ID | ID |
| | | | NO: 100) | NO: 600) |
| cds_0 | RHO-2 | 0.134454179 | CCGA | CCGA |
| | | | AGAC | AGAC |
| | | | GAAG | GAAG |
| | | | UAUC | TATC |
| | | | CAUG | CATG |
| | | | CA | CA |
| | | | (SEQ | (SEQ |
| | | | ID | ID |
| | | | NO: 101) | NO: 601) |
| cds_0 | RHO-3 | 0.174725089 | AGUA | AGTA |
| | | | UCCA | TCCA |
| | | | UGCA | TGCA |
| | | | GAGA | GAGA |
| | | | GGUG | GGTG |
| | | | UA | TA |
| | | | (SEQ | (SEQ |
| | | | ID | ID |
| | | | NO: 102) | NO: 602) |
| cds_0 | RHO-4 | 0.093809401 | CUAG | CTAG |
| | | | GUUG | GTTG |
| | | | AGCA | AGCA |
| | | | GGAU | GGAT |
| | | | GUAG | GTAG |
| | | | UU | TT |
| | | | (SEQ | (SEQ |
| | | | ID | ID |
| | | | NO: 103) | NO: 603) |
| cds_0 | RHO-5 | 0.109343522 | CAUG | CATG |
| | | | GCUC | GCTC |
| | | | AGCC | AGCC |
| | | | AGGU | AGGT |
| | | | AGUA | AGTA |
| | | | CU | CT |
| | | | (SEQ | (SEQ |
| | | | ID | ID |
| | | | NO: 104) | NO: 604) |
| cds_0 | RHO-6 | 0.112374147 | ACGG | ACGG |
| | | | GUGU | GTGT |
| | | | GGUA | GGTA |
| | | | CGCA | CGCA |
| | | | GCCC | GCCC |
| | | | CU | CT |
| | | | (SEQ | (SEQ |
| | | | ID | ID |
| | | | NO: 105) | NO: 605) |
| cds_0; | RHO-7 | 0.297946972 | CCCA | CCCA |
| intron_0 | | | CACC | CACC |
| | | | CGGC | CGGC |
| | | | UCAU | TCAT |
| | | | ACCG | ACCG |
| | | | CC | CC |
| | | | (SEQ | (SEQ |
| | | | ID | ID |
| | | | NO: 106) | NO: 606) |
| cds_0; | RHO-8 | 0.118235744 | CCCU | CCCT |
| intron_0 | | | GGGC | GGGC |
| | | | GGUA | GGTA |
| | | | UGAG | TGAG |
| | | | CCGG | CCGG |
| | | | GU | GT |
| | | | (SEQ | (SEQ |
| | | | ID | ID |
| | | | NO: 107) | NO: 607) |
| cds_1 | RHO-9 | 0.270630335 | CCAU | CCAT |
| | | | CAUG | CATG |
| | | | GGCG | GGCG |
| | | | UUGC | TrGCCT |
| | | | CUUC | TCAC |
| | | | AC | |
| | | | (SEQ | (SEQ |
| | | | ID | ID |
| | | | NO: 108) | NO: 608) |
| cds_1; | RHO-10 | 0.567902679 | GUGC | GTGC |
| intron_1 | | | CAUU | CATT |
| | | | ACCU | ACCT |
| | | | GGAC | GGAC |
| | | | CAGC | CAGC |
| | | | CG | CG |
| | | | (SEQ | (SEQ |
| | | | ID | ID |
| | | | NO: 109) | NO: 609) |
| cds_1; | RHO-11 | 0.106516652 | UUAC | TTAC |
| intron_1 | | | CUGG | CTGG |
| | | | ACCA | ACCA |
| | | | GCCG | GCCG |
| | | | GCGA | GCGA |
| | | | GU | GT |
| | | | (SEQ | (SEQ |
| | | | ID | ID |
| | | | NO: 110) | NO: 610) |

Table 2 provides targeting domains for a 5′UTR RHO target position in the RHO gene selected according to the second-tier parameters. The targeting domains were selected based on cutting in the 5′ UTR region of the RHO gene and exhibiting >10% editing in T-cells. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases).

TABLE 2
Tier 2

| | | | | Target- |
|---|---|---|---|---|
| | | | Target- | ing |
| Location | | Indel | ing | Domain |
| in RHO | gRNA | Fraction | Domain | (DNA)/ |
| gene | ID | Window | (RNA) | Protospacer |
| utr5_0 | RHO-12 | 0.459024462 | GCAUUCU | GCATTCT |
| | | | UGGGUGG | TGGGTGG |
| | | | GAGCAGC | GAGCAGC |
| | | | C | C |
| | | | (SEQ ID | (SEQ ID |
| | | | NO: 111) | NO: 611) |
| utr5_0 | RHO-13 | 0.20572897 | GCUCAGC | GCTCAGC |
| | | | CACUCAG | CACTCAG |
| | | | GGCUCCA | GGCTCCA |
| | | | G | G |
| | | | (SEQ ID | (SEQ ID |
| | | | NO: 112) | NO: 612) |
| utr5_0 | RHO-14 | 0.409641098 | UGACCCG | TGACCCG |
| | | | UGGCUGC | TGGCTGC |
| | | | UCCCACC | TCCCACC |
| | | | C | C |
| | | | (SEQ ID | (SEQ ID |
| | | | NO: 113) | NO: 613) |
| utr5_0 | RHO-15 | 0.134736551 | AGCUCAG | AGCTCAG |
| | | | GCCUUCG | GCCTTCG |
| | | | CAGCAUU | CAGCATT |
| | | | C | C |
| | | | (SEQ ID | (SEQ ID |
| | | | NO: 114) | NO: 614) |

Table 3 provides targeting domains for an intron 1 RHO target position in the RHO gene selected according to the third-tier parameters. The targeting domains were selected based on cutting in intron 1 of the RHO gene and exhibiting >10% editing in T-cells. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases).

TABLE 3
Tier 3

| Location | | lndel | | Targeting |
|---|---|---|---|---|
| in | | Fraction | Targeting | Domain/ |
| RHO | gRNA | Window | Domain | Protospacer |
| gene | ID | Average | (RNA) | (DNA) |
| intron_0 | RHO-16 | 0.107449452 | UAGCA | TAGCA |
| | | | GAAGA | GAAGA |
| | | | AUGCA | ATGCA |
| | | | UCCUA | TCCTA |
| | | | AU | AT |
| | | | (SEQ | (SEQ |
| | | | ID | ID |
| | | | NO: | NO: |
| | | | 115) | 615) |
| intron_0 | RHO-17 | 0.107559427 | ACACG | ACACG |
| | | | CUGAG | CTGAG |
| | | | GAGAG | GAGAG |
| | | | CUGGG | CTGGG |
| | | | CA | CA |
| | | | (SEQ | (SEQ |
| | | | ID | ID |
| | | | NO: | NO: |
| | | | 116) | 616) |
| intron_0 | RHO-18 | 0.116786532 | GCAAA | GCAAA |
| | | | UAACU | TAACT |
| | | | UCCCC | TCCCC |
| | | | CAUUC | CATTC |
| | | | CC | CC |
| | | | (SEQ | (SEQ |
| | | | ID | ID |
| | | | NO: | NO: |
| | | | 117) | 617) |
| intron_0 | RHO-19 | 0.129975835 | AGACC | AGACC |
| | | | CAGGC | CAGGC |
| | | | UGGGC | TGGGC |
| | | | ACUGA | ACTGA |
| | | | GG | GG |
| | | | (SEQ | (SEQ |
| | | | ID | ID |
| | | | NO: | NO: |
| | | | 118) | 618) |
| intron_0 | RHO-20 | 0.130270513 | CUAGG | CTAGG |
| | | | UCUCC | TCTCC |
| | | | UGGCU | TGGCT |
| | | | GUGAU | GTGAT |
| | | | CC | CC |
| | | | (SEQ | (SEQ |
| | | | ID | ID |
| | | | NO: | NO: |
| | | | 119) | 619) |
| intron_0 | RHO-21 | 0.132448578 | CCAGA | CCAGA |
| | | | AGGUG | AGGTG |
| | | | GGUGU | GGTGT |
| | | | GCCAC | GCCAC |
| | | | UU | TT |
| | | | (SEQ | (SEQ |
| | | | ID | ID |
| | | | NO: | NO: |
| | | | 120) | 620) |
| intron_0 | RHO-22 | 0.140129895 | AACAA | AACAA |
| | | | GGAAC | GGAAC |
| | | | UCUGC | TCTGC |
| | | | CCCAC | CCCAC |
| | | | AU | AT |
| | | | (SEQ | (SEQ |
| | | | ID | ID |
| | | | NO: | NO: |
| | | | 121) | 621) |
| intron_0 | RHO-23 | 0.142141636 | CAGGA | CAGGA |
| | | | UUGAA | TTGAA |
| | | | CUGGG | CTGGG |
| | | | AACCC | AACCC |
| | | | GG | GG |
| | | | (SEQ | (SEQ |
| | | | ID | ID |
| | | | NO: | NO: |
| | | | 122) | 622) |
| intron_0 | RHO-24 | 0.147082642 | GGGCG | GGGCG |
| | | | UCACA | TCACA |
| | | | CAGGG | CAGGG |
| | | | ACGGG | ACGGG |
| | | | TG | TG |
| | | | (SEQ | (SEQ |
| | | | ID | ID |
| | | | NO: | NO: |
| | | | 123) | 623) |
| intron_0 | RHO-25 | 0.14820997 | CUGUG | CTGTG |
| | | | AUCCA | ATCCA |
| | | | GGAAU | GGAAT |
| | | | AUCUC | ATCTC |
| | | | UG | TG |
| | | | (SEQ | (SEQ |
| | | | ID | ID |
| | | | NO: | NO: |
| | | | 124) | 624) |
| intron_0 | RHO-26 | 0.150900653 | UUGCA | TTGCA |
| | | | UUUAA | TTTAA |
| | | | CAGGA | CAGGA |
| | | | AAACA | AAACA |
| | | | GA | GA |
| | | | (SEQ | (SEQ |
| | | | ID | ID |
| | | | NO: | NO: |
| | | | 125) | 625) |
| intron_0 | RHO-27 | 0.151929784 | GGAGU | GGAGT |
| | | | GCACC | GCACC |
| | | | CUCCU | CTCCT |
| | | | UAGGC | TAGGC |
| | | | AG | AG |
| | | | (SEQ | (SEQ |
| | | | ID | ID |
| | | | NO: | NO: |
| | | | 126) | 626) |
| intron_0 | RHO-28 | 0.152980769 | CAUCU | CATCT |
| | | | GUCCU | GTCCT |
| | | | GCUCA | GCTCA |
| | | | CCACC | CCACC |
| | | | CC | CC |
| | | | (SEQ | (SEQ |
| | | | ID | ID |
| | | | NO: | NO: |
| | | | 127) | 627) |
| intron_0 | RHO-29 | 0.156913097 | GAGGG | GAGGG |
| | | | GAGGC | GAGGC |
| | | | AGAGG | AGAGG |
| | | | AUGCC | ATGCC |
| | | | AG | AG |
| | | | (SEQ | (SEQ |
| | | | ID | ID |
| | | | NO: | NO: |
| | | | 128) | 628) |
| intron_0 | RHO-30 | 0.166237876 | CUCAG | CTCAG |
| | | | GGAAU | GGAAT |
| | | | CUCUG | CTCTG |
| | | | GCCAU | GCCAT |
| | | | UG | TG |
| | | | (SEQ | (SEQ |
| | | | ID | ID |
| | | | NO: | NO: |
| | | | 129) | 629) |
| intron_0 | RHO-31 | 0.166367333 | UGCAC | TGCAC |
| | | | UCCCC | TCCCC |
| | | | CCUAG | CCTAG |
| | | | ACAGG | ACAGG |
| | | | GA | GA |
| | | | (SEQ | (SEQ |
| | | | ID | ID |
| | | | NO: | NO: |
| | | | 130) | 630) |
| intron_0 | RHO-32 | 0.172983706 | UGCUG | TGCTG |
| | | | UUUGU | TTTGT |
| | | | GCAGG | GCAGG |
| | | | GCUGG | GCTGG |
| | | | CA | CA |
| | | | (SEQ | (SEQ |
| | | | ID | ID |
| | | | NO: | NO: |
| | | | 131) | 63l) |
| intron_0 | RHO-33 | 0.185512517 | ACUGG | ACTGG |
| | | | GACAU | GACAT |
| | | | UCCUA | TCCTA |
| | | | ACAGU | ACAGT |
| | | | GA | GA |
| | | | (SEQ | (SEQ |
| | | | ID | ID |
| | | | NO: | NO: |
| | | | 132) | 632) |
| intron_0 | RHO-34 | 0.190420346 | AUCAG | ATCAG |
| | | | GGGGU | GGGGT |
| | | | CAGGA | CAGGA |
| | | | UUGAA | TTGAA |
| | | | CU | CT |
| | | | (SEQ | (SEQ |
| | | | ID | ID |
| | | | NO: | NO: |
| | | | 133) | 633) |
| intron_0 | RHO-35 | 0.194765615 | CUCCU | CTCCT |
| | | | CUCUG | CTCTG |
| | | | GGGGC | GGGGC |
| | | | CCAAG | CCAAG |
| | | | CU | CT |
| | | | (SEQ | (SEQ |
| | | | ID | ID |
| | | | NO: | NO: |
| | | | 134) | 634) |
| intron_0 | RHO-36 | 0.197589827 | CUGCA | CTGCA |
| | | | UCUCA | TCTCA |
| | | | GCAGA | GCAGA |
| | | | GAUAU | GATAT |
| | | | UC | TC |
| | | | (SEQ | (SEQ |
| | | | ID | ID |
| | | | NO: | NO: |
| | | | 135) | 635) |
| intron_0 | RHO-37 | 0.199499884 | UGUUU | TGTTT |
| | | | CCCUU | CCCTT |
| | | | GGAGC | GGAGC |
| | | | AGCUG | AGCTG |
| | | | UG | TG |
| | | | (SEQ | (SEQ |
| | | | ID | ID |
| | | | NO: | NO: |
| | | | 136) | 636) |
| intron_0 | RHO-38 | 0.212418288 | GCGCU | GCGCT |
| | | | CUGGG | CTGGG |
| | | | CCCAU | CCCAT |
| | | | AAGGG | AAGGG |
| | | | AC | AC |
| | | | (SEQ | (SEQ |
| | | | ID | ID |
| | | | NO: | NO: |
| | | | 137) | 637) |
| intron_0 | RHO-39 | 0.215235707 | AGGAU | AGGAT |
| | | | UGAAC | TGAAC |
| | | | UGGGA | TGGGA |
| | | | ACCCG | ACCCG |
| | | | GU | GT |
| | | | (SEQ | (SEQ |
| | | | ID | ID |
| | | | NO: | NO: |
| | | | 138) | 638) |
| intron_0 | RHO-40 | 0.21710799 | CCUAG | CCTAG |
| | | | GAGAG | GAGAG |
| | | | GCCCC | GCCCC |
| | | | CACAU | CACAT |
| | | | GU | GT |
| | | | (SEQ | (SEQ |
| | | | ID | ID |
| | | | NO: | NO: |
| | | | 139) | 639) |
| intron_0 | RHO-41 | 0.217881646 | AUCAC | ATCAC |
| | | | UCAGU | TCAGT |
| | | | UCUGG | TCTGG |
| | | | CCAGA | CCAGA |
| | | | AG | AG |
| | | | (SEQ | (SEQ |
| | | | ID | ID |
| | | | NO: | NO: |
| | | | 140) | 640) |
| intron_0 | RHO-42 | 0.227315789 | AGAGC | AGAGC |
| | | | UGGGC | TGGGC |
| | | | AAAGA | AAAGA |
| | | | AAUUC | AATTC |
| | | | CA | CA |
| | | | (SEQ | (SEQ |
| | | | ID | ID |
| | | | NO: | NO: |
| | | | 141) | 641) |
| intron_0 | RHO-43 | 0.230358178 | CCACC | CCACC |
| | | | CCAUG | CCATG |
| | | | AAGUU | AAGTT |
| | | | CCAUA | CCATA |
| | | | GG | GG |
| | | | (SEQ | (SEQ |
| | | | ID | ID |
| | | | NO: | NO: |
| | | | 142) | 642) |
| intron_0 | RHO-44 | 0.231888098 | CCACC | CCACC |
| | | | CUGAG | CTGAG |
| | | | CUUGG | CTTGG |
| | | | GCCCC | GCCCC |
| | | | CA | CA |
| | | | (SEQ | (SEQ |
| | | | ID | ID |
| | | | NO: | NO: |
| | | | 143) | 643) |
| intron_0 | RHO-45 | 0.234285631 | CAGAG | CAGAG |
| | | | GAAGA | GAAGA |
| | | | AGAAG | AGAAG |
| | | | GAAAU | GAAAT |
| | | | GA | GA |
| | | | (SEQ | (SEQ |
| | | | ID | ID |
| | | | NO: | NO: |
| | | | 144) | 644) |
| intron_0 | RHO-46 | 0.240341645 | AAACA | AAACA |
| | | | GCAGC | GCAGC |
| | | | CCGGC | CCGGC |
| | | | UAUCA | TATCA |
| | | | CC | CC |
| | | | (SEQ | (SEQ |
| | | | ID | ID |
| | | | NO: | NO: |
| | | | 145) | 645) |
| intron_0 | RHO-47 | 0.242233765 | GGAUU | GGATT |
| | | | GAACU | GAACT |
| | | | GGGAA | GGGAA |
| | | | CCCGG | CCCGG |
| | | | UA | TA |
| | | | (SEQ | (SEQ |
| | | | ID | ID |
| | | | NO: | NO: |
| | | | 146) | 646) |
| intron_0 | RHO-48 | 0.242660421 | UGUGU | TGTGT |
| | | | GUGUG | GTGTG |
| | | | UGUGU | TGTGT |
| | | | UUAGC | TTAGC |
| | | | AG | AG |
| | | | (SEQ | (SEQ |
| | | | ID | ID |
| | | | NO: | NO: |
| | | | 147) | 647) |
| intron_0 | RHO-49 | 0.251755576 | UCACA | TCACA |
| | | | CAGGG | CAGGG |
| | | | ACGGG | ACGGG |
| | | | UGCAG | TGCAG |
| | | | AG | AG |
| | | | (SEQ | (SEQ |
| | | | ID | ID |
| | | | NO: | NO: |
| | | | 148) | 648) |
| intron_0 | RHO-50 | 0.252241304 | GUGUG | GTGTG |
| | | | UGUGU | TGTGT |
| | | | GUGUG | GTGTG |
| | | | UGUUU | TGTTT |
| | | | AG | AG |
| | | | (SEQ | (SEQ |
| | | | ID | ID |
| | | | NO: | NO: |
| | | | 149) | 649) |
| intron_0 | RHO-51 | 0.255029622 | UGAGC | TGAGC |
| | | | UUGGG | TTGGG |
| | | | CCCCC | CCCCC |
| | | | AGAGA | AGAGA |
| | | | GG | GG |
| | | | (SEQ | (SEQ |
| | | | ID | ID |
| | | | NO: | NO: |
| | | | 150) | 650) |
| intron_0 | RHO-52 | 0.263525952 | AAUAU | AATAT |
| | | | CUCUG | CTCTG |
| | | | CUGAG | CTGAG |
| | | | AUGCA | ATGCA |
| | | | GG | GG |
| | | | (SEQ | (SEQ |
| | | | ID | ID |
| | | | NO: | NO: |
| | | | 151) | 651) |
| intron_0 | RHO-53 | 0.2666129 | GGAGA | GGAGA |
| | | | GGGGA | GGGGA |
| | | | AGAGA | AGAGA |
| | | | CUCAU | CTCAT |
| | | | UU | TT |
| | | | (SEQ | (SEQ |
| | | | ID | ID |
| | | | NO: | NO: |
| | | | 152) | 652) |
| intron_0 | RHO-54 | 0.287053205 | AGAAC | AGAAC |
| | | | UGAGU | TGAGT |
| | | | GAUCU | GATCT |
| | | | GUGAU | GTGAT |
| | | | UA | TA |
| | | | (SEQ | (SEQ |
| | | | ID | ID |
| | | | NO: | NO: |
| | | | 153) | 653) |
| intron_0 | RHO-55 | 0.291326632 | CCACU | CCACT |
| | | | CUCCC | CTCCC |
| | | | UAUGG | TATGG |
| | | | AACUU | AACTT |
| | | | CA | CA |
| | | | (SEQ | (SEQ |
| | | | ID | ID |
| | | | NO: | NO: |
| | | | 154) | 654) |
| intron_0 | RHO-56 | 0.292218928 | AUAAG | ATAAG |
| | | | GGACA | GGACA |
| | | | CGAAU | CGAAT |
| | | | CAGAU | CAGAT |
| | | | CA | CA |
| | | | (SEQ | (SEQ |
| | | | ID | ID |
| | | | NO: | NO: |
| | | | 155) | 655) |
| intron_0 | RHO-57 | 0.305482452 | UGGAU | TGGAT |
| | | | UUUCC | TTTCC |
| | | | AUUCU | ATTCT |
| | | | CCAGU | CCAGT |
| | | | CA | CA |
| | | | (SEQ | (SEQ |
| | | | ID | ID |
| | | | NO: | NO: |
| | | | 156) | 656) |
| intron_0 | RHO-58 | 0.310447227 | GUGCA | GTGCA |
| | | | GGAGC | GGAGC |
| | | | CCGGG | CCGGG |
| | | | AGCAU | AGCAT |
| | | | GG | GG |
| | | | (SEQ | (SEQ |
| | | | ID | ID |
| | | | NO: | NO: |
| | | | 157) | 657) |
| intron_0 | RHO-59 | 0.31581459 | GGGUG | GGGTG |
| | | | GUGAG | GTGAG |
| | | | CAGGA | CAGGA |
| | | | CAGAU | CAGAT |
| | | | GU | GT |
| | | | (SEQ | (SEQ |
| | | | ID | ID |
| | | | NO: | NO: |
| | | | 158) | 658) |
| intron_0 | RHO-60 | 0.329433399 | CAGCU | CAGCT |
| | | | CUCCC | CTCCC |
| | | | UCAGU | TCAGT |
| | | | GCCCA | GCCCA |
| | | | GC | GC |
| | | | (SEQ | (SEQ |
| | | | ID | ID |
| | | | NO: | NO: |
| | | | 159) | 659) |
| intron_0 | RHO-61 | 0.337601649 | CCUGC | CCTGC |
| | | | UGGGG | TGGGG |
| | | | CGUCA | CGTCA |
| | | | CACAG | CACAG |
| | | | GG | GG |
| | | | (SEQ | (SEQ |
| | | | ID | ID |
| | | | NO: | NO: |
| | | | 160) | 660) |
| intron_0 | RHO-62 | 0.341369802 | CACAC | CACAC |
| | | | ACACA | ACACA |
| | | | CAAAA | CAAAA |
| | | | CUCCC | CTCCC |
| | | | UA | TA |
| | | | (SEQ | (SEQ |
| | | | ID | ID |
| | | | NO: | NO: |
| | | | 161) | 661) |
| intron_0 | RHO-63 | 0.342930279 | ACUUA | ACTTA |
| | | | CGGGU | CGGGT |
| | | | GGUUG | GGTTG |
| | | | UUCUC | TTCTC |
| | | | UG | TG |
| | | | (SEQ | (SEQ |
| | | | ID | ID |
| | | | NO: | NO: |
| | | | 162) | 662) |
| intron_0 | RHO-64 | 0.347123022 | CACAG | CACAG |
| | | | GGAAG | GGAAG |
| | | | ACCCA | ACCCA |
| | | | AUGAC | ATGAC |
| | | | UG | TG |
| | | | (SEQ | (SEQ |
| | | | ID | ID |
| | | | NO: | NO: |
| | | | 163) | 663) |
| intron_0 | RHO-65 | 0.3604802 | AGCAC | AGCAC |
| | | | AGACC | AGACC |
| | | | CCACU | CCACT |
| | | | GCCUA | GCCTA |
| | | | AG | AG |
| | | | (SEQ | (SEQ |
| | | | ID | ID |
| | | | NO: | NO: |
| | | | 164) | 664) |
| intron_0 | RHO-66 | 0.396256305 | ACCUG | ACCTG |
| | | | AGGAC | AGGAC |
| | | | AGGGG | AGGGG |
| | | | CUGAG | CTGAG |
| | | | AG | AG |
| | | | (SEQ | (SEQ |
| | | | ID | ID |
| | | | NO: | NO: |
| | | | 165) | 665) |
| intron_0 | RHO-67 | 0.397224629 | CAACA | CAACA |
| | | | AUGGC | ATGGC |
| | | | CAGAG | CAGAG |
| | | | AUUCC | ATTCC |
| | | | CU | CT |
| | | | (SEQ | (SEQ |
| | | | ID | ID |
| | | | NO: | NO: |
| | | | 166) | 666) |
| intron_0 | RHO-68 | 0.40353484 | UGCUG | TGCTG |
| | | | CCUCG | CCTCG |
| | | | GUCCC | GTCCC |
| | | | AUUCU | ATTCT |
| | | | CA | CA |
| | | | (SEQ | (SEQ |
| | | | ID | ID |
| | | | NO: | NO: |
| | | | 167) | 667) |
| intron_0 | RHO-69 | 0.416729506 | UGCUG | TGCTG |
| | | | CCUGG | CCTGG |
| | | | CCACA | CCACA |
| | | | UCCCU | TCCCT |
| | | | AA | AA |
| | | | (SEQ | (SEQ |
| | | | ID | ID |
| | | | NO: | NO: |
| | | | 168) | 668) |

### III. RNA-Guided Nucleases

RNA-guided nucleases according to the present disclosure include, without limitation, naturally-occurring Class 2 CRISPR nucleases such as Cas9, and Cpfl, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpfl), species (e.g., S. pyogenes vs. S. aureus) or variation (e.g., full-length vs. truncated or split; naturally-occurring PAM specificity vs. engineered PAM specificity).

Turning to the PAM sequence, this structure takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally-occurring RNA-guided nucleases typically form DSBs in target nucleic acids, but engineered variants have been produced that generate only SSBs (discussed above; see also Ran 2013, incorporated by reference herein), or that do not cut at all.

The terms “RNA-guided nuclease” and “RNA-guided nuclease molecule” are used interchangeably herein. In some embodiments, the RNA-guided nuclease is a RNA-guided DNA endonuclease enzyme. In some embodiments, the RNA-guided nuclease is a CRISPR nuclease. Examples of RNA-guided nucleases suitable for use in the context of the methods, strategies, and treatment modalities provided herein are listed in Table 4 below, and the methods, compositions, and treatment modalities disclosed herein can, in some embodiments, make use of any combination of RNA-guided nucleases disclosed herein, or known to those of ordinary skill in the art.

TABLE 4
RNA-Guided Nucleases

| | Length | | |
|---|---|---|---|
| Nuclease | (a.a.) | PAM | Reference |
| SpCas9 | 1368 | NGG | Cong et al., Science. 2013; 339(6121): 819-23 |
| SaCas9 | 1053 | NNGRRT | Ran etal., Nature. 2015; 520(7546): 186-91. |
| (KKH) | 1067 | NNNRRT | Kleinstiver et al., Nat Biotechnol. |
| SaCas9 | | | 2015; 33(12): 1293-1298 |
| AsCpf1 | 1353 | TTTV | Zetsche et al., Nat Biotechnol. 2017; 35(1): 31-34. |
| (AsCas12a) | | | |
| LbCpf1 | 1274 | TTTV | Zetsche et al., Cell. 2015; 163(3): 759-71. |
| (LbCas12a) | | | |
| CasX | 980 | TTC | Burstein et al., Nature. 2017; 542(7640): 237-241. |
| CasY | 1200 | TA | Burstein et al., Nature. 2017; 542(7640): 237-241. |
| Cas12h1 | 870 | RTR | Yan et al., Science. 2019; 363(6422): 88-91. |
| Cas12i1 | 1093 | TTN | Yan et al., Science. 2019; 363(6422): 88-91. |
| Cas12c1 | unknown | TG | Yan et al., Science. 2019; 363(6422): 88-91. |
| Cas12c2 | unknown | TN | Yan et al., Science. 2019; 363(6422): 88-91. |
| eSpCas9 | 1423 | NGG | Chen et al., Nature. 2017; 550(7676): 407-410. |
| Cas9-HF1 | 1367 | NGG | Chen et al., Nature. 2017; 550(7676): 407-410. |
| HypaCas9 | 1404 | NGG | Chen et al., Nature. 2017; 550(7676): 407-410. |
| dCas9-Fokl | 1623 | NGG | U.S. Pat. No. 9,322,037 |
| Sniper-Cas9 | 1389 | NGG | Lee et al., Nat Commun. 2018; 9(1): 3048. |
| xCas9 | 1786 | NGG, NG, | Wang et al., Plant Biotechnol J. 2018; pbi.13053. |
| | | GAA, | |
| | | GAT | |
| AaCas12b | 1129 | TTN | Teng et al. Cell Discov. 2018; 4: 63. |
| evoCas9 | 1423 | NGG | Casini et al., Nat Biotechnol. 2018; 36(3): 265-271. |
| SpCas9-NG | 1423 | NG | Nishimasu et al. Science. 2018; 361(6408): 1259- |
| | | | 1262. |
| VRQR | 1368 | NGA | Li et al., The CRISPR Journal, 2018; 01: 01 |
| VRER | 1372 | NGCG | Kleinstiver et al., Nature. 2016; 529(7587): 490-5. |
| NmeCas9 | 1082 | NNNNGA | Amrani et al., Genome Biol. 2018; 19(1): 214. |
| | | TT | |
| CjCas9 | 984 | NNNNRY | Kim et al., Nat Commun. 2017; 8: 14500. |
| | | AC | |
| BhCas12b | 1108 | ATTN | Strecker et al., Nat Commun. 2019 Jan. |
| | | | 22; 10(1): 212. |
| BhCas12b | 1108 | ATTN | Strecker etal., Nat Commun. 2019 Jan. |
| V4 | | | 22; 10(1): 212. |

In one embodiment, the RNA-guided nuclease is a Acidaminococcus sp. Cpfl RR variant (AsCpfl-RR). In another embodiment, the RNA-guided nuclease is a Cpfl RVR variant

Exemplary suitable methods for designing targeting domains and guide RNAs, as well as for the use of the various Cas nucleases in the context of genome editing approaches, are known to those of skill in the art. Some exemplary methods are disclosed herein, and additional suitable methods will be apparent to the skilled artisan based on the present disclosure. The disclosure is not limited in this respect.

### IV. RHO Genomic Sequence and Complementary DNA Sequences

The RHO genomic sequence is known to those of ordinary skill in the art. An exemplary RHO genomic sequence is provided below for ease of reference:

(SEQ ID NO: 1)
AGAGTCATCCAGCTGGAGCCCTGAGTGGCTGAGCTCAGGCCTTCGCAGCATTCTTGGGTGGG
AGCAGCCACGGGTCAGCCACAAGGGCCACAGCCATGAATGGCACAGAAGGCCCTAACTTCTA
CGTGCCCTTCTCCAATGCGACGGGTGTGGTACGCAGCCCCTTCGAGTACCCACAGTACTACC
TGGCTGAGCCATGGCAGTTCTCCATGCTGGCCGCCTACATGTTTCTGCTGATCGTGCTGGGC
TTCCCCATCAACTTCCTCACGCTCTACGTCACCGTCCAGCACAAGAAGCTGCGCACGCCTCT
CAACTACATCCTGCTCAACCTAGCCGTGGCTGACCTCTTCATGGTCCTAGGTGGCTTCACCA
GCACCCTCTACACCTCTCTGCATGGATACTTCGTCTTCGGGCCCACAGGATGCAATTTGGAG
GGCTTCTTTGCCACCCTGGGCGGTATGAGCCGGGTGTGGGTGGGGTGTGCAGGAGCCCGGGA
GCATGGAGGGGTCTGGGAGAGTCCCGGGCTTGGCGGTGGTGGCTGAGAGGCCTTCTCCCTTC
TCCTGTCCTGTCAATGTTATCCAAAGCCCTCATATATTCAGTCAACAAACACCATTCATGGT
GATAGCCGGGCTGCTGTTTGTGCAGGGCTGGCACTGAACACTGCCTTGATCTTATTTGGAGC
AATATGCGCTTGTCTAATTTCACAGCAAGAAAACTGAGCTGAGGCTCAAAGAAGTCAAGCGC
CCTGCTGGGGCGTCACACAGGGACGGGTGCAGAGTTGAGTTGGAAGCCCGCATCTATCTCGG
GCCATGTTTGCAGCACCAAGCCTCTGTTTCCCTTGGAGCAGCTGTGCTGAGTCAGACCCAGG
CTGGGCACTGAGGGAGAGCTGGGCAAGCCAGACCCCTCCTCTCTGGGGGCCCAAGCTCAGGG
TGGGAAGTGGATTTTCCATTCTCCAGTCATTGGGTCTTCCCTGTGCTGGGCAATGGGCTCGG
TCCCCTCTGGCATCCTCTGCCTCCCCTCTCAGCCCCTGTCCTCAGGTGCCCCTCCAGCCTCC
CTGCCGCGTTCCAAGTCTCCTGGTGTTGAGAACCGCAAGCAGCCGCTCTGAAGCAGTTCCTT
TTTGCTTTAGAATAATGTCTTGCATTTAACAGGAAAACAGATGGGGTGCTGCAGGGATAACA
GATCCCACTTAACAGAGAGGAAAACTGAGGCAGGGAGAGGGGAAGAGACTCATTTAGGGATG
TGGCCAGGCAGCAACAAGAGCCTAGGTCTCCTGGCTGTGATCCAGGAATATCTCTGCTGAGA
TGCAGGAGGAGACGCTAGAAGCAGCCATTGCAAAGCTGGGTGACGGGGAGAGCTTACCGCCA
GCCACAAGCGTCTCTCTGCCAGCCTTGCCCTGTCTCCCCCATGTCCAGGCTGCTGCCTCGGT
CCCATTCTCAGGGAATCTCTGGCCATTGTTGGGTGTTTGTTGCATTCAATAATCACAGATCA
CTCAGTTCTGGCCAGAAGGTGGGTGTGCCACTTACGGGTGGTTGTTCTCTGCAGGGTCAGTC
CCAGTTTACAAATATTGTCCCTTTCACTGTTAGGAATGTCCCAGTTTGGTTGATTAACTATA
TGGCCACTCTCCCTATGGAACTTCATGGGGTGGTGAGCAGGACAGATGTCTGAATTCCATCA
TTTCCTTCTTCTTCCTCTGGGCAAAACATTGCACATTGCTTCATGGCTCCTAGGAGAGGCCC
CCACATGTCCGGGTTATTTCATTTCCCGAGAAGGGAGAGGGAGGAAGGACTGCCAATTCTGG
GTTTCCACCACCTCTGCATTCCTTCCCAACAAGGAACTCTGCCCCACATTAGGATGCATTCT
TCTGCTAAACACACACACACACACACACACACACAACACACACACACACACACACACACACA
CACACACAAAACTCCCTACCGGGTTCCCAGTTCAATCCTGACCCCCTGATCTGATTCGTGTC
CCTTATGGGCCCAGAGCGCTAAGCAAATAACTTCCCCCATTCCCTGGAATTTCTTTGCCCAG
CTCTCCTCAGCGTGTGGTCCCTCTGCCCCTTCCCCCTCCTCCCAGCACCAAGCTCTCTCCTT
CCCCAAGGCCTCCTCAAATCCCTCTCCCACTCCTGGTTGCCTTCCTAGCTACCCTCTCCCTG
TCTAGGGGGGAGTGCACCCTCCTTAGGCAGTGGGGTCTGTGCTGACCGCCTGCTGACTGCCT
TGCAGGTGAAATTGCCCTGTGGTCCTTGGTGGTCCTGGCCATCGAGCGGTACGTGGTGGTGT
GTAAGCCCATGAGCAACTTCCGCTTCGGGGAGAACCATGCCATCATGGGCGTTGCCTTCACC
TGGGTCATGGCGCTGGCCTGCGCCGCACCCCCACTCGCCGGCTGGTCCAGGTAATGGCACTG
AGCAGAAGGGAAGAAGCTCCGGGGGCTCTTTGTAGGGTCCTCCAGTCAGGACTCAAACCCAG
TAGTGTCTGGTTCCAGGCACTGACCTTGTATGTCTCCTGGCCCAAATGCCCACTCAGGGTAG
GGGTGTAGGGCAGAAGAAGAAACAGACTCTAATGTTGCTACAAGGGCTGGTCCCATCTCCTG
AGCCCCATGTCAAACAGAATCCAAGACATCCCAACCCTTCACCTTGGCTGTGCCCCTAATCC
TCAACTAAGCTAGGCGCAAATTCCAATCCTCTTTGGTCTAGTACCCCGGGGGCAGCCCCCTC
TAACCTTGGGCCTCAGCAGCAGGGGAGGCCACACCTTCCTAGTGCAGGTGGCCATATTGTGG
CCCCTTGGAACTGGGTCCCACTCAGCCTCTAGGCGATTGTCTCCTAATGGGGCTGAGATGAG
ACACAGTGGGGACAGTGGTTTGGACAATAGGACTGGTGACTCTGGTCCCCAGAGGCCTCATG
TCCCTCTGTCTCCAGAAAATTCCCACTCTCACTTCCCTTTCCTCCTCAGTCTTGCTAGGGTC
CATTTCTTACCCCTTGCTGAATTTGAGCCCACCCCCTGGACTTTTTCCCCATCTTCTCCAAT
CTGGCCTAGTTCTATCCTCTGGAAGCAGAGCCGCTGGACGCTCTGGGTTTCCTGAGGCCCGT
CCACTGTCACCAATATCAGGAACCATTGCCACGTCCTAATGACGTGCGCTGGAAGCCTCTAG
TTTCCAGAAGCTGCACAAAGATCCCTTAGATACTCTGTGTGTCCATCTTTGGCCTGGAAAAT
ACTCTCACCCTGGGGCTAGGAAGACCTCGGTTTGTACAAACTTCCTCAAATGCAGAGCCTGA
GGGCTCTCCCCACCTCCTCACCAACCCTCTGCGTGGCATAGCCCTAGCCTCAGCGGGCAGTG
GATGCTGGGGCTGGGCATGCAGGGAGAGGCTGGGTGGTGTCATCTGGTAACGCAGCCACCAA
ACAATGAAGCGACACTGATTCCACAAGGTGCATCTGCATCCCCATCTGATCCATTCCATCCT
GTCACCCAGCCATGCAGACGTTTATGATCCCCTTTTCCAGGGAGGGAATGTGAAGCCCCAGA
AAGGGCCAGCGCTCGGCAGCCACCTTGGCTGTTCCCAAGTCCCTCACAGGCAGGGTCTCCCT
ACCTGCCTGTCCTCAGGTACATCCCCGAGGGCCTGCAGTGCTCGTGTGGAATCGACTACTAC
ACGCTCAAGCCGGAGGTCAACAACGAGTCTTTTGTCATCTACATGTTCGTGGTCCACTTCAC
CATCCCCATGATTATCATCTTTTTCTGCTATGGGCAGCTCGTCTTCACCGTCAAGGAGGTAC
GGGCCGGGGGGTGGGCGGCCTCACGGCTCTGAGGGTCCAGCCCCCAGCATGCATCTGCGGCT
CCTGCTCCCTGGAGGAGCCATGGTCTGGACCCGGGTCCCGTGTCCTGCAGGCCGCTGCCCAG
CAGCAGGAGTCAGCCACCACACAGAAGGCAGAGAAGGAGGTCACCCGCATGGTCATCATCAT
GGTCATCGCTTTCCTGATCTGCTGGGTGCCCTACGCCAGCGTGGCATTCTACATCTTCACCC
ACCAGGGCTCCAACTTCGGTCCCATCTTCATGACCATCCCAGCGTTCTTTGCCAAGAGCGCC
GCCATCTACAACCCTGTCATCTATATCATGATGAACAAGCAGGTGCCTACTGCGGGTGGGAG
GGCCCCAGTGCCCCAGGCCACAGGCGCTGCCTGCCAAGGACAAGCTACTTCCCAGGGCAGGG
GAGGGGGCTCCATCAGGGTTACTGGCAGCAGTCTTGGGTCAGCAGTCCCAATGGGGAGTGTG
TGAGAAATGCAGATTCCTGGCCCCACTCAGAACTGCTGAATCTCAGGGTGGGCCCAGGAACC
TGCATTTCCAGCAAGCCCTCCACAGGTGGCTCAGATGCTCACTCAGGTGGGAGAAGCTCCAG
TCAGCTAGTTCTGGAAGCCCAATGTCAAAGTCAGAAGGACCCAAGTCGGGAATGGGATGGGC
CAGTCTCCATAAAGCTGAATAAGGAGCTAAAAAGTCTTATTCTGAGGGGTAAAGGGGTAAAG
GGTTCCTCGGAGAGGTACCTCCGAGGGGTAAACAGTTGGGTAAACAGTCTCTGAAGTCAGCT
CTGCCATTTTCTAGCTGTATGGCCCTGGGCAAGTCAATTTCCTTCTCTGTGCTTTGGTTTCC
TCATCCATAGAAAGGTAGAAAGGGCAAAACACCAAACTCTTGGATTACAAGAGATAATTTAC
AGAACACCCTTGGCACACAGAGGGCACCATGAAATGTCACGGGTGACACAGCCCCCTTGTGC
TCAGTCCCTGGCATCTCTAGGGGTGAGGAGCGTCTGCCTAGCAGGTTCCCTCCAGGAAGCTG
GATTTGAGTGGATGGGGCGCTGGAATCGTGAGGGGCAGAAGCAGGCAAAGGGTCGGGGCGAA
CCTCACTAACGTGCCAGTTCCAAGCACACTGTGGGCAGCCCTGGCCCTGACTCAAGCCTCTT
GCCTTCCAGTTCCGGAACTGCATGCTCACCACCATCTGCTGCGGCAAGAACCCACTGGGTGA
CGATGAGGCCTCTGCTACCGTGTCCAAGACGGAGACGAGCCAGGTGGCCCCGGCCTAAGACC
TGCCTAGGACTCTGTGGCCGACTATAGGCGTCTCCCATCCCCTACACCTTCCCCCAGCCACA
GCCATCCCACCAGGAGCAGCGCCTGTGCAGAATGAACGAAGTCACATAGGCTCCTTAATTTT
TTTTTTTTTTTTAAGAAATAATTAATGAGGCTCCTCACTCACCTGGGACAGCCTGAGAAGGG
ACATCCACCAAGACCTACTGATCTGGAGTCCCACGTTCCCCAAGGCCAGCGGGATGTGTGCC
CCTCCTCCTCCCAACTCATCTTTCAGGAACACGAGGATTCTTGCTTTCTGGAAAAGTGTCCC
AGCTTAGGGATAAGTGTCTAGCACAGAATGGGGCACACAGTAGGTGCTTAATAAATGCTGGA
TGGATGCAGGAAGGAATGGAGGAATGAATGGGAAGGGAGAACATATCTATCCTCTCAGACCC
TCGCAGCAGCAGCAACTCATACTTGGCTAATGATATGGAGCAGTTGTTTTTCCCTCCCTGGG
CCTCACTTTCTTCTCCTATAAAATGGAAATCCCAGATCCCTGGTCCTGCCGACACGCAGCTA
CTGAGAAGACCAAAAGAGGTGTGTGTGTGTCTATGTGTGTGTTTCAGCACTTTGTAAATAGC
AAGAAGCTGTACAGATTCTAGTTAATGTTGTGAATAACATCAATTAATGTAACTAGTTAATT
ACTATGATTATCACCTCCTGATAGTGAACATTTTGAGATTGGGCATTCAGATGATGGGGTTT
CACCCAACCTTGGGGCAGGTTTTTAAAAATTAGCTAGGCATCAAGGCCAGACCAGGGCTGGG
GGTTGGGCTGTAGGCAGGGACAGTCACAGGAATGCAGAATGCAGTCATCAGACCTGAAAAAA
CAACACTGGGGGAGGGGGACGGTGAAGGCCAAGTTCCCAATGAGGGTGAGATTGGGCCTGGG
GTCTCACCCCTAGTGTGGGGCCCCAGGTCCCGTGCCTCCCCTTCCCAATGTGGCCTATGGAG
AGACAGGCCTTTCTCTCAGCCTCTGGAAGCCACCTGCTCTTTTGCTCTAGCACCTGGGTCCC
AGCATCTAGAGCATGGAGCCTCTAGAAGCCATGCTCACCCGCCCACATTTAATTAACAGCTG
AGTCCCTGATGTCATCCTTATCTCGAAGAGCTTAGAAACAAAGAGTGGGAAATTCCACTGGG
CCTACCTTCCTTGGGGATGTTCATGGGCCCCAGTTTCCAGTTTCCCTTGCCAGACAAGCCCA
TCTTCAGCAGTTGCTAGTCCATTCTCCATTCTGGAGAATCTGCTCCAAAAAGCTGGCCACAT
CTCTGAGGTGTCAGAATTAAGCTGCCTCAGTAACTGCTCCCCCTTCTCCATATAAGCAAAGC
CAGAAGCTCTAGCTTTACCCAGCTCTGCCTGGAGACTAAGGCAAATTGGGCCATTAAAAGCT
CAGCTCCTATGTTGGTATTAACGGTGGTGGGTTTTGTTGCTTTCACACTCTATCCACAGGAT
AGATTGAAACTGCCAGCTTCCACCTGATCCCTGACCCTGGGATGGCTGGATTGAGCAATGAG
CAGAGCCAAGCAGCACAGAGTCCCCTGGGGCTAGAGGTGGAGGAGGCAGTCCTGGGAATGGG
AAAAACCCCA

The RHO genomic sequence can be annotated as follows:

mRNA 1..456,2238..2406,3613..3778,3895..4134,4970..6706

CDS 96..456,2238..2406,3613..3778,3895..4134,4970..5080

Exemplary target domains, described in more detail elsewhere herein, are provided below in Table 5 for the purpose of illustration:

TABLE 5

| | Position of target domain in RHO genomic |
|---|---|
| Reference ID | sequence (SEQ ID NO: 1) |
| RHO-1 | 74 . . . 95 |
| RHO-2 | 391 . . . 412 |
| RHO-3 | 381 . . . 402 |
| RHO-4 | 312 . . . 333 |
| RHO-5 | 178 . . . 199 |
| RHO-6 | 144 . . . 165 |
| RHO-7 | 453 . . . 474 |
| RHO-8 | 448 . . . 469 |
| RHO-9 | 2334 . . . 2355 |
| RHO-10 | 2395 . . . 2416 |
| RHO-11 | 2389 . . . 2410 |

A variety of RHO cDNA sequences may be used herein. In certain embodiments, the RHO cDNA may be delivered to provide an exogenous functional RHO cDNA.

Provided below is an exemplary nucleic acid sequence of a wild-type RHO cDNA:

(SEQ ID NO: 2)
ATGAATGGCACAGAAGGCCCTAACTTCTACGTGCCCTTCTCCAATGCGAC
GGGTGTGGTACGCAGCCCCTTCGAGTACCCACAGTACTACCTGGCTGAGC
CATGGCAGTTCTCCATGCTGGCCGCCTACATGTTTCTGCTGATCGTGCTG
GGCTTCCCCATCAACTTCCTCACGCTCTACGTCACCGTCCAGCACAAGAA
GCTGCGCACGCCTCTCAACTACATCCTGCTCAACCTAGCCGTGGCTGACC
TCTTCATGGTCCTAGGTGGCTTCACCAGCACCCTCTACACCTCTCTGCAT
GGATACTTCGTCTTCGGGCCCACAGGATGCAATTTGGAGGGCTTCTTTGC
CACCCTGGGCGGTGAAATTGCCCTGTGGTCCTTGGTGGTCCTGGCCATCG
AGCGGTACGTGGTGGTGTGTAAGCCCATGAGCAACTTCCGCTTCGGGGAG
AACCATGCCATCATGGGCGTTGCCTTCACCTGGGTCATGGCGCTGGCCTG
CGCCGCACCCCCACTCGCCGGCTGGTCCAGGTACATCCCCGAGGGCCTGC
AGTGCTCGTGTGGAATCGACTACTACACGCTCAAGCCGGAGGTCAACAAC
GAGTCTTTTGTCATCTACATGTTCGTGGTCCACTTCACCATCCCCATGAT
TATCATCTTTTTCTGCTATGGGCAGCTCGTCTTCACCGTCAAGGAGGCCG
CTGCCCAGCAGCAGGAGTCAGCCACCACACAGAAGGCAGAGAAGGAGGTC
ACCCGCATGGTCATCATCATGGTCATCGCTTTCCTGATCTGCTGGGTGCC
CTACGCCAGCGTGGCATTCTACATCTTCACCCACCAGGGCTCCAACTTCG
GTCCCATCTTCATGACCATCCCAGCGTTCTTTGCCAAGAGCGCCGCCATC
TACAACCCTGTCATCTATATCATGATGAACAAGCAGTTCCGGAACTGCAT
GCTCACCACCATCTGCTGCGGCAAGAACCCACTGGGTGACGATGAGGCCT
CTGCTACCGTGTCCAAGACGGAGACGAGCCAGGTGGCCCCGGCCTAA

In certain embodiments, the RHO cDNA may be codon-optimized to increase expression. In certain embodiments, the RHO cDNA may be codon-modified to be resistant to hybridization with a gRNA targeting domain. In certain embodiments, the RHO cDNA is not codon-modified to be resistant to hybridization with a gRNA targeting domain.

Provided below are exemplary nucleic acid sequences of codon optimized RHO cDNA:

Codon optimized RHO-encoding sequence 1 (Codon 1):
(SEQ ID NO: 13)
ATGAACGGCACCGAGGGCCCCAACTTCTACGTCCCCTTCAGCAACGCCACCGGCGTCGTCCG
CAGCCCCTTCGAGTACCCCCAGTACTACCTGGCCGAGCCCTGGCAGTTCAGCATGCTGGCCG
CCTACATGTTCCTGCTGATCGTCCTGGGCTTCCCCATCAACTTCCTGACCCTGTACGTCACC
GTCCAGCACAAGAAGCTGCGCACCCCCCTGAACTACATCCTGCTGAACCTGGCCGTCGCCGA
CCTGTTCATGGTCCTGGGCGGCTTCACCAGCACCCTGTACACCAGCCTGCACGGCTACTTCG
TCTTCGGCCCCACCGGCTGCAACCTGGAGGGCTTCTTCGCCACCCTGGGCGGCGAGATCGCC
CTGTGGAGCCTGGTCGTCCTGGCCATCGAGCGCTACGTCGTCGTCTGCAAGCCCATGAGCAA
CTTCCGCTTCGGCGAGAACCACGCCATCATGGGCGTCGCCTTCACCTGGGTCATGGCCCTGG
CCTGCGCCGCCCCCCCCCTGGCCGGCTGGAGCCGCTACATCCCCGAGGGCCTGCAGTGCAGC
TGCGGCATCGACTACTACACCCTGAAGCCCGAGGTCAACAACGAGAGCTTCGTCATCTACAT
GTTCGTCGTCCACTTCACCATCCCCATGATCATCATCTTCTTCTGCTACGGCCAGCTGGTCT
TCACCGTCAAGGAGGCCGCCGCCCAGCAGCAGGAGAGCGCCACCACCCAGAAGGCCGAGAAG
GAGGTCACCCGCATGGTCATCATCATGGTCATCGCCTTCCTGATCTGCTGGGTCCCCTACGC
CAGCGTCGCCTTCTACATCTTCACCCACCAGGGCAGCAACTTCGGCCCCATCTTCATGACCA
TCCCCGCCTTCTTCGCCAAGAGCGCCGCCATCTACAACCCCGTCATCTACATCATGATGAAC
AAGCAGTTCCGCAACTGCATGCTGACCACCATCTGCTGCGGCAAGAACCCCCTGGGCGACGA
CGAGGCCAGCGCCACCGTCAGCAAGACCGAGACCAGCCAGGTCGCCCCCGCCTAA
Codon optimized RHO-encoding sequence 2 (Codon 2):
(SEQ ID NO: 14)
ATGAACGGCACCGAGGGCCCCAACTTCTACGTGCCCTTCTCCAACGCCACCGGCGTGGTGCG
CTCCCCCTTCGAGTACCCCCAGTACTACCTGGCCGAGCCCTGGCAGTTCTCCATGCTGGCCG
CCTACATGTTCCTGCTGATCGTGCTGGGCTTCCCCATCAACTTCCTGACCCTGTACGTGACC
GTGCAGCACAAGAAGCTGCGCACCCCCCTGAACTACATCCTGCTGAACCTGGCCGTGGCCGA
CCTGTTCATGGTGCTGGGCGGCTTCACCTCCACCCTGTACACCTCCCTGCACGGCTACTTCG
TGTTCGGCCCCACCGGCTGCAACCTGGAGGGCTTCTTCGCCACCCTGGGCGGCGAGATCGCC
CTGTGGTCCCTGGTGGTGCTGGCCATCGAGCGCTACGTGGTGGTGTGCAAGCCCATGTCCAA
CTTCCGCTTCGGCGAGAACCACGCCATCATGGGCGTGGCCTTCACCTGGGTGATGGCCCTGG
CCTGCGCCGCCCCCCCCCTGGCCGGCTGGTCCCGCTACATCCCCGAGGGCCTGCAGTGCTCC
TGCGGCATCGACTACTACACCCTGAAGCCCGAGGTGAACAACGAGTCCTTCGTGATCTACAT
GTTCGTGGTGCACTTCACCATCCCCATGATCATCATCTTCTTCTGCTACGGCCAGCTGGTGT
TCACCGTGAAGGAGGCCGCCGCCCAGCAGCAGGAGTCCGCCACCACCCAGAAGGCCGAGAAG
GAGGTGACCCGCATGGTGATCATCATGGTGATCGCCTTCCTGATCTGCTGGGTGCCCTACGC
CTCCGTGGCCTTCTACATCTTCACCCACCAGGGCTCCAACTTCGGCCCCATCTTCATGACCA
TCCCCGCCTTCTTCGCCAAGTCCGCCGCCATCTACAACCCCGTGATCTACATCATGATGAAC
AAGCAGTTCCGCAACTGCATGCTGACCACCATCTGCTGCGGCAAGAACCCCCTGGGCGACGA
CGAGGCCTCCGCCACCGTGTCCAAGACCGAGACCTCCCAGGTGGCCCCCGCCTAA
Codon Optimized RHO-encoding sequence 3 (Codon 3):
(SEQ ID NO: 15)
ATGAACGGCACCGAGGGCCCCAACTTCTACGTCCCCTTCAGCAACGCCACCGGCGTCGTCCG
CAGCCCCTTCGAGTACCCCCAGTACTACCTGGCCGAGCCCTGGCAGTTCTCTATGCTGGCCG
CCTACATGTTCCTGCTGATCGTCCTGGGCTTCCCTATCAACTTCCTCACCCTCTACGTCACC
GTCCAGCACAAGAAGCTCCGCACCCCTCTCAACTACATCCTCCTTAACCTTGCCGTCGCCGA
CCTTTTCATGGTCCTTGGCGGCTTCACCTCTACTCTTTACACTTCTTTGCACGGGTACTTCG
TGTTCGGTCCTACTGGTTGCAACTTGGAGGGTTTCTTCGCCACTTTGGGTGGTGAGATCGCC
TTGTGGTCGTTGGTGGTGTTAGCTATCGAGCGATACGTGGTGGTGTGCAAGCCTATGTCGAA
CTTCCGGTTCGGTGAGAATCATGCTATCATGGGAGTGGCTTTTACTTGGGTGATGGCTTTAG
CTTGCGCTGCTCCTCCGTTAGCTGGATGGTCGCGTTATATCCCGGAGGGATTACAGTGCTCA
TGCGGAATCGACTATTATACTCTAAAGCCGGAAGTTAATAATGAATCATTTGTTATTTATAT
GTTTGTTGTTCATTTTACAATTCCGATGATTATTATTTTTTTTTGTTATGGACAGCTAGTTT
TTACAGTTAAGGAAGCAGCAGCACAGCAACAAGAATCAGCAACAACACAAAAGGCAGAAAAA
GAAGTTACAAGGATGGTTATTATTATGGTAATTGCATTTCTAATATGTTGGGTACCGTATGC
ATCCGTAGCATTTTATATATTTACACATCAAGGGTCCAATTTTGGGCCAATATTTATGACGA
TACCAGCGTTTTTTGCGAAATCCGCGGCGATATATAATCCAGTAATATATATAATGATGAAT
AAACAATTTAGAAATTGTATGCTAACGACGATATGTTGTGGGAAAAATCCACTAGGGGATGA
TGAAGCGAGTGCGACGGTAAGTAAAACGGAAACGAGTCAAGTAGCGCCAGCGTAA
Codon Optimized RHO-encoding sequence 4 (Codon 4):
(SEQ ID NO: 16)
ATGAACGGCACCGAGGGTCCCAATTTCTACGTCCCATTTTCCAACGCCACGGGGGTGGTACG
CAGCCCTTTCGAATATCCGCAGTACTATCTGGCTGAGCCCTGGCAGTTTTCTATGCTCGCAG
CGTACATGTTCTTGCTAATCGTTCTGGGATTTCCAATTAATTTCCTCACATTGTATGTCACC
GTGCAGCACAAGAAGCTACGGACGCCTCTGAACTACATCCTCTTGAATCTAGCCGTCGCTGA
CCTGTTTATGGTTCTCGGCGGTTTCACATCGACCTTGTATACGTCACTACATGGGTACTTTG
TCTTCGGACCGACAGGCTGCAACCTGGAAGGTTTTTTCGCAACCCTCGGGGGAGAGATTGCG
TTGTGGTCCCTAGTGGTACTGGCCATCGAAAGGTATGTTGTCGTGTGTAAGCCCATGAGCAA
TTTTCGCTTCGGCGAGAACCACGCTATTATGGGTGTAGCATTTACGTGGGTTATGGCGCTCG
CCTGCGCTGCACCACCTTTGGCGGGGTGGTCTCGGTACATCCCGGAAGGACTACAGTGTTCG
TGCGGCATTGATTATTACACACTGAAGCCCGAGGTCAATAACGAATCATTCGTGATCTATAT
GTTTGTAGTTCATTTCACCATTCCAATGATCATTATCTTTTTCTGTTACGGTCAGCTCGTCT
TTACGGTGAAGGAGGCCGCTGCACAGCAGCAGGAATCCGCGACAACCCAGAAGGCCGAGAAG
GAAGTAACGAGGATGGTTATTATCATGGTCATTGCTTTCTTGATCTGCTGGGTGCCTTATGC
AAGCGTAGCGTTTTACATTTTCACACACCAGGGGTCTAATTTTGGACCGATCTTCATGACCA
TTCCCGCCTTTTTCGCTAAGTCGGCAGCGATCTATAACCCAGTTATTTACATCATGATGAAT
AAGCAGTTTCGCAACTGTATGCTAACGACAATTTGCTGTGGCAAGAATCCTCTGGGTGACGA
TGAGGCCTCAGCTACCGTCTCCAAGACGGAAACAAGCCAGGTGGCACCGGCGTAA
Codon Optimized RHO-encoding sequence 5 (Codon 5):
(SEQ ID NO: 17)
ATGAATGGGACTGAAGGACCTAATTTCTATGTGCCATTTAGCAATGCTACTGGCGTTGTCAG
AAGCCCCTTCGAATATCCACAATACTATCTGGCCGAACCTTGGCAGTTCAGCATGCTCGCTG
CCTATATGTTTCTGCTGATTGTGCTGGGCTTTCCCATAAATTTCCTCACCCTGTATGTTACT
GTTCAACACAAAAAGCTGCGGACGCCTCTGAACTACATACTGCTGAACCTGGCCGTCGCCGA
CCTGTTTATGGTCCTGGGAGGCTTTACAAGCACTCTGTATACAAGCCTGCACGGCTACTTCG
TGTTCGGCCCCACAGGCTGCAACCTCGAAGGCTTCTTTGCCACCCTCGGAGGAGAGATTGCC
CTGTGGAGCCTGGTGGTGCTGGCCATCGAAAGGTATGTGGTGGTGTGTAAACCCATGTCCAA
TTTTCGGTTCGGCGAGAACCACGCTATTATGGGAGTGGCTTTCACTTGGGTGATGGCCCTGG
CCTGCGCCGCCCCACCACTGGCCGGGTGGAGCCGGTACATCCCAGAGGGGCTGCAATGTAGC
TGCGGAATCGACTATTATACCCTGAAACCAGAGGTGAACAACGAGAGCTTTGTGATTTATAT
GTTTGTGGTGCATTTTACAATTCCTATGATTATCATTTTCTTCTGTTACGGGCAACTGGTGT
TTACCGTGAAGGAAGCCGCCGCTCAACAGCAGGAGAGCGCCACAACCCAAAAGGCCGAGAAG
GAGGTGACCAGAATGGTGATTATTATGGTGATCGCTTTTCTGATTTGCTGGGTGCCATACGC
TAGCGTCGCTTTCTATATTTTCACTCACCAGGGGAGCAACTTCGGCCCCATTTTCATGACAA
TCCCTGCCTTTTTTGCTAAAAGCGCCGCCATCTATAACCCAGTGATCTACATCATGATGAAC
AAACAGTTTAGGAACTGTATGCTCACAACAATCTGCTGTGGAAAGAACCCCCTCGGCGATGA
CGAAGCCAGCGCCACCGTCAGCAAGACAGAAACAAGCCAGGTGGCCCCTGCCTAA
Codon Optimized RHO-encoding sequence 6 (Codon 6):
(SEQ ID NO: 18)
ATGAATGGCACAGAGGGCCCTAACTTCTACGTGCCCTTTAGCAATGCCACAGGCGTCGTGCG
GAGCCCTTTTGAGTACCCTCAGTACTATCTGGCCGAGCCTTGGCAGTTTAGCATGCTGGCCG
CCTACATGTTCCTGCTGATCGTGCTGGGCTTCCCCATCAACTTTCTGACCCTGTACGTGACC
GTGCAGCACAAGAAGCTGCGGACCCCTCTGAACTACATCCTGCTGAATCTGGCCGTGGCCGA
CCTGTTTATGGTGCTCGGCGGCTTTACCAGCACACTGTACACAAGCCTGCACGGCTACTTCG
TGTTTGGCCCCACCGGCTGCAATCTGGAAGGCTTTTTTGCCACACTCGGCGGCGAAATTGCT
CTGTGGTCACTGGTGGTGCTGGCCATCGAGAGATACGTGGTCGTGTGCAAGCCCATGAGCAA
CTTCAGATTCGGCGAGAACCACGCCATCATGGGCGTCGCCTTTACATGGGTTATGGCCCTGG
CTTGTGCAGCTCCTCCTCTTGCCGGCTGGTCCAGATATATTCCTGAGGGCCTGCAGTGCAGC
TGCGGCATCGATTACTACACCCTGAAGCCTGAAGTGAACAACGAGAGCTTCGTGATCTACAT
GTTTGTGGTGCACTTCACGATCCCCATGATCATCATATTCTTTTGCTACGGCCAGCTGGTGT
TCACCGTGAAAGAAGCCGCTGCTCAGCAGCAAGAGAGCGCCACAACACAGAAAGCCGAGAAA
GAAGTGACCCGGATGGTCATTATCATGGTTATCGCCTTTCTGATCTGTTGGGTGCCCTACGC
CAGCGTGGCCTTCTACATCTTTACCCACCAAGGCAGCAACTTCGGCCCCATCTTTATGACAA
TCCCCGCCTTCTTTGCCAAGAGCGCCGCCATCTACAACCCCGTGATCTATATCATGATGAAC
AAGCAGTTCCGCAACTGCATGCTGACCACCATCTGCTGCGGAAAGAACCCTCTGGGAGATGA
TGAGGCCAGCGCCACCGTGTCTAAGACCGAAACATCTCAGGTGGCCCCTGCATGA

In certain embodiments, the RHO cDNA may include a modified 5′ UTR, a modified 3′UTR, or a combination thereof. For example, in certain embodiments, the RHO cDNA may include a truncated 5′ UTR, a truncated 3′UTR, or a combination thereof. In certain embodiments, the RHO cDNA may include a 3′UTR from a known stable messenger RNA (mRNA). For example, in certain embodiments, the RHO cDNA may include a heterologous 3′-UTR downstream of the RHO coding sequence. For example, in some embodiments, the RHO cDNA may include an α-globin 3′ UTR. In certain embodiments, the RHO cDNA may include a β-globin 3′ UTR. In certain embodiments, the RHO cDNA may include one or more introns. In certain embodiments, the RHO cDNA may include a truncation of one or more introns.

Exemplary suitable heterologous 3′-UTRs that can be used to stabilize the transcript of the RHO cDNA include, but are not limited, to the following:

HBA1 3′UTR:
(SEQ ID NO: 38)
GCTGGAGCCTCGGTGGCCATGCTTCTTGCCCCTTGGGCCTCCCCCCAGCCCCTCCTCCCCTT
CCTGCACCCGTACCCCCGTGGTCTTTGAATAAAGTCTGAGTGGGCGGCA
short HBA1 3′UTR:
(SEQ ID NO: 39)
GCTGGAGCCTCGGTGGCCATGCTTCTTGCCCCTTGGGCCTCCCCCCAGCCCCTCCTCCCCTT
CCTGCACCCGTACCCCCGTGGTCTTTGAATAAAGTCTGA
TH 3′UTR:
(SEQ ID NO: 40)
GTGCACGGCGTCCCTGAGGGCCCTTCCCAACCTCCCCTGGTCCTGCACTGTCCCGGAGCTCA
GGCCCTGGTGAGGGGCTGGGTCCCGGGTGCCCCCCATGCCCTCCCTGCTGCCAGGCTCCCAC
TGCCCCTGCACCTGCTTCTCAGCGCAACAGCTGTGTGTGCCCGTGGTGAGGTTGTGCTGCCT
GTGGTGAGGTCCTGTCCTGGCTCCCAGGGTCCTGGGGGCTGCTGCACTGCCCTCCGCCCTTC
CCTGACACTGTCTGCTGCCCCAATCACCGTCACAATAAAAGAAACTGTGGTCTCTA
COL1A1 3′UTR:
(SEQ ID NO: 41)
ACTCCCTCCATCCCAACCTGGCTCCCTCCCACCCAACCAACTTTCCCCCCAACCCGGAAACA
GACAAGCAACCCAAACTGAACCCCCTCAAAAGCCAAAAAATGGGAGACAATTTCACATGGAC
TTTGGAAAATATTTTTTTCCTTTGCATTCATCTCTCAAACTTAGTTTTTATCTTTGACCAAC
CGAACATGACCAAAAACCAAAAGTGCATTCAACCTTACCAAAAAAAAAAAAAAAAAAAGAAT
AAATAAATAACTTTTTAAAAAAGGAAGCTTGGTCCACTTGCTTGAAGACCCATGCGGGGGTA
AGTCCCTTTCTGCCCGTTGGGCTTATGAAACCCCAATGCTGCCCTTTCTGCTCCTTTCTCCA
CACCCCCCTTGGGGCCTCCCCTCCACTCCTTCCCAAATCTGTCTCCCCAGAAGACACAGGAA
ACAATGTATTGTCTGCCCAGCAATCAAAGGCAATGCTCAAACACCCAAGTGGCCCCCACCCT
CAGCCCGCTCCTGCCCGCCCAGCACCCCCAGGCCCTGGGGGACCTGGGGTTCTCAGACTGCC
AAAGAAGCCTTGCCATCTGGCGCTCCCATGGCTCTTGCAACATCTCCCCTTCGTTTTTGAGG
GGGTCATGCCGGGGGAGCCACCAGCCCCTCACTGGGTTCGGAGGAGAGTCAGGAAGGGCCAC
GACAAAGCAGAAACATCGGATTTGGGGAACGCGTGTCAATCCCTTGTGCCGCAGGGCTGGGC
GGGAGAGACTGTTCTGTTCCTTGTGTAACTGTGTTGCTGAAAGACTACCTCGTTCTTGTCTT
GATGTGTCACCGGGGCAACTGCCTGGGGGCGGGGATGGGGGCAGGGTGGAAGCGGCTCCCCA
TTTTATACCAAAGGTGCTACATCTATGTGATGGGTGGGGTGGGGAGGGAATCACTGGTGCTA
TAGAAATTGAGATGCCCCCCCAGGCCAGCAAATGTTCCTTTTTGTTCAAAGTCTATTTTTAT
TCCTTGATATTTTTCTTTTTTTTTTTTTTTTTTTGTGGATGGGGACTTGTGAATTTTTCTAA
AGGTGCTATTTAACATGGGAGGAGAGCGTGTGCGGCTCCAGCCCAGCCCGCTGCTCACTTTC
CACCCTCTCTCCACCTGCCTCTGGCTTCTCAGGCCTCTGCTCTCCGACCTCTCTCCTCTGAA
ACCCTCCTCCACAGCTGCAGCCCATCCTCCCGGCTCCCTCCTAGTCTGTCCTGCGTCCTCTG
TCCCCGGGTTTCAGAGACAACTTCCCAAAGCACAAAGCAGTTTTTCCCCCTAGGGGTGGGAG
GAAGCAAAAGACTCTGTACCTATTTTGTATGTGTATAATAATTTGAGATGTTTTTAATTATT
TTGATTGCTGGAATAAAGCATGTGGAAATGACCCAAACATAA
ALOX15 3′UTR:
(SEQ ID NO: 42)
GCGTCGCCACCCTTTGGTTATTTCAGCCCCCATCACCCAAGCCACAAGCTGACCCCTTCGTG
GTTATAGCCCTGCCCTCCCAAGTCCCACCCTCTTCCCATGTCCCACCCTCCCTAGAGGGGCA
CCTTTTCATGGTCTCTGCACCCAGTGAACACATTTTACTCTAGAGGCATCACCTGGGACCTT
ACTCCTCTTTCCTTCCTTCCTCCTTTCCTATCTTCCTTCCTCTCTCTCTTCCTCTTTCTTCA
TTCAGATCTATATGGCAAATAGCCACAATTATATAAATCATTTCAAGACTAGAATAGGGGGA
TATAATACATATTACTCCACACCTTTTATGAATCAAATATGATTTTTTTGTTGTTGTTAAGA
CAGAGTCTCACTTTGACACCCAGGCTGGAGTGCAGTGGTGCCATCACCACGGCTCACTGCAG
CCTCAGCGTCCTGGGCTCAAATGATCCTCCCACCTCAGCCTCCTGAGTAGCTGGGACTACAG
GCTCATGCCATCATGCCCAGCTAATATTTTTTTATTTTCGTGGAGACGGGGCCTCACTATGT
TGCCTAGGCTGGAAATAGGATTTTGAACCCAAATTGAGTTTAACAATAATAAAAAGTTGTTT
TACGCTAAAGATGGAAAAGAACTAGGACTGAACTATTTTAAATAAAATATTGGCAAAAGAA

In certain embodiments, the RHO cDNA may include one or more introns. In certain embodiments, the RHO cDNA may include a truncation of one or more introns.

Table 6 below provides exemplary sequences of RHO cDNA containing introns.

TABLE 6

| cDNA Identifier | RHO cDNA sequence |
|---|---|
| RHO cDNA | ATGAATGGCACAGAAGGCCCTAACTTCTACGTGCCCTTCTCCAATGCGACGGGTGTGG |
| with intron 1 | TACGCAGCCCCTTCGAGTACCCACAGTACTACCTGGCTGAGCCATGGCAGTTCTCCAT |
| | GCTGGCCGCCTACATGTTTCTGCTGATCGTGCTGGGCTTCCCCATCAACTTCCTCACG |
| | CTCTACGTCACCGTCCAGCACAAGAAGCTGCGCACGCCTCTCAACTACATCCTGCTCA |
| | ACCTAGCCGTGGCTGACCTCTTCATGGTCCTAGGTGGCTTCACCAGCACCCTCTACAC |
| | CTCTCTGCATGGATACTTCGTCTTCGGGCCCACAGGATGCAATTTGGAGGGCTTCTTT |
| | GCCACCCTGGGCGGTATGAGCCGGGTGTGGGTGGGGTGTGCAGGAGCCCGGGAGCATG |
| | GAGGGGTCTGGGAGAGTCCCGGGCTTGGCGGTGGTGGCTGAGAGGCCTTCTCCCTTCT |
| | CCTGTCCTGTCAATGTTATCCAAAGCCCTCATATATTCAGTCAACAAACACCATTCAT |
| | GGTGATAGCCGGGCTGCTGTTTGTGCAGGGCTGGCACTGAACACTGCCTTGATCTTAT |
| | TTGGAGCAATATGCGCTTGTCTAATTTCACAGCAAGAAAACTGAGCTGAGGCTCAAAG |
| | AAGTCAAGCGCCCTGCTGGGGCGTCACACAGGGACGGGTGCAGAGTTGAGTTGGAAGC |
| | CCGCATCTATCTCGGGCCATGTTTGCAGCACCAAGCCTCTGTTTCCCTTGGAGCAGCT |
| | GTGCTGAGTCAGACCCAGGCTGGGCACTGAGGGAGAGCTGGGCAAGCCAGACCCCTCC |
| | TCTCTGGGGGCCCAAGCTCAGGGTGGGAAGTGGATTTTCCATTCTCCAGTCATTGGGT |
| | CTTCCCTGTGCTGGGCAATGGGCTCGGTCCCCTCTGGCATCCTCTGCCTCCCCTCTCA |
| | GCCCCTGTCCTCAGGTGCCCCTCCAGCCTCCCTGCCGCGTTCCAAGTCTCCTGGTGTT |
| | GAGAACCGCAAGCAGCCGCTCTGAAGCAGTTCCTTTTTGCTTTAGAATAATGTCTTGC |
| | ATTTAACAGGAAAACAGATGGGGTGCTGCAGGGATAACAGATCCCACTTAACAGAGAG |
| | GAAAACTGAGGCAGGGAGAGGGGAAGAGACTCATTTAGGGATGTGGCCAGGCAGCAAC |
| | AAGAGCCTAGGTCTCCTGGCTGTGATCCAGGAATATCTCTGCTGAGATGCAGGAGGAG |
| | ACGCTAGAAGCAGCCATTGCAAAGCTGGGTGACGGGGAGAGCTTACCGCCAGCCACAA |
| | GCGTCTCTCTGCCAGCCTTGCCCTGTCTCCCCCATGTCCAGGCTGCTGCCTCGGTCCC |
| | ATTCTCAGGGAATCTCTGGCCATTGTTGGGTGTTTGTTGCATTCAATAATCACAGATC |
| | ACTCAGTTCTGGCCAGAAGGTGGGTGTGCCACTTACGGGTGGTTGTTCTCTGCAGGGT |
| | CAGTCCCAGTTTACAAATATTGTCCCTTTCACTGTTAGGAATGTCCCAGTTTGGTTGA |
| | TTAACTATATGGCCACTCTCCCTATGGAACTTCATGGGGTGGTGAGCAGGACAGATGT |
| | CTGAATTCCATCATTTCCTTCTTCTTCCTCTGGGCAAAACATTGCACATTGCTTCATG |
| | GCTCCTAGGAGAGGCCCCCACATGTCCGGGTTATTTCATTTCCCGAGAAGGGAGAGGG |
| | AGGAAGGACTGCCAATTCTGGGTTTCCACCACCTCTGCATTCCTTCCCAACAAGGAAC |
| | TCTGCCCCACATTAGGATGCATTCTTCTGCTAAACACACACACACACACACACACACA |
| | CAACACACACACACACACACACACACACACACACACAAAACTCCCTACCGGGTTCCCA |
| | GTTCAATCCTGACCCCCTGATCTGATTCGTGTCCCTTATGGGCCCAGAGCGCTAAGCA |
| | AATAACTTCCCCCATTCCCTGGAATTTCTTTGCCCAGCTCTCCTCAGCGTGTGGTCCC |
| | TCTGCCCCTTCCCCCTCCTCCCAGCACCAAGCTCTCTCCTTCCCCAAGGCCTCCTCAA |
| | ATCCCTCTCCCACTCCTGGTTGCCTTCCTAGCTACCCTCTCCCTGTCTAGGGGGGAGT |
| | GCACCCTCCTTAGGCAGTGGGGTCTGTGCTGACCGCCTGCTGACTGCCTTGCAGGTGA |
| | AATTGCCCTGTGGTCCTTGGTGGTCCTGGCCATCGAGCGGTACGTGGTGGTGTGTAAG |
| | CCCATGAGCAACTTCCGCTTCGGGGAGAACCATGCCATCATGGGCGTTGCCTTCACCT |
| | GGGTCATGGCGCTGGCCTGCGCCGCACCCCCACTCGCCGGCTGGTCCAGGTACATCCC |
| | CGAGGGCCTGCAGTGCTCGTGTGGAATCGACTACTACACGCTCAAGCCGGAGGTCAAC |
| | AACGAGTCTTTTGTCATCTACATGTTCGTGGTCCACTTCACCATCCCCATGATTATCA |
| | TCTTTTTCTGCTATGGGCAGCTCGTCTTCACCGTCAAGGAGGCCGCTGCCCAGCAGCA |
| | GGAGTCAGCCACCACACAGAAGGCAGAGAAGGAGGTCACCCGCATGGTCATCATCATG |
| | GTCATCGCTTTCCTGATCTGCTGGGTGCCCTACGCCAGCGTGGCATTCTACATCTTCA |
| | CCCACCAGGGCTCCAACTTCGGTCCCATCTTCATGACCATCCCAGCGTTCTTTGCCAA |
| | GAGCGCCGCCATCTACAACCCTGTCATCTATATCATGATGAACAAGCAGTTCCGGAAC |
| | TGCATGCTCACCACCATCTGCTGCGGCAAGAACCCACTGGGTGACGATGAGGCCTCTG |
| | CTACCGTGTCCAAGACGGAGACGAGCCAGGTGGCCCCGGCCTAA |
| | (SEQ ID NO: 4) |
| RHO cDNA with | ATGAATGGCACAGAAGGCCCTAACTTCTACGTGCCCTTCTCCAATGCGACGGGTGTG |
| intron 2 | GTACGCAGCCCCTTCGAGTACCCACAGTACTACCTGGCTGAGCCATGGCAGTTCTCC |
| | ATGCTGGCCGCCTACATGTTTCTGCTGATCGTGCTGGGCTTCCCCATCAACTTCCTC |
| | ACGCTCTACGTCACCGTCCAGCACAAGAAGCTGCGCACGCCTCTCAACTACATCCTG |
| | CTCAACCTAGCCGTGGCTGACCTCTTCATGGTCCTAGGTGGCTTCACCAGCACCCTC |
| | TACACCTCTCTGCATGGATACTTCGTCTTCGGGCCCACAGGATGCAATTTGGAGGGC |
| | TTCTTTGCCACCCTGGGCGGTGAAATTGCCCTGTGGTCCTTGGTGGTCCTGGCCATC |
| | GAGCGGTACGTGGTGGTGTGTAAGCCCATGAGCAACTTCCGCTTCGGGGAGAACCAT |
| | GCCATCATGGGCGTTGCCTTCACCTGGGTCATGGCGCTGGCCTGCGCCGCACCCCCA |
| | CTCGCCGGCTGGTCCAGGTAATGGCACTGAGCAGAAGGGAAGAAGCTCCGGGGGCTC |
| | TTTGTAGGGTCCTCCAGTCAGGACTCAAACCCAGTAGTGTCTGGTTCCAGGCACTGA |
| | CCTTGTATGTCTCCTGGCCCAAATGCCCACTCAGGGTAGGGGTGTAGGGCAGAAGAA |
| | GAAACAGACTCTAATGTTGCTACAAGGGCTGGTCCCATCTCCTGAGCCCCATGTCAA |
| | ACAGAATCCAAGACATCCCAACCCTTCACCTTGGCTGTGCCCCTAATCCTCAACTAA |
| | GCTAGGCGCAAATTCCAATCCTCTTTGGTCTAGTACCCCGGGGGCAGCCCCCTCTAA |
| | CCTTGGGCCTCAGCAGCAGGGGAGGCCACACCTTCCTAGTGCAGGTGGCCATATTGT |
| | GGCCCCTTGGAACTGGGTCCCACTCAGCCTCTAGGCGATTGTCTCCTAATGGGGCTG |
| | AGATGAGACACAGTGGGGACAGTGGTTTGGACAATAGGACTGGTGACTCTGGTCCCC |
| | AGAGGCCTCATGTCCCTCTGTCTCCAGAAAATTCCCACTCTCACTTCCCTTTCCTCC |
| | TCAGTCTTGCTAGGGTCCATTTCTTACCCCTTGCTGAATTTGAGCCCACCCCCTGGA |
| | CTTTTTCCCCATCTTCTCCAATCTGGCCTAGTTCTATCCTCTGGAAGCAGAGCCGCT |
| | GGACGCTCTGGGTTTCCTGAGGCCCGTCCACTGTCACCAATATCAGGAACCATTGCC |
| | ACGTCCTAATGACGTGCGCTGGAAGCCTCTAGTTTCCAGAAGCTGCACAAAGATCCC |
| | TTAGATACTCTGTGTGTCCATCTTTGGCCTGGAAAATACTCTCACCCTGGGGCTAGG |
| | AAGACCTCGGTTTGTACAAACTTCCTCAAATGCAGAGCCTGAGGGCTCTCCCCACCT |
| | CCTCACCAACCCTCTGCGTGGCATAGCCCTAGCCTCAGCGGGCAGTGGATGCTGGGG |
| | CTGGGCATGCAGGGAGAGGCTGGGTGGTGTCATCTGGTAACGCAGCCACCAAACAAT |
| | GAAGCGACACTGATTCCACAAGGTGCATCTGCATCCCCATCTGATCCATTCCATCCT |
| | GTCACCCAGCCATGCAGACGTTTATGATCCCCTTTTCCAGGGAGGGAATGTGAAGCC |
| | CCAGAAAGGGCCAGCGCTCGGCAGCCACCTTGGCTGTTCCCAAGTCCCTCACAGGCA |
| | GGGTCTCCCTACCTGCCTGTCCTCAGGTACATCCCCGAGGGCCTGCAGTGCTCGTGT |
| | GGAATCGACTACTACACGCTCAAGCCGGAGGTCAACAACGAGTCTTTTGTCATCTAC |
| | ATGTTCGTGGTCCACTTCACCATCCCCATGATTATCATCTTTTTCTGCTATGGGCAG |
| | CTCGTCTTCACCGTCAAGGAGGCCGCTGCCCAGCAGCAGGAGTCAGCCACCACACAG |
| | AAGGCAGAGAAGGAGGTCACCCGCATGGTCATCATCATGGTCATCGCTTTCCTGATC |
| | TGCTGGGTGCCCTACGCCAGCGTGGCATTCTACATCTTCACCCACCAGGGCTCCAAC |
| | TTCGGTCCCATCTTCATGACCATCCCAGCGTTCTTTGCCAAGAGCGCCGCCATCTAC |
| | AACCCTGTCATCTATATCATGATGAACAAGCAGTTCCGGAACTGCATGCTCACCACC |
| | ATCTGCTGCGGCAAGAACCCACTGGGTGACGATGAGGCCTCTGCTACCGTGTCCAAG |
| | ACGGAGACGAGCCAGGTGGCCCCGGCCTAA |
| | (SEQ ID NO: 5) |
| RHO cDNA | ATGAATGGCACAGAAGGCCCTAACTTCTACGTGCCCTTCTCCAATGCGACGGGTGTG |
| with intron 3 | GTACGCAGCCCCTTCGAGTACCCACAGTACTACCTGGCTGAGCCATGGCAGTTCTCC |
| | ATGCTGGCCGCCTACATGTTTCTGCTGATCGTGCTGGGCTTCCCCATCAACTTCCTC |
| | ACGCTCTACGTCACCGTCCAGCACAAGAAGCTGCGCACGCCTCTCAACTACATCCTG |
| | CTCAACCTAGCCGTGGCTGACCTCTTCATGGTCCTAGGTGGCTTCACCAGCACCCTC |
| | TACACCTCTCTGCATGGATACTTCGTCTTCGGGCCCACAGGATGCAATTTGGAGGGC |
| | TTCTTTGCCACCCTGGGCGGTGAAATTGCCCTGTGGTCCTTGGTGGTCCTGGCCATC |
| | GAGCGGTACGTGGTGGTGTGTAAGCCCATGAGCAACTTCCGCTTCGGGGAGAACCAT |
| | GCCATCATGGGCGTTGCCTTCACCTGGGTCATGGCGCTGGCCTGCGCCGCACCCCCA |
| | CTCGCCGGCTGGTCCAGGTACATCCCCGAGGGCCTGCAGTGCTCGTGTGGAATCGAC |
| | TACTACACGCTCAAGCCGGAGGTCAACAACGAGTCTTTTGTCATCTACATGTTCGTG |
| | GTCCACTTCACCATCCCCATGATTATCATCTTTTTCTGCTATGGGCAGCTCGTCTTC |
| | ACCGTCAAGGAGGTACGGGCCGGGGGGTGGGCGGCCTCACGGCTCTGAGGGTCCAGC |
| | CCCCAGCATGCATCTGCGGCTCCTGCTCCCTGGAGGAGCCATGGTCTGGACCCGGGT |
| | CCCGTGTCCTGCAGGCCGCTGCCCAGCAGCAGGAGTCAGCCACCACACAGAAGGCAG |
| | AGAAGGAGGTCACCCGCATGGTCATCATCATGGTCATCGCTTTCCTGATCTGCTGGG |
| | TGCCCTACGCCAGCGTGGCATTCTACATCTTCACCCACCAGGGCTCCAACTTCGGTC |
| | CCATCTTCATGACCATCCCAGCGTTCTTTGCCAAGAGCGCCGCCATCTACAACCCTG |
| | TCATCTATATCATGATGAACAAGCAGTTCCGGAACTGCATGCTCACCACCATCTGCT |
| | GCGGCAAGAACCCACTGGGTGACGATGAGGCCTCTGCTACCGTGTCCAAGACGGAGA |
| | CGAGCCAGGTGGCCCCGGCCTAA |
| | (SEQ ID NO: 6) |
| RHO cDNA | ATGAATGGCACAGAAGGCCCTAACTTCTACGTGCCCTTCTCCAATGCGACGGGTGTG |
| with intron 4 | GTACGCAGCCCCTTCGAGTACCCACAGTACTACCTGGCTGAGCCATGGCAGTTCTCC |
| | ATGCTGGCCGCCTACATGTTTCTGCTGATCGTGCTGGGCTTCCCCATCAACTTCCTC |
| | ACGCTCTACGTCACCGTCCAGCACAAGAAGCTGCGCACGCCTCTCAACTACATCCTG |
| | CTCAACCTAGCCGTGGCTGACCTCTTCATGGTCCTAGGTGGCTTCACCAGCACCCTC |
| | TACACCTCTCTGCATGGATACTTCGTCTTCGGGCCCACAGGATGCAATTTGGAGGGC |
| | TTCTTTGCCACCCTGGGCGGTGAAATTGCCCTGTGGTCCTTGGTGGTCCTGGCCATC |
| | GAGCGGTACGTGGTGGTGTGTAAGCCCATGAGCAACTTCCGCTTCGGGGAGAACCAT |
| | GCCATCATGGGCGTTGCCTTCACCTGGGTCATGGCGCTGGCCTGCGCCGCACCCCCA |
| | CTCGCCGGCTGGTCCAGGTACATCCCCGAGGGCCTGCAGTGCTCGTGTGGAATCGAC |
| | TACTACACGCTCAAGCCGGAGGTCAACAACGAGTCTTTTGTCATCTACATGTTCGTG |
| | GTCCACTTCACCATCCCCATGATTATCATCTTTTTCTGCTATGGGCAGCTCGTCTTC |
| | ACCGTCAAGGAGGCCGCTGCCCAGCAGCAGGAGTCAGCCACCACACAGAAGGCAGAG |
| | AAGGAGGTCACCCGCATGGTCATCATCATGGTCATCGCTTTCCTGATCTGCTGGGTG |
| | CCCTACGCCAGCGTGGCATTCTACATCTTCACCCACCAGGGCTCCAACTTCGGTCCC |
| | ATCTTCATGACCATCCCAGCGTTCTTTGCCAAGAGCGCCGCCATCTACAACCCTGTC |
| | ATCTATATCATGATGAACAAGCAGGTGCCTACTGCGGGTGGGAGGGCCCCAGTGCCC |
| | CAGGCCACAGGCGCTGCCTGCCAAGGACAAGCTACTTCCCAGGGCAGGGGAGGGGGC |
| | TCCATCAGGGTTACTGGCAGCAGTCTTGGGTCAGCAGTCCCAATGGGGAGTGTGTGA |
| | GAAATGCAGATTCCTGGCCCCACTCAGAACTGCTGAATCTCAGGGTGGGCCCAGGAA |
| | CCTGCATTTCCAGCAAGCCCTCCACAGGTGGCTCAGATGCTCACTCAGGTGGGAGAA |
| | GCTCCAGTCAGCTAGTTCTGGAAGCCCAATGTCAAAGTCAGAAGGACCCAAGTCGGG |
| | AATGGGATGGGCCAGTCTCCATAAAGCTGAATAAGGAGCTAAAAAGTCTTATTCTGA |
| | GGGGTAAAGGGGTAAAGGGTTCCTCGGAGAGGTACCTCCGAGGGGTAAACAGTTGGG |
| | TAAACAGTCTCTGAAGTCAGCTCTGCCATTTTCTAGCTGTATGGCCCTGGGCAAGTC |
| | AATTTCCTTCTCTGTGCTTTGGTTTCCTCATCCATAGAAAGGTAGAAAGGGCAAAAC |
| | ACCAAACTCTTGGATTACAAGAGATAATTTACAGAACACCCTTGGCACACAGAGGGC |
| | ACCATGAAATGTCACGGGTGACACAGCCCCCTTGTGCTCAGTCCCTGGCATCTCTAG |
| | GGGTGAGGAGCGTCTGCCTAGCAGGTTCCCTCCAGGAAGCTGGATTTGAGTGGATGG |
| | GGCGCTGGAATCGTGAGGGGCAGAAGCAGGCAAAGGGTCGGGGCGAACCTCACTAAC |
| | GTGCCAGTTCCAAGCACACTGTGGGCAGCCCTGGCCCTGACTCAAGCCTCTTGCCTT |
| | CCAGTTCCGGAACTGCATGCTCACCACCATCTGCTGCGGCAAGAACCCACTGGGTGA |
| | CGATGAGGCCTCTGCTACCGTGTCCAAGACGGAGACGAGCCAGGTGGCCCCGGCCTA |
| | A |
| | (SEQ ID NO: 7) |

### V. Genome Editing Approaches

In some embodiments, the RHO gene is altered using one of the approaches discussed herein.

### NHEJ-Mediated Knock-Out of RHO

Some aspects of this disclosure provide strategies, methods, compositions, and treatment modalities that are characterized by targeting an RNA-guided nuclease, e.g., a Cas9 or Cpfl nuclease to a RHO target sequence, e.g., a target sequence described herein and/or using a guide RNA described herein, wherein the RNA-guided nuclease cuts the RHO genomic DNA at or near the RHO target sequence, resulting in NHEJ-mediated repair of the cut genomic DNA. The outcome of this NHEJ-mediated repair is typically the creation of an indel at the cut site, which in turn results in a loss-of-function of the cut RHO gene. A loss-of-function can be characterized by a decrease or a complete abolishment of expression of a gene product, e.g., in the case of the RHO gene: a RHO gene product, for example, a RHO transcript or a RHO protein, or by expression of a gene product that does not exhibit a function of the wild-type gene product. In some embodiments, a loss-of-function of the RHO gene is characterized by expression of a lower level of functional RHO protein. In some embodiments, a loss-of-function of the RHO gene is characterized by abolishment of expression of RHO protein from the RHO gene. In some embodiments, a loss-of-function of a mutant RHO gene or allele is characterized by decreased expression, or abolishment of expression, of the encoded mutant RHO protein.

As described herein, nuclease-induced non-homologous end-joining (NHEJ) can be used to introduce indels at a target position. Nuclease-induced NHEJ can also be used to remove (e.g., delete) genomic sequence including the mutation at a target position in a gene of interest.

While not wishing to be bound by theory, it is believed that, in an embodiment, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair.

The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs as long as the generation of a specific final sequence is not required. If a double-strand break is targeted near to a specific sequence motif, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of deletion.

Both double strand cleaving RNA-guided nucleases and single strand, or nickase, RNA-guided nucleases can be used in the methods and compositions described herein to generate break-induced indels.

Some exemplary methods featuring NHEJ-mediated knock-out of the RHO gene are provided herein, as are some exemplary suitable guide RNAs, RNA-guided nucleases, delivery methods, and other aspects related to such methods. Additional suitable methods, guide RNAs, RNA-guided nucleases, delivery methods, etc., will be apparent to those of ordinary skill in the art based on the present disclosure.

### HDR Repair and Template Nucleic Acids

As described herein, in certain embodiments, nuclease-induced homology directed repair (HDR) can be used to alter a target position of a mutant RHO gene (e.g., knock out) and replace the mutant RHO gene with a wild-type RHO sequence. While not wishing to be bound by theory, it is believed that alteration of the target position occurs by homology-directed repair (HDR) with a donor template or template nucleic acid. For example, the donor template or the template nucleic acid provides for alteration of the target position. It is contemplated that a plasmid donor can be used as a template for homologous recombination. It is further contemplated that a single stranded donor template can be used as a template for alteration of the target position by alternate methods of homology directed repair (e.g., single strand annealing) between the cut sequence and the donor template. Donor template-effected alteration of a target sequence depends on cleavage by an RNA-guided nuclease molecule. Cleavage by RNA-guided nuclease molecule can comprise a double strand break or two single strand breaks.

Mutant RHO genes that can be replaced with wild-type RHO by HDR using a template nucleic acid include mutant RHO genes comprising point mutations, mutation hotspots or sequence insertions. In an embodiment, a mutant RHO gene having a point mutation or a mutation hotspot (e.g., a mutation hotspot of less than about 30 bp, e.g., less than 25, 20, 15, 10 or 5 bp) can be altered (e.g., knocked out) by either a single double-strand break or two single strand breaks. In an embodiment, a mutant RHO gene having a point mutation or a mutation hotspot (e.g., a mutation hotspot greater than about 30 bp, e.g., more than 35, 40, 45, 50, 75, 100, 150, 200, 250, 300, 400 or 500 bp) or an insertion can be altered (e.g., knocked out) by (1) a single double-strand break, (2) two single strand breaks, (3) two double stranded breaks with a break occurring on each side of the target position, or (4) four single stranded breaks with a pair of single stranded breaks occurring on each side of the target position.

Mutant RHO genes that can be altered (e.g., knocked out) by HDR and replaced with a template nucleic acid include, but are not limited to, those in Table A, such as P23, e.g., P23H or P23L, T58, e.g., T58R and P347, e.g., P347T, P347A, P347S, P347G, P347L or P347R.

### Double Strand Break Mediated Alteration

In an embodiment, double strand cleavage is affected by an RNA-guided nuclease. In certain embodiments, the RNA-guided nuclease may be a Cas9 molecule having cleavage activity associated with an HNH-like domain and cleavage activity associated with anRuvC-like domain, e.g., an N-terminal RuvC-like domain, e.g., a wild type Cas9. Such embodiments require only a single gRNA.

### Single Strand Break Mediated Alteration

In other embodiments, two single strand breaks, or nicks, are affected by a Cas9 molecule having nickase activity, e.g., cleavage activity associated with an HNH-like domain or cleavage activity associated with an N-terminal RuvC-like domain. Such embodiments require two gRNAs, one for placement of each single strand break. In an embodiment, the Cas9 molecule having nickase activity cleaves the strand to which the gRNA hybridizes, but not the strand that is complementary to the strand to which the gRNA hybridizes. In an embodiment, the Cas9 molecule having nickase activity does not cleave the strand to which the gRNA hybridizes, but rather cleaves the strand that is complementary to the strand to which the gRNA hybridizes.

In an embodiment, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (the complementary strand, which does not have the NGG PAM on it). In other embodiments, a Cas9 molecule having an H840, e.g., an H840A, mutation can be used as a nickase. H840A inactivates HNH; therefore, the Cas9 nickase has (only) RuvC activity and cuts on the non-complementary strand (the strand that has the NGG PAM and whose sequence is identical to the gRNA).

In an embodiment, in which a nickase and two gRNAs are used to position two single strand nicks, one nick is on the +strand and one nick is on the - strand of the target nucleic acid. The PAMs are outwardly facing. The gRNAs can be selected such that the gRNAs are separated by, from about 0-50, 0-100, or 0-200 nucleotides. In an embodiment, there is no overlap between the target domains that are complementary to the targeting domains of the two gRNAs. In an embodiment, the gRNAs do not overlap and are separated by as much as 50, 100, or 200 nucleotides. In an embodiment, the use of two gRNAs can increase specificity, e.g., by decreasing off-target binding (Ran 2013).

In an embodiment, a single nick can be used to induce HDR. It is contemplated herein that a single nick can be used to increase the ratio of HR to NHEJ at a given cleavage site.

### Placement of the Double Strand Break or a Single Strand Break Relative to the Target Position

The double strand break or single strand break in one of the strands should be sufficiently close to the target position such that alteration occurs. In an embodiment, the distance is not more than 50, 100, 200, 300, 350 or 400 nucleotides. While not wishing to be bound by theory, it is believed that the break should be sufficiently close to the target position such that the break is within the region that is subject to exonuclease-mediated removal during end resection.

In an embodiment, in which a gRNA (unimolecular (or chimeric) or modular gRNA) and RNA-guided nuclease induce a double strand break for the purpose of inducing HDR-mediated replacement, the cleavage site is between 0-200 bp (e.g., 0-175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position. In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In an embodiment, in which two gRNAs (independently, unimolecular (or chimeric) or modular gRNA) complexing with Cas9 nickases induce two single strand breaks for the purpose of inducing HDR-mediated replacement, the closer nick is between 0-200 bp (e.g., 0-175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position and the two nicks will ideally be within 25-55 bp of each other (e.g., 25 to 50, 25 to 45, 25 to 40, 25 to 35, 25 to 30, 30 to 55, 30 to 50, 30 to 45, 30 to 40, 30 to 35, 35 to 55, 35 to 50, 35 to 45, 35 to 40, 40 to 55, 40 to 50, 40 to 45 bp) and no more than 100 bp away from each other (e.g., no more than 90, 80, 70, 60, 50, 40, 30, 20, 10 or 5 bp away from each other). In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In one embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

### Length of the Homology Arms

The homology arm should extend at least as far as the region in which end resection may occur, e.g., in order to allow the resected single stranded overhang to find a complementary region within the donor template. The overall length could be limited by parameters such as plasmid size or viral packaging limits. In an embodiment, a homology arm does not extend into repeated elements, e.g., ALU repeats, LINE repeats.

Exemplary homology arm lengths include a least 50, 100, 250, 500, 750 or 1000 nucleotides.

Target position, as used herein, refers to a site on a target nucleic acid (e.g., the RHO gene) that is modified by a Cas9 molecule-dependent process. For example, the target position can be a modified Cas9 molecule cleavage of the target nucleic acid and template nucleic acid directed modification, e.g., alteration, of the target position. In an embodiment, a target position can be a site between two nucleotides, e.g., adjacent nucleotides, on the target nucleic acid into which one or more nucleotides is added. The target position may comprise one or more nucleotides that are altered, e.g., knocked out, by a template nucleic acid. In an embodiment, the target position is within a target domain (e.g., the sequence to which the gRNA binds). In an embodiment, a target position is upstream or downstream of a target domain (e.g., the sequence to which the gRNA binds).

A template nucleic acid, as that term is used herein, refers to a nucleic acid sequence which can be used in conjunction with an RNA-guided nuclease molecule and a gRNA molecule to alter the structure of a target position. In an embodiment, the target nucleic acid is modified to have some or all of the sequence of the template nucleic acid, typically at or near cleavage site(s). In an embodiment, the template nucleic acid is single stranded. In an alternate embodiment, the template nucleic acid is double stranded. In an embodiment, the template nucleic acid is DNA, e.g., double stranded DNA. In an alternate embodiment, the template nucleic acid is single stranded DNA. In an embodiment, the template nucleic acid is encoded on the same vector backbone, e.g. AAV genome, plasmid DNA, as the Cas9 and gRNA. In an embodiment, the template nucleic acid is excised from a vector backbone in vivo, e.g., it is flanked by gRNA recognition sequences.

In an embodiment, the template nucleic acid alters the structure of the target position by participating in a homology directed repair event. In an embodiment, the template nucleic acid alters the sequence of the target position. In an embodiment, the template nucleic acid results in the incorporation of a modified, or non-naturally occurring base into the target nucleic acid.

Typically, the template sequence undergoes a breakage-mediated or -catalyzed recombination with the target sequence. In an embodiment, the template nucleic acid includes a sequence that corresponds to a site on the target sequence that is cleaved by an eaCas9 mediated cleavage event. In an embodiment, the template nucleic acid includes a sequence that corresponds to both, a first site on the target sequence that is cleaved in a first Cas9 mediated event, and a second site on the target sequence that is cleaved in a second Cas9 mediated event.

In an embodiment, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation.

In other embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in the RHO gene can be used to alter the structure of a target sequence. The template sequence can be used to alter an unwanted structure, e.g., an unwanted or mutant nucleotide.

A template nucleic acid comprises the following components:

[5′ homology arm]-[replacement sequence]-[3′ homology arm].

The homology arms provide for recombination into the chromosome, thus replacing the undesired element, e.g., a mutation or signature, with the replacement sequence. In an embodiment, the homology arms flank the most distal cleavage sites.

In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence.

In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

### Exemplary Template Nucleic Acids

Exemplary template nucleic acids (also referred to herein as donor constructs) comprise one or more nucleotides of a RHO gene. In certain embodiments, the template nucleic acid comprises a RHO cDNA molecule. In certain embodiments, the template nucleic acid sequence may be codon modified to be resistant to hybridization with a gRNA molecule.

Table 7 below provides exemplary template nucleic acids. In an embodiment, the template nucleic acid includes the 5′ homology arm and the 3′ homology arm of a row from Table 7. In other embodiments, a 5′ homology arm from the first column can be combined with a 3′ homology arm from Table 7. In each embodiment, a combination of the 5′ and 3′ homology arms include a replacement sequence, e.g., a cytosine (C) residue.

TABLE 7

| 5′ homology arm (the number | Re- | 3′ homology arm (the number |
|---|---|---|
| of nucleotides from SEQ ID | placement | of nucleotides from SEQ ID |
| NO: 5′H, beginning at the 3′ | Sequence = | NO: 3′H, beginning at the 5′ |
| end of SEQ ID NO: 5′H) | C | end of SEQ ID NO: 3'H) |
| 10 or more | | 10 or more |
| 20 or more | | 20 or more |
| 50 or more | | 50 or more |
| 100 or more | | 100 or more |
| 150 or more | | 150 or more |
| 200 or more | | 200 or more |
| 250 or more | | 250 or more |
| 300 or more | | 300 or more |
| 350 or more | | 350 or more |
| 400 or more | | 400 or more |
| 450 or more | | 450 or more |
| 500 or more | | 500 or more |
| 550 or more | | 550 or more |
| 600 or more | | 600 or more |
| 650 or more | | 650 or more |
| 700 or more | | 700 or more |
| 750 or more | | 750 or more |
| 800 or more | | 800 or more |
| 850 or more | | 850 or more |
| 900 or more | | 900 or more |
| 1000 or more | | 1000 or more |
| 1100 or more | | 1100 or more |
| 1200 or more | | 1200 or more |
| 1300 or more | | 1300 or more |
| 1400 or more | | 1400 or more |
| 1500 or more | | 1500 or more |
| 1600 or more | | 1600 or more |
| 1700 or more | | 1700 or more |
| 1800 or more | | 1800 or more |
| 1900 or more | | 1900 or more |
| 1200 or more | | 1200 or more |
| At least 50 but not long | | At least 50 but not long |
| enough to include a | | enough to include a |
| repeated element. | | repeated element. |
| At least 100 but not long | | At least 100 but not long |
| enough to include a | | enough to include a |
| repeated element. | | repeated element. |
| At least 150 but not long | | At least 150 but not long |
| enough to include a | | enough to include a |
| repeated element. | | repeated element. |
| 5 to 100 nucleotides | | 5 to 100 nucleotides |
| 10 to 150 nucleotides | | 10 to 150 nucleotides |
| 20 to 150 nucleotides | | 20 to 150 nucleotides |

Examples of gRNAs in Genome Editing Methods

gRNA molecules as described herein can be used with RNA-guided nuclease molecules (e.g., Cas9 or Cpfl molecules) that generate a double strand break or a single strand break to alter the sequence of a target nucleic acid, e.g., a target position or target genetic signature. The skilled artisan will be able to ascertain additional suitable gRNA molecules that can be used in conjunction with the methods and treatment modalities disclosed herein based on the present disclosure. Suitable gRNA molecules include, without limitations, those described in U.S. Patent Application No. US 2017/0073674 A1 and International Publication No. WO 2017/165862 A1, the entire contents of each of which are incorporated by reference herein.

### VI. Target Cells

RNA-guided nuclease molecules (e.g., Cas9 or Cpfl molecules) and gRNA molecules, e.g., a Cas9 or Cpfl molecule/gRNA molecule complex can be used to manipulate a cell, e.g., to edit a target nucleic acid, in a wide variety of cells

In some embodiments, a cell is manipulated by editing (e.g., altering) one or more target genes, e.g., as described herein. In some embodiments, the expression of one or more target genes (e.g., one or more target genes described herein) is modulated, e.g., in vivo. In other embodiments, the expression of one or more target genes (e.g., one or more target genes described herein) is modulated, e.g., ex vivo.

The RNA-guided nuclease molecules (e.g., Cas9 or Cpfl molecules), gRNA molecules, and RHO cDNA molecules described herein can be delivered to a target cell. In an embodiment, the target cell is a cell from the eye, e.g., a retinal cell, e.g., a photoreceptor cell. In an embodiment, the target cell is a cone photoreceptor cell or cone cell. In an embodiment, the target cell is a rod photoreceptor cell or rod cell. In an embodiment, the target cell is a macular cone photoreceptor cell. In an exemplary embodiment, cone photoreceptors in the macula are targeted, i.e., cone photoreceptors in the macula are the target cells.

A suitable cell can also include a stem cell such as, by way of example, an embryonic stem cell, an induced pluripotent stem cell, a hematopoietic stem cell, a neuronal stem cell and a mesenchymal stem cell. In an embodiment, the cell is an induced pluripotent stem cells (iPS) cell or a cell derived from an iPS cell, e.g., an iPS cell generated from the subject, modified to alter (e.g., knock out) the mutant RHO gene and deliver exogenous RHO cDNA to the cell and differentiated into a retinal progenitor cell or a retinal cell, e.g., retinal photoreceptor, and injected into the eye of the subject, e.g., subretinally, e.g., in the submacular region of the retina.

### VII. Delivery, Formulations and Routes of Administration

The components, e.g., an RNA-guided nuclease molecule (e.g., Cas9 or Cpfl molecule), gRNA molecule, and RHO cDNA molecule can be delivered or formulated in a variety of forms, see, e.g., Tables 8-9. In an embodiment, one RNA-guided nuclease molecule (e.g., Cas9 or Cpfl molecule), one or more (e.g., 1, 2, 3, 4, or more) gRNA molecules, and the sequence of the RHO cDNA molecule are delivered, e.g., by an AAV vector. In an embodiment, the sequence encoding the RNA-guided nuclease molecule (e.g., Cas9 or Cpfl molecule), the sequence(s) encoding the one or more (e.g., 1, 2, 3, 4, or more) gRNA molecules, and the sequence of the RHO cDNA molecule are present on the same nucleic acid molecule, e.g., an AAV vector. In an embodiment, the sequence encoding the RNA-guided nuclease molecule (e.g., Cas9 or Cpfl molecule) is present on a first nucleic acid molecule, e.g., an AAV vector, and the sequence(s) encoding the one or more (e.g., 1, 2, 3, 4, or more) gRNA molecules and the sequence of the RHO cDNA molecule are present on a second nucleic acid molecule, e.g., an AAV vector. In an embodiment, the sequence encoding the RNA-guided nuclease molecule (e.g., Cas9 or Cpfl molecule) is present on a first nucleic acid molecule, e.g., an AAV vector, and the sequence(s) encoding the one or more (e.g., 1, 2, 3, 4, or more) gRNA molecules are present on a second nucleic acid molecule, e.g., an AAV vector, and the sequence of the RHO cDNA molecule is present on a third nucleic acid molecule, e.g., an AAV vector.

When an RNA-guided nuclease molecule (e.g., Cas9 or Cpfl molecule), gRNA, or RHO cDNA component is delivered encoded in DNA the DNA will typically include a control region, e.g., comprising a promoter, to effect expression. Useful promoters for RNA-guided nuclease molecule (e.g., Cas9 or Cpfl molecule) sequences include CMV, EFS, EF-1a, MSCV, PGK, CAG, hGRK1, hCRX, hNRL, and hRCVRN control promoters. Useful promoters for gRNAs include H1, EF-1a and U6 promoters. Useful promoters for RHO cDNA sequences include CMV, EFS, EF-1a, MSCV, PGK, CAG, hGRK1, hCRX, hNRL, and hRCVRN control promoters. In certain embodiments, useful promoters for RHO cDNA and RNA-guided nuclease molecule sequences include a RHO promoter sequence. In certain embodiments, the RHO promoter sequence may be a minimal RHO promoter sequence. In certain embodiments, a minimal RHO promoter sequence may comprise the sequence set forth in SEQ ID NO:44. In some embodiments, a minimal RHO promoter comprises no more than 100 bp, no more than 200 bp, no more than 250 bp, no more than 300 bp, no more than 400 bp, no more than 500 bp, no more than 600 bp, no more than 700 bp, no more than 800 bp, no more than 900 bp, or no more than 1000 bp of the endogenous RHO promoter region, e.g., the region of up to 3000 bp upstream from the RHO transcription start site. In some embodiments, the minimal RHO promoter comprises no more than 100 bp, no more than 200 bp, no more than 250 bp, no more than 300 bp, no more than 400 bp, no more than 500 bp, or no more than 600 bp of the sequence proximal to the transcription start site of the endogenous RHO gene, and the distal enhancer region of the RHO promoter, or a fragment thereof. In certain embodiments, the minimal RHO cDNA promoter may be a rod-specific promoter. In certain embodiments, the RHO cDNA promoter may be a human opsin promoter. RHO promoters, and engineered promoter variants, suitable for use in the context of the methods, compositions, and treatment modalities provided herein include, for example, those described in Pellissier 2014; and those described in International Patent Applications PCT/NL2014/050549, PCT/US2016/050809, and PCT/US2016/019725, the entire contents of each of which are incorporated by reference herein.

In an embodiment, the promoter is a constitutive promoter. In another embodiment, the promoter is a tissue specific promoter. Promoters with similar or dissimilar strengths can be selected to tune the expression of components. Sequences encoding an RNA-guided nuclease molecule can comprise a nuclear localization signal (NLS), e.g., an SV40 NLS. In an embodiment, the sequence encoding an RNA-guided nuclease molecule comprises at least two nuclear localization signals. In an embodiment, a promoter for an RNA-guided nuclease molecule, a gRNA molecule, or a RHO cDNA molecule can be, independently, inducible, tissue specific, or cell specific. To detect the expression of an RNA-guided nuclease, an affinity tag can be used. Useful affinity tag sequences include, but are not limited to, 3×Flag tag, single Flag tag, HA tag, Myc tag or HIS tag. Exemplary affinity tag sequences are disclosed in Table 12. To regulate RNA-guided nuclease expression, e.g., in mammalian cells, polyadenylation signals (poly(A) signals) can be used. Exemplary polyadenylation signals are disclosed in Table 13.

Table 8 provides examples of the form in which the components can be delivered to a target cell.

TABLE 8
Elements

| RNA-guided | | | |
|---|---|---|---|
| nuclease | gRNA | | |
| molecule(s) | molecule(s) | RHO cDNA | Comments |
| DNA | DNA | DNA | In this embodiment, an RNA-guided |
| | | | nuclease and a gRNA are transcribed from |
| | | | DNA. In this embodiment, they are encoded |
| | | | on separate molecules. In this embodiment, |
| | | | the RHO cDNA is provided as a separate |
| | | | DNA molecule. |
| DNA | DNA | | In this embodiment, an RNA-guided |
| | | | nuclease and a gRNA are transcribed from |
| | | | DNA. In this embodiment, they are encoded |
| | | | on separate molecules. In this embodiment, |
| | | | the RHO cDNA is provided on the same |
| | | | DNA molecule that encodes the gRNA. |
| DNA | | DNA | In this embodiment, an RNA-guided |
| | | | nuclease and a gRNA are transcribed from |
| | | | DNA, here from a single molecule. In this |
| | | | embodiment, the RHO cDNA is provided as |
| | | | a separate DNA molecule. |
| DNA | DNA | DNA | In this embodiment, an RNA-guided |
| | | | nuclease and a gRNA are transcribed from |
| | | | DNA. In this embodiment, they are encoded |
| | | | on separate molecules. In this embodiment, |
| | | | the RHO cDNA is provided on the same |
| | | | DNA molecule that encodes the RNA- |
| | | | guided nuclease. |
| DNA | RNA | DNA | In this embodiment, an RNA-guided |
| | | | nuclease, is transcribed from DNA, and a |
| | | | gRNA is provided as in vitro transcribed or |
| | | | synthesized RNA. In this embodiment, the |
| | | | RHO cDNA is provided as a separate DNA |
| | | | molecule. |
| DNA | RNA | DNA | In this embodiment, an RNA-guided |
| | | | nuclease is transcribed from DNA, and a |
| | | | gRNA is provided as in vitro transcribed or |
| | | | synthesized RNA. In this embodiment, the |
| | | | RHO cDNA is provided on the same DNA |
| | | | molecule that encodes the RNA-guided |
| | | | nuclease. |
| mRNA | RNA | DNA | In this embodiment, an RNA-guided |
| | | | nuclease is translated from in vitro |
| | | | transcribed mRNA, and a gRNA is provided |
| | | | as in vitro transcribed or synthesized RNA. |
| | | | In this embodiment, the RHO cDNA is |
| | | | provided as a DNA molecule. |
| mRNA | DNA | DNA | In this embodiment, an RNA-guided |
| | | | nuclease is translated from in vitro |
| | | | transcribed mRNA, and a gRNA is |
| | | | transcribed from DNA. In this embodiment, |
| | | | the RHO cDNA is provided as a separate |
| | | | DNA molecule. |
| mRNA | DNA | | In this embodiment, an RNA-guided |
| | | | nuclease is translated from in vitro |
| | | | transcribed mRNA, and a gRNA is |
| | | | transcribed from DNA. In this embodiment, |
| | | | the RHO cDNA is provided on the same |
| | | | DNA molecule that encodes the gRNA. |
| Protein | DNA | DNA | In this embodiment, an RNA-guided |
| | | | nuclease is provided as a protein, and a |
| | | | gRNA is transcribed from DNA. In this |
| | | | embodiment, the RHO cDNA is provided as |
| | | | a separate DNA molecule. |
| Protein | DNA | | In this embodiment, an RNA-guided |
| | | | nuclease is provided as a protein, and a |
| | | | gRNA is transcribed from DNA. In this |
| | | | embodiment, the RHO cDNA is provided on |
| | | | the same DNA molecule that encodes the |
| | | | gRNA. |
| Protein | RNA | DNA | In this embodiment, an RNA-guided |
| | | | nuclease is provided as a protein, and a |
| | | | gRNA is provided as transcribed or |
| | | | synthesized RNA. In this embodiment, the |
| | | | RHO cDNA is provided as a DNA molecule. |

Table 9 summarizes various delivery methods for the components of an RNA-guided nuclease system, e.g., the Cas9 or Cpfl molecule component, the gRNA molecule component, and the RHO cDNA molecule component as described herein.

TABLE 9

| | | Delivery | | | |
|---|---|---|---|---|---|
| | | into Non- | Duration | | Type of |
| | | Dividing | of | Genome | Molecule |
| Delivery Vector/Mode | | Cells | Expression | Integration | Delivered |
| Physical (e.g., | | YES | Transient | NO | Nucleic |
| electroporation, particle | | | | | Acids and |
| gun, Calcium Phosphate | | | | | Proteins |
| transfection) | | | | | |
| Viral | Retrovirus | NO | Stable | YES | RNA |
| | Lentivirus | YES | Stable | YES/NO | RNA |
| | | | | with | |
| | | | | mod- | |
| | | | | ifications | |
| | Adenovirus | YES | Transient | NO | DNA |
| | Adeno- | YES | Stable | NO | DNA |
| | Associated | | | | |
| | Virus (AAV) | | | | |
| | Vaccinia | YES | Very | NO | DNA |
| | Virus | | Transient | | |
| | Herpes | YES | Stable | NO | DNA |
| | Simplex Virus | | | | |
| Non-Viral | Cationic | YES | Transient | Depends | Nucleic |
| | Liposomes | | | on what is | Acids and |
| | | | | delivered | Proteins |
| | Polymeric | YES | Transient | Depends | Nucleic |
| | Nanoparticles | | | on what is | Acids and |
| | | | | delivered | Proteins |
| Biological | Attenuated | YES | Transient | NO | Nucleic |
| Non-Viral | Bacteria | | | | Acids |
| Delively | Engineered | YES | Transient | NO | Nucleic |
| Vehicles | Bacteriophages | | | | Acids |
| | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

Table 10 describes exemplary promoter sequences that can be used in AAV vectors for RNA-guided nuclease (e.g., Cas9 or Cpfl) expression.

TABLE 10
RNA-Guided Nuclease Promoter Sequences

| Promoter | Length (bp) | DNA Sequence |
|---|---|---|
| CMV | 617 | CATTGATTATTGACTAGTTATTAATAGTAATCAAT |
| | | TACGGGGTCATTAGTTCATAGCCCATATATGGAGT |
| | | TCCGCGTTACATAACTTACGGTAAATGGCCCGCCT |
| | | GGCTGACCGCCCAACGACCCCCGCCCATTGACGTC |
| | | AATAATGACGTATGTTCCCATAGTAACGCCAATAG |
| | | GGACTTTCCATTGACGTCAATGGGTGGACTATTTA |
| | | CGGTAAACTGCCCACTTGGCAGTACATCAAGTGTA |
| | | TCATATGCCAAGTACGCCCCCTATTGACGTCAATG |
| | | ACGGTAAATGGCCCGCCTGGCATTATGCCCAGTAC |
| | | ATGACCTTATGGGACTTTCCTACTTGGCAGTACAT |
| | | CTACGTATTAGTCATCGCTATTACCATGGTGATGC |
| | | GGTTTTGGCAGTACATCAATGGGCGTGGATAGCGG |
| | | TTTGACTCACGGGGATTTCCAAGTCTCCACCCCAT |
| | | TGACGTCAATGGGAGTTTGTTTTGGCACCAAAATC |
| | | AACGGGACTTTCCAAAATGTCGTAACAACTCCGCC |
| | | CCATTGACGCAAATGGGCGGTAGGCGTGTACGGTG |
| | | GGAGGTCTATATAAGCAGAGCTGGTTTAGTGAACC |
| | | GTCAGATCCGCTAGAGATCCGC |
| | | (SEQ ID NO: 45) |
| EFS | 252 | TCGAGTGGCTCCGGTGCCCGTCAGTGGGCAGAGCG |
| | | CACATCGCCCACAGTCCCCGAGAAGTTGGGGGGAG |
| | | GGGTCGGCAATTGAACCGGTGCCTAGAGAAGGTGG |
| | | CGCGGGGTAAACTGGGAAAGTGATGTCGTGTACTG |
| | | GCTCCGCCTTTTTCCCGAGGGTGGGGGAGAACCGT |
| | | ATATAAGTGCAGTAGTCGCCGTGAACGTTCTTTTT |
| | | CGCAACGGGTTTGCCGCCAGAACACAGGTGTCGTG |
| | | ACCGCGG |
| | | (SEQ ID NO: 46) |
| Human GRK1 | 292 | GGGCCCCAGAAGCCTGGTGGTTGTTTGTCCTTCTC |
| (rhodopsin kinase) | | AGGGGAAAAGTGAGGCGGCCCCTTGGAGGAAGGGG |
| | | CCGGGCAGAATGATCTAATCGGATTCCAAGCAGCT |
| | | CAGGGGATTGTCTTTTTCTAGCACCTTCTTGCCAC |
| | | TCCTAAGCGTCCTCCGTGACCCCGGCTGGGATTTC |
| | | GCCTGGTGCTGTGTCAGCCCCGGTCTCCCAGGGGC |
| | | TTCCCAGTGGTCCCCAGGAACCCTCGACAGGGCCC |
| | | GGTCTCTCTCGTCCAGCAAGGGCAGGGACGGGCCA |
| | | CAGGCCAAGGGC |
| | | (SEQ ID NO: 47) |
| Human CRX (cone | 113 | GCCTGTAGCC TTAATCTCTC CTAGCAGGGG |
| rod homeobox | | GTTTGGGGGA GGGAGGAGGA GAAAGAAAGG |
| transcription factor) | | GCCCCTTATG GCTGAGACAC AATGACCCAG |
| | | CCACAAGGAG GGATTACCGG GCG |
| | | (SEQ ID NO: 48) |
| Human NRL (neural | 281 | AGGTAGGAAG TGGCCTTTAA CTCCATAGAC |
| retina leucine zipper | | CCTATTTAAA CAGCTTCGGA CAGGTTTAAA |
| transcription factor | | CATCTCCTTG GATAATTCCT AGTATCCCTG |
| enhance upstream of | | TTCCCACTCC TACTCAGGGA TGATAGCTCT |
| the human TK | | AAGAGGTGTT AGGGGATTAG GCTGAAAATG |
| terminal promoter) | | TAGGTCACCC CTCAGCCATC TGGGAACTAG |
| | | AATGAGTGAG AGAGGAGAGA GGGGCAGAGA |
| | | CACACACATT CGCATATTAA GGTGACGCGT |
| | | GTGGCCTCGA ACACCGAGCG ACCCTGCAGC |
| | | GACCCGCTTA A |
| | | (SEQ ID NO: 49) |
| Human RCVRN | 235 | ATTTTAATCT CACTAGGGTT CTGGGAGCAC |
| (recoverin) | | CCCCCCCCAC CGCTCCCGCC CTCCACAAAG |
| | | CTCCTGGGCC CCTCCTCCCT TCAAGGATTG |
| | | CGAAGAGCTG GTCGCAAATC CTCCTAAGCC |
| | | ACCAGCATCT CGGTCTTCAG CTCACACCAG |
| | | CCTTGAGCCC AGCCTGCGGC CAGGGGACCA |
| | | CGCACGTCCC ACCCACCCAG CGACTCCCCA |
| | | GCCGCTGCCC ACTCTTCCTC ACTCA |
| | | (SEQ ID NO: 50) |
| Human rhodopsin | 516 | CCACGTCAGA ATCAAACCCT CACCTTAACC |
| promoter | | TCATTAGCGT TGGGCATAAT CACCAGGCCA |
| | | AGCGCCTTAA ACTACGAGAG GCCCCATCCC |
| | | ACCCGCCCTG CCTTAGCCCT GCCACGTGTG |
| | | CCAAACGCTG TTAGACCCAA CACCACCCAG |
| | | GCCAGGTAGG GGGCTGGAGC CCAGGTGGGC |
| | | ATTTGAGTCA CCAACCCCCA GGCAGTCTCC |
| | | CTTTTCCTGG ATCCTGAGTA CCTCTCCTCC |
| | | CTGACCTCAG GCTTCCTCCT AGTGTCACCT |
| | | TGGCCCCTCT TAGAAGCCAA TTAGGCCCTC |
| | | AGTTTCTGCA GCGGGGATTA ATATGATTAT |
| | | GAACACCCCC AATCTCCCAG ATGCTGATTC |
| | | AGCCAGGAGC TTAGGAGGGG GAGGTCACTT |
| | | TATAAGGGTC TGGGGGGGTC AGAACCCAGA |
| | | GTCATCCAGC TGGAGCCCTG AGTGGCTGAG |
| | | CTCAGGCCTT CGCAGCATTC TTGGGTGGGA |
| | | GCAGCCACGG GTCAGCCACA AGGGCCACCA |
| | | CCATGG |
| | | (SEQ ID NO: 43) |
| Minimal Human | 249 | GTCACCTTGGCCCCTCTTAGAAGCCAATTAGGCCC |
| rhodopsin promoter | | TCAGTTTCTGCAGCGGGGATTAATATGATTATGAA |
| | | CACCCCCAATCTCCCAGATGCTGATTCAGCCAGGA |
| | | GCTTAGGAGGGGGAGGTCACTTTATAAGGGTCTGG |
| | | GGGGGTCAGAACCCAGAGTCATCCAGCTGGAGCCC |
| | | TGAGTGGCTGAGCTCAGGCCTTCGCAGCATTCTTG |
| | | GGTGGGAGCAGCCACGGGTCAGCCACAAGGGCCAC |
| | | AGCC |
| | | (SEQ ID NO: 44) |

Table 11 describes exemplary promoter sequences that can be used in AAV vectors for RHO cDNA.

TABLE 11
RHO cDNA Promoter Sequences

| Promoter | Length (bp) | DNA Sequence |
|---|---|---|
| CMV | 617 | CATTGATTATTGACTAGTTATTAATAGTAATCAATTA |
| | | CGGGGTCATTAGTTCATAGCCCATATATGGAGTTCCG |
| | | CGTTACATAACTTACGGTAAATGGCCCGCCTGGCTGA |
| | | CCGCCCAACGACCCCCGCCCATTGACGTCAATAATGA |
| | | CGTATGTTCCCATAGTAACGCCAATAGGGACTTTCCA |
| | | TTGACGTCAATGGGTGGACTATTTACGGTAAACTGCC |
| | | CACTTGGCAGTACATCAAGTGTATCATATGCCAAGTA |
| | | CGCCCCCTATTGACGTCAATGACGGTAAATGGCCCGC |
| | | CTGGCATTATGCCCAGTACATGACCTTATGGGACTTT |
| | | CCTACTTGGCAGTACATCTACGTATTAGTCATCGCTA |
| | | TTACCATGGTGATGCGGTTTTGGCAGTACATCAATGG |
| | | GCGTGGATAGCGGTTTGACTCACGGGGATTTCCAAGT |
| | | CTCCACCCCATTGACGTCAATGGGAGTTTGTTTTGGC |
| | | ACCAAAATCAACGGGACTTTCCAAAATGTCGTAACAA |
| | | CTCCGCCCCATTGACGCAAATGGGCGGTAGGCGTGTA |
| | | CGGTGGGAGGTCTATATAAGCAGAGCTGGTTTAGTGA |
| | | ACCGTCAGATCCGCTAGAGATCCGC |
| | | (SEQ ID NO: 45) |
| EFS | 252 | TCGAGTGGCTCCGGTGCCCGTCAGTGGGCAGAGCGCA |
| | | CATCGCCCACAGTCCCCGAGAAGTTGGGGGGAGGGGT |
| | | CGGCAATTGAACCGGTGCCTAGAGAAGGTGGCGCGGG |
| | | GTAAACTGGGAAAGTGATGTCGTGTACTGGCTCCGCC |
| | | TTTTTCCCGAGGGTGGGGGAGAACCGTATATAAGTGC |
| | | AGTAGTCGCCGTGAACGTTCTTTTTCGCAACGGGTTT |
| | | GCCGCCAGAACACAGGTGTCGTGACCGCGG |
| | | (SEQ ID NO: 46) |
| Human GRK1 | 292 | GGGCCCCAGAAGCCTGGTGGTTGTTTGTCCTTCTCAG |
| (rhodopsin | | GGGAAAAGTGAGGCGGCCCCTTGGAGGAAGGGGCCGG |
| kinase) | | GCAGAATGATCTAATCGGATTCCAAGCAGCTCAGGGG |
| | | ATTGTCTTTTTCTAGCACCTTCTTGCCACTCCTAAGC |
| | | GTCCTCCGTGACCCCGGCTGGGATTTCGCCTGGTGCT |
| | | GTGTCAGCCCCGGTCTCCCAGGGGCTTCCCAGTGGTC |
| | | CCCAGGAACCCTCGACAGGGCCCGGTCTCTCTCGTCC |
| | | AGCAAGGGCAGGGACGGGCCACAGGCCAAGGGC |
| | | (SEQ ID NO: 47) |
| Human CRX | 113 | GCCTGTAGCC TTAATCTCTC CTAGCAGGGG |
| (cone rod | | GTTTGGGGGA GGGAGGAGGA GAAAGAAAGG |
| homeobox | | GCCCCTTATG GCTGAGACAC AATGACCCAG |
| transcription | | CCACAAGGAG GGATTACCGG GCG |
| factor) | | (SEQ ID NO: 48) |
| Human NRL | 281 | AGGTAGGAAG TGGCCTTTAA CTCCATAGAC |
| (neural retina | | CCTATTTAAA CAGCTTCGGA CAGGTTTAAA |
| leucine zipper | | CATCTCCTTG GATAATTCCT AGTATCCCTG |
| transcription | | TTCCCACTCC TACTCAGGGA TGATAGCTCT |
| factor enhance | | AAGAGGTGTT AGGGGATTAG GCTGAAAATG |
| upstream of the | | TAGGTCACCC CTCAGCCATC TGGGAACTAG |
| human TK | | AATGAGTGAG AGAGGAGAGA GGGGCAGAGA |
| terminal | | CACACACATT CGCATATTAA GGTGACGCGT |
| promoter) | | GTGGCCTCGA ACACCGAGCG ACCCTGCAGC |
| | | GACCCGCTTA A |
| | | (SEQ ID NO: 49) |
| Human RCVRN | 235 | ATTTTAATCT CACTAGGGTT CTGGGAGCAC |
| (recoverin) | | CCCCCCCCAC CGCTCCCGCC CTCCACAAAG |
| | | CTCCTGGGCC CCTCCTCCCT TCAAGGATTG |
| | | CGAAGAGCTG GTCGCAAATC CTCCTAAGCC |
| | | ACCAGCATCT CGGTCTTCAG CTCACACCAG |
| | | CCTTGAGCCC AGCCTGCGGC CAGGGGACCA |
| | | CGCACGTCCC ACCCACCCAG CGACTCCCCA |
| | | GCCGCTGCCC ACTCTTCCTC ACTCA |
| | | (SEQ ID NO: 50) |
| Human | 516 | CCACGTCAGA ATCAAACCCT CACCTTAACC |
| rhodopsin | | TCATTAGCGT TGGGCATAAT CACCAGGCCA |
| promoter | | AGCGCCTTAA ACTACGAGAG GCCCCATCCC |
| | | ACCCGCCCTG CCTTAGCCCT GCCACGTGTG |
| | | CCAAACGCTG TTAGACCCAA CACCACCCAG |
| | | GCCAGGTAGG GGGCTGGAGC CCAGGTGGGC |
| | | ATTTGAGTCA CCAACCCCCA GGCAGTCTCC |
| | | CTTTTCCTGG ATCCTGAGTA CCTCTCCTCC |
| | | CTGACCTCAG GCTTCCTCCT AGTGTCACCT |
| | | TGGCCCCTCT TAGAAGCCAA TTAGGCCCTC |
| | | AGTTTCTGCA GCGGGGATTA ATATGATTAT |
| | | GAACACCCCC AATCTCCCAG ATGCTGATTC |
| | | AGCCAGGAGC TTAGGAGGGG GAGGTCACTT |
| | | TATAAGGGTC TGGGGGGGTC AGAACCCAGA |
| | | GTCATCCAGC TGGAGCCCTG AGTGGCTGAG |
| | | CTCAGGCCTT CGCAGCATTC TTGGGTGGGA |
| | | GCAGCCACGG GTCAGCCACA AGGGCCACCA |
| | | CCATGG |
| | | (SEQ ID NO: 43) |
| Minimal Human | 249 | GTCACCTTGGCCCCTCTTAGAAGCCAATTAGGCCCTC |
| rhodopsin | | AGTTTCTGCAGCGGGGATTAATATGATTATGAACACC |
| promoter | | CCCAATCTCCCAGATGCTGATTCAGCCAGGAGCTTAG |
| | | GAGGGGGAGGTCACTTTATAAGGGTCTGGGGGGGTCA |
| | | GAACCCAGAGTCATCCAGCTGGAGCCCTGAGTGGCTG |
| | | AGCTCAGGCCTTCGCAGCATTCTTGGGTGGGAGCAGC |
| | | CACGGGTCAGCCACAAGGGCCACAGCC |
| | | (SEQ ID NO: 44) |

Table 12 describes exemplary affinity tag sequences that can be used in AAV vectors, e.g., for RNA-guided nuclease (e.g., Cas9 or Cpfl) expression.

TABLE 12
Exemplary Affinity Tag Sequences

| Affinity tag | Amino Acid Sequence |
|---|---|
| 3XFlag tag | DYKDHDGDYKDHDIDYKDDDDK |
| | (SEQ ID NO: 51) |
| Flag tag (single) | DYKDDDDK |
| | (SEQ ID NO: 52) |
| HA tag | YPYDVPDYA |
| | (SEQ ID NO: 53) |
| Myc tag | EQKLISEEDL |
| | (SEQ ID NO: 54) |
| HIS tag | HHHHHH |
| | (SEQ ID NO: 55) |

Table 13 describes exemplary polyadenylation (polyA) sequences that can be used in AAV vectors, e.g., for RNA-guided nuclease (e.g., Cas9 or Cpfl) expression.

TABLE 13
Exemplary PolyA Sequences

| PolyA | DNA sequence |
|---|---|
| Mini poly A | TAGCAATAAA GGATCGTTTA TTTTCATTGG |
| | AAGCGTGTGT TGGTTTTTTG ATCAGGCGCG |
| | (SEQ ID NO: 56) |
| bGH polyA | GCTGCAGGAT GACCGGTCAT CATCACCATC |
| | ACCATTGAGT TTAAACCCGC TGATCAGCCT |
| | CGACTGTGCC TTCTAGTTGC CAGCCATCTG |
| | TTGTTTGCCC CTCCCCCGTG CCTTCCTTGA |
| | CCCTGGAAGG TGCCACTCCC ACTGTCCTTT |
| | CCTAATAAAA TGAGGAAATT GCATCGCATT |
| | GTCTGAGTAG GTGTCATTCT ATTCTGGGGG |
| | GTGGGGTGGG GCAGGACA |
| | (SEQ ID NO: 57) |
| SV40 polyA | ATGCTTTATT TGTGAAATTT GTGATGCTAT |
| | TGCTTTATTT GTAACCATTA TAAGCTGCAA |
| | TAAACAAGTT AACAACAACA ATTGCATTCA |
| | TTTTATGTTT CAGGTTCAGG GGGAGGTGTG |
| | GGAGGTTTTT TAAA |
| | (SEQ ID NO: 58) |

Table 14 describes exemplary Inverted Terminal Repeat (ITR) sequences that can be used in AAV vectors.

TABLE 14
Sequences of ITRs from Exemplary AAV Serotypes

| AAV | | |
|---|---|---|
| Sero- | | |
| type | Left ITR Sequence | Right ITR Sequence |
| AAV1 | TTGCCCACTC CCTCTCTGCG | TTACCCCTAG TGATGGAGTT |
| | CGCTCGCTCG CTCGGTGGGG | GCCCACTCCC TCTCTGCGCG |
| | CCTGCGGACC AAAGGTCCGC | CTCGCTCGCT CGGTGGGGCC |
| | AGACGGCAGA GCTCTGCTCT | GGCAGAGCAG AGCTCTGCCG |
| | GCCGGCCCCA CCGAGCGAGC | TCTGCGGACC TTTGGTCCGC |
| | GAGCGCGCAG AGAGGGAGTG | AGGCCCCACC GAGCGAGCGA |
| | GGCAACTCCA TCACTAGGGG | GCGCGCAGAG AGGGAGTGGG |
| | TAA (SEQ ID NO: 59) | CAA |
| | | (SEQ ID NO: 68) |
| AAV2 | TTGGCCACTC CCTCTCTGCG | AGGAACCCCT AGTGATGGAG |
| | CGCTCGCTCG CTCACTGAGG | TTGGCCACTC CCTCTCTGCG |
| | CCGGGCGACC AAAGGTCGCC | CGCTCGCTCG CTCACTGAGG |
| | CGACGCCCGG GCTTTGCCCG | CCGCCCGGGC AAAGCCCGGG |
| | GGCGGCCTCA GTGAGCGAGC | CGTCGGGCGA CCTTTGGTCG |
| | GAGCGCGCAG AGAGGGAGTG | CCCGGCCTCA GTGAGCGAGC |
| | GCCAACTCCA TCACTAGGGG | GAGCGCGCAG AGAGGGAGTG |
| | TTCCT | GCCAA |
| | (SEQ ID NO: 60) | (SEQ ID NO: 69) |
| AAV3B | TGGCCACTCC CTCTATGCGC | ATACCTCTAG TGATGGAGTT |
| | ACTCGCTCGC TCGGTGGGGC | GGCCACTCCC TCTATGCGCA |
| | CTGGCGACCA AAGGTCGCCA | CTCGCTCGCT CGGTGGGGCC |
| | GACGGACGTG CTTTGCACGT | GGACGTGCAA AGCACGTCCG |
| | CCGGCCCCAC CGAGCGAGCG | TCTGGCGACC TTTGGTCGCC |
| | AGTGCGCATA GAGGGAGTGG | AGGCCCCACC GAGCGAGCGA |
| | CCAACTCCAT CACTAGAGGT | GTGCGCATAG AGGGAGTGGC |
| | AT | CA |
| | (SEQ ID NO: 61) | (SEQ ID NO: 70) |
| AAV4 | TTGGCCACTC CCTCTATGCG | GGGCAAACCT AGATGATGGA |
| | CGCTCGCTCA CTCACTCGGC | GTTGGCCACT CCCTCTATGC |
| | CCTGGAGACC AAAGGTCTCC | GCGCTCGCTC ACTCACTCGG |
| | AGACTGCCGG CCTCTGGCCG | CCCTGCCGGC CAGAGGCCGG |
| | GCAGGGCCGA GTGAGTGAGC | CAGTCTGGAG ACCTTTGGTC |
| | GAGCGCGCAT AGAGGGAGTG | TCCAGGGCCG AGTGAGTGAG |
| | GCCAACTCCA TCATCTAGGT | CGAGCGCGCA TAGAGGGAGT |
| | TTGCCC | GGCCAA |
| | (SEQ ID NO: 62) | (SEQ ID NO: 71) |
| AAV5 | CTCTCCCCCC TGTCGCGTTC | TTGCTTGAGA GTGTGGCACT |
| | GCTCGCTCGC TGGCTCGTTT | CTCCCCCCTG TCGCGTTCGC |
| | GGGGGGGTGG CAGCTCAAAG | TCGCTCGCTG GCTCGTTTGG |
| | AGCTGCCAGA CGACGGCCCT | GGGGGCGACG GCCAGAGGGC |
| | CTGGCCGTCG CCCCCCCAAA | CGTCGTCTGG CAGCTCTTTG |
| | CGAGCCAGCG AGCGAGCGAA | AGCTGCCACC CCCCCAAACG |
| | CGCGACAGGG GGGAGAGTGC | AGCCAGCGAG CGAGCGAACG |
| | CACACTCTCA | CGACAGGGGG GAGAG |
| | AGCAA | (SEQ ID NO: 72) |
| | (SEQ ID NO: 63) | |
| AAV6 | ATACCCCTAG TGATGGAGTT | TTGCCCACTC CCTCTATGCG |
| | GCCCACTCCC TCTATGCGCG | CGCTCGCTCG CTCGGTGGGG |
| | CTCGCTCGCT CGGTGGGGCC | CCTGCGGACC AAAGGTCCGC |
| | GGCAGAGCAG AGCTCTGCCG | AGACGGCAGA GCTCTGCTCT |
| | TCTGCGGACC TTTGGTCCGC | GCCGGCCCCA CCGAGCGAGC |
| | AGGCCCCACC GAGCGAGCGA | GAGCGCGCAT AGAGGGAGTG |
| | GCGCGCATAG AGGGAGTGGG | GGCAACTCCA TCACTAGGGG |
| | CAA | TAT |
| | (SEQ ID NO: 64) | (SEQ ID NO: 73) |
| AAV7 | TTGGCCACTC CCTCTATGCG | CGGTACCCCT AGTGATGGAG |
| | CGCTCGCTCG CTCGGTGGGG | TTGGCCACTC CCTCTATGCG |
| | CCTGCGGACC AAAGGTCCGC | CGCTCGCTCG CTCGGTGGGG |
| | AGACGGCAGA GCTCTGCTCT | CCGGCAGAGC AGAGCTCTGC |
| | GCCGGCCCCA CCGAGCGAGC | CGTCTGCGGA CCTTTGGTCC |
| | GAGCGCGCAT AGAGGGAGTG | GCAGGCCCCA CCGAGCGAGC |
| | GCCAACTCCA TCACTAGGGG | GAGCGCGCAT AGAGGGAGTG |
| | TACCG | GCCAA |
| | (SEQ ID NO: 65) | (SEQ ID NO: 74) |
| AAV8 | CAGAGAGGGA GTGGCCAACT | GGTGTCGCAA AATGCCGCAA |
| | CCATCACTAG GGGTAGCGCG | AAGCACTCAC GTGACAGCTA |
| | AAGCGCCTCC CACGCTGCCG | ATACAGGACC ACTCCCCTAT |
| | CGTCAGCGCT GACGTAAATT | GACGTAATTT ACGTCAGCGC |
| | ACGTCATAGG GGAGTGGTCC | TGACGCGGCA GCGTGGGAGG |
| | TGTATTAGCT GTCACGTGAG | CGCTTCGCGC TACCCCTAGT |
| | TGCTTTTGCG GCATTTTGCG | GATGGAGTTG GCCACTCCCT |
| | ACACC | CTCTG |
| | (SEQ ID NO: 66) | (SEQ ID NO: 75) |
| AAV9 | CAGAGAGGGA GTGGCCAACT | GTGTCGCAAA ATGTCGCAAA |
| | CCATCACTAG GGGTAATCGC | AGCACTCACG TGACAGCTAA |
| | GAAGCGCCTC CCACGCTGCC | TACAGGACCA CTCCCCTATG |
| | GCGTCAGCGC TGACGTAGAT | ACGTAATCTA CGTCAGCGCT |
| | TACGTCATAG GGGAGTGGTC | GACGCGGCAG CGTGGGAGGC |
| | CTGTATTAGC TGTCACGTGA | GCTTCGCGAT TACCCCTAGT |
| | GTGCTTTTGC GACATTTTGC | GATGGAGTTG GCCACTCCCT |
| | GACAC | CTCTG |
| | (SEQ ID NO: 67) | (SEQ ID NO: 76) |
| AAV | TGCAGGCAGCTGCGCGCTCGC | AGGAACCCCTAGTGATGGAGT |
| | TCGCTCACTGAGGCCGCCCGG | TGGCCACTCCCTCTCTGCGCG |
| | GCAAAGCCCGGGCGTCGGGCG | CTCGCTCGCTCACTGAGGCCG |
| | ACCTTTGGTCGCCCGGCCTCA | GGCGACCAAAGGTCGCCCGAC |
| | GTGAGCGAGCGAGCGCGCAGA | GCCCGGGCTTTGCCCGGGCGG |
| | GAGGGAGTGGCCAACTCCATC | CCTCAGTGAGCGAGCGAGCGC |
| | ACTAGGGGTTCCT | GCAGCTGCCTGCA |
| | (SEQ ID NO: 92) | (SEQ ID NO: 93) |

Additional exemplary sequences for the recombinant AAV genome components described herein are provided below.

Exemplary U6 promoter sequence:
(SEQ ID NO: 78)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACGAAACACC.

Exemplary gRNA targeting domain sequences are described herein, e.g., in Tables 1-3, and 18.

Skilled artisans will understand that it may be advantageous in some embodiments to add a 5′ G to a gRNA targeting domain sequence, e.g., when the gRNA is driven by a U6 promoter.

Exemplary gRNA scaffold domain sequences:
(SEQ ID NO: 79)
GTTTTAGTACTCTGGAAACAGAATCTACTAAAACAAGGCAAAATGCCGTG
TTTATCTCGTCAACTTGTTGGCGAGATTTTTT;
(SEQ ID NO: 12)
GTTATAGTACTCTGGAAACAGAATCTACTATAACAAGGCAAAATGCCGTG
TTTATCTCGTCAACTTGTTGGCGAGA.
Exemplary N-ter NLS nucleotide sequence:
(SEQ ID NO: 81)
CCGAAGAAAAAGCGCAAGGTCGAAGCGTCC.
Exemplary N-ter NLS amino acid sequence:
(SEQ ID NO: 82)
PKKKRKV.
Exemplary Cas9 nucleotide sequences as described
herein.
Exemplary Cas9 amino acid sequences as described
herein.
Exemplary Cpf1 nucleotide sequences as described
herein.
Exemplary Cpf1 amino acid sequences as described
herein.
Exemplary C-ter NLS sequence:
(SEQ ID NO: 83)
CCCAAGAAGAAGAGGAAAGTC.
Exemplary C-ter NLS amino acid sequence:
(SEQ ID NO: 84)
PKKKRKV.
Exemplary poly(A) signal sequence:
(SEQ ID NO: 56)
TAGCAATAAAGGATCGTTTATTTTCATTGGAAGCGTGTGTTGGTTTTTTG
ATCAGGCGCG.
Exemplary 3xFLAG nucleotide sequence:
(SEQ ID NO: 86)
GACTACAAAGACCATGACGGTGATTATAAAGATCATGACATCGATTACAA
GGATGACGATGACAAG.
Exemplary 3xFLAG amino acid sequence:
(SEQ ID NO: 51)
DYKDHDGDYKDHDIDYKDDDDK.
Exemplary spacer sequences:
(SEQ ID NO: 77)
CAGATCTGAATTCGGTACC;
(SEQ ID NO: 80)
GGTACCGCTAGCGCTTAAGTCGCGATGTACGGGCCAGATATACGCGTTG
A;
(SEQ ID NO: 85)
TCCAAGCTTCGCAGGAAAGAACATGTGAGCAAAAGGCCAGCAAAAGGCGT
TAACTCTAGATTTAAATGCATGCTGGGGAGAGATCT;
(SEQ ID NO: 87)
CGACTTAGTTCGATCGAAGG.
Exemplary SV40 intron sequence:
(SEQ ID NO: 94)
TCTAGAGGATCCGGTACTCGAGGAACTGAAAAACCAGAAAGTTAACTGGT
AAGTTTAGTCTTTTTGTCTTTTATTTCAGGTCCCGGATCCGGTGGTGGTG
CAAATCAAAGAACTGCTCCTCAGTGGATGTTGCCTTTACTTCTAGGCCTG
TACGGAAGTGTTAC.

In certain aspects, the present disclosure focuses on AAV vectors encoding CRISPR/RNA-guided nuclease genome editing systems and a RHO cDNA molecule, and on the use of such vectors to treat adRP. Exemplary AAV vector genomes are schematized in FIG. 2, which illustrate certain fixed and variable elements of these vectors: a first AAV vector comprising ITRs, an RNA-guided nuclease (e.g., Cas9) coding sequence and a promoter to drive its expression, with the RNA-guided nuclease coding sequence flanked by NLS sequences; and a second AAV vector comprising ITRs, one RHO cDNA sequence and a minimal RHO promoter to drive its expression and one gRNA sequence and promoter sequences to drive its expression. Additional exemplary AAV vector genomes are also set forth in FIGS. 3 and 16-18. Exemplary AAV vector genome sequences are set forth in SEQ ID NOs: 8-11.

Turning first to the gRNA utilized in the nucleic acids or AAV vectors of the present disclosure, one or more gRNAs may be used to cut the 5′ region of a mutant RHO gene (e.g., 5′ UTR, exon 1, exon 2, intron 1, exon 1/intron border). In certain embodiments, cutting in the 5′ region of the mutant RHO gene results in knocking out or loss of function of the mutant RHO gene. In certain embodiments, one or more gRNAs may be used to cut the coding region of a mutant RHO gene (e.g., exon 1, exon 2, exon 3, exon 4, exon 5) or the non-coding region of a mutant RHO gene (e.g., 5′ UTR, introns, 3′ UTR). In certain embodiments, cutting in the coding region or non-coding region of the mutant RHO gene may result in knocking out or loss of function of the mutant RHO gene.

Targeting domain sequences of exemplary guides (both DNA and RNA sequences) are presented in Tables 1-3 and 18.

In some embodiments, the gRNAs used in the present disclosure may be derived from S. aureus gRNAs and can be unimolecular or modular, as described below. Exemplary DNA and RNA sequences corresponding to unimolecular S. aureus gRNAs are shown below:

DNA: [N]₁₆₋₂₄
(SEQ ID NO: 88)
GTTTTAGTACTCTGGAAACAGAATCTACTAAAACAAGGCAAAATGCC
GTGTTTATCTCGTCAACTTGTTGGCGAGATTTTTT
and
RNA: [N]₁₆₋₂₄
(SEQ ID NO: 89)
GUUUUAGUACUCUGGAAACAGAAUCUACUAAAACAAGGCAAAAUGCC
GUGUUUAUCUCGUCAACUUGUUGGCGAGAUUUUUU.
DNA: [N]₁₆₋₂₄
(SEQ ID NO: 90)
GTTATAGTACTCTGGAAACAGAATCTACTATAACAAGGCAAAATGCC
GTGTTTATCTCGTCAACTTGTTGGCGAGATTTTTT
and
RNA: [N]₁₆₋₂₄
(SEQ ID NO: 91)
GUUAUAGUACUCUGGAAACAGAAUCUACUAUAACAAGGCAAAAUGCC
GUGUUUAUCUCGUCAACUUGUUGGCGAGAUUUUUU.

It should be noted that the targeting domain can have any suitable length. gRNAs used in the various embodiments of this disclosure preferably include targeting domains of between 16 and 24 (inclusive) bases in length at their 5′ ends, and optionally include a 3′ U6 termination sequence as illustrated.

In some instances, modular guides can be used. In the exemplary unimolecular gRNA sequences above, a 5′ portion corresponding to a crRNA (underlined) is connected by a GAAA linker to a 3′ portion corresponding to a tracrRNA (double underlined). Skilled artisans will appreciate that two-part modular gRNAs can be used that correspond to the underlined and double underlined sections.

Skilled artisans will appreciate that the exemplary gRNA designs set forth herein can be modified in a variety of ways, which are described below or are known in the art; the incorporation of such modifications is within the scope of this disclosure.

Expression of the one or more gRNAs in the AAV vector may be driven by a pair of U6 promoters, such as a human U6 promoter. An exemplary U6 promoter sequence, as set forth in Maeder, is SEQ ID NO:78.

Turning next to RNA-guided nucleases, in some embodiments the RNA-guided nuclease may be a Cas9 or Cpfl protein. In certain embodiments, the Cas9 protein is S. pyogenes Cas9. In certain embodiments, the Cas9 protein is S. aureus Cas9. In further embodiments of this disclosure an Cas9 sequence is modified to include two nuclear localization sequences (NLSs) at the C- and N-termini of the Cas9 protein, and a mini-polyadenylation signal (or Poly-A sequence). Exemplary Cas9 sequences and Cpfl sequences are provided herein. These sequences are exemplary in nature and are not intended to be limiting. The skilled artisan will appreciate that modifications of these sequences may be possible or desirable in certain applications; such modifications are described below, or are known in the art, and are within the scope of this disclosure.

Skilled artisans will also appreciate that polyadenylation signals are widely used and known in the art, and that any suitable polyadenylation signal can be used in the embodiments of this disclosure. Exemplary polyadenylation signals are set forth in SEQ ID NOs:56-58.

Cas9 expression may be driven, in certain vectors of this disclosure, by one of three promoters: cytomegalovirus (CMV) (i.e., SEQ ID NO:45), elongation factor-1 (EFS) (i.e., SEQ ID NO:46), or human g-protein receptor coupled kinase-1 (hGRK1) (i.e., SEQ ID NO:47), which is specifically expressed in retinal photoreceptor cells. Modifications of the sequences of the promoters may be possible or desirable in certain applications, and such modifications are within the scope of this disclosure. In certain embodiments, Cas9 expression may be driven by a RHO promoter described herein (e.g., a minimum RHO Promoter (250 bp) SEQ ID NO:44).

Turning next to RHO cDNA, in some embodiments the RHO cDNA molecule may be wild-type RHO cDNA (e.g., SEQ ID NO:2). In certain embodiments, the RHO cDNA molecule may be a codon-modified cDNA to be resistant to hybridizing with a gRNA. In certain embodiments, the RHO cDNA molecule is not codon-modified to be resistant to hybridizing with a gRNA. In certain embodiments, the RHO cDNA molecule may be a codon-optimized cDNA to provide increased expression of rhodopsin protein (e.g., SEQ ID NOs:13-18). In certain embodiments, the RHO cDNA may comprise a modified 3′ UTR, for example, a 3′ UTR from a highly expressed, stable transcript, such as alpha- or beta-globin. Exemplarily 3′ UTRs are set forth in SEQ ID NOs:38-42. In certain embodiments, the RHO cDNA may include one or more introns (e.g., SEQ ID NOs:4-7). In certain embodiments, the RHO cDNA may include a truncation of one or more introns.

In certain embodiments, RHO cDNA expression may be driven by a rod-specific promoter. In certain embodiments, RHO cDNA expression may be driven by a RHO promoter described herein (e.g., a minimum RHO Promoter (250 bp) SEQ ID NO:44).

AAV genomes according to the present disclosure generally incorporate inverted terminal repeats (ITRs) derived from the AAV5 serotype. Exemplary left and right ITRs are SEQ ID NO:63 (AAV5 Left ITR) and SEQ ID NO:72 (AAV5 Right ITR), respectively. In certain embodiments, exemplary left and right ITRs are SEQ ID NO:92 (AAV Left ITR) and SEQ ID NO:93 (AAV Right ITR), respectively. It should be noted, however, that numerous modified versions of the AAV5 ITRs are used in the field, and the ITR sequences shown herein are exemplary and are not intended to be limiting. Modifications of these sequences are known in the art, or will be evident to skilled artisans, and are thus included in the scope of this disclosure.

The gRNA, RNA-guided nuclease, and RHO cDNA promoters are variable and can be selected from the lists presented herein. For clarity, this disclosure encompasses nucleic acids and/or AAV vectors comprising any combination of these elements, though certain combinations may be preferred for certain applications.

In various embodiments, a first nucleic acid or AAV vector may encode the following: left and right AAV ITR sequences (e.g., AAV5 ITRs), a promoter (e.g., CMV, hGRK1, EFS, RHO promoter) to drive expression of an RNA-guided nuclease (e.g., Cas9 encoded by a Cas9 nucleic acid molecule or Cpfl encoded by a Cpfl nucleic acid), NLS sequences flanking the RNA-guided nuclease nucleic acid molecule, and a second nucleic acid or AAV vector may encode the following: left and right AAV ITR sequences (e.g., AAV5 ITRs), a U6 promoter to drive expression of a guide RNA comprising a targeting domain sequence (e.g., a sequence according to a sequence in Tables 1-3 or 18), and a RHO promoter (e.g., minimal RHO promoter) to drive expression of a RHO cDNA molecule.

The nucleic acid or AAV vector may also comprise a Simian virus 40 (SV40) splice donor/splice acceptor (SD/SA) sequence element. In certain embodiments, the SV40 SD/SA element may be positioned between the promoter and the RNA-guided nuclease gene (e.g., Cas9 or Cpfl gene). In certain embodiments, a Kozak consensus sequence may precede the start codon of the RNA-guided nuclease (e.g., Cas9 or Cpfl) to ensure robust RNA-guided nuclease (e.g., Cas9 or Cpfl) expression.

In some embodiments, the nucleic acid or AAV vector shares at least 80%, 85%, 90%, 95%, 96%, 97%, 98%, 99% or greater sequence identity with one of the nucleic acids or AAV vectors recited above.

It should be noted that these sequences described above are exemplary and can be modified in ways that do not disrupt the operating principles of elements they encode. Such modifications, some of which are discussed below, are within the scope of this disclosure. Without limiting the foregoing, skilled artisans will appreciate that the DNA, RNA or protein sequences of the elements of this disclosure may be varied in ways that do not interrupt their function, and that a variety of similar sequences that are substantially similar (e.g., greater than 90%, 95%, 96%, 97%, 98% or 99% sequence similarity, or in the case of short sequences such as gRNA targeting domains, sequences that differ by no more than 1, 2 or 3 nucleotides) can be utilized in the various systems, methods and AAV vectors described herein. Such modified sequences are within the scope of this disclosure.

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween20) and/or a thermosensitive or reverse-thermosensitive polymer (e.g., pluronic). Other pharmaceutical formulation elements known in the art may also be suitable for use in the compositions described here.

Compositions comprising AAV vectors according to this disclosure can be administered to subjects by any suitable means, including without limitation injection, for example, subretinal injection. The concentration of AAV vector within the composition is selected to ensure, among other things, that a sufficient AAV dose is administered to the retina of the subject, taking account of dead volume within the injection apparatus and the relatively limited volume that can be safely administered to the retina. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal space. In some instances, the volume is selected to form a bleb in the subretinal space, for example 1 microliter, 10 microliters, 50 microliters, 100 microliters, 150 microliters, 200 microliters, 250 microliters, 300 microliters, etc.

Any region of the retina may be targeted, though the fovea (which extends approximately 1 degree out from the center of the eye) may be preferred in certain instances due to its role in central visual acuity and the relatively high concentration of cone photoreceptors there relative to peripheral regions of the retina. Alternatively or additionally, injections may be targeted to parafoveal regions (extending between approximately 2 and 10 degrees off center), which are characterized by the presence of both rod and cone photoreceptor cells. In addition, injections into the parafoveal region may be made at comparatively acute angles using needle paths that cross the midline of the retina. For instance, injection paths may extend from the nasal aspect of the sclera near the limbus through the vitreal chamber and into the parafoveal retina on the temporal side, from the temporal aspect of the sclera to the parafoveal retina on the nasal side, from a portion of the sclera located superior to the cornea to an inferior parafoveal position, and/or from an inferior portion of the sclera to a superior parafoveal position. The use of relatively small angles of injection relative to the retinal surface may advantageously reduce or limit the potential for spillover of vector from the bleb into the vitreous body and, consequently, reduce the loss of the vector during delivery. In other cases, the macula (inclusive of the fovea) can be targeted, and in other cases, additional retinal regions can be targeted, or can receive spillover doses.

To mitigate ocular inflammation and associated discomfort, one or more corticosteroids may be administered before, during, and/or after administration of the composition comprising AAV vectors. In certain embodiments, the corticosteroid may be an oral corticosteroid. In certain embodiments, the oral corticosteroid may be prednisone. In certain embodiments, the corticosteroid may be administered as a prophylactic, prior to administration of the composition comprising AAV vectors. For example, the corticosteroid may be administered the day prior to administration, or 2 days, 3 days, 4 days, 5 days, 6 days, 7 days, 8 days, 9 days, 10 days, 11 days, 12 days, 13 days, or 14 days prior to administration of the composition comprising AAV vectors. In certain embodiments, the corticosteroid may be administered for 1 week to 10 weeks after administration of the composition comprising AAV vectors (e.g., 1 week, 2 weeks, 3 weeks, 4 weeks, 5 weeks, 6 weeks, 7 weeks, 8 weeks, 9 weeks, or 10 weeks after administration of the composition comprising AAV vectors). In certain embodiments, the corticosteroid treatment may be administered prior to (e.g., the day prior to administration, or 2 days, 3 days, 4 days, 5 days, 6 days, 7 days, 8 days, 9 days, 10 days, 11 days, 12 days, 13 days, or 14 days prior to administration) and after administration of the composition comprising AAV vectors (e.g., 1 week, 2 weeks, 3 weeks, 4 weeks, 5 weeks, 6 weeks, 7 weeks, 8 weeks, 9 weeks, or 10 weeks after administration). For example, the corticosteroid treatment may be administered beginning 3 days prior to until 6 weeks after administration of the AAV vector.

Suitable doses of corticosteroids may include, for example, 0.1 mg/kg/day to 10 mg/kd/day (e.g., 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, or 1.0 mg/kg/day). In certain embodiments, the corticosteroid may be administered at an elevated dose during the corticosteroid treatment, followed by a tapered dose of the corticosteroid. For example, 0.5 mg/kg/day corticosteroid may be administered for 4 weeks, followed by a 15-day taper (0.4 mg/kg/day for 5 days, and then 0.2 mg/kg/day for 5 days, and then 0.1 mg/kg/day for 5 days). The corticosteroid dose may be increased if there is an increase in vitreous inflammation by 1+ on the grading scale following surgery (e.g., within 4 weeks after surgery). For example, if there is an increase in vitreous inflammation by 1+ on the grading scale while the patient is receiving a 0.5 mg/kg/day dose (e.g., within 4 weeks after surgery), the corticosteroid dose may be may be increased to 1 mg/kg/day. If any inflammation is present within 4 weeks after surgery, the taper may be delayed.

For pre-clinical development purposes, systems, compositions, nucleotides and vectors according to this disclosure can be evaluated ex vivo using a retinal explant system, or in vivo using an animal model such as a mouse, rabbit, pig, nonhuman primate, etc. Retinal explants are optionally maintained on a support matrix, and AAV vectors can be delivered by injection into the space between the photoreceptor layer and the support matrix, to mimic subretinal injection. Tissue for retinal explanation can be obtained from human or animal subjects, for example mouse.

Explants are particularly useful for studying the expression of gRNAs, RNA-guided nucleases, and rhodopsin protein following viral transduction, and for studying genome editing over comparatively short intervals. These models also permit higher throughput than may be possible in animal models and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

DNA-Based Delivery of an RNA-Guided Nuclease Molecule, a gRNA Molecule, and/or a RHO Expression Cassette

DNA encoding RNA-guided nuclease molecules (e.g., Cas9 or Cpfl molecules), gRNA molecules, and/or RHO cDNA molecules can be administered to subjects or delivered into cells by art-known methods or as described herein. For example, RNA-guided nuclease (e.g., Cas9 or Cpfl) encoding DNA, gRNA-encoding DNA, and/or RHO cDNA can be delivered, e.g., by vectors (e.g., viral or non-viral vectors), non-vector based methods (e.g., using naked DNA or DNA complexes), or a combination thereof.

In some embodiments, the RNA-guided nuclease (e.g., Cas9 or Cpfl)-encoding DNA, gRNA-encoding DNA, and/or RHO cDNA is delivered by a vector (e.g., viral vector/virus or plasmid).

A vector can comprise a sequence that encodes an RNA-guided nuclease-encoding DNA, gRNA-encoding DNA, and/or RHO cDNA molecule. A vector can also comprise a sequence encoding a signal peptide (e.g., for nuclear localization, nucleolar localization, mitochondrial localization), fused, e.g., to an RNA-guided nuclease sequence. For example, a vector can comprise a nuclear localization sequence (e.g., from SV40) fused to the sequence encoding the RNA-guided nuclease (e.g., Cas9 or Cpfl) molecule.

One or more regulatory/control elements, e.g., a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is recognized by RNA polymerase II (e.g., a CMV promoter). In other embodiments, the promoter is recognized by RNA polymerase III (e.g., a U6 promoter). In some embodiments, the promoter is a regulated promoter (e.g., inducible promoter). In other embodiments, the promoter is a constitutive promoter. In some embodiments, the promoter is a tissue specific promoter. In some embodiments, the promoter is a viral promoter. In other embodiments, the promoter is a non-viral promoter.

In some embodiments, the vector or delivery vehicle is a viral vector (e.g., for generation of recombinant viruses). In some embodiments, the virus is a DNA virus (e.g., dsDNA or ssDNA virus). In other embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in human. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the RNA-guided nuclease molecule, the gRNA molecule, and/or the RHO cDNA molecule. In other embodiments, the virus causes long-lasting, e.g., at least 1 week, 2 weeks, 1 month, 2 months, 3 months, 6 months, 9 months, 1 year, 2 years, or permanent expression, of the RNA-guided nuclease molecule, the gRNA molecule, and/or the RHO cDNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the RNA-guided nuclease-encoding DNA, gRNA-encoding DNA, and/or RHO cDNA is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the RNA-guided nuclease-encoding DNA, gRNA-encoding DNA, and/or RHO cDNA is delivered by a recombinant lentivirus. For example, the lentivirus is replication-defective, e.g., does not comprise one or more genes required for viral replication.

In some embodiments, the RNA-guided nuclease-encoding DNA, gRNA-encoding DNA, and/or RHO cDNA is delivered by a recombinant adenovirus. In some embodiments, the adenovirus is engineered to have reduced immunity in human.

In some embodiments, the RNA-guided nuclease-encoding DNA, gRNA-encoding DNA, and/or RHO cDNA is delivered by a recombinant AAV. In some embodiments, the AAV can incorporate its genome into that of a host cell, e.g., a target cell as described herein.

In some embodiments, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods.

In some embodiments, the RNA-guided nuclease-encoding DNA, gRNA-encoding DNA, and/or RHO cDNA is delivered by a hybrid virus, e.g., a hybrid of one or more of the viruses described herein.

A Packaging cell is used to form a virus particle that is capable of infecting a host or target cell. Such a cell includes a 293 cell, which can package adenovirus, and a w2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In an embodiment, the viral vector has the ability of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In an embodiment, the viral vector achieves cell type specific expression. For example, a tissue-specific promoter can be constructed to restrict expression of the transgene (Cas 9 and gRNA) in only the target cell. The specificity of the vector can also be mediated by microRNA-dependent control of transgene expression. In an embodiment, the viral vector has increased efficiency of fusion of the viral vector and a target cell membrane. For example, a fusion protein such as fusion-competent hemagglutin (HA) can be incorporated to increase viral uptake into cells. In an embodiment, the viral vector has the ability of nuclear localization. For example, a virus that requires the breakdown of the cell wall (during cell division) and therefore will not infect a non-diving cell can be altered to incorporate a nuclear localization peptide in the matrix protein of the virus thereby enabling the transduction of non-proliferating cells.

In some embodiments, the RNA-guided nuclease-encoding DNA, gRNA-encoding DNA, and/or RHO cDNA is delivered by a non-vector based method (e.g., using naked DNA or DNA complexes). For example, the DNA can be delivered, e.g., by organically modified silica or silicate (Ormosil), electroporation, gene gun, sonoporation, magnetofection, lipid-mediated transfection, dendrimers, inorganic nanoparticles, calcium phosphates, or a combination thereof.

In some embodiments, the RNA-guided nuclease-encoding DNA, gRNA-encoding DNA, and/or RHO cDNA is delivered by a combination of a vector and a non-vector based method. For example, a virosome comprises a liposome combined with an inactivated virus (e.g., HIV or influenza virus), which can result in more efficient gene transfer, e.g., in a respiratory epithelial cell than either a viral or a liposomal method alone.

In an embodiment, the delivery vehicle is a non-viral vector. In an embodiment, the non-viral vector is an inorganic nanoparticle (e.g., attached to the payload to the surface of the nanoparticle). Exemplary inorganic nanoparticles include, e.g., magnetic nanoparticles (e.g., Fe₃MnO₂), or silica. The outer surface of the nanoparticle can be conjugated with a positively charged polymer (e.g., polyethylenimine, polylysine, polyserine) which allows for attachment (e.g., conjugation or entrapment) of payload. In an embodiment, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids for gene transfer are shown below in Table 15.

TABLE 15
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP—DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl- | DOSPA | Cationic |
| 1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |

Exemplary polymers for gene transfer are shown below in Table 16.

TABLE 16
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS—PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly(2-(dimethylamino)ethyl methacrylate) | pDMAEMA |
| Poly(2-aminoethyl propylene phosphate) | PPE—EA |
| Chitosan | |
| Galactosylated chitosan | |
| N-Dodacylated chitosan | |
| Histone | |
| Collagen | |
| Dextran-spermine | D—SPM |

In an embodiment, the vehicle has targeting modifications to increase target cell update of nanoparticles and liposomes, e.g., cell specific antigens, monoclonal antibodies, single chain antibodies, aptamers, polymers, sugars, and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimuli-cleavable polymer is used, e.g., for release in a cellular compartment. For example, disulfide-based cationic polymers that are cleaved in the reducing cellular environment can be used.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In an embodiment, the vehicle is a genetically modified bacteriophage (e.g., engineered phages having large packaging capacity, less immunogenic, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovesicle (30-100 nm) of endocytic origin (e.g., can be produced from various cell types and can therefore be taken up by cells without the need of for targeting ligands).

In an embodiment, one or more nucleic acid molecules (e.g., DNA molecules) other than the components of an RNA-guided nuclease system, e.g., the Cas9 or Cpfl molecule component, the gRNA molecule component, and/or the RHO cDNA molecule component described herein, are delivered. In an embodiment, the nucleic acid molecule is delivered at the same time as one or more of the components of the RNA-guided nuclease system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the RNA-guided nuclease system are delivered. In an embodiment, the nucleic acid molecule is delivered by a different means than one or more of the components of the RNA-guided nuclease system, e.g., the Cas9 or Cpfl molecule component, the gRNA molecule component, and/or the RHO cDNA molecule component are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component, the gRNA molecule component, and/or the RHO cDNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

### Delivery of RNA Encoding an RNA-Guided Nuclease Molecule

RNA encoding RNA-guided nuclease molecules (e.g., Cas9 or Cpfl molecules described herein), gRNA molecules, and/or RHO cDNA molecules can be delivered into cells, e.g., target cells described herein, by art-known methods or as described herein. For example, RNA-guided nuclease molecules (e.g., Cas9 or Cpfl molecules described herein), gRNA molecules, and/or RHO cDNA molecules can be delivered, e.g., by microinjection, electroporation, lipid-mediated transfection, peptide-mediated delivery, or a combination thereof

### Delivery RNA-Guided Nuclease Molecule Protein

RNA-guided nuclease molecules (e.g., Cas9 or Cpfl molecules described herein) can be delivered into cells by art-known methods or as described herein. For example, RNA-guided nuclease protein molecules can be delivered, e.g., by microinjection, electroporation, lipid-mediated transfection, peptide-mediated delivery, or a combination thereof. Delivery can be accompanied by DNA encoding a gRNA and/or RHO cDNA or by a gRNA and/or RHO cDNA.

### Routes of Administration

Systemic modes of administration include oral and parenteral routes. Parenteral routes include, by way of example, intravenous, intraarterial, intraosseous, intramuscular, intradermal, subcutaneous, intranasal and intraperitoneal routes. Components administered systemically may be modified or formulated to target the components to the eye.

Local modes of administration include, by way of example, intraocular, intraorbital, subconjuctival, intravitreal, subretinal or transscleral routes. In an embodiment, significantly smaller amounts of the components (compared with systemic approaches) may exert an effect when administered locally (for example, intravitreally) compared to when administered systemically (for example, intravenously). Local modes of administration can reduce or eliminate the incidence of potentially toxic side effects that may occur when therapeutically effective amounts of a component are administered systemically.

In an embodiment, components described herein are delivered by subretinally, e.g., by subretinal injection. Subretinal injections may be made directly into the macular, e.g., submacular injection.

In an embodiment, components described herein are delivered by intravitreal injection. Intravitreal injection has a relatively low risk of retinal detachment risk. In an embodiment, nanoparticle or viral, e.g., AAV vector, e.g., an AAV5 vector, e.g., a modified AAV5 vector, an AAV2 vector, e.g., a modified AAV2 vector, is delivered intravitreally.

Methods for administration of agents to the eye are known in the medical arts and can be used to administer components described herein. Exemplary methods include intraocular injection (e.g., retrobulbar, subretinal, submacular, intravitreal and intrachoridal), iontophoresis, eye drops, and intraocular implantation (e.g., intravitreal, sub-Tenons and sub-conjunctival).

Administration may be provided as a periodic bolus (for example, subretinally, intravenously or intravitreally) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag). Components may be administered locally, for example, by continuous release from a sustained release drug delivery device immobilized to an inner wall of the eye or via targeted transscleral controlled release into the choroid (see, for example, PCT/US00/00207, PCT/US02/14279, Ambati 2000a, and Ambati 2000b. A variety of devices suitable for administering components locally to the inside of the eye are known in the art. See, for example, U.S. Pat. Nos. 6,251,090, 6,299,895, 6,416,777, 6,413,540, and PCT/US00/28187.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used for intraocular injection. Typically the microspheres are composed of a polymer of lactic acid and glycolic acid, which are structured to form hollow spheres. The spheres can be approximately 15-30 microns in diameter and can be loaded with components described herein.

### Bi-Modal or Differential Delivery of Components

Separate delivery of the components of an RNA-guided nuclease system, e.g., the RNA-guided nuclease molecule component (e.g., Cas9 or Cpfl molecule component), the gRNA molecule component, and the RHO cDNA molecule component, and more particularly, delivery of the components by differing modes, can enhance performance, e.g., by improving tissue specificity and safety.

In an embodiment, the RNA-guided nuclease molecule component, the gRNA molecule component, and the RHO cDNA molecule component, are delivered by different modes, or as sometimes referred to herein as differential modes. Different or differential modes, as used herein, refer modes of delivery that confer different pharmacodynamic or pharmacokinetic properties on the subject component molecule, e.g., n RNA-guided nuclease molecule, gRNA molecule, or RHO cDNA molecule. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., adeno-associated virus or lentivirus, delivery.

By way of example, the components, e.g., an RNA-guided nuclease molecule, a gRNA molecule, and a RHO cDNA molecule can be delivered by modes that differ in terms of resulting half-life or persistent of the delivered component the body, or in a particular compartment, tissue or organ. In an embodiment, a gRNA molecule can be delivered by such modes. The RNA-guided nuclease molecule component can be delivered by a mode which results in less persistence or less exposure to the body or a particular compartment or tissue or organ. The RHO cDNA molecule component may be delivered by a mode that difference from that mode of the gRNA molecule component and the RNA-guided nuclease molecule component.

More generally, in an embodiment, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In an embodiment, the first pharmacodynamic or pharmacokinetic property, e.g., distribution, persistence or exposure, is more limited than the second pharmacodynamic or pharmacokinetic property.

In an embodiment, the first mode of delivery is selected to optimize, e.g., minimize, a pharmacodynamic or pharmacokinetic property, e.g., distribution, persistence or exposure.

In an embodiment, the second mode of delivery is selected to optimize, e.g., maximize, a pharmacodynamic or pharmcokinetic property, e.g., distribution, persistence or exposure.

In an embodiment, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In an embodiment, the second mode of delivery comprises a relatively transient element, e.g., an RNA or protein.

In an embodiment, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, an RNA-guided nuclease molecule, is delivered in a transient manner, for example as mRNA or as protein, ensuring that the full RNA-guided nuclease molecule/gRNA molecule complex is only present and active for a short period of time.

Furthermore, the components can be delivered in different molecular form or with different delivery vectors that complement one another to enhance safety and tissue specificity.

Use of differential delivery modes can enhance performance, safety and efficacy. E.g., the likelihood of an eventual off-target modification can be reduced. Delivery of immunogenic components, e.g., RNA-guided nuclease molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in an embodiment, a first component, e.g., a gRNA molecule is delivered by a first delivery mode that results in a first spatial, e.g., tissue, distribution. A second component, e.g., an RNA-guided nuclease molecule is delivered by a second delivery mode that results in a second spatial, e.g., tissue, distribution. In an embodiment, the first mode comprises a first element selected from a liposome, nanoparticle, e.g., polymeric nanoparticle, and a nucleic acid, e.g., viral vector. The second mode comprises a second element selected from the group. In an embodiment, the first mode of delivery comprises a first targeting element, e.g., a cell specific receptor or an antibody, and the second mode of delivery does not include that element. In embodiment, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule and the RNA-guided nuclease molecule are packaged in separated delivery vehicles with distinct but overlapping tissue tropism, the fully functional complex is only formed in the tissue that is targeted by both vectors.

### Ex Vivo Delivery

In some embodiments, components described in Table 8 are introduced into cells which are then introduced into the subject. Methods of introducing the components can include, e.g., any of the delivery methods described in Table 9.

### VIII. Modified Nucleosides, Nucleotides, and Nucleic Acids

In some embodiments of the present disclosure, modified nucleosides and/or modified nucleotides can be present in nucleic acids, e.g., in a gRNA molecule provided herein. Some exemplary nucleoside, nucleotide, and nucleic acid modifications useful in the context of the present RNA-guided nuclease technology are provided herein, and the skilled artisan will be able to ascertain additional suitable modifications that can be used in conjunction with the nucleosides, nucleotides, and nucleic acids and treatment modalities disclosed herein based on the present disclosure. Suitable nucleoside, nucleotide, and nucleic acid modifications include, without limitation, those described in U.S. Patent Application No. US 2017/0073674 A1 and International Publication No. WO 2017/165862 A1, the entire contents of each of which are incorporated by reference herein.

### EXAMPLES

The following Examples are merely illustrative and are not intended to limit the scope or content of the disclosure in any way.

### Example 1: Screening of gRNAs for Editing RHO Alleles in T Cells

Approximately 430 gRNAs targeting various positions within the RHO gene for use with Cas9 were designed and screened for editing activity in T cells. Briefly, SA Cas9 and guide RNA were complexed at a 1:2 ratio (RNP complex) and delivered to T cells via electroporation. Three days after electroporation, gDNA was extracted from T cells and the target site was PCR amplified from the gDNA. Sequencing analysis of the RHO PCR gene product was evaluated by next generation sequencing (NGS). Table 18 below provides the RNA and DNA sequences of the targeting domains of the gRNAs that exhibited >0.1% editing in T cells. These data indicate that gRNA comprising targeting domains set forth in Table 18 and Cas9 support editing of the RHO gene.

### Example 2: Dose-Dependent Editing of RHO Alleles in HEK293 Cells

Three gRNAs whose target sites are predicted to be within exon 1 or exon 2 of the RHO gene, RHO-3, RHO-7, and RHO-10 (Table 17), were selected for further optimization and testing for dose-dependent editing with Cas9. Briefly, increasing concentrations of control plasmid (expressing Cas9 with scrambled gRNA that does not target a sequence within the human genome) or plasmids expressing Cas9 and gRNA were delivered to HEK293 cells by electroporation. Three days after electroporation, gDNA was extracted from HEK293 cells and the gRNA target site was PCR amplified from the gDNA.

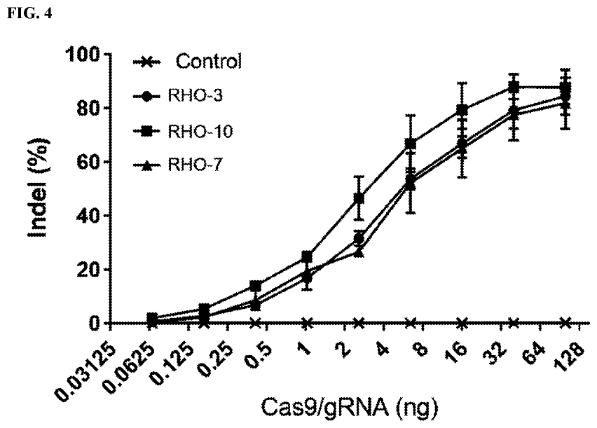

Sequencing analysis of the RHO PCR gene product was evaluated by NGS. The increasing concentration of Cas9/gRNA plasmid supported an increase in indels at the RHO gene to 80% (FIG. 4). Sequencing analysis indicated that increasing the plasmid concentration resulted in an increase in indels.

TABLE 17
gRNAs Targeting RHO Gene

| | Targeting | Targeting Domain |
|---|---|---|
| gRNA ID | Domain (RNA) | (DNA)/Protospacer |
| RHO-3 | AGUAUCCAUGCAGAGAGG | AGTATCCATGCAGAGAGG |
| | UGUA(SEQ ID NO: 102) | TGTA (SEQ ID NO: |
| | | 602) |
| RHO-7 | CCCACACCCGGCUCAUAC | CCCACACCCGGCTCATAC |
| | CGCC(SEQ ID NO: 106) | CGCC (SEQ ID NO: |
| | | 606) |
| RHO-10 | GUGCCAUUACCUGGACCA | GTGCCATTACCTGGACCA |
| | (GCCGSEQ ID NO: 109) | GCCG (SEQ ID NO: |
| | | 609) |

Specificity of the gRNA (i.e., RHO-3, RHO-7, RHO-10) and Cas9 ribonucleoprotein complexes was evaluated using two different assays that are well-known to skilled artisans for profiling CRISPR-Cas9 specificity, the Digenome-seq (digested genome sequencing) and GUIDE-seq assays. No apparent off target editing was detected under physiological conditions for RNP comprising RHO-3, RHO-7, or RHO-10 gRNA complexed with Cas9 (data not shown).

### Example 3: Characterization of Novel RHO Alleles Generated by Simulation of On-Targeted Editing by RHO-3, RHO-7, and RHO-10 gRNAs

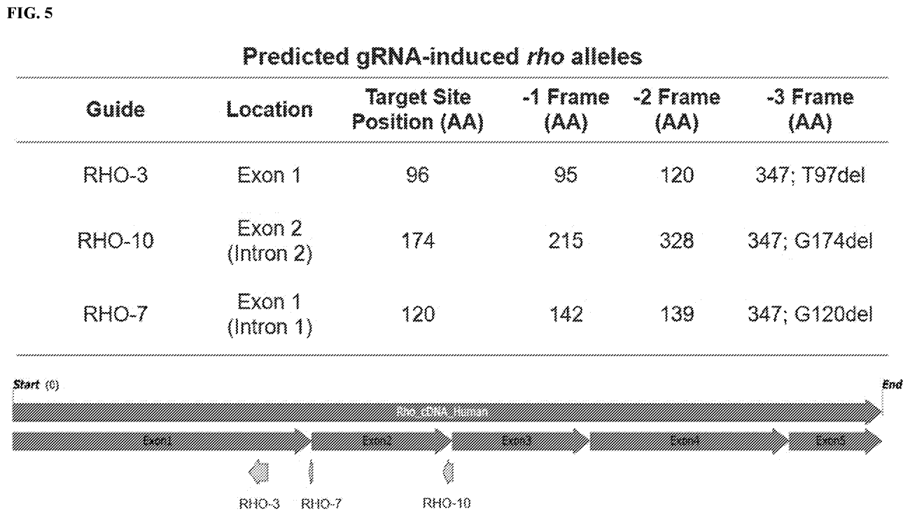

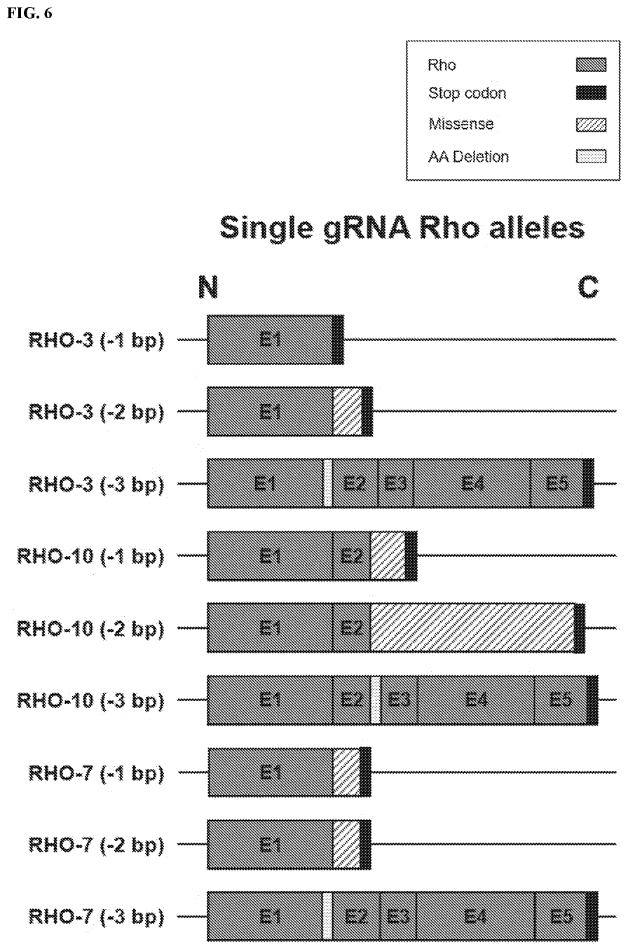

The cut sites generated by on-targeted editing of RHO-3, RHO-7, or RHO-10 gRNA (see targeting domains in Table 17) of RHO alleles were predicted. FIG. 5 illustrates the predicted cutting locations of RHO-3, RHO-7, or RHO-10 gRNAs on the RHO human cDNA and resulting lengths of RHO protein. RHO-3 is predicted to target Exon 1, RHO-10 is predicted to target the boundary of Exon 2 and Intron 2, and RHO-7 is predicted to target the boundary of Exon 1 and Intron 1 of RHO cDNA. Deletions of 1 or 2 base pairs at the RHO-3, RHO-10, or RHO-7 target sites are predicted to cause frameshifts in the RHO cDNA resulting in abnormal RHO proteins. FIG. 6 shows schematics of the predicted RHO alleles resulting from editing by RHO-3, RHO-10, or RHO-7 gRNAs.

The effects of the alleles generated by on-targeted editing by RHO-3, RHO-7, or RHO-10 gRNA were characterized to determine whether editing using these gRNAs could result in potentially deleterious RHO alleles. Briefly, wild-type (WT) or mock-edited RHO alleles were cloned into mammalian expression plasmids under the control of a CMV promoter and lipofected into HEK293 cells. Mock-edited RHO alleles included each of the mutated alleles shown in FIG. 6 (i.e., RHO-3 (−1, −2, or −3 bp), RHO-10 (−1, −2, or −3 bp), or RHO-7 (−1 bp, −2 bp, −3 bp)). The well-known P23H RHO variant leading to a dominant form of retinitis pigmentosa was also cloned and tested. After 48 hours of overexpression, cell viability for WT and each mock-edited allele was assessed using ATPLite Luminescence Assay (Perkin Elmer).

While WT RHO overexpression induced relatively no cytotoxicity with respect to the vector control (pUC19 plasmid, upper dotted line), P23H RHO resulted in 50% cell death (lower dotted line), as expected (FIG. 7A). Furthermore, expression of the frameshifting of one- or two-base pair deletions at the RHO-3, RHO-7, or RHO-10 gRNA target sites did not induce significant loss in cell viability with respect to WT RHO (FIG. 7A, see RHO-3 1 and 2 bp del; RHO-10 1 and 2 bp del; and RHO-7 1 and 2 bp del). However, for in-frame three-base pair deletions at RHO-3 and RHO-10 target sites, there was a significant loss in cell viability, resulting in levels of cell death comparable to that of P23H RHO (FIG. 7A, see RHO-3 3 bp del and RHO-10 3 bp del). This was not the case for all gRNAs as a three-base pair deletion at the RHO-7 sequence resulted in a non-cytotoxic RHO allele (FIG. 7A, see RHO-7 3 bp del).

Next, to determine whether the RHO-3, RHO-7, and RHO-10 mock-edited RHO alleles could reduce toxicity of the P23H variant of RHO, mock-edited RHO-3, RHO-7, and RHO-10 RHO alleles shown in FIG. 6 and containing the P23H mutation were cloned into mammalian expression plasmids under the control of a CMV promoter and lipofected into HEK293 cells. After 48 hours of overexpression, cell viability for WT and each mock-edited allele was assessed using ATPLite Luminescence Assay (Perkin Elmer).

Expression of the frameshifting of one- or two-base pair deletions at the RHO-3, RHO-7, or RHO-10 gRNA target sites reduced toxicity of the P23H variant of RHO and did not induce significant loss in cell viability with respect to WT RHO (FIG. 7B, see RHO-3 1 and 2 bp del, RHO-10 1 and 2 bp del and RHO-7 1 and 2 bp del). The in-frame three-base pair deletions at RHO-3 and RHO-10 target sites did not reduce toxicity of the P23H variant of RHO as there was a significant loss in cell viability, resulting in levels of cell death comparable to that of P23H RHO (FIG. 7B, see RHO-3 3 bp del and RHO-10 3 bp del). However, the three-base pair deletion at the RHO-7 target sequence reduced toxicity of the P23H variant of RHO and resulted in a non-cytotoxic RHO allele (FIG. 7B, see RHO-7 3 bp del).

These data indicate that out-of-frame RHO edits produced by RHO-3, RHO-7, or RHO-10 gRNA were productive and non-toxic while the effect of in-frame edits were gRNA/locus dependent.

### Example 4: Editing of Non-Human Primate Explants by Ribonucleoproteins Comprising Cas9 and gRNA Targeting the RHO Gene

The ability of ribonucleoproteins comprising RHO-9 gRNA targeting the RHO gene and Cas9 to edit explants from non-human primates (NHP) was assessed. The RHO-9 gRNA (comprising the targeting domain sequence set forth in SEQ ID NO:108 (RNA) (SEQ ID NO:608 (DNA), Table 1) is cross-reactive and can edit both human and NHP RHO sequences.

Briefly, retinal explants from NHP donors were harvested and transferred to a membrane on a trans-well chamber in a 24 well plate. 300 μl of retinal media was added to the 24 well plate (i.e., Neurobasal-A media (no phenol red) (470 mL) containing B27 (with VitA) 50× (20 mL), Antibiotic-Antimycotic (5 mL), and GlutaMAX 1% (5 mL)). Transduction with dual AAV comprising RHO-9 gRNA, SA Cas9, and Replacement RHO occurred after 24-48 hours. AAVs were diluted to the desired titer (10¹² vg/ml)) with the retinal media to obtain the final concentration in a total of 100 μl. The diluted/titered AAV was added dropwise on top of the explant in the 24 well plate. 300 μl of retinal media was replenished every 72 hours. After 2-4 weeks, explants were lysed to obtain DNA, RNA and protein for molecular biology analysis. To measure the percentage of rods in the explants, a rod-specific mRNA (neural retina leucine zipper (NRL)) was extracted from the explants and measured. The housekeeping RNA (beta actin (ACTB)) was also measured to determine the total number of cells.

As shown in FIG. 8, each data point represents a single explant, which can contain differing numbers of rod photoreceptors. The x-axis shows the delta between ACTB and NRL RNA levels as measured by RT-qPCR, which is a measure for the percentage of rods in the explant at the time of lysing the explants. A correlation between significant editing and high percentage of rods was shown, demonstrating that robust editing levels can be achieved in explants with a substantial number of rods (FIG. 8). These data show that gRNA targeting RHO can efficiently edit non-human primate explants.

### Example 5: Optimization of RHO Replacement Vector

Various components of the RHO replacement vector (e.g., promoter, UTRs, RHO sequence) were optimized to identify the optimal RHO replacement vector for maximal expression of RHO mRNA and RHO protein. First, a dual luciferase system was designed to test the impact that different lengths of the RHO promoter have on RHO expression. The components of the luciferase system included a Renilla luciferase driven by CMV in the backbone to normalize for plasmid concentrations and transfection efficiencies (FIG. 9).

Briefly, plasmids containing different lengths of the RHO promoter and the RHO gene tagged with a firefly luciferase separated by a self-cleaving T2A peptide (100 ng/10,000 cells) were transfected into HEK293 cells along with a plasmid expressing NRL, CRX, and NONo (100 ng/10,000) to turn on expression from the RHO promoters (see Yadav 2014, the entire contents of which are incorporated herein by reference). 72 hours later the cells were lysed and both transfection efficiency (Firefly) and experimental variable (NanoLuc) were analyzed. The Nano-Glo® Dual-Luciferase® Reporter Assay System (Promega Corporation, Cat #N1521) was used to measure luminescence. Luminescence from both Firefly and NanoLuc were measured. As shown in FIG. 10, promoters of different lengths were shown to be functional, including the minimal 250 bp RHO promoter (SEQ ID NO:44).

Next, varying 3′ UTRs were tested to determine whether 3′ UTRs can improve expression of RHO mRNA and RHO protein. Briefly, 3′ UTRs from highly stable transcripts and genes were cloned downstream of CMV RHO (i.e., HBA1 3′ UTR (SEQ ID NO:38), short HBA1 3′ UTR (SEQ ID NO:39), TH 3′ UTR (SEQ ID NO:40), COL1A1 3′UTR (SEQ ID NO:41), ALOX15 3′UTR (SEQ ID NO:42), and minUTR (SEQ ID NO:56)). Vectors (500 ng) were transfected into HEK293 cells (80,000 cells/well). 72 hours later the cells were lysed, and RHO mRNA and protein expression levels were determined using RHO RT-qPCR and RHO ELISA assays, respectively. FIG. 11A shows that incorporation of 3′ UTRs from stable transcripts into the RHO replacement vector improved RHO mRNA expression levels. FIG. 11B shows that incorporation of 3′ UTRs from stable transcripts into the RHO replacement vector also improved RHO protein expression levels.

Next, incorporation of sequences of RHO introns 1, 2, 3, or 4 were added to RHO cDNA (i.e., SEQ ID NOs:4-7, respectively) in the RHO replacement vector to determine the impact on RHO protein expression. Vectors (500 and 250 ng) were transfected into HEK293 cells (80,000/well). 72 hours later the cells were lysed, and RHO protein expression was determined using RHO ELISA. FIG. 12 shows that addition of introns affects RHO protein expression.

Lastly, different codon optimized RHO cDNA constructs (i.e., SEQ ID NOs:13-18) were tested to determine the impact of codon optimization on RHO expression. Vectors (500 and 250 ng) were transfected into HEK293 cells (80,000/well). 72 hours later the cells were lysed and RHO protein expression was determined using a RHO ELISA. FIG. 13 shows that codon optimization of the RHO cDNA impacts RHO protein expression.

### Example 6: In Vivo Editing Using Self-Limiting Cas9 Vector System to Reduce Cas9 Levels after Successful Editing

The ability of a dual vector system expressing Cas9 and gRNAs to edit the RHO genome and to render Cas9 vector expression non-functional was tested in vivo. The self-limiting vector system has previously been published (see WO2018/106693, published on Jun. 14, 2018, and entitled Systems and Methods for One-Shot guide RNA (ogRNA) Targeting of Endogenous and Source DNA, the entire contents of which are incorporated herein by reference). Briefly, a Cas9 vector system was generated in which the Cas9 vector comprised a target site for the RHO gRNA within the Cas9 cDNA (SD Cas9). Six weeks after administration of the SD Cas9 and RHO vectors, Cas9 protein levels, Cas9 AAV, and editing of RHO was assessed.

FIG. 14A indicates that the SD Cas9 vector system demonstrated successful silencing of Cas9 levels. FIG. 14B indicates that the vector system carrying the SD Cas9 system resulted in robust editing at the RHO locus, albeit at slightly lower levels as compared to a vector system encoding a wild-type Cas9 sequence.

### Example 7: Editing of Human Explants by Ribonucleoproteins Comprising gRNA Targeting the RHO Gene and Cas9

The ability of ribonucleoproteins comprising RHO-9 gRNA (Table 1) targeting the RHO gene and Cas9 to edit human explants was assessed. Briefly, retinal explants from one human donor were harvested and transferred to a membrane on a trans-well chamber in a 24 well plate. 300 μl of retinal media was added to the 24 well plate (i.e., Neurobasal-A media (no phenol red) (470 mL) containing B27 (with VitA) 50× (20 mL), Antibiotic- Antimycotic (5 mL), and GlutaMAX 1% (5 mL)). Different “knock-down and replace” strategies were compared: “shRNA”: transduction of retinal explants with shRNA targeting the RHO gene and a replacement vector providing a RHO cDNA (as published in Cideciyan 2018); “Vector A”: a two-vector system (Vector 1 comprising saCas9 driven by the minimal RHO promoter (250 bp), and Vector 2 comprising a codon-optimized RHO cDNA (Codon 6 (SEQ ID NO:18)) and comprising a HBA1 3′ UTR under the control of the minimal 250 bp RHO promoter, as well as a the RHO-9 gRNA under the control of a U6 promoter); “Vector B”: a two-vector system identical to “Vector A” except for Vector 2 comprising a wt RHO cDNA; and “UTC”: untransduced control. The respective AAVs were diluted to the desired titer (1×10¹² vg/ml) with the retinal media to obtain the final concentration in a total of 100 μl. The diluted/titered AAV was added dropwise on top of the explant in the 24 well plate. 300 μl of retinal media was replenished every 72 hours. After 4 weeks, explants were lysed to obtain protein for molecular biology analysis. The ratio of RHO protein:total protein was measured. Data indicate that Vector A (comprising the minimal 250 bp promoter, RHO cDNA, HBA1 3′ UTR, and RHO-9 gRNA), resulted in robust expression of RHO protein (FIG. 15).

### Example 8: Administration of a Gene Editing System to a Patient in Need Thereof

A human patient presenting with adRP is administered a gene editing system comprising two AAV5-based expression vectors.

Vector 1 comprises a nucleic acid sequence encoding an S. aureus Cas9 protein, flanked on each site by a nuclear localization sequence under the control of a GRK1 promoter or under the control of a RHO minimal promoter (e.g., 250 bp RHO promoter).

Vector 2 comprises a nucleic acid sequence encoding one or more guide RNAs, each under the control of a U6 promoter. The targeting domain of the one or more guide RNAs, independently, is selected from the following sequences:

RHO-1
(SEQ ID NO: 100)
GUCAGCCACAAGGGCCACAGCC
RHO-2:
(SEQ ID NO: 101)
CCGAAGACGAAGUAUCCAUGCA
RHO-3:
(SEQ ID NO: 102)
AGUAUCCAUGCAGAGAGGUGUA
RHO-4:
(SEQ ID NO: 103)
CUAGGUUGAGCAGGAUGUAGUU
RHO-5:
(SEQ ID NO: 104)
CAUGGCUCAGCCAGGUAGUACU
RHO-6:
(SEQ ID NO: 105)
ACGGGUGUGGUACGCAGCCCCU
RHO-7:
(SEQ ID NO: 106)
CCCACACCCGGCUCAUACCGCC
RHO-8:
(SEQ ID NO: 107)
CCCUGGGCGGUAUGAGCCGGGU
RHO-9:
(SEQ ID NO: 108)
CCAUCAUGGGCGUUGCCUUCAC
RHO-10:
(SEQ ID NO: 109)
GUGCCAUUACCUGGACCAGCCG
RHO-11:
(SEQ ID NO: 110)
UUACCUGGACCAGCCGGCGAGU

The nucleic acid sequence encoding the guide RNA is under the control of a U6 promoter. Vector 2 further comprises a nucleic acid comprising an upstream sequence encoding a RHO 5′-UTR, a RHO cDNA, and a downstream sequence encoding an HBA1 3′-UTR under the control of a minimal RHO promoter sequence that comprises a portion of the RHO distal enhancer and a portion of the RHO proximal promoter region. The [promoter]-[5′UTR]-[cDNA]-[3′UTR] sequence of Vector 2 is as follows:

(SEQ ID NO: 8)
CCACGTCAGAATCAAACCCTCACCTTAACCTCATTAGCGTTGGGCATAAT
CACCAGGCCAAGCGCCTTAAACTACGAGAGGCCCCATCCCACCCGCCCTG
CCTTAGCCCTGCCACGTGTGCCAAACGCTGTTAGACCCAACACCACCCAG
GCCAGGTAGGGGGCTGGAGCCCAGGTGGGCATTTGAGTCACCAACCCCCA
GGCAGTCTCCCTTTTCCTGGATCCTGAGTACCTCTCCTCCCTGACCTCAG
GCTTCCTCCTAGTGTCACCTTGGCCCCTCTTAGAAGCCAATTAGGCCCTC
AGTTTCTGCAGCGGGGATTAATATGATTATGAACACCCCCAATCTCCCAG
ATGCTGATTCAGCCAGGAGCTTAGGAGGGGGAGGTCACTTTATAAGGGTC
TGGGGGGGTCAGAACCCAGAGTCATCCAGCTGGAGCCCTGAGTGGCTGAG
CTCAGGCCTTCGCAGCATTCTTGGGTGGGAGCAGCCACGGGTCAGCCACA
AGGGCCACCACCATGAATGGCACAGAAGGCCCTAACTTCTACGTGCCCTT
CTCCAATGCGACGGGTGTGGTACGCAGCCCCTTCGAGTACCCACAGTACT
ACCTGGCTGAGCCATGGCAGTTCTCCATGCTGGCCGCCTACATGTTTCTG
CTGATCGTGCTGGGCTTCCCCATCAACTTCCTCACGCTCTACGTCACCGT
CCAGCACAAGAAGCTGCGCACGCCTCTCAACTACATCCTGCTCAACCTAG
CCGTGGCTGACCTCTTCATGGTCCTAGGTGGCTTCACCAGCACCCTCTAC
ACCTCTCTGCATGGATACTTCGTCTTCGGGCCCACAGGATGCAATTTGGA
GGGCTTCTTTGCCACCCTGGGCGGTGAAATTGCCCTGTGGTCCTTGGTGG
TCCTGGCCATCGAGCGGTACGTGGTGGTGTGTAAGCCCATGAGCAACTTC
CGCTTCGGGGAGAACCATGCCATCATGGGCGTTGCCTTCACCTGGGTCAT
GGCGCTGGCCTGCGCCGCACCCCCACTCGCCGGCTGGTCCAGGTACATCC
CCGAGGGCCTGCAGTGCTCGTGTGGAATCGACTACTACACGCTCAAGCCG
GAGGTCAACAACGAGTCTTTTGTCATCTACATGTTCGTGGTCCACTTCAC
CATCCCCATGATTATCATCTTTTTCTGCTATGGGCAGCTCGTCTTCACCG
TCAAGGAGGCCGCTGCCCAGCAGCAGGAGTCAGCCACCACACAGAAGGCA
GAGAAGGAGGTCACCCGCATGGTCATCATCATGGTCATCGCTTTCCTGAT
CTGCTGGGTGCCCTACGCCAGCGTGGCATTCTACATCTTCACCCACCAGG
GCTCCAACTTCGGTCCCATCTTCATGACCATCCCAGCGTTCTTTGCCAAG
AGCGCCGCCATCTACAACCCTGTCATCTATATCATGATGAACAAGCAGTT
CCGGAACTGCATGCTCACCACCATCTGCTGCGGCAAGAACCCACTGGGTG
ACGATGAGGCCTCTGCTACCGTGTCCAAGACGGAGACGAGCCAGGTGGCC
CCGGCCTAAGCTGGAGCCTCGGTGGCCATGCTTCTTGCCCCTTGGGCCTC
CCCCCAGCCCCTCCTCCCCTTCCTGCACCCGTACCCCCGTGGTCTTTGAA
TAAAGTCTGAGTGGGCGGCA

Where a guide RNA is used that comprises a targeting domain that binds to a wild-type RHO sequence present in the RHO cDNA, a codon-modified version of the RHO cDNA may be substituted for the RHO cDNA comprised in the nucleic acid construct above.

Vector 1 and Vector 2 are packaged into viral particles according to methods known in the art, and delivered to the patient via subretinal injection at a dose of about 300 microliters of 1×10¹¹-3×10¹¹ viral genomes (vg)/mL. The patient is monitored post-administration, and periodically subjected to an assessment of one or more symptoms associated with adRP. For example, the patient is periodically subjected to an assessment of rod photoreceptor function, e.g., by scotopic microperimetry. About one year after administration of Vector 1 and Vector 2, the patient shows an amelioration of at least one adRP associated symptom, e.g. a stabilization of rod function, characterized by improved rod function compared to the expected level of rod function in the patient, or in an appropriate control group, in the absence of a clinical intervention.

TABLE 18
gRNAs Providing >0.1% Editing of RHO Alleles in
HEK293T Cells

| gRNA | | Targeting Domain(DNA)/ |
|---|---|---|
| ID | Targeting Domain (RNA) | Protospacer |
| RHO-1 | GUCAGCCACAAGGGCCACAGCC | GTCAGCCACAAGGGCCACAGCC |
| | (SEQ ID NO: 100) | (SEQ ID NO: 600) |
| RHO-2 | CCGAAGACGAAGUAUCCAUGCA | CCGAAGACGAAGTATCCATGCA |
| | (SEQ ID NO: 101) | (SEQ ID NO: 601) |
| RHO-3 | AGUAUCCAUGCAGAGAGGUGUA | AGTATCCATGCAGAGAGGTGTA |
| | (SEQ ID NO: 102) | (SEQ ID NO: 602) |
| RHO-4 | CUAGGUUGAGCAGGAUGUAGUU | CTAGGTTGAGCAGGATGTAGTT |
| | (SEQ ID NO: 103) | (SEQ ID NO: 603) |
| RHO-5 | CAUGGCUCAGCCAGGUAGUACU | CATGGCTCAGCCAGGTAGTACT |
| | (SEQ ID NO: 104) | (SEQ ID NO: 604) |
| RHO-6 | ACGGGUGUGGUACGCAGCCCCU | ACGGGTGTGGTACGCAGCCCCT |
| | (SEQ ID NO: 105) | (SEQ ID NO: 605) |
| RHO-7 | CCCACACCCGGCUCAUACCGCC | CCCACACCCGGCTCATACCGCC |
| | (SEQ ID NO: 106) | (SEQ ID NO: 606) |
| RHO-8 | CCCUGGGCGGUAUGAGCCGGGU | CCCTGGGCGGTATGAGCCGGGT |
| | (SEQ ID NO: 107) | (SEQ ID NO: 607) |
| RHO-9 | CCAUCAUGGGCGUUGCCUUCAC | CCATCATGGGCGTTGCCTTCAC |
| | (SEQ ID NO: 108) | (SEQ ID NO: 608) |
| RHO-10 | GUGCCAUUACCUGGACCAGCCG | GTGCCATTACCTGGACCAGCCG |
| | (SEQ ID NO: 109) | (SEQ ID NO: 609) |
| RHO-11 | UUACCUGGACCAGCCGGCGAGU | TTACCTGGACCAGCCGGCGAGT |
| | (SEQ ID NO: 110) | (SEQ ID NO: 610) |
| RHO-12 | GCAUUCUUGGGUGGGAGCAGCC | GCATTCTTGGGTGGGAGCAGCC |
| | (SEQ ID NO: 111) | (SEQ ID NO: 611) |
| RHO-13 | GCUCAGCCACUCAGGGCUCCAG | GCTCAGCCACTCAGGGCTCCAG |
| | (SEQ ID NO: 112) | (SEQ ID NO: 612) |
| RHO-14 | UGACCCGUGGCUGCUCCCACCC | TGACCCGTGGCTGCTCCCACCC |
| | (SEQ ID NO: 113) | (SEQ ID NO: 613) |
| RHO-15 | AGCUCAGGCCUUCGCAGCAUUC | AGCTCAGGCCTTCGCAGCATTC |
| | (SEQ ID NO: 114) | (SEQ ID NO: 614) |
| RHO-17 | ACACGCUGAGGAGAGCUGGGCA | ACACGCTGAGGAGAGCTGGGCA |
| | (SEQ ID NO: 116) | (SEQ ID NO: 616) |
| RHO-18 | GCAAAUAACUUCCCCCAUUCCC | GCAAATAACTTCCCCCATTCCC |
| | (SEQ ID NO: 117) | (SEQ ID NO: 617) |
| RHO-19 | AGACCCAGGCUGGGCACUGAGG | AGACCCAGGCTGGGCACTGAGG |
| | (SEQ ID NO: 118) | (SEQ ID NO: 618) |
| RHO-20 | CUAGGUCUCCUGGCUGUGAUCC | CTAGGTCTCCTGGCTGTGATCC |
| | (SEQ ID NO: 119) | (SEQ ID NO: 619) |
| RHO-21 | CCAGAAGGUGGGUGUGCCACUU | CCAGAAGGTGGGTGTGCCACTT |
| | (SEQ ID NO: 120) | (SEQ ID NO: 620) |
| RHO-24 | GGGCGUCACACAGGGACGGGUG | GGGCGTCACACAGGGACGGGTG |
| | (SEQ ID NO: 123) | (SEQ ID NO: 623) |
| RHO-25 | CUGUGAUCCAGGAAUAUCUCUG | CTGTGATCCAGGAATATCTCTG |
| | (SEQ ID NO: 124) | (SEQ ID NO: 624) |
| RHO-26 | UUGCAUUUAACAGGAAAACAGA | TTGCATTTAACAGGAAAACAGA |
| | (SEQ ID NO: 125) | (SEQ ID NO: 625) |
| RHO-27 | GGAGUGCACCCUCCUUAGGCAG | GGAGTGCACCCTCCTTAGGCAG |
| | (SEQ ID NO: 126) | (SEQ ID NO: 626) |
| RHO-28 | CAUCUGUCCUGCUCACCACCCC | CATCTGTCCTGCTCACCACCCC |
| | (SEQ ID NO: 127) | (SEQ ID NO: 627) |
| RHO-29 | GAGGGGAGGCAGAGGAUGCCAG | GAGGGGAGGCAGAGGATGCCAG |
| | (SEQ ID NO: 128) | (SEQ ID NO: 628) |
| RHO-30 | CUCAGGGAAUCUCUGGCCAUUG | CTCAGGGAATCTCTGGCCATTG |
| | (SEQ ID NO: 129) | (SEQ ID NO: 629) |
| RHO-31 | UGCACUCCCCCCUAGACAGGGA | TGCACTCCCCCCTAGACAGGGA |
| | (SEQ ID NO: 130) | (SEQ ID NO: 630) |
| RHO-32 | UGCUGUUUGUGCAGGGCUGGCA | TGCTGTTTGTGCAGGGCTGGCA |
| | (SEQ ID NO: 131) | (SEQ ID NO: 631) |
| RHO-33 | ACUGGGACAUUCCUAACAGUGA | ACTGGGACATTCCTAACAGTGA |
| | (SEQ ID NO: 132) | (SEQ ID NO: 632) |
| RHO-35 | CUCCUCUCUGGGGGCCCAAGCU | CTCCTCTCTGGGGGCCCAAGCT |
| | (SEQ ID NO: 134) | (SEQ ID NO: 634) |
| RHO-36 | CUGCAUCUCAGCAGAGAUAUUC | CTGCATCTCAGCAGAGATATTC |
| | (SEQ ID NO: 135) | (SEQ ID NO: 635) |
| RHO-37 | UGUUUCCCUUGGAGCAGCUGUG | TGTTTCCCTTGGAGCAGCTGTG |
| | (SEQ ID NO: 136) | (SEQ ID NO: 636) |
| RHO-40 | CCUAGGAGAGGCCCCCACAUGU | CCTAGGAGAGGCCCCCACATGT |
| | (SEQ ID NO: 139) | (SEQ ID NO: 639) |
| RHO-41 | AUCACUCAGUUCUGGCCAGAAG | ATCACTCAGTTCTGGCCAGAAG |
| | (SEQ ID NO: 140) | (SEQ ID NO: 640) |
| RHO-42 | AGAGCUGGGCAAAGAAAUUCCA | AGAGCTGGGCAAAGAAATTCCA |
| | (SEQ ID NO: 141) | (SEQ ID NO: 641) |
| RHO-43 | CCACCCCAUGAAGUUCCAUAGG | CCACCCCATGAAGTTCCATAGG |
| | (SEQ ID NO: 142) | (SEQ ID NO: 642) |
| RHO-44 | CCACCCUGAGCUUGGGCCCCCA | CCACCCTGAGCTTGGGCCCCCA |
| | (SEQ ID NO: 143) | (SEQ ID NO: 643) |
| RHO-45 | CAGAGGAAGAAGAAGGAAAUGA | CAGAGGAAGAAGAAGGAAATGA |
| | (SEQ ID NO: 144) | (SEQ ID NO: 644) |
| RHO-46 | AAACAGCAGCCCGGCUAUCACC | AAACAGCAGCCCGGCTATCACC |
| | (SEQ ID NO: 145) | (SEQ ID NO: 645) |
| RHO-49 | UCACACAGGGACGGGUGCAGAG | TCACACAGGGACGGGTGCAGAG |
| | (SEQ ID NO: 148) | (SEQ ID NO: 648) |
| RHO-51 | UGAGCUUGGGCCCCCAGAGAGG | TGAGCTTGGGCCCCCAGAGAGG |
| | (SEQ ID NO: 150) | (SEQ ID NO: 650) |
| RHO-52 | AAUAUCUCUGCUGAGAUGCAGG | AATATCTCTGCTGAGATGCAGG |
| | (SEQ ID NO: 151) | (SEQ ID NO: 651) |
| RHO-53 | GGAGAGGGGAAGAGACUCAUUU | GGAGAGGGGAAGAGACTCATTT |
| | (SEQ ID NO: 152) | (SEQ ID NO: 652) |
| RHO-54 | AGAACUGAGUGAUCUGUGAUUA | AGAACTGAGTGATCTGTGATTA |
| | (SEQ ID NO: 153) | (SEQ ID NO: 653) |
| RHO-55 | CCACUCUCCCUAUGGAACUUCA | CCACTCTCCCTATGGAACTTCA |
| | (SEQ ID NO: 154) | (SEQ ID NO: 654) |
| RHO-57 | UGGAUUUUCCAUUCUCCAGUCA | TGGATTTTCCATTCTCCAGTCA |
| | (SEQ ID NO: 156) | (SEQ ID NO: 656) |
| RHO-58 | GUGCAGGAGCCCGGGAGCAUGG | GTGCAGGAGCCCGGGAGCATGG |
| | (SEQ ID NO: 157) | (SEQ ID NO: 657) |
| RHO-59 | GGGUGGUGAGCAGGACAGAUGU | GGGTGGTGAGCAGGACAGATGT |
| | (SEQ ID NO: 158) | (SEQ ID NO: 658) |
| RHO-60 | CAGCUCUCCCUCAGUGCCCAGC | CAGCTCTCCCTCAGTGCCCAGC |
| | (SEQ ID NO: 159) | (SEQ ID NO: 659) |
| RHO-61 | CCUGCUGGGGCGUCACACAGGG | CCTGCTGGGGCGTCACACAGGG |
| | (SEQ ID NO: 160) | (SEQ ID NO: 660) |
| RHO-63 | ACUUACGGGUGGUUGUUCUCUG | ACTTACGGGTGGTTGTTCTCTG |
| | (SEQ ID NO: 162) | (SEQ ID NO: 662) |
| RHO-64 | CACAGGGAAGACCCAAUGACUG | CACAGGGAAGACCCAATGACTG |
| | (SEQ ID NO: 163) | (SEQ ID NO: 663) |
| RHO-65 | AGCACAGACCCCACUGCCUAAG | AGCACAGACCCCACTGCCTAAG |
| | (SEQ ID NO: 164) | (SEQ ID NO: 664) |
| RHO-66 | ACCUGAGGACAGGGGCUGAGAG | ACCTGAGGACAGGGGCTGAGAG |
| | (SEQ ID NO: 165) | (SEQ ID NO: 665) |
| RHO-67 | CAACAAUGGCCAGAGAUUCCCU | CAACAATGGCCAGAGATTCCCT |
| | (SEQ ID NO: 166) | (SEQ ID NO: 666) |
| RHO-68 | UGCUGCCUCGGUCCCAUUCUCA | TGCTGCCTCGGTCCCATTCTCA |
| | (SEQ ID NO: 167) | (SEQ ID NO: 667) |
| RHO-69 | UGCUGCCUGGCCACAUCCCUAA | TGCTGCCTGGCCACATCCCTAA |
| | (SEQ ID NO: 168) | (SEQ ID NO: 668) |
| RHO-70 | GCCACUCUCCCUAUGGAACUUC | GCCACTCTCCCTATGGAACTTC |
| | (SEQ ID NO: 169) | (SEQ ID NO: 669) |
| RHO-71 | GAGGGAGGAAGGACUGCCAAUU | GAGGGAGGAAGGACTGCCAATT |
| | (SEQ ID NO: 170) | (SEQ ID NO: 670) |
| RHO-72 | GAGGGUAGCUAGGAAGGCAACC | GAGGGTAGCTAGGAAGGCAACC |
| | (SEQ ID NO: 171) | (SEQ ID NO: 671) |
| RHO-73 | GGAAGGCAACCAGGAGUGGGAG | GGAAGGCAACCAGGAGTGGGAG |
| | (SEQ ID NO: 172) | (SEQ ID NO: 672) |
| RHO-74 | GCUGAGAUGCAGGAGGAGACGC | GCTGAGATGCAGGAGGAGACGC |
| | (SEQ ID NO: 173) | (SEQ ID NO: 673) |
| RHO-75 | AGGCUGGAGGGGCACCUGAGGA | AGGCTGGAGGGGCACCTGAGGA |
| | (SEQ ID NO: 174) | (SEQ ID NO: 674) |
| RHO-76 | AGGAAGGCAACCAGGAGUGGGA | AGGAAGGCAACCAGGAGTGGGA |
| | (SEQ ID NO: 175) | (SEQ ID NO: 675) |
| RHO-77 | CCGGGAGCAUGGAGGGGUCUGG | CCGGGAGCATGGAGGGGTCTGG |
| | (SEQ ID NO: 176) | (SEQ ID NO: 676) |
| RHO-78 | GGAUAACAGAUCCCACUUAACA | GGATAACAGATCCCACTTAACA |
| | (SEQ ID NO: 177) | (SEQ ID NO: 677) |
| RHO-79 | AGGCAGAGGAUGCCAGAGGGGA | AGGCAGAGGATGCCAGAGGGGA |
| | (SEQ ID NO: 178) | (SEQ ID NO: 678) |
| RHO-80 | GGGCCCAAGCUCAGGGUGGGAA | GGGCCCAAGCTCAGGGTGGGAA |
| | (SEQ ID NO: 179) | (SEQ ID NO: 679) |
| RHO-81 | UAACUAUAUGGCCACUCUCCCU | TAACTATATGGCCACTCTCCCT |
| | (SEQ ID NO: 180) | (SEQ ID NO: 680) |
| RHO-82 | UCCCACUUAACAGAGAGGAAAA | TCCCACTTAACAGAGAGGAAAA |
| | (SEQ ID NO: 181) | (SEQ ID NO: 681) |
| RHO-83 | GAAUGCAGAGGUGGUGGAAACC | GAATGCAGAGGTGGTGGAAACC |
| | (SEQ ID NO: 182) | (SEQ ID NO: 682) |
| RHO-84 | GGGAGACAGGGCAAGGCUGGCA | GGGAGACAGGGCAAGGCTGGCA |
| | (SEQ ID NO: 183) | (SEQ ID NO: 683) |
| RHO-85 | CACCACCCCAUGAAGUUCCAUA | CACCACCCCATGAAGTTCCATA |
| | (SEQ ID NO: 184) | (SEQ ID NO: 684) |
| RHO-86 | GCCAUAUAGUUAAUCAACCAAA | GCCATATAGTTAATCAACCAAA |
| | (SEQ ID NO: 185) | (SEQ ID NO: 685) |
| RHO-87 | GUAGCUAGGAAGGCAACCAGGA | GTAGCTAGGAAGGCAACCAGGA |
| | (SEQ ID NO: 186) | (SEQ ID NO: 686) |
| RHO-88 | CACAUUGCUUCAUGGCUCCUAG | CACATTGCTTCATGGCTCCTAG |
| | (SEQ ID NO:187) | (SEQ ID NO: 687) |
| RHO-89 | CUGAGCUUGGGCCCCCAGAGAG | CTGAGCTTGGGCCCCCAGAGAG |
| | (SEQ ID NO:188) | (SEQ ID NO: 688) |
| RHO-90 | ACCGAGCCCAUUGCCCAGCACA | ACCGAGCCCATTGCCCAGCACA |
| | (SEQ ID NO: 189) | (SEQ ID NO: 689) |
| RHO-91 | CUCAAAGAAGUCAAGCGCCCUG | CTCAAAGAAGTCAAGCGCCCTG |
| | (SEQ ID NO: 190) | (SEQ ID NO: 690) |
| RHO-92 | GCUACCCUCUCCCUGUCUAGGG | GCTACCCTCTCCCTGTCTAGGG |
| | (SEQ ID NO: 191) | (SEQ ID NO: 691) |
| RHO-93 | ACCCUGAGCUUGGGCCCCCAGA | ACCCTGAGCTTGGGCCCCCAGA |
| | (SEQ ID NO: 192) | (SEQ ID NO: 692) |
| RHO-94 | GGCAGAGGGACCACACGCUGAG | GGCAGAGGGACCACACGCTGAG |
| | (SEQ ID NO: 193) | (SEQ ID NO: 693) |
| RHO-95 | UCUGACUCAGCACAGCUGCUCC | TCTGACTCAGCACAGCTGCTCC |
| | (SEQ ID NO: 194) | (SEQ ID NO: 694) |
| RHO-96 | CUCUCAGCCACCACCGCCAAGC | CTCTCAGCCACCACCGCCAAGC |
| | (SEQ ID NO: 195) | (SEQ ID NO: 695) |
| RHO-97 | AGGGAUGUGGCCAGGCAGCAAC | AGGGATGTGGCCAGGCAGCAAC |
| | (SEQ ID NO: 196) | (SEQ ID NO: 696) |
| RHO-98 | CACCUGAGGACAGGGGCUGAGA | CACCTGAGGACAGGGGCTGAGA |
| | (SEQ ID NO: 197) | (SEQ ID NO: 697) |
| RHO-99 | GCCCAUGAUGGCAUGGUUCUCC | GCCCATGATGGCATGGTTCTCC |
| | (SEQ ID NO: 198) | (SEQ ID NO: 698) |
| RHO-100 | GAAGGGGCAGAGGGACCACACG | GAAGGGGCAGAGGGACCACACG |
| | (SEQ ID NO: 199) | (SEQ ID NO: 699) |
| RHO-101 | AGCACCCUCUACACCUCUCUGC | AGCACCCTCTACACCTCTCTGC |
| | (SEQ ID NO: 200) | (SEQ ID NO: 700) |
| RHO-102 | CUUUGGAUAACAUUGACAGGAC | CTTTGGATAACATTGACAGGAC |
| | (SEQ ID NO: 201) | (SEQ ID NO: 701) |
| RHO-103 | GGUGAAGCCACCUAGGACCAUG | GGTGAAGCCACCTAGGACCATG |
| | (SEQ ID NO: 202) | (SEQ ID NO: 702) |
| RHO-104 | UAACAUUGACAGGACAGGAGAA | TAACATTGACAGGACAGGAGAA |
| | (SEQ ID NO: 203) | (SEQ ID NO: 703) |
| RHO-105 | GGGAGAGGGGAAGAGACUCAUU | GGGAGAGGGGAAGAGACTCATT |
| | (SEQ ID NO: 204) | (SEQ ID NO: 704) |
| RHO-106 | GCUGUGCUGAGUCAGACCCAGG | GCTGTGCTGAGTCAGACCCAGG |
| | (SEQ ID NO: 205) | (SEQ ID NO: 705) |
| RHO-107 | UUGAGGAGGCCUUGGGGAAGGA | TTGAGGAGGCCTTGGGGAAGGA |
| | (SEQ ID NO: 206) | (SEQ ID NO: 706) |
| RHO-108 | GCCCGGGAGCAUGGAGGGGUCU | GCCCGGGAGCATGGAGGGGTCT |
| | (SEQ ID NO: 207) | (SEQ ID NO: 707) |
| RHO-109 | GUAAACUGGGACUGACCCUGCA | GTAAACTGGGACTGACCCTGCA |
| | (SEQ ID NO: 208) | (SEQ ID NO: 708) |
| RHO-110 | AUAACAUUGACAGGACAGGAGA | ATAACATTGACAGGACAGGAGA |
| | (SEQ ID NO: 209) | (SEQ ID NO: 709) |
| RHO-111 | GGCAGGGAGGCUGGAGGGGCAC | GGCAGGGAGGCTGGAGGGGCAC |
| | (SEQ ID NO: 210) | (SEQ ID NO: 710) |
| RHO-112 | GCAAACAUGGCCCGAGAUAGAU | GCAAACATGGCCCGAGATAGAT |
| | (SEQ ID NO: 211) | (SEQ ID NO: 711) |
| RHO-113 | GGACCGAGCCCAUUGCCCAGCA | GGACCGAGCCCATTGCCCAGCA |
| | (SEQ ID NO: 212) | (SEQ ID NO: 712) |
| RHO-114 | GCUCUACGUCACCGUCCAGCAC | GCTCTACGTCACCGTCCAGCAC |
| | (SEQ ID NO: 213) | (SEQ ID NO: 713) |
| RHO-115 | AGCACAGCUGCUCCAAGGGAAA | AGCACAGCTGCTCCAAGGGAAA |
| | (SEQ ID NO: 214) | (SEQ ID NO: 714) |
| RHO-116 | CUAAAGCAAAAAGGAACUGCUU | CTAAAGCAAAAAGGAACTGCTT |
| | (SEQ ID NO: 215) | (SEQ ID NO: 715) |
| RHO-117 | GAGAGGAAAACUGAGGCAGGGA | GAGAGGAAAACTGAGGCAGGGA |
| | (SEQ ID NO: 216) | (SEQ ID NO: 716) |
| RHO-118 | CAUUGCAAAGCUGGGUGACGGG | CATTGCAAAGCTGGGTGACGGG |
| | (SEQ ID NO: 217) | (SEQ ID NO: 717) |
| RHO-119 | UUGCCACCCUGGGCGGUAUGAG | TTGCCACCCTGGGCGGTATGAG |
| | (SEQ ID NO: 218) | (SEQ ID NO: 718) |
| RHO-120 | AGCUAGGAAGGCAACCAGGAGU | AGCTAGGAAGGCAACCAGGAGT |
| | (SEQ ID NO: 219) | (SEQ ID NO: 719) |
| RHO-121 | UCUCUGGGGGCCCAAGCUCAGG | TCTCTGGGGGCCCAAGCTCAGG |
| | (SEQ ID NO: 220) | (SEQ ID NO: 720) |
| RHO-122 | AGCACAGGGAAGACCCAAUGAC | AGCACAGGGAAGACCCAATGAC |
| | (SEQ ID NO: 221) | (SEQ ID NO: 721) |
| RHO-123 | GUUGACUGAAUAUAUGAGGGCU | GTTGACTGAATATATGAGGGCT |
| | (SEQ ID NO: 222) | (SEQ ID NO: 722) |
| RHO-124 | UUGUAAACUGGGACUGACCCUG | TTGTAAACTGGGACTGACCCTG |
| | (SEQ ID NO: 223) | (SEQ ID NO: 723) |
| RHO-125 | CACACCCACCUUCUGGCCAGAA | CACACCCACCTTCTGGCCAGAA |
| | (SEQ ID NO: 224) | (SEQ ID NO: 724) |
| RHO-126 | CCAGAGGAAGAAGAAGGAAAUG | CCAGAGGAAGAAGAAGGAAATG |
| | (SEQ ID NO: 225) | (SEQ ID NO: 725) |
| RHO-127 | GAGAUAUUCCUGGAUCACAGCC | GAGATATTCCTGGATCACAGCC |
| | (SEQ ID NO: 226) | (SEQ ID NO: 726) |
| RHO-128 | AGGGGCAGAGGGACCACACGCU | AGGGGCAGAGGGACCACACGCT |
| | (SEQ ID NO: 227) | (SEQ ID NO: 727) |
| RHO-129 | AACUAUAUGGCCACUCUCCCUA | AACTATATGGCCACTCTCCCTA |
| | (SEQ ID NO: 228) | (SEQ ID NO: 728) |
| RHO-130 | GCUGCUUGCGGUUCUCAACACC | GCTGCTTGCGGTTCTCAACACC |
| | (SEQ ID NO: 229) | (SEQ ID NO: 729) |
| RHO-131 | CACCAUGAAUGGUGUUUGUUGA | CACCATGAATGGTGTTTGTTGA |
| | (SEQ ID NO: 230) | (SEQ ID NO: 730) |
| RHO-132 | GCAGCCAUUGCAAAGCUGGGUG | GCAGCCATTGCAAAGCTGGGTG |
| | (SEQ ID NO: 231) | (SEQ ID NO: 731) |
| RHO-133 | UGACUCAGCACAGCUGCUCCAA | TGACTCAGCACAGCTGCTCCAA |
| | (SEQ ID NO: 232) | (SEQ ID NO: 732) |
| RHO-134 | CUGGGAGGAGGGGGAAGGGGCA | CTGGGAGGAGGGGGAAGGGGCA |
| | (SEQ ID NO: 233) | (SEQ ID NO: 733) |
| RHO-135 | GAUAACAUUGACAGGACAGGAG | GATAACATTGACAGGACAGGAG |
| | (SEQ ID NO: 234) | (SEQ ID NO: 734) |
| RHO-136 | CCAAACUGGGACAUUCCUAACA | CCAAACTGGGACATTCCTAACA |
| | (SEQ ID NO: 235) | (SEQ ID NO: 735) |
| RHO-137 | AGGAAAACAGAUGGGGUGCUGC | AGGAAAACAGATGGGGTGCTGC |
| | (SEQ ID NO: 236) | (SEQ ID NO: 736) |
| RHO-138 | CGGACAUGUGGGGGCCUCUCCU | CGGACATGTGGGGGCCTCTCCT |
| | (SEQ ID NO: 237) | (SEQ ID NO: 737) |
| RHO-139 | GCAAAGAAAUUCCAGGGAAUGG | GCAAAGAAATTCCAGGGAATGG |
| | (SEQ ID NO: 238) | (SEQ ID NO: 738) |
| RHO-140 | CCAGGAGACUUGGAACGCGGCA | CCAGGAGACTTGGAACGCGGCA |
| | (SEQ ID NO: 239) | (SEQ ID NO: 739) |
| RHO-141 | UGGUCCUUGGUGGUCCUGGCCA | TGGTCCTTGGTGGTCCTGGCCA |
| | (SEQ ID NO: 240) | (SEQ ID NO: 740) |
| RHO-142 | AAUGGAAAAUCCACUUCCCACC | AATGGAAAATCCACTTCCCACC |
| | (SEQ ID NO: 241) | (SEQ ID NO: 741) |
| RHO-143 | GCCCGAAGACGAAGUAUCCAUG | GCCCGAAGACGAAGTATCCATG |
| | (SEQ ID NO: 242) | (SEQ ID NO: 742) |
| RHO-144 | GUGCUGGACGGUGACGUAGAGC | GTGCTGGACGGTGACGTAGAGC |
| | (SEQ ID NO: 243) | (SEQ ID NO: 743) |
| RHO-145 | AGAAACAUGUAGGCGGCCAGCA | AGAAACATGTAGGCGGCCAGCA |
| | (SEQ ID NO: 244) | (SEQ ID NO: 744) |
| RHO-146 | CCGCUCGAUGGCCAGGACCACC | CCGCTCGATGGCCAGGACCACC |
| | (SEQ ID NO: 245) | (SEQ ID NO: 745) |
| RHO-147 | UCAGCACAGACCCCACUGCCUA | TCAGCACAGACCCCACTGCCTA |
| | (SEQ ID NO: 246) | (SEQ ID NO: 746) |
| RHO-148 | GAAUAUCUCUGCUGAGAUGCAG | GAATATCTCTGCTGAGATGCAG |
| | (SEQ ID NO: 247) | (SEQ ID NO: 747) |
| RHO-149 | GAGUACCCACAGUACUACCUGG | GAGTACCCACAGTACTACCTGG |
| | (SEQ ID NO: 248) | (SEQ ID NO: 748) |
| RHO-150 | CAACCAGGAGUGGGAGAGGGAU | CAACCAGGAGTGGGAGAGGGAT |
| | (SEQ ID NO: 249) | (SEQ ID NO: 749) |
| RHO-151 | UUGAGAACCGCAAGCAGCCGCU | TTGAGAACCGCAAGCAGCCGCT |
| | (SEQ ID NO: 250) | (SEQ ID NO: 750) |
| RHO-152 | GCAAGCCAGACCCCUCCUCUCU | GCAAGCCAGACCCCTCCTCTCT |
| | (SEQ ID NO: 251) | (SEQ ID NO: 751) |
| RHO-153 | GAGAGCUGGGCAAAGAAAUUCC | GAGAGCTGGGCAAAGAAATTCC |
| | (SEQ ID NO: 252) | (SEQ ID NO: 752) |
| RHO-154 | CGAGGCAGCAGCCUGGACAUGG | CGAGGCAGCAGCCTGGACATGG |
| | (SEQ ID NO: 253) | (SEQ ID NO: 753) |
| RHO-155 | AGGAAUAUCUCUGCUGAGAUGC | AGGAATATCTCTGCTGAGATGC |
| | (SEQ ID NO: 254) | (SEQ ID NO: 754) |
| RHO-156 | UUCCCGAGAAGGGAGAGGGAGG | TTCCCGAGAAGGGAGAGGGAGG |
| | (SEQ ID NO: 255) | (SEQ ID NO: 755) |
| RHO-157 | UCCUUCCUCCCUCUCCCUUCUC | TCCTTCCTCCCTCTCCCTTCTC |
| | (SEQ ID NO: 256) | (SEQ ID NO: 756) |
| RHO-158 | UGUUUUGCCCAGAGGAAGAAGA | TGTTTTGCCCAGAGGAAGAAGA |
| | (SEQ ID NO: 257) | (SEQ ID NO: 757) |
| RHO-159 | CCGGCUGGUCCAGGUAAUGGCA | CCGGCTGGTCCAGGTAATGGCA |
| | (SEQ ID NO: 258) | (SEQ ID NO: 758) |
| RHO-160 | CAGCACAGGGAAGACCCAAUGA | CAGCACAGGGAAGACCCAATGA |
| | (SEQ ID NO: 259) | (SEQ ID NO: 759) |
| RHO-161 | ACCAGGAGUGGGAGAGGGAUUU | ACCAGGAGTGGGAGAGGGATTT |
| | (SEQ ID NO: 260) | (SEQ ID NO: 760) |
| RHO-162 | GCUGGUGAAGCCACCUAGGACC | GCTGGTGAAGCCACCTAGGACC |
| | (SEQ ID NO: 261) | (SEQ ID NO: 761) |
| RHO-163 | GGCGGUAUGAGCCGGGUGUGGG | GGCGGTATGAGCCGGGTGTGGG |
| | (SEQ ID NO: 262) | (SEQ ID NO: 762) |
| RHO-164 | CAGCCAUUGCAAAGCUGGGUGA | CAGCCATTGCAAAGCTGGGTGA |
| | (SEQ ID NO: 263) | (SEQ ID NO: 763) |
| RHO-165 | ACAUUGACAGGACAGGAGAAGG | ACATTGACAGGACAGGAGAAGG |
| | (SEQ ID NO: 264) | (SEQ ID NO: 764) |
| RHO-166 | UGGGUCUUCCCUGUGCUGGGCA | TGGGTCTTCCCTGTGCTGGGCA |
| | (SEQ ID NO: 265) | (SEQ ID NO: 765) |
| RHO-167 | GUACGUGGUGGUGUGUAAGCCC | GTACGTGGTGGTGTGTAAGCCC |
| | (SEQ ID NO: 266) | (SEQ ID NO: 766) |
| RHO-168 | AGCAAAUAACUUCCCCCAUUCC | AGCAAATAACTTCCCCCATTCC |
| | (SEQ ID NO: 267) | (SEQ ID NO: 767) |
| RHO-169 | GGAUUUGAGGAGGCCUUGGGGA | GGATTTGAGGAGGCCTTGGGGA |
| | (SEQ ID NO: 268) | (SEQ ID NO: 768) |
| RHO-170 | CCCUGAGCUUGGGCCCCCAGAG | CCCTGAGCTTGGGCCCCCAGAG |
| | (SEQ ID NO: 269) | (SEQ ID NO: 769) |
| RHO-171 | CAGAGAUUCCCUGAGAAUGGGA | CAGAGATTCCCTGAGAATGGGA |
| | (SEQ ID NO: 270) | (SEQ ID NO: 770) |
| RHO-172 | GAGUUGGAAGCCCGCAUCUAUC | GAGTTGGAAGCCCGCATCTATC |
| | (SEQ ID NO: 271) | (SEQ ID NO: 771) |
| RHO-173 | AGUCCUUCCUCCCUCUCCCUUC | AGTCCTTCCTCCCTCTCCCTTC |
| | (SEQ ID NO: 272) | (SEQ ID NO: 772) |
| RHO-174 | GUUAUUUCAUUUCCCGAGAAGG | GTTATTTCATTTCCCGAGAAGG |
| | (SEQ ID NO: 273) | (SEQ ID NO: 773) |
| RHO-175 | AUUUCAUUUCCCGAGAAGGGAG | ATTTCATTTCCCGAGAAGGGAG |
| | (SEQ ID NO: 274) | (SEQ ID NO: 774) |
| RHO-176 | GACGUAGAGCGUGAGGAAGUUG | GACGTAGAGCGTGAGGAAGTTG |
| | (SEQ ID NO: 275) | (SEQ ID NO: 775) |
| RHO-177 | CAUUUCCCGAGAAGGGAGAGGG | CATTTCCCGAGAAGGGAGAGGG |
| | (SEQ ID NO: 276) | (SEQ ID NO: 776) |
| RHO-178 | GUAGAGCGUGAGGAAGUUGAUG | GTAGAGCGTGAGGAAGTTGATG |
| | (SEQ ID NO: 277) | (SEQ ID NO: 777) |
| RHO-179 | CAGGCCUUCGCAGCAUUCUUGG | CAGGCCTTCGCAGCATTCTTGG |
| | (SEQ ID NO: 278) | (SEQ ID NO: 778) |
| RHO-180 | AGGUAGUACUGUGGGUACUCGA | AGGTAGTACTGTGGGTACTCGA |
| | (SEQ ID NO: 279) | (SEQ ID NO: 779) |
| RHO-181 | AAACAUGUAGGCGGCCAGCAUG | AAACATGTAGGCGGCCAGCATG |
| | (SEQ ID NO: 280) | (SEQ ID NO: 780) |
| RHO-182 | UUUCAUUUCCCGAGAAGGGAGA | TTTCATTTCCCGAGAAGGGAGA |
| | (SEQ ID NO: 281) | (SEQ ID NO: 781) |
| RHO-183 | GGGAAGACCCAAUGACUGGAGA | GGGAAGACCCAATGACTGGAGA |
| | (SEQ ID NO: 282) | (SEQ ID NO: 782) |
| RHO-184 | AAAACUGAGGCAGGGAGAGGGG | AAAACTGAGGCAGGGAGAGGGG |
| | (SEQ ID NO: 283) | (SEQ ID NO: 783) |
| RHO-185 | UGAGUCAGACCCAGGCUGGGCA | TGAGTCAGACCCAGGCTGGGCA |
| | (SEQ ID NO: 284) | (SEQ ID NO: 784) |
| RHO-186 | GGGAUUUGAGGAGGCCUUGGGG | GGGATTTGAGGAGGCCTTGGGG |
| | (SEQ ID NO: 285) | (SEQ ID NO: 785) |
| RHO-187 | UCUGGGGGCCCAAGCUCAGGGU | TCTGGGGGCCCAAGCTCAGGGT |
| | (SEQ ID NO: 286) | (SEQ ID NO: 786) |
| RHO-188 | CGGGCCCACAGGAUGCAAUUUG | CGGGCCCACAGGATGCAATTTG |
| | (SEQ ID NO: 287) | (SEQ ID NO: 787) |
| RHO-189 | ACGUAGAGCGUGAGGAAGUUGA | ACGTAGAGCGTGAGGAAGTTGA |
| | (SEQ ID NO: 288) | (SEQ ID NO: 788) |
| RHO-190 | GACCGAGGCAGCAGCCUGGACA | GACCGAGGCAGCAGCCTGGACA |
| | (SEQ ID NO: 289) | (SEQ ID NO: 789) |
| RHO-191 | CAGGCUGGGCACUGAGGGAGAG | CAGGCTGGGCACTGAGGGAGAG |
| | (SEQ ID NO: 290) | (SEQ ID NO: 790) |
| RHO-192 | UAUUUCAUUUCCCGAGAAGGGA | TATTTCATTTCCCGAGAAGGGA |
| | (SEQ ID NO: 291) | (SEQ ID NO: 791) |
| RHO-193 | GUCCCGGGCUUGGCGGUGGUGG | GTCCCGGGCTTGGCGGTGGTGG |
| | (SEQ ID NO: 292) | (SEQ ID NO: 792) |
| RHO-194 | CUGCUGCCUCGGUCCCAUUCUC | CTGCTGCCTCGGTCCCATTCTC |
| | (SEQ ID NO: 293) | (SEQ ID NO: 793) |
| RHO-195 | AGCGUCUCCUCCUGCAUCUCAG | AGCGTCTCCTCCTGCATCTCAG |
| | (SEQ ID NO: 294) | (SEQ ID NO: 794) |
| RHO-196 | UCAGACCCAGGCUGGGCACUGA | TCAGACCCAGGCTGGGCACTGA |
| | (SEQ ID NO: 295) | (SEQ ID NO: 795) |
| RHO-197 | AGCUACCCUCUCCCUGUCUAGG | AGCTACCCTCTCCCTGTCTAGG |
| | (SEQ ID NO: 296) | (SEQ ID NO: 796) |
| RHO-198 | CAGAGAGGAAAACUGAGGCAGG | CAGAGAGGAAAACTGAGGCAGG |
| | (SEQ ID NO: 297) | (SEQ ID NO: 797) |
| RHO-199 | GGAGAGGGAUUUGAGGAGGCCU | GGAGAGGGATTTGAGGAGGCCT |
| | (SEQ ID NO: 298) | (SEQ ID NO: 798) |
| RHO-200 | GUCCUUCCUCCCUCUCCCUUCU | GTCCTTCCTCCCTCTCCCTTCT |
| | (SEQ ID NO: 299) | (SEQ ID NO: 799) |
| RHO-201 | AGAGAGCUUGGUGCUGGGAGGA | AGAGAGCTTGGTGCTGGGAGGA |
| | (SEQ ID NO: 300) | (SEQ ID NO: 800) |
| RHO-202 | CCUUCUCGGGAAAUGAAAUAAC | CCTTCTCGGGAAATGAAATAAC |
| | (SEQ ID NO: 301) | (SEQ ID NO: 801) |
| RHO-203 | GCGGUUCUCAACACCAGGAGAC | GCGGTTCTCAACACCAGGAGAC |
| | (SEQ ID NO: 302) | (SEQ ID NO: 802) |
| RHO-204 | CUCUGGGGGCCCAAGCUCAGGG | CTCTGGGGGCCCAAGCTCAGGG |
| | (SEQ ID NO: 303) | (SEQ ID NO: 803) |
| RHO-205 | UGUGCAGGAGCCCGGGAGCAUG | TGTGCAGGAGCCCGGGAGCATG |
| | (SEQ ID NO: 304) | (SEQ ID NO: 804) |
| RHO-206 | CAGAGAGGUGUAGAGGGUGCUG | CAGAGAGGTGTAGAGGGTGCTG |
| | (SEQ ID NO: 305) | (SEQ ID NO: 805) |
| RHO-207 | CUCCCCGAAGCGGAAGUUGCUC | CTCCCCGAAGCGGAAGTTGCTC |
| | (SEQ ID NO: 306) | (SEQ ID NO: 806) |
| RHO-208 | GCUAGAAGCAGCCAUUGCAAAG | GCTAGAAGCAGCCATTGCAAAG |
| | (SEQ ID NO: 307) | (SEQ ID NO: 807) |
| RHO-209 | CAAACACCAUUCAUGGUGAUAG | CAAACACCATTCATGGTGATAG |
| | (SEQ ID NO: 308) | (SEQ ID NO: 808) |
| RHO-210 | UCAUUUCCCGAGAAGGGAGAGG | TCATTTCCCGAGAAGGGAGAGG |
| | (SEQ ID NO: 309) | (SEQ ID NO: 809) |
| RHO-211 | UCACCACCCCAUGAAGUUCCAU | TCACCACCCCATGAAGTTCCAT |
| | (SEQ ID NO: 310) | (SEQ ID NO: 810) |
| RHO-212 | GGGAGUGCACCCUCCUUAGGCA | GGGAGTGCACCCTCCTTAGGCA |
| | (SEQ ID NO: 311) | (SEQ ID NO: 811) |
| RHO-213 | AAUGGCCAGAGAUUCCCUGAGA | AATGGCCAGAGATTCCCTGAGA |
| | (SEQ ID NO: 312) | (SEQ ID NO: 812) |
| RHO-214 | AGAAUGGGACCGAGGCAGCAGC | AGAATGGGACCGAGGCAGCAGC |
| | (SEQ ID NO: 313) | (SEQ ID NO: 813) |
| RHO-215 | GGCAAGCCAGACCCCUCCUCUC | GGCAAGCCAGACCCCTCCTCTC |
| | (SEQ ID NO: 314) | (SEQ ID NO: 814) |
| RHO-216 | CCCGGGCUUGGCGGUGGUGGCU | CCCGGGCTTGGCGGTGGTGGCT |
| | (SEQ ID NO: 315) | (SEQ ID NO: 815) |
| RHO-217 | AGCCCGGGAGCAUGGAGGGGUC | AGCCCGGGAGCATGGAGGGGTC |
| | (SEQ ID NO: 316) | (SEQ ID NO: 816) |
| RHO-218 | CCGGGUUAUUUCAUUUCCCGAG | CCGGGTTATTTCATTTCCCGAG |
| | (SEQ ID NO: 317) | (SEQ ID NO: 817) |
| RHO-219 | GGUGUUUGUUGACUGAAUAUAU | GGTGTTTGTTGACTGAATATAT |
| | (SEQ ID NO: 318) | (SEQ ID NO: 818) |
| RHO-220 | CCGUCCCUGUGUGACGCCCCAG | CCGTCCCTGTGTGACGCCCCAG |
| | (SEQ ID NO: 319) | (SEQ ID NO: 819) |
| RHO-221 | GGACAGGGGCUGAGAGGGGAGG | GGACAGGGGCTGAGAGGGGAGG |
| | (SEQ ID NO: 320) | (SEQ ID NO: 820) |
| RHO-222 | AGAGGGUGCUGGUGAAGCCACC | AGAGGGTGCTGGTGAAGCCACC |
| | (SEQ ID NO: 321) | (SEQ ID NO: 821) |
| RHO-223 | AUUGCAUCCUGUGGGCCCGAAG | ATTGCATCCTGTGGGCCCGAAG |
| | (SEQ ID NO: 322) | (SEQ ID NO: 822) |
| RHO-224 | CGGGUUAUUUCAUUUCCCGAGA | CGGGTTATTTCATTTCCCGAGA |
| | (SEQ ID NO: 323) | (SEQ ID NO: 823) |
| RHO-225 | GGAAAUGAAAUAACCCGGACAU | GGAAATGAAATAACCCGGACAT |
| | (SEQ ID NO: 324) | (SEQ ID NO: 824) |
| RHO-226 | CUGACUCAGCACAGCUGCUCCA | CTGACTCAGCACAGCTGCTCCA |
| | (SEQ ID NO: 325) | (SEQ ID NO: 825) |
| RHO-227 | GGCACCUGAGGACAGGGGCUGA | GGCACCTGAGGACAGGGGCTGA |
| | (SEQ ID NO: 326) | (SEQ ID NO: 826) |
| RHO-228 | GGAGAGCUGGGCAAAGAAAUUC | GGAGAGCTGGGCAAAGAAATTC |
| | (SEQ ID NO: 327) | (SEQ ID NO: 827) |
| RHO-229 | GGGCGGUAUGAGCCGGGUGUGG | GGGCGGTATGAGCCGGGTGTGG |
| | (SEQ ID NO: 328) | (SEQ ID NO: 828) |
| RHO-230 | CCUCCCUCUCCCUUCUCGGGAA | CCTCCCTCTCCCTTCTCGGGAA |
| | (SEQ ID NO: 329) | (SEQ ID NO: 829) |
| RHO-231 | UCCAGGUAAUGGCACUGAGCAG | TCCAGGTAATGGCACTGAGCAG |
| | (SEQ ID NO: 330) | (SEQ ID NO: 830) |
| RHO-232 | GUGGGGGCCUCUCCUAGGAGCC | GTGGGGGCCTCTCCTAGGAGCC |
| | (SEQ ID NO: 331) | (SEQ ID NO: 831) |
| RHO-233 | GAUGGCAUGGUUCUCCCCGAAG | GATGGCATGGTTCTCCCCGAAG |
| | (SEQ ID NO: 332) | (SEQ ID NO: 832) |
| RHO-234 | CGUCGCAUUGGAGAAGGGCACG | CGTCGCATTGGAGAAGGGCACG |
| | (SEQ ID NO: 333) | (SEQ ID NO: 833) |
| RHO-235 | UGGGUGGGGUGUGCAGGAGCCC | TGGGTGGGGTGTGCAGGAGCCC |
| | (SEQ ID NO: 334) | (SEQ ID NO: 834) |
| RHO-236 | CUGGACGGUGACGUAGAGCGUG | CTGGACGGTGACGTAGAGCGTG |
| | (SEQ ID NO: 335) | (SEQ ID NO: 835) |
| RHO-237 | GAGGAAAACUGAGGCAGGGAGA | GAGGAAAACTGAGGCAGGGAGA |
| | (SEQ ID NO: 336) | (SEQ ID NO: 836) |
| RHO-238 | CUGAACACUGCCUUGAUCUUAU | CTGAACACTGCCTTGATCTTAT |
| | (SEQ ID NO: 337) | (SEQ ID NO: 837) |
| RHO-239 | CAUUACCUGGACCAGCCGGCGA | CATTACCTGGACCAGCCGGCGA |
| | (SEQ ID NO: 338) | (SEQ ID NO: 838) |
| RHO-240 | GGAGAGAGCUUGGUGCUGGGAG | GGAGAGAGCTTGGTGCTGGGAG |
| | (SEQ ID NO: 339) | (SEQ ID NO: 839) |
| RHO-241 | AGAAUAAUGUCUUGCAUUUAAC | AGAATAATGTCTTGCATTTAAC |
| | (SEQ ID NO: 340) | (SEQ ID NO: 840) |
| RH0-242 | CUAGGAAGGCAACCAGGAGUGG | CTAGGAAGGCAACCAGGAGTGG |
| | (SEQ ID NO: 341) | (SEQ ID NO: 841) |
| RHO-243 | UCUCCCAGACCCCUCCAUGCUC | TCTCCCAGACCCCTCCATGCTC |
| | (SEQ ID NO: 342) | (SEQ ID NO: 842) |
| RH0-244 | ACAGGGGCUGAGAGGGGAGGCA | ACAGGGGCTGAGAGGGGAGGCA |
| | (SEQ ID NO: 343) | (SEQ ID NO: 843) |
| RHO-245 | GGGGCAGAGGGACCACACGCUG | GGGGCAGAGGGACCACACGCTG |
| | (SEQ ID NO: 344) | (SEQ ID NO: 844) |
| RHO-246 | AGGGGAGGCAGAGGAUGCCAGA | AGGGGAGGCAGAGGATGCCAGA |
| | (SEQ ID NO: 345) | (SEQ ID NO: 845) |
| RHO-247 | UGGUCCAGGUAAUGGCACUGAG | TGGTCCAGGTAATGGCACTGAG |
| | (SEQ ID NO: 346) | (SEQ ID NO: 846) |
| RHO-248 | CCGGACAUGUGGGGGCCUCUCC | CCGGACATGTGGGGGCCTCTCC |
| | (SEQ ID NO: 347) | (SEQ ID NO: 847) |
| RHO-249 | GCAGGCCAGCGCCAUGACCCAG | GCAGGCCAGCGCCATGACCCAG |
| | (SEQ ID NO: 348) | (SEQ ID NO: 848) |
| RHO-250 | CUAGCUACCCUCUCCCUGUCUA | CTAGCTACCCTCTCCCTGTCTA |
| | (SEQ ID NO: 349) | (SEQ ID NO: 849) |
| RHO-251 | GCUUUGGAUAACAUUGACAGGA | GCTTTGGATAACATTGACAGGA |
| | (SEQ ID NO: 350) | (SEQ ID NO: 850) |
| RHO-252 | GCCAUUGCAAAGCUGGGUGACG | GCCATTGCAAAGCTGGGTGACG |
| | (SEQ ID NO: 351) | (SEQ ID NO: 851) |
| RHO-253 | CCUAGGUCUCCUGGCUGUGAUC | CCTAGGTCTCCTGGCTGTGATC |
| | (SEQ ID NO: 352) | (SEQ ID NO: 852) |
| RHO-254 | AACAGAGAGGAAAACUGAGGCA | AACAGAGAGGAAAACTGAGGCA |
| | (SEQ ID NO: 353) | (SEQ ID NO: 853) |
| RHO-255 | AUUACCUGGACCAGCCGGCGAG | ATTACCTGGACCAGCCGGCGAG |
| | (SEQ ID NO: 354) | (SEQ ID NO: 854) |
| RHO-256 | GAGGGGCACCUGAGGACAGGGG | GAGGGGCACCTGAGGACAGGGG |
| | (SEQ ID NO: 355) | (SEQ ID NO: 855) |
| RHO-257 | GGGUUAUUUCAUUUCCCGAGAA | GGGTTATTTCATTTCCCGAGAA |
| | (SEQ ID NO: 356) | (SEQ ID NO: 856) |
| RHO-258 | AGGGUGCACUCCCCCCUAGACA | AGGGTGCACTCCCCCCTAGACA |
| | (SEQ ID NO: 357) | (SEQ ID NO: 857) |
| RHO-259 | CCAGGAGUGGGAGAGGGAUUUG | CCAGGAGTGGGAGAGGGATTTG |
| | (SEQ ID NO: 358) | (SEQ ID NO: 858) |
| RHO-260 | AGAGGGGAGGCAGAGGAUGCCA | AGAGGGGAGGCAGAGGATGCCA |
| | (SEQ ID NO: 359) | (SEQ ID NO: 859) |
| RHO-261 | CCGCCUGCUGACUGCCUUGCAG | CCGCCTGCTGACTGCCTTGCAG |
| | (SEQ ID NO: 360) | (SEQ ID NO: 860) |
| RHO-262 | GGCUUGGUGCUGCAAACAUGGC | GGCTTGGTGCTGCAAACATGGC |
| | (SEQ ID NO: 361) | (SEQ ID NO: 861) |
| RHO-263 | CAGGUAAUGGCACUGAGCAGAA | CAGGTAATGGCACTGAGCAGAA |
| | (SEQ ID NO: 362) | (SEQ ID NO: 862) |
| RHO-264 | UUGGAACGCGGCAGGGAGGCUG | TTGGAACGCGGCAGGGAGGCTG |
| | (SEQ ID NO: 363) | (SEQ ID NO: 863) |
| RHO-265 | UGUCCGGGUUAUUUCAUUUCCC | TGTCCGGGTTATTTCATTTCCC |
| | (SEQ ID NO: 364) | (SEQ ID NO: 864) |
| RHO-266 | CAGGUAGUACUGUGGGUACUCG | CAGGTAGTACTGTGGGTACTCG |
| | (SEQ ID NO: 365) | (SEQ ID NO: 865) |
| RHO-267 | AUAACAGAUCCCACUUAACAGA | ATAACAGATCCCACTTAACAGA |
| | (SEQ ID NO: 366) | (SEQ ID NO: 866) |
| RHO-268 | AGGGACGGGUGCAGAGUUGAGU | AGGGACGGGTGCAGAGTTGAGT |
| | (SEQ ID NO: 367) | (SEQ ID NO: 867) |
| RHO-269 | GAAGGAGAGAGCUUGGUGCUGG | GAAGGAGAGAGCTTGGTGCTGG |
| | (SEQ ID NO: 368) | (SEQ ID NO: 868) |
| RHO-270 | GGUCAGCCACGGCUAGGUUGAG | GGTCAGCCACGGCTAGGTTGAG |
| | (SEQ ID NO: 369) | (SEQ ID NO: 869) |
| RHO-271 | AUUUCACAGCAAGAAAACUGAG | ATTTCACAGCAAGAAAACTGAG |
| | (SEQ ID NO: 370) | (SEQ ID NO: 870) |
| RHO-272 | UCAAAGAAGUCAAGCGCCCUGC | TCAAAGAAGTCAAGCGCCCTGC |
| | (SEQ ID NO: 371) | (SEQ ID NO: 871) |
| RHO-273 | GCUGCUCCCACCCAAGAAUGCU | GCTGCTCCCACCCAAGAATGCT |
| | (SEQ ID NO: 372) | (SEQ ID NO: 872) |
| RHO-274 | GCAACAAACACCCAACAAUGGC | GCAACAAACACCCAACAATGGC |
| | (SEQ ID NO: 373) | (SEQ ID NO: 873) |
| RHO-275 | AAAUCCACUUCCCACCCUGAGC | AAATCCACTTCCCACCCTGAGC |
| | (SEQ ID NO: 374) | (SEQ ID NO: 874) |
| RHO-276 | CAGGGAGGCUGGAGGGGCACCU | CAGGGAGGCTGGAGGGGCACCT |
| | (SEQ ID NO: 375) | (SEQ ID NO: 875) |
| RHO-277 | GGGCAAGCCAGACCCCUCCUCU | GGGCAAGCCAGACCCCTCCTCT |
| | (SEQ ID NO: 376) | (SEQ ID NO: 876) |
| RHO-278 | CAGGAAAACAGAUGGGGUGCUG | CAGGAAAACAGATGGGGTGCTG |
| | (SEQ ID NO: 377) | (SEQ ID NO: 877) |
| RHO-279 | UUGGAGAAGGGCACGUAGAAGU | TTGGAGAAGGGCACGTAGAAGT |
| | (SEQ ID NO: 378) | (SEQ ID NO: 878) |
| RHO-280 | AGAGCUUGGUGCUGGGAGGAGG | AGAGCTTGGTGCTGGGAGGAGG |
| | (SEQ ID NO: 379) | (SEQ ID NO: 879) |
| RHO-281 | UAGCUAGGAAGGCAACCAGGAG | TAGCTAGGAAGGCAACCAGGAG |
| | (SEQ ID NO: 380) | (SEQ ID NO: 880) |
| RHO-282 | GGCUAGGUUGAGCAGGAUGUAG | GGCTAGGTTGAGCAGGATGTAG |
| | (SEQ ID NO: 381) | (SEQ ID NO: 881) |
| RHO-283 | CUCACCACCCCAUGAAGUUCCA | CTCACCACCCCATGAAGTTCCA |
| | (SEQ ID NO: 382) | (SEQ ID NO: 882) |
| RHO-284 | AAGCAAUGUGCAAUGUUUUGCC | AAGCAATGTGCAATGTTTTGCC |
| | (SEQ ID NO: 383) | (SEQ ID NO: 883) |
| RHO-285 | GGAAGACCCAAUGACUGGAGAA | GGAAGACCCAATGACTGGAGAA |
| | (SEQ ID NO: 384) | (SEQ ID NO: 884) |
| RHO-286 | UGGCCAGGACCACCAAGGACCA | TGGCCAGGACCACCAAGGACCA |
| | (SEQ ID NO: 385) | (SEQ ID NO: 885) |
| RHO-287 | AAAUAUUGUCCCUUUCACUGUU | AAATATTGTCCCTTTCACTGTT |
| | (SEQ ID NO: 386) | (SEQ ID NO: 886) |
| RHO-288 | CAUGAGCAACUUCCGCUUCGGG | CATGAGCAACTTCCGCTTCGGG |
| | (SEQ ID NO: 387) | (SEQ ID NO: 887) |
| RHO-289 | AGAGAUAUUCCUGGAUCACAGC | AGAGATATTCCTGGATCACAGC |
| | (SEQ ID NO: 388) | (SEQ ID NO: 888) |
| RHO-290 | CAUGGAGGGGUCUGGGAGAGUC | CATGGAGGGGTCTGGGAGAGTC |
| | (SEQ ID NO: 389) | (SEQ ID NO: 889) |
| RHO-291 | AUGUUUUGCCCAGAGGAAGAAG | ATGTTTTGCCCAGAGGAAGAAG |
| | (SEQ ID NO: 390) | (SEQ ID NO: 890) |
| RHO-292 | GUGGGUGGGGUGUGCAGGAGCC | GTGGGTGGGGTGTGCAGGAGCC |
| | (SEQ ID NO: 391) | (SEQ ID NO: 891) |
| RHO-293 | CCAGGUAAUGGCACUGAGCAGA | CCAGGTAATGGCACTGAGCAGA |
| | (SEQ ID NO: 392) | (SEQ ID NO: 892) |
| RHO-294 | CCCAACAAUGGCCAGAGAUUCC | CCCAACAATGGCCAGAGATTCC |
| | (SEQ ID NO: 393) | (SEQ ID NO: 893) |
| RHO-295 | GCACCUGAGGACAGGGGCUGAG | GCACCTGAGGACAGGGGCTGAG |
| | (SEQ ID NO: 394) | (SEQ ID NO: 894) |
| RHO-296 | GUCAGACCCAGGCUGGGCACUG | GTCAGACCCAGGCTGGGCACTG |
| | (SEQ ID NO: 395) | (SEQ ID NO: 895) |
| RHO-297 | GGGGCACCUGAGGACAGGGGCU | GGGGCACCTGAGGACAGGGGCT |
| | (SEQ ID NO: 396) | (SEQ ID NO: 896) |
| RHO-298 | AGAGGAAAACUGAGGCAGGGAG | AGAGGAAAACTGAGGCAGGGAG |
| | (SEQ ID NO: 397) | (SEQ ID NO: 897) |
| RHO-299 | AGGGAUAACAGAUCCCACUUAA | AGGGATAACAGATCCCACTTAA |
| | (SEQ ID NO: 398) | (SEQ ID NO: 898) |
| RHO-300 | CUUGGUGCUGGGAGGAGGGGGA | CTTGGTGCTGGGAGGAGGGGGA |
| | (SEQ ID NO: 399) | (SEQ ID NO: 899) |
| RHO-301 | AGAGGGUAGCUAGGAAGGCAAC | AGAGGGTAGCTAGGAAGGCAAC |
| | (SEQ ID NO: 400) | (SEQ ID NO: 900) |
| RHO-302 | UUGCACAUUGCUUCAUGGCUCC | TTGCACATTGCTTCATGGCTCC |
| | (SEQ ID NO: 401) | (SEQ ID NO: 901) |
| RHO-303 | GACCGAGCCCAUUGCCCAGCAC | GACCGAGCCCATTGCCCAGCAC |
| | (SEQ ID NO: 402) | (SEQ ID NO: 902) |
| RHO-304 | UGAACACUGCCUUGAUCUUAUU | TGAACACTGCCTTGATCTTATT |
| | (SEQ ID NO: 403) | (SEQ ID NO: 903) |
| RHO-305 | GGUGCACUCCCCCCUAGACAGG | GGTGCACTCCCCCCTAGACAGG |
| | (SEQ ID NO: 404) | (SEQ ID NO: 904) |
| RHO-306 | GCUUGGUGCUGGGAGGAGGGGG | GCTTGGTGCTGGGAGGAGGGGG |
| | (SEQ ID NO: 405) | (SEQ ID NO: 905) |
| RHO-307 | GGAUACUUCGUCUUCGGGCCCA | GGATACTTCGTCTTCGGGCCCA |
| | (SEQ ID NO: 406) | (SEQ ID NO: 906) |
| RHO-308 | AGUCAGACCCAGGCUGGGCACU | AGTCAGACCCAGGCTGGGCACT |
| | (SEQ ID NO: 407) | (SEQ ID NO: 907) |
| RHO-309 | AGCACCAAGCCUCUGUUUCCCU | AGCACCAAGCCTCTGTTTCCCT |
| | (SEQ ID NO: 408) | (SEQ ID NO: 908) |
| RHO-310 | UGGGCAAAGAAAUUCCAGGGAA | TGGGCAAAGAAATTCCAGGGAA |
| | (SEQ ID NO: 409) | (SEQ ID NO: 909) |
| RHO-311 | AGAGGGAUUUGAGGAGGCCUUG | AGAGGGATTTGAGGAGGCCTTG |
| | (SEQ ID NO: 410) | (SEQ ID NO: 910) |
| RHO-312 | GCAAUGUUUUGCCCAGAGGAAG | GCAATGTTTTGCCCAGAGGAAG |
| | (SEQ ID NO: 411) | (SEQ ID NO: 911) |
| RHO-313 | CAUGUCCGGGUUAUUUCAUUUC | CATGTCCGGGTTATTTCATTTC |
| | (SEQ ID NO: 412) | (SEQ ID NO: 912) |
| RHO-314 | AAGCCCAUGAGCAACUUCCGCU | AAGCCCATGAGCAACTTCCGCT |
| | (SEQ ID NO: 413) | (SEQ ID NO: 913) |
| RHO-315 | UCCCACCCUGAGCUUGGGCCCC | TCCCACCCTGAGCTTGGGCCCC |
| | (SEQ ID NO: 414) | (SEQ ID NO: 914) |
| RHO-316 | GAGAGAGCUUGGUGCUGGGAGG | GAGAGAGCTTGGTGCTGGGAGG |
| | (SEQ ID NO: 415) | (SEQ ID NO: 915) |
| RHO-317 | CUACGUGCCCUUCUCCAAUGCG | CTACGTGCCCTTCTCCAATGCG |
| | (SEQ ID NO: 416) | (SEQ ID NO: 916) |
| RHO-318 | CUUGCAUUUAACAGGAAAACAG | CTTGCATTTAACAGGAAAACAG |
| | (SEQ ID NO: 417) | (SEQ ID NO: 917) |
| RHO-319 | GAAAUGAAAUAACCCGGACAUG | GAAATGAAATAACCCGGACATG |
| | (SEQ ID NO: 418) | (SEQ ID NO: 918) |
| RHO-320 | CGAAGGCCUGAGCUCAGCCACU | CGAAGGCCTGAGCTCAGCCACT |
| | (SEQ ID NO: 419) | (SEQ ID NO: 919) |
| RHO-321 | GGAGGGUGCACUCCCCCCUAGA | GGAGGGTGCACTCCCCCCTAGA |
| | (SEQ ID NO: 420) | (SEQ ID NO: 920) |
| RHO-322 | CAGCACCAAGCCUCUGUUUCCC | CAGCACCAAGCCTCTGTTTCCC |
| | (SEQ ID NO: 421) | (SEQ ID NO: 921) |
| RHO-323 | GGGCAAAGAAAUUCCAGGGAAU | GGGCAAAGAAATTCCAGGGAAT |
| | (SEQ ID NO: 422) | (SEQ ID NO: 922) |
| RHO-324 | CUUCGGGGAGAACCAUGCCAUC | CTTCGGGGAGAACCATGCCATC |
| | (SEQ ID NO: 423) | (SEQ ID NO: 923) |
| RHO-325 | UGGGAGGAGGGGGAAGGGGCAG | TGGGAGGAGGGGGAAGGGGCAG |
| | (SEQ ID NO: 424) | (SEQ ID NO: 924) |
| RHO-326 | CCUAGACAGGGAGAGGGUAGCU | CCTAGACAGGGAGAGGGTAGCT |
| | (SEQ ID NO: 425) | (SEQ ID NO: 925) |
| RHO-327 | UAACAGAGAGGAAAACUGAGGC | TAACAGAGAGGAAAACTGAGGC |
| | (SEQ ID NO: 426) | (SEQ ID NO: 926) |
| RHO-328 | UCUCAGCCACCACCGCCAAGCC | TCTCAGCCACCACCGCCAAGCC |
| | (SEQ ID NO: 427) | (SEQ ID NO: 927) |
| RHO-329 | GUCAGCACAGACCCCACUGCCU | GTCAGCACAGACCCCACTGCCT |
| | (SEQ ID NO: 428) | (SEQ ID NO: 928) |
| RHO-330 | AGGAAAACUGAGGCAGGGAGAG | AGGAAAACTGAGGCAGGGAGAG |
| | (SEQ ID NO: 429) | (SEQ ID NO: 929) |
| RHO-331 | AGCCAUUGCAAAGCUGGGUGAC | AGCCATTGCAAAGCTGGGTGAC |
| | (SEQ ID NO: 430) | (SEQ ID NO: 930) |
| RHO-332 | AAAUGAAAUAACCCGGACAUGU | AAATGAAATAACCCGGACATGT |
| | (SEQ ID NO: 431) | (SEQ ID NO: 931) |
| RHO-333 | UAGCUACCCUCUCCCUGUCUAG | TAGCTACCCTCTCCCTGTCTAG |
| | (SEQ ID NO: 432) | (SEQ ID NO: 932) |
| RHO-334 | UGUGGGUGGGGUGUGCAGGAGC | TGTGGGTGGGGTGTGCAGGAGC |
| | (SEQ ID NO: 433) | (SEQ ID NO: 933) |
| RHO-335 | UGGGGAAGGAGAGAGCUUGGUG | TGGGGAAGGAGAGAGCTTGGTG |
| | (SEQ ID NO: 434) | (SEQ ID NO: 934) |
| RHO-336 | GACUUGGAACGCGGCAGGGAGG | GACTTGGAACGCGGCAGGGAGG |
| | (SEQ ID NO: 435) | (SEQ ID NO: 935) |
| RHO-337 | AAGGAGAGAGCUUGGUGCUGGG | AAGGAGAGAGCTTGGTGCTGGG |
| | (SEQ ID NO: 436) | (SEQ ID NO: 936) |
| RHO-338 | GGGAAGGAGAGAGCUUGGUGCU | GGGAAGGAGAGAGCTTGGTGCT |
| | (SEQ ID NO: 437) | (SEQ ID NO: 937) |
| RHO-339 | AUUUGAGGAGGCCUUGGGGAAG | ATTTGAGGAGGCCTTGGGGAAG |
| | (SEQ ID NO: 438) | (SEQ ID NO: 938) |
| RHO-340 | AUCCAGCUGGAGCCCUGAGUGG | ATCCAGCTGGAGCCCTGAGTGG |
| | (SEQ ID NO: 439) | (SEQ ID NO: 939) |
| RHO-341 | GAGAGCUUGGUGCUGGGAGGAG | GAGAGCTTGGTGCTGGGAGGAG |
| | (SEQ ID NO: 440) | (SEQ ID NO: 940) |
| RHO-342 | UCCUAGCUACCCUCUCCCUGUC | TCCTAGCTACCCTCTCCCTGTC |
| | (SEQ ID NO: 441) | (SEQ ID NO: 941) |
| RHO-343 | CCGAGGCAGCAGCCUGGACAUG | CCGAGGCAGCAGCCTGGACATG |
| | (SEQ ID NO: 442) | (SEQ ID NO: 942) |
| RHO-344 | GGGGAAGGAGAGAGCUUGGUGC | GGGGAAGGAGAGAGCTTGGTGC |
| | (SEQ ID NO: 443) | (SEQ ID NO: 943) |
| RHO-345 | UGCUGGGAGGAGGGGGAAGGGG | TGCTGGGAGGAGGGGGAAGGGG |
| | (SEQ ID NO: 444) | (SEQ ID NO: 944) |
| RHO-346 | CUUCUUGUGCUGGACGGUGACG | CTTCTTGTGCTGGACGGTGACG |
| | (SEQ ID NO: 445) | (SEQ ID NO: 945) |
| RHO-347 | UACCACACCCGUCGCAUUGGAG | TACCACACCCGTCGCATTGGAG |
| | (SEQ ID NO: 446) | (SEQ ID NO: 946) |
| RHO-348 | AGCAGCCUGGACAUGGGGGAGA | AGCAGCCTGGACATGGGGGAGA |
| | (SEQ ID NO: 447) | (SEQ ID NO: 947) |
| RHO-349 | AGCCAGGUAGUACUGUGGGUAC | AGCCAGGTAGTACTGTGGGTAC |
| | (SEQ ID NO: 448) | (SEQ ID NO: 948) |
| RHO-350 | GGCUGCUUGCGGUUCUCAACAC | GGCTGCTTGCGGTTCTCAACAC |
| | (SEQ ID NO: 449) | (SEQ ID NO: 949) |
| RHO-351 | GGACCGAGGCAGCAGCCUGGAC | GGACCGAGGCAGCAGCCTGGAC |
| | (SEQ ID NO: 450) | (SEQ ID NO: 950) |
| RHO-352 | CUGGGCAAAGAAAUUCCAGGGA | CTGGGCAAAGAAATTCCAGGGA |
| | (SEQ ID NO: 451) | (SEQ ID NO: 951) |
| RHO-353 | UGAGAGGGGAGGCAGAGGAUGC | TGAGAGGGGAGGCAGAGGATGC |
| | (SEQ ID NO: 452) | (SEQ ID NO: 952) |
| RHO-354 | GAGGGUGCACUCCCCCCUAGAC | GAGGGTGCACTCCCCCCTAGAC |
| | (SEQ ID NO: 453) | (SEQ ID NO: 953) |
| RHO-355 | CGGUUCUCAACACCAGGAGACU | CGGTTCTCAACACCAGGAGACT |
| | (SEQ ID NO: 454) | (SEQ ID NO: 954) |
| RHO-356 | UGUGCAAUGUUUUGCCCAGAGG | TGTGCAATGTTTTGCCCAGAGG |
| | (SEQ ID NO: 455) | (SEQ ID NO: 955) |
| RHO-357 | GGGGGAGACAGGGCAAGGCUGG | GGGGGAGACAGGGCAAGGCTGG |
| | (SEQ ID NO: 456) | (SEQ ID NO: 956) |
| RHO-358 | GCCGGGUGUGGGUGGGGUGUGC | GCCGGGTGTGGGTGGGGTGTGC |
| | (SEQ ID NO: 457) | (SEQ ID NO: 957) |
| RHO-359 | CUGCGUACCACACCCGUCGCAU | CTGCGTACCACACCCGTCGCAT |
| | (SEQ ID NO: 458) | (SEQ ID NO: 958) |
| RHO-360 | CACCCAAGAAUGCUGCGAAGGC | CACCCAAGAATGCTGCGAAGGC |
| | (SEQ ID NO: 459) | (SEQ ID NO: 959) |
| RHO-361 | CCUAGCUACCCUCUCCCUGUCU | CCTAGCTACCCTCTCCCTGTCT |
| | (SEQ ID NO: 460) | (SEQ ID NO: 960) |
| RHO-362 | CACCAGGAGACUUGGAACGCGG | CACCAGGAGACTTGGAACGCGG |
| | (SEQ ID NO: 461) | (SEQ ID NO: 961) |
| RHO-363 | UUGGAUAACAUUGACAGGACAG | TTGGATAACATTGACAGGACAG |
| | (SEQ ID NO: 462) | (SEQ ID NO: 962) |
| RHO-364 | UUCGGGCCCACAGGAUGCAAUU | TTCGGGCCCACAGGATGCAATT |
| | (SEQ ID NO: 463) | (SEQ ID NO: 963) |
| RHO-365 | GAAGUAUCCAUGCAGAGAGGUG | GAAGTATCCATGCAGAGAGGTG |
| | (SEQ ID NO: 464) | (SEQ ID NO: 964) |
| RHO-366 | GGUGUGCAGGAGCCCGGGAGCA | GGTGTGCAGGAGCCCGGGAGCA |
| | (SEQ ID NO: 465) | (SEQ ID NO: 965) |
| RHO-367 | GGAGCAGCCACGGGUCAGCCAC | GGAGCAGCCACGGGTCAGCCAC |
| | (SEQ ID NO: 466) | (SEQ ID NO: 966) |
| RHO-368 | AGCGCCCUGCUGGGGCGUCACA | AGCGCCCTGCTGGGGCGTCACA |
| | (SEQ ID NO: 467) | (SEQ ID NO: 967) |
| RHO-369 | GAGCCCGGGAGCAUGGAGGGGU | GAGCCCGGGAGCATGGAGGGGT |
| | (SEQ ID NO: 468) | (SEQ ID NO: 968) |
| RHO-370 | AGGGCCACAGCCAUGAAUGGCA | AGGGCCACAGCCATGAATGGCA |
| | (SEQ ID NO: 469) | (SEQ ID NO: 969) |
| RHO-371 | GCAAUGUGCAAUGUUUUGCCCA | GCAATGTGCAATGTTTTGCCCA |
| | (SEQ ID NO: 470) | (SEQ ID NO: 970) |
| RHO-372 | GAAGAGGUCAGCCACGGCUAGG | GAAGAGGTCAGCCACGGCTAGG |
| | (SEQ ID NO: 471) | (SEQ ID NO: 971) |
| RHO-373 | GGCCUUCGCAGCAUUCUUGGGU | GGCCTTCGCAGCATTCTTGGGT |
| | (SEQ ID NO: 472) | (SEQ ID NO: 972) |
| RHO-374 | UUAACAGAGAGGAAAACUGAGG | TTAACAGAGAGGAAAACTGAGG |
| | (SEQ ID NO: 473) | (SEQ ID NO: 973) |
| RHO-375 | UGAUGGCAUGGUUCUCCCCGAA | TGATGGCATGGTTCTCCCCGAA |
| | (SEQ ID NO: 474) | (SEQ ID NO: 974) |
| RHO-376 | ACCGAGGCAGCAGCCUGGACAU | ACCGAGGCAGCAGCCTGGACAT |
| | (SEQ ID NO: 475) | (SEQ ID NO: 975) |
| RHO-377 | AGGGACCACACGCUGAGGAGAG | AGGGACCACACGCTGAGGAGAG |
| | (SEQ ID NO: 476) | (SEQ ID NO: 976) |
| RHO-378 | UGGAACGCGGCAGGGAGGCUGG | TGGAACGCGGCAGGGAGGCTGG |
| | (SEQ ID NO: 477) | (SEQ ID NO: 977) |
| RHO-379 | UGCACAUUGCUUCAUGGCUCCU | TGCACATTGCTTCATGGCTCCT |
| | (SEQ ID NO: 478) | (SEQ ID NO: 978) |
| RHO-380 | GCGUUCCAAGUCUCCUGGUGUU | GCGTTCCAAGTCTCCTGGTGTT |
| | (SEQ ID NO: 479) | (SEQ ID NO: 979) |
| RHO-381 | GGGUGUGCAGGAGCCCGGGAGC | GGGTGTGCAGGAGCCCGGGAGC |
| | (SEQ ID NO: 480) | (SEQ ID NO: 980) |
| RHO-382 | GGCAAAGAAAUUCCAGGGAAUG | GGCAAAGAAATTCCAGGGAATG |
| | (SEQ ID NO: 481) | (SEQ ID NO: 981) |
| RHO-383 | GGCUGGAGGGGCACCUGAGGAC | GGCTGGAGGGGCACCTGAGGAC |
| | (SEQ ID NO: 482) | (SEQ ID NO: 982) |
| RHO-384 | GCGCCCUGCUGGGGCGUCACAC | GCGCCCTGCTGGGGCGTCACAC |
| | (SEQ ID NO: 483) | (SEQ ID NO: 983) |
| RHO-385 | GCGUACCACACCCGUCGCAUUG | GCGTACCACACCCGTCGCATTG |
| | (SEQ ID NO: 484) | (SEQ ID NO: 984) |
| RHO-386 | ACCAGGAGACUUGGAACGCGGC | ACCAGGAGACTTGGAACGCGGC |
| | (SEQ ID NO: 485) | (SEQ ID NO: 985) |
| RHO-387 | GCUGCUGCCUCGGUCCCAUUCU | GCTGCTGCCTCGGTCCCATTCT |
| | (SEQ ID NO: 486) | (SEQ ID NO: 986) |
| RHO-388 | GAAGCCCUCCAAAUUGCAUCCU | GAAGCCCTCCAAATTGCATCCT |
| | (SEQ ID NO: 487) | (SEQ ID NO: 987) |
| RHO-389 | CGUAGAGCGUGAGGAAGUUGAU | CGTAGAGCGTGAGGAAGTTGAT |
| | (SEQ ID NO: 488) | (SEQ ID NO: 988) |
| RHO-390 | CUGAAGCAGUUCCUUUUUGCUU | CTGAAGCAGTTCCTTTTTGCTT |
| | (SEQ ID NO: 489) | (SEQ ID NO: 989) |
| RHO-391 | GCUGGACGGUGACGUAGAGCGU | GCTGGACGGTGACGTAGAGCGT |
| | (SEQ ID NO: 490) | (SEQ ID NO: 990) |
| RHO-392 | UGAGGGCUUUGGAUAACAUUGA | TGAGGGCTTTGGATAACATTGA |
| | (SEQ ID NO: 491) | (SEQ ID NO: 991) |
| RHO-393 | AGCCGGGUGUGGGUGGGGUGUG | AGCCGGGTGTGGGTGGGGTGTG |
| | (SEQ ID NO: 492) | (SEQ ID NO: 992) |
| RHO-394 | CUCAGUUUUCCUCUCUGUUAAG | CTCAGTTTTCCTCTCTGTTAAG |
| | (SEQ ID NO: 493) | (SEQ ID NO: 993) |
| RHO-395 | CAAGACAUUAUUCUAAAGCAAA | CAAGACATTATTCTAAAGCAAA |
| | (SEQ ID NO: 494) | (SEQ ID NO: 994) |
| RHO-396 | UGGAACUUCAUGGGGUGGUGAG | TGGAACTTCATGGGGTGGTGAG |
| | (SEQ ID NO: 495) | (SEQ ID NO: 995) |
| RHO-397 | GAGAGGGAUUUGAGGAGGCCUU | GAGAGGGATTTGAGGAGGCCTT |
| | (SEQ ID NO: 496) | (SEQ ID NO: 996) |
| RHO-398 | CUUCGGGCCCACAGGAUGCAAU | CTTCGGGCCCACAGGATGCAAT |
| | (SEQ ID NO: 497) | (SEQ ID NO: 997) |
| RHO-399 | ACUUGGAACGCGGCAGGGAGGC | ACTTGGAACGCGGCAGGGAGGC |
| | (SEQ ID NO: 498) | (SEQ ID NO: 998) |
| RHO-400 | AUGGCCAGAGAUUCCCUGAGAA | ATGGCCAGAGATTCCCTGAGAA |
| | (SEQ ID NO: 499) | (SEQ ID NO: 999) |
| RHO-401 | CCUCAGUUUUCCUCUCUGUUAA | CCTCAGTTTTCCTCTCTGTTAA |
| | (SEQ ID NO: 500) | (SEQ ID NO: 1000) |
| RHO-402 | UAACAGAUCCCACUUAACAGAG | TAACAGATCCCACTTAACAGAG |
| | (SEQ ID NO: 501) | (SEQ ID NO: 1001) |
| RHO-403 | GGGAGAGGGAUUUGAGGAGGCC | GGGAGAGGGATTTGAGGAGGCC |
| | (SEQ ID NO: 502) | (SEQ ID NO: 1002) |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

### Additional Sequences

Exemplary sequences that may be used in certain embodiments are set forth below:

AAV ITR
(SEQ ID NO: 92)
TGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCA
TCACTAGGGGTTCCT
U6 Promoter:
(SEQ ID NO: 78)
AAGGTCGGGCAGGAAGAGGGCCTATTTCCCATGATTCCTTCATATTTGCATATACGATACAA
GGCTGTTAGAGAGATAATTAGAATTAATTTGACTGTAAACACAAAGATATTAGTACAAAATA
CGTGACGTAGAAAGTAATAATTTCTTGGGTAGTTTGCAGTTTTAAAATTATGTTTTAAAATG
GACTATCATATGCTTACCGTAACTTGAAAGTATTTCGATTTCTTGGCTTTATATATCTTGTG
GAAAGGACGAAACACC
Exemplary saCas9 gRNA protospacer:
(SEQ ID NO: 606)
CCCACACCCGGCTCATACCGCC
Guide RNA scaffold sequence:
(SEQ ID NO: 12)
GTTATAGTACTCTGGAAACAGAATCTACTATAACAAGGCAAAATGCCGTGTTTATCTCGTCA
ACTTGTTGGCGAGA
Minimal RHO Promoter (250 bp):
(SEQ ID NO: 44)
GTCACCTTGGCCCCTCTTAGAAGCCAATTAGGCCCTCAGTTTCTGCAGCGGGGATTAATATG
ATTATGAACACCCCCAATCTCCCAGATGCTGATTCAGCCAGGAGCTTAGGAGGGGGAGGTCA
CTTTATAAGGGTCTGGGGGGGTCAGAACCCAGAGTCATCCAGCTGGAGCCCTGAGTGGCTGA
GCTCAGGCCTTCGCAGCATTCTTGGGTGGGAGCAGCCACGGGTCAGCCACAAGGGCCACAGC
C
SV40 Intron:
(SEQ ID NO: 94)
TCTAGAGGATCCGGTACTCGAGGAACTGAAAAACCAGAAAGTTAACTGGTAAGTTTAGTCTT
TTTGTCTTTTATTTCAGGTCCCGGATCCGGTGGTGGTGCAAATCAAAGAACTGCTCCTCAGT
GGATGTTGCCTTTACTTCTAGGCCTGTACGGAAGTGTTAC
Codon Optimized RHO-encoding sequence 1 (Codon 1):
(SEQ ID NO: 13)
ATGAACGGCACCGAGGGCCCCAACTTCTACGTCCCCTTCAGCAACGCCACCGGCGTCGTCCG
CAGCCCCTTCGAGTACCCCCAGTACTACCTGGCCGAGCCCTGGCAGTTCAGCATGCTGGCCG
CCTACATGTTCCTGCTGATCGTCCTGGGCTTCCCCATCAACTTCCTGACCCTGTACGTCACC
GTCCAGCACAAGAAGCTGCGCACCCCCCTGAACTACATCCTGCTGAACCTGGCCGTCGCCGA
CCTGTTCATGGTCCTGGGCGGCTTCACCAGCACCCTGTACACCAGCCTGCACGGCTACTTCG
TCTTCGGCCCCACCGGCTGCAACCTGGAGGGCTTCTTCGCCACCCTGGGCGGCGAGATCGCC
CTGTGGAGCCTGGTCGTCCTGGCCATCGAGCGCTACGTCGTCGTCTGCAAGCCCATGAGCAA
CTTCCGCTTCGGCGAGAACCACGCCATCATGGGCGTCGCCTTCACCTGGGTCATGGCCCTGG
CCTGCGCCGCCCCCCCCCTGGCCGGCTGGAGCCGCTACATCCCCGAGGGCCTGCAGTGCAGC
TGCGGCATCGACTACTACACCCTGAAGCCCGAGGTCAACAACGAGAGCTTCGTCATCTACAT
GTTCGTCGTCCACTTCACCATCCCCATGATCATCATCTTCTTCTGCTACGGCCAGCTGGTCT
TCACCGTCAAGGAGGCCGCCGCCCAGCAGCAGGAGAGCGCCACCACCCAGAAGGCCGAGAAG
GAGGTCACCCGCATGGTCATCATCATGGTCATCGCCTTCCTGATCTGCTGGGTCCCCTACGC
CAGCGTCGCCTTCTACATCTTCACCCACCAGGGCAGCAACTTCGGCCCCATCTTCATGACCA
TCCCCGCCTTCTTCGCCAAGAGCGCCGCCATCTACAACCCCGTCATCTACATCATGATGAAC
AAGCAGTTCCGCAACTGCATGCTGACCACCATCTGCTGCGGCAAGAACCCCCTGGGCGACGA
CGAGGCCAGCGCCACCGTCAGCAAGACCGAGACCAGCCAGGTCGCCCCCGCCTAA
Codon Optimized RHO-encoding sequence 2 (Codon 2):
(SEQ ID NO: 14)
ATGAACGGCACCGAGGGCCCCAACTTCTACGTGCCCTTCTCCAACGCCACCGGCGTGGTGCG
CTCCCCCTTCGAGTACCCCCAGTACTACCTGGCCGAGCCCTGGCAGTTCTCCATGCTGGCCG
CCTACATGTTCCTGCTGATCGTGCTGGGCTTCCCCATCAACTTCCTGACCCTGTACGTGACC
GTGCAGCACAAGAAGCTGCGCACCCCCCTGAACTACATCCTGCTGAACCTGGCCGTGGCCGA
CCTGTTCATGGTGCTGGGCGGCTTCACCTCCACCCTGTACACCTCCCTGCACGGCTACTTCG
TGTTCGGCCCCACCGGCTGCAACCTGGAGGGCTTCTTCGCCACCCTGGGCGGCGAGATCGCC
CTGTGGTCCCTGGTGGTGCTGGCCATCGAGCGCTACGTGGTGGTGTGCAAGCCCATGTCCAA
CTTCCGCTTCGGCGAGAACCACGCCATCATGGGCGTGGCCTTCACCTGGGTGATGGCCCTGG
CCTGCGCCGCCCCCCCCCTGGCCGGCTGGTCCCGCTACATCCCCGAGGGCCTGCAGTGCTCC
TGCGGCATCGACTACTACACCCTGAAGCCCGAGGTGAACAACGAGTCCTTCGTGATCTACAT
GTTCGTGGTGCACTTCACCATCCCCATGATCATCATCTTCTTCTGCTACGGCCAGCTGGTGT
TCACCGTGAAGGAGGCCGCCGCCCAGCAGCAGGAGTCCGCCACCACCCAGAAGGCCGAGAAG
GAGGTGACCCGCATGGTGATCATCATGGTGATCGCCTTCCTGATCTGCTGGGTGCCCTACGC
CTCCGTGGCCTTCTACATCTTCACCCACCAGGGCTCCAACTTCGGCCCCATCTTCATGACCA
TCCCCGCCTTCTTCGCCAAGTCCGCCGCCATCTACAACCCCGTGATCTACATCATGATGAAC
AAGCAGTTCCGCAACTGCATGCTGACCACCATCTGCTGCGGCAAGAACCCCCTGGGCGACGA
CGAGGCCTCCGCCACCGTGTCCAAGACCGAGACCTCCCAGGTGGCCCCCGCCTAA
Codon Optimized RHO-encoding sequence 3 (Codon 3):
(SEQ ID NO: 15)
ATGAACGGCACCGAGGGCCCCAACTTCTACGTCCCCTTCAGCAACGCCACCGGCGTCGTCCG
CAGCCCCTTCGAGTACCCCCAGTACTACCTGGCCGAGCCCTGGCAGTTCTCTATGCTGGCCG
CCTACATGTTCCTGCTGATCGTCCTGGGCTTCCCTATCAACTTCCTCACCCTCTACGTCACC
GTCCAGCACAAGAAGCTCCGCACCCCTCTCAACTACATCCTCCTTAACCTTGCCGTCGCCGA
CCTTTTCATGGTCCTTGGCGGCTTCACCTCTACTCTTTACACTTCTTTGCACGGGTACTTCG
TGTTCGGTCCTACTGGTTGCAACTTGGAGGGTTTCTTCGCCACTTTGGGTGGTGAGATCGCC
TTGTGGTCGTTGGTGGTGTTAGCTATCGAGCGATACGTGGTGGTGTGCAAGCCTATGTCGAA
CTTCCGGTTCGGTGAGAATCATGCTATCATGGGAGTGGCTTTTACTTGGGTGATGGCTTTAG
CTTGCGCTGCTCCTCCGTTAGCTGGATGGTCGCGTTATATCCCGGAGGGATTACAGTGCTCA
TGCGGAATCGACTATTATACTCTAAAGCCGGAAGTTAATAATGAATCATTTGTTATTTATAT
GTTTGTTGTTCATTTTACAATTCCGATGATTATTATTTTTTTTTGTTATGGACAGCTAGTTT
TTACAGTTAAGGAAGCAGCAGCACAGCAACAAGAATCAGCAACAACACAAAAGGCAGAAAAA
GAAGTTACAAGGATGGTTATTATTATGGTAATTGCATTTCTAATATGTTGGGTACCGTATGC
ATCCGTAGCATTTTATATATTTACACATCAAGGGTCCAATTTTGGGCCAATATTTATGACGA
TACCAGCGTTTTTTGCGAAATCCGCGGCGATATATAATCCAGTAATATATATAATGATGAAT
AAACAATTTAGAAATTGTATGCTAACGACGATATGTTGTGGGAAAAATCCACTAGGGGATGA
TGAAGCGAGTGCGACGGTAAGTAAAACGGAAACGAGTCAAGTAGCGCCAGCGTAA
Codon Optimized RHO-encoding sequence 4 (Codon 4):
(SEQ ID NO: 16)
ATGAACGGCACCGAGGGTCCCAATTTCTACGTCCCATTTTCCAACGCCACGGGGGTGGTACG
CAGCCCTTTCGAATATCCGCAGTACTATCTGGCTGAGCCCTGGCAGTTTTCTATGCTCGCAG
CGTACATGTTCTTGCTAATCGTTCTGGGATTTCCAATTAATTTCCTCACATTGTATGTCACC
GTGCAGCACAAGAAGCTACGGACGCCTCTGAACTACATCCTCTTGAATCTAGCCGTCGCTGA
CCTGTTTATGGTTCTCGGCGGTTTCACATCGACCTTGTATACGTCACTACATGGGTACTTTG
TCTTCGGACCGACAGGCTGCAACCTGGAAGGTTTTTTCGCAACCCTCGGGGGAGAGATTGCG
TTGTGGTCCCTAGTGGTACTGGCCATCGAAAGGTATGTTGTCGTGTGTAAGCCCATGAGCAA
TTTTCGCTTCGGCGAGAACCACGCTATTATGGGTGTAGCATTTACGTGGGTTATGGCGCTCG
CCTGCGCTGCACCACCTTTGGCGGGGTGGTCTCGGTACATCCCGGAAGGACTACAGTGTTCG
TGCGGCATTGATTATTACACACTGAAGCCCGAGGTCAATAACGAATCATTCGTGATCTATAT
GTTTGTAGTTCATTTCACCATTCCAATGATCATTATCTTTTTCTGTTACGGTCAGCTCGTCT
TTACGGTGAAGGAGGCCGCTGCACAGCAGCAGGAATCCGCGACAACCCAGAAGGCCGAGAAG
GAAGTAACGAGGATGGTTATTATCATGGTCATTGCTTTCTTGATCTGCTGGGTGCCTTATGC
AAGCGTAGCGTTTTACATTTTCACACACCAGGGGTCTAATTTTGGACCGATCTTCATGACCA
TTCCCGCCTTTTTCGCTAAGTCGGCAGCGATCTATAACCCAGTTATTTACATCATGATGAAT
AAGCAGTTTCGCAACTGTATGCTAACGACAATTTGCTGTGGCAAGAATCCTCTGGGTGACGA
TGAGGCCTCAGCTACCGTCTCCAAGACGGAAACAAGCCAGGTGGCACCGGCGTAA
Codon Optimized RHO-encoding sequence 5 (Codon 5):
(SEQ ID NO: 17)
ATGAATGGGACTGAAGGACCTAATTTCTATGTGCCATTTAGCAATGCTACTGGCGTTGTCAG
AAGCCCCTTCGAATATCCACAATACTATCTGGCCGAACCTTGGCAGTTCAGCATGCTCGCTG
CCTATATGTTTCTGCTGATTGTGCTGGGCTTTCCCATAAATTTCCTCACCCTGTATGTTACT
GTTCAACACAAAAAGCTGCGGACGCCTCTGAACTACATACTGCTGAACCTGGCCGTCGCCGA
CCTGTTTATGGTCCTGGGAGGCTTTACAAGCACTCTGTATACAAGCCTGCACGGCTACTTCG
TGTTCGGCCCCACAGGCTGCAACCTCGAAGGCTTCTTTGCCACCCTCGGAGGAGAGATTGCC
CTGTGGAGCCTGGTGGTGCTGGCCATCGAAAGGTATGTGGTGGTGTGTAAACCCATGTCCAA
TTTTCGGTTCGGCGAGAACCACGCTATTATGGGAGTGGCTTTCACTTGGGTGATGGCCCTGG
CCTGCGCCGCCCCACCACTGGCCGGGTGGAGCCGGTACATCCCAGAGGGGCTGCAATGTAGC
TGCGGAATCGACTATTATACCCTGAAACCAGAGGTGAACAACGAGAGCTTTGTGATTTATAT
GTTTGTGGTGCATTTTACAATTCCTATGATTATCATTTTCTTCTGTTACGGGCAACTGGTGT
TTACCGTGAAGGAAGCCGCCGCTCAACAGCAGGAGAGCGCCACAACCCAAAAGGCCGAGAAG
GAGGTGACCAGAATGGTGATTATTATGGTGATCGCTTTTCTGATTTGCTGGGTGCCATACGC
TAGCGTCGCTTTCTATATTTTCACTCACCAGGGGAGCAACTTCGGCCCCATTTTCATGACAA
TCCCTGCCTTTTTTGCTAAAAGCGCCGCCATCTATAACCCAGTGATCTACATCATGATGAAC
AAACAGTTTAGGAACTGTATGCTCACAACAATCTGCTGTGGAAAGAACCCCCTCGGCGATGA
CGAAGCCAGCGCCACCGTCAGCAAGACAGAAACAAGCCAGGTGGCCCCTGCCTAA
Codon Optimized RHO-encoding sequence 6 (Codon 6):
(SEQ ID NO: 18)
ATGAATGGCACAGAGGGCCCTAACTTCTACGTGCCCTTTAGCAATGCCACAGGCGTCGTGCG
GAGCCCTTTTGAGTACCCTCAGTACTATCTGGCCGAGCCTTGGCAGTTTAGCATGCTGGCCG
CCTACATGTTCCTGCTGATCGTGCTGGGCTTCCCCATCAACTTTCTGACCCTGTACGTGACC
GTGCAGCACAAGAAGCTGCGGACCCCTCTGAACTACATCCTGCTGAATCTGGCCGTGGCCGA
CCTGTTTATGGTGCTCGGCGGCTTTACCAGCACACTGTACACAAGCCTGCACGGCTACTTCG
TGTTTGGCCCCACCGGCTGCAATCTGGAAGGCTTTTTTGCCACACTCGGCGGCGAAATTGCT
CTGTGGTCACTGGTGGTGCTGGCCATCGAGAGATACGTGGTCGTGTGCAAGCCCATGAGCAA
CTTCAGATTCGGCGAGAACCACGCCATCATGGGCGTCGCCTTTACATGGGTTATGGCCCTGG
CTTGTGCAGCTCCTCCTCTTGCCGGCTGGTCCAGATATATTCCTGAGGGCCTGCAGTGCAGC
TGCGGCATCGATTACTACACCCTGAAGCCTGAAGTGAACAACGAGAGCTTCGTGATCTACAT
GTTTGTGGTGCACTTCACGATCCCCATGATCATCATATTCTTTTGCTACGGCCAGCTGGTGT
TCACCGTGAAAGAAGCCGCTGCTCAGCAGCAAGAGAGCGCCACAACACAGAAAGCCGAGAAA
GAAGTGACCCGGATGGTCATTATCATGGTTATCGCCTTTCTGATCTGTTGGGTGCCCTACGC
CAGCGTGGCCTTCTACATCTTTACCCACCAAGGCAGCAACTTCGGCCCCATCTTTATGACAA
TCCCCGCCTTCTTTGCCAAGAGCGCCGCCATCTACAACCCCGTGATCTATATCATGATGAAC
AAGCAGTTCCGCAACTGCATGCTGACCACCATCTGCTGCGGAAAGAACCCTCTGGGAGATGA
TGAGGCCAGCGCCACCGTGTCTAAGACCGAAACATCTCAGGTGGCCCCTGCATGA
Hemoglobin A1 (HBA1) 3′ UTR:
(SEQ ID NO: 38)
GCTGGAGCCTCGGTGGCCATGCTTCTTGCCCCTTGGGCCTCCCCCCAGCCCCTCCTCCCCTT
CCTGCACCCGTACCCCCGTGGTCTTTGAATAAAGTCTGAGTGGGCGGCA
Minimal UTR (minPolyA):
(SEQ ID NO: 56)
TAGCAATAAAGGATCGTTTATTTTCATTGGAAGCGTGTGTTGGTTTTTTGATCAGGCGCG
Inverted ITR sequence:
(SEQ ID NO: 93)
AGGAACCCCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCGAGCGAGC
GCGCAGCTGCCTGCA
Exemplary replacement vector (250 bp minimal RHO promoter
driving codon-optimized RHO cDNA; U6 promoter driving gRNA
targeting RHO) (see FIG. 16 for feature annotation):
(SEQ ID NO: 11)
TGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCA
TCACTAGGGGTTCCTGCGGCCGCGGTTCCTCAGATCTGAATTCGGTACCAAGGTCGGGCAGG
AAGAGGGCCTATTTCCCATGATTCCTTCATATTTGCATATACGATACAAGGCTGTTAGAGAG
ATAATTAGAATTAATTTGACTGTAAACACAAAGATATTAGTACAAAATACGTGACGTAGAAA
GTAATAATTTCTTGGGTAGTTTGCAGTTTTAAAATTATGTTTTAAAATGGACTATCATATGC
TTACCGTAACTTGAAAGTATTTCGATTTCTTGGCTTTATATATCTTGTGGAAAGGACGAAAC
ACCGCCCACACCCGGCTCATACCGCCGTTATAGTACTCTGGAAACAGAATCTACTATAACAA
GGCAAAATGCCGTGTTTATCTCGTCAACTTGTTGGCGAGATTTTTTCGACTTAGTTCGATCG
AAGGAAGGTCGGGCAGGAAGAGGGCCTATTTCCCATGATTCCTTCATATTTGCATATACGAT
ACAAGGCTGTTAGAGAGATAATTAGAATTAATTTGACTGTAAACACAAAGATATTAGTACAA
AATACGTGACGTAGAAAGTAATAATTTCTTGGGTAGTTTGCAGTTTTAAAATTATGTTTTAA
AATGGACTATCATATGCTTACCGTAACTTGAAAGTATTTCGATTTCTTGGCTTTATATATCT
TGTGGAAAGGACGAAACACCGCCCACACCCGGCTCATACCGCCGTTATAGTACTCTGGAAAC
AGAATCTACTATAACAAGGCAAAATGCCGTGTTTATCTCGTCAACTTGTTGGCGAGATTTTT
TGGTACCGCTAGCGCTGTCACCTTGGCCCCTCTTAGAAGCCAATTAGGCCCTCAGTTTCTGC
AGCGGGGATTAATATGATTATGAACACCCCCAATCTCCCAGATGCTGATTCAGCCAGGAGCT
TAGGAGGGGGAGGTCACTTTATAAGGGTCTGGGGGGGTCAGAACCCAGAGTCATCCAGCTGG
AGCCCTGAGTGGCTGAGCTCAGGCCTTCGCAGCATTCTTGGGTGGGAGCAGCCACGGGTCAG
CCACAAGGGCCACAGCCTCTAGAGGATCCGGTACTCGAGGAACTGAAAAACCAGAAAGTTAA
CTGGTAAGTTTAGTCTTTTTGTCTTTTATTTCAGGTCCCGGATCCGGTGGTGGTGCAAATCA
AAGAACTGCTCCTCAGTGGATGTTGCCTTTACTTCTAGGCCTGTACGGAAGTGTTACTCCGC
CACCATGAATGGCACAGAGGGCCCTAACTTCTACGTGCCCTTTAGCAATGCCACAGGCGTCG
TGCGGAGCCCTTTTGAGTACCCTCAGTACTATCTGGCCGAGCCTTGGCAGTTTAGCATGCTG
GCCGCCTACATGTTCCTGCTGATCGTGCTGGGCTTCCCCATCAACTTTCTGACCCTGTACGT
GACCGTGCAGCACAAGAAGCTGCGGACCCCTCTGAACTACATCCTGCTGAATCTGGCCGTGG
CCGACCTGTTTATGGTGCTCGGCGGCTTTACCAGCACACTGTACACAAGCCTGCACGGCTAC
TTCGTGTTTGGCCCCACCGGCTGCAATCTGGAAGGCTTTTTTGCCACACTCGGCGGCGAAAT
TGCTCTGTGGTCACTGGTGGTGCTGGCCATCGAGAGATACGTGGTCGTGTGCAAGCCCATGA
GCAACTTCAGATTCGGCGAGAACCACGCCATCATGGGCGTCGCCTTTACATGGGTTATGGCC
CTGGCTTGTGCAGCTCCTCCTCTTGCCGGCTGGTCCAGATATATTCCTGAGGGCCTGCAGTG
CAGCTGCGGCATCGATTACTACACCCTGAAGCCTGAAGTGAACAACGAGAGCTTCGTGATCT
ACATGTTTGTGGTGCACTTCACGATCCCCATGATCATCATATTCTTTTGCTACGGCCAGCTG
GTGTTCACCGTGAAAGAAGCCGCTGCTCAGCAGCAAGAGAGCGCCACAACACAGAAAGCCGA
GAAAGAAGTGACCCGGATGGTCATTATCATGGTTATCGCCTTTCTGATCTGTTGGGTGCCCT
ACGCCAGCGTGGCCTTCTACATCTTTACCCACCAAGGCAGCAACTTCGGCCCCATCTTTATG
ACAATCCCCGCCTTCTTTGCCAAGAGCGCCGCCATCTACAACCCCGTGATCTATATCATGAT
GAACAAGCAGTTCCGCAACTGCATGCTGACCACCATCTGCTGCGGAAAGAACCCTCTGGGAG
ATGATGAGGCCAGCGCCACCGTGTCTAAGACCGAAACATCTCAGGTGGCCCCTGCATGAGCT
GGAGCCTCGGTGGCCATGCTTCTTGCCCCTTGGGCCTCCCCCCAGCCCCTCCTCCCCTTCCT
GCACCCGTACCCCCGTGGTCTTTGAATAAAGTCTGAGTGGGCGGCACATGCTGGGGAGAGAT
CTGCGGCCGCAGGAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCTCAGTGA
GCGAGCGAGCGCGCAGCTGCCTGCA
Cas9 Vector 2 (250 bp minimal RHO promoter driving Cas9
w/alpha globin UTR) (see FIG. 17 for feature annotation):
(SEQ ID NO: 10)
CCTGCAGGCAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAACTC
CATCACTAGGGGTTCCTAAGCGGCCGCGGTTCCTCAGATCTGAATTCGGTACCTGTCACCTT
GGCCCCTCTTAGAAGCCAATTAGGCCCTCAGTTTCTGCAGCGGGGATTAATATGATTATGAA
CACCCCCAATCTCCCAGATGCTGATTCAGCCAGGAGCTTAGGAGGGGGAGGTCACTTTATAA
GGGTCTGGGGGGGTCAGAACCCAGAGTCATCCAGCTGGAGCCCTGAGTGGCTGAGCTCAGGC
CTTCGCAGCATTCTTGGGTGGGAGCAGCCACGGGTCAGCCACAAGGGCCACAGCCTCTAGAG
GATCCGGTACTCGAGGAACTGAAAAACCAGAAAGTTAACTGGTAAGTTTAGTCTTTTTGTCT
TTTATTTCAGGTCCCGGATCCGGTGGTGGTGCAAATCAAAGAACTGCTCCTCAGTGGATGTT
GCCTTTACTTCTAGGCCTGTACGGAAGTGTTACTCCGCCACCATGGGACCGAAGAAAAAGCG
CAAGGTCGAAGCGTCCATGAAAAGGAACTACATTCTGGGGCTGGACATCGGGATTACAAGCG
TGGGGTATGGGATTATTGACTATGAAACAAGGGACGTGATCGACGCAGGCGTCAGACTGTTC
AAGGAGGCCAACGTGGAAAACAATGAGGGACGGAGAAGCAAGAGGGGAGCCAGGCGCCTGAA
ACGACGGAGAAGGCACAGAATCCAGAGGGTGAAGAAACTGCTGTTCGATTACAACCTGCTGA
CCGACCATTCTGAGCTGAGTGGAATTAATCCTTATGAAGCCAGGGTGAAAGGCCTGAGTCAG
AAGCTGTCAGAGGAAGAGTTTTCCGCAGCTCTGCTGCACCTGGCTAAGCGCCGAGGAGTGCA
TAACGTCAATGAGGTGGAAGAGGACACCGGCAACGAGCTGTCTACAAAGGAACAGATCTCAC
GCAATAGCAAAGCTCTGGAAGAGAAGTATGTCGCAGAGCTGCAGCTGGAACGGCTGAAGAAA
GATGGCGAGGTGAGAGGGTCAATTAATAGGTTCAAGACAAGCGACTACGTCAAAGAAGCCAA
GCAGCTGCTGAAAGTGCAGAAGGCTTACCACCAGCTGGATCAGAGCTTCATCGATACTTATA
TCGACCTGCTGGAGACTCGGAGAACCTACTATGAGGGACCAGGAGAAGGGAGCCCCTTCGGA
TGGAAAGACATCAAGGAATGGTACGAGATGCTGATGGGACATTGCACCTATTTTCCAGAAGA
GCTGAGAAGCGTCAAGTACGCTTATAACGCAGATCTGTACAACGCCCTGAATGACCTGAACA
ACCTGGTCATCACCAGGGATGAAAACGAGAAACTGGAATACTATGAGAAGTTCCAGATCATC
GAAAACGTGTTTAAGCAGAAGAAAAAGCCTACACTGAAACAGATTGCTAAGGAGATCCTGGT
CAACGAAGAGGACATCAAGGGCTACCGGGTGACAAGCACTGGAAAACCAGAGTTCACCAATC
TGAAAGTGTATCACGATATTAAGGACATCACAGCACGGAAAGAAATCATTGAGAACGCCGAA
CTGCTGGATCAGATTGCTAAGATCCTGACTATCTACCAGAGCTCCGAGGACATCCAGGAAGA
GCTGACTAACCTGAACAGCGAGCTGACCCAGGAAGAGATCGAACAGATTAGTAATCTGAAGG
GGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGA
CCTGAGTCAGCAGAAAGAGATCCCAACCACACTGGTGGACGATTTCATTCTGTCACCCGTGG
TCAAGCGGAGCTTCATCCAGAGCATCAAAGTGATCAACGCCATCATCAAGAAGTACGGCCTG
CCCAATGATATCATTATCGAGCTGGCTAGGGAGAAGAACAGCAAGGACGCACAGAAGATGAT
CAATGAGATGCAGAAACGAAACCGGCAGACCAATGAACGCATTGAAGAGATTATCCGAACTA
CCGGGAAAGAGAACGCAAAGTACCTGATTGAAAAAATCAAGCTGCACGATATGCAGGAGGGA
AAGTGTCTGTATTCTCTGGAGGCCATCCCCCTGGAGGACCTGCTGAACAATCCATTCAACTA
CGAGGTCGATCATATTATCCCCAGAAGCGTGTCCTTCGACAATTCCTTTAACAACAAGGTGC
TGGTCAAGCAGGAAGAGAACTCTAAAAAGGGCAATAGGACTCCTTTCCAGTACCTGTCTAGT
TCAGATTCCAAGATCTCTTACGAAACCTTTAAAAAGCACATTCTGAATCTGGCCAAAGGAAA
GGGCCGCATCAGCAAGACCAAAAAGGAGTACCTGCTGGAAGAGCGGGACATCAACAGATTCT
CCGTCCAGAAGGATTTTATTAACCGGAATCTGGTGGACACAAGATACGCTACTCGCGGCCTG
ATGAATCTGCTGCGATCCTATTTCCGGGTGAACAATCTGGATGTGAAAGTCAAGTCCATCAA
CGGCGGGTTCACATCTTTTCTGAGGCGCAAATGGAAGTTTAAAAAGGAGCGCAACAAAGGGT
ACAAGCACCATGCCGAAGATGCTCTGATTATCGCAAATGCCGACTTCATCTTTAAGGAGTGG
AAAAAGCTGGACAAAGCCAAGAAAGTGATGGAGAACCAGATGTTCGAAGAGAAGCAGGCCGA
ATCTATGCCCGAAATCGAGACAGAACAGGAGTACAAGGAGATTTTCATCACTCCTCACCAGA
TCAAGCATATCAAGGATTTCAAGGACTACAAGTACTCTCACCGGGTGGATAAAAAGCCCAAC
AGAGAGCTGATCAATGACACCCTGTATAGTACAAGAAAAGACGATAAGGGGAATACCCTGAT
TGTGAACAATCTGAACGGACTGTACGACAAAGATAATGACAAGCTGAAAAAGCTGATCAACA
AAAGTCCCGAGAAGCTGCTGATGTACCACCATGATCCTCAGACATATCAGAAACTGAAGCTG
ATTATGGAGCAGTACGGCGACGAGAAGAACCCACTGTATAAGTACTATGAAGAGACTGGGAA
CTACCTGACCAAGTATAGCAAAAAGGATAATGGCCCCGTGATCAAGAAGATCAAGTACTATG
GGAACAAGCTGAATGCCCATCTGGACATCACAGACGATTACCCTAACAGTCGCAACAAGGTG
GTCAAGCTGTCACTGAAGCCATACAGATTCGATGTCTATCTGGACAACGGCGTGTATAAATT
TGTGACTGTCAAGAATCTGGATGTCATCAAAAAGGAGAACTACTATGAAGTGAATAGCAAGT
GCTACGAAGAGGCTAAAAAGCTGAAAAAGATTAGCAACCAGGCAGAGTTCATCGCCTCCTTT
TACAACAACGACCTGATTAAGATCAATGGCGAACTGTATAGGGTCATCGGGGTGAACAATGA
TCTGCTGAACCGCATTGAAGTGAATATGATTGACATCACTTACCGAGAGTATCTGGAAAACA
TGAATGATAAGCGCCCCCCTCGAATTATCAAAACAATTGCCTCTAAGACTCAGAGTATCAAA
AAGTACTCAACCGACATTCTGGGAAACCTGTATGAGGTGAAGAGCAAAAAGCACCCTCAGAT
TATCAAAAAGGGCGGATCCCCCAAGAAGAAGAGGAAAGTCTCGAGCTAGGCTGGAGCCTCGG
TGGCCATGCTTCTTGCCCCTTGGGCCTCCCCCCAGCCCCTCCTCCCCTTCCTGCACCCGTAC
CCCCGTGGTCTTTGAATAAAGTCTGAGTGGGCGGCACATGCTGGGGAGAGATCTGCGGCCGC
CTAGCAATAAAGGATCGTTTATTTTCATTGGAAGCGTGTGTTGGTTTTTTGATCAGGCGCGA
GGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCG
CGCAGCTGCCTGCAGG
Cas9 Vector 1 (550 bp minimal RHO promoter driving wt Cas9
with SV40 polyA signal) (see FIG. 18 for feature annotation):
(SEQ ID NO: 9)
CCTGCAGGCAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAACTC
CATCACTAGGGGTTCCTAAGCGGCCGCGGTTCCTCAGATCTGAATTCTCATGTTACAGGCAG
GGAGACGGGCACAAAACACAAATAAAAAGCTTCCATGCTGTCAGAAGCACTATGCAAAAAGC
AAGATGCTGAGGTCATGGAGCTCCTCCTGTCAGAGGAGTGTGGGGACTGGATGACTCCAGAG
GTAACTTGTGGGGGAACGAACAGGTAAGGGGCTGTGTGACGAGATGAGAGACTGGGAGAATA
AACCAGAAAGTCTCTAGCTGTCCAGAGGACATAGCACAGAGGCCCATGGTCCCTATTTCAAA
CCCAGGCCACCAGACTGAGCTGGGACCTTGGGACAGACAAGTCATGCAGAAGTTAGGGGACC
TTCTCCTCCCTTTTCCTGGATCCTGAGTACCTCTCCTCCCTGACCTCAGGCTTCCTCCTAGT
GTCACCTTGGCCCCTCTTAGAAGCCAATTAGGCCCTCAGTTTCTGCAGCGGGGATTAATATG
ATTATGAACACCCCCAATCTCCCAGATGCTGATTCAGCCAGGAGCTTAGGAGGGGGAGGTCA
CTTTATAAGGGTCTGGGGGGGTCAGAACCCAGAGTCATCCAGCTGGAGCCCTGAGTGGCTGA
GCTCAGGCCTTCGCAGCATTCTTGGGTGGGAGCAGCCACGGGTCAGCCACAATCTAGAGGAT
CCGGTACTCGAGGAACTGAAAAACCAGAAAGTTAACTGGTAAGTTTAGTCTTTTTGTCTTTT
ATTTCAGGTCCCGGATCCGGTGGTGGTGCAAATCAAAGAACTGCTCCTCAGTGGATGTTGCC
TTTACTTCTAGGCCTGTACGGAAGTGTTACGCGGCCGCCACCATGGGACCGAAGAAAAAGCG
CAAGGTCGAAGCGTCCATGAAAAGGAACTACATTCTGGGGCTGGACATCGGGATTACAAGCG
TGGGGTATGGGATTATTGACTATGAAACAAGGGACGTGATCGACGCAGGCGTCAGACTGTTC
AAGGAGGCCAACGTGGAAAACAATGAGGGACGGAGAAGCAAGAGGGGAGCCAGGCGCCTGAA
ACGACGGAGAAGGCACAGAATCCAGAGGGTGAAGAAACTGCTGTTCGATTACAACCTGCTGA
CCGACCATTCTGAGCTGAGTGGAATTAATCCTTATGAAGCCAGGGTGAAAGGCCTGAGTCAG
AAGCTGTCAGAGGAAGAGTTTTCCGCAGCTCTGCTGCACCTGGCTAAGCGCCGAGGAGTGCA
TAACGTCAATGAGGTGGAAGAGGACACCGGCAACGAGCTGTCTACAAAGGAACAGATCTCAC
GCAATAGCAAAGCTCTGGAAGAGAAGTATGTCGCAGAGCTGCAGCTGGAACGGCTGAAGAAA
GATGGCGAGGTGAGAGGGTCAATTAATAGGTTCAAGACAAGCGACTACGTCAAAGAAGCCAA
GCAGCTGCTGAAAGTGCAGAAGGCTTACCACCAGCTGGATCAGAGCTTCATCGATACTTATA
TCGACCTGCTGGAGACTCGGAGAACCTACTATGAGGGACCAGGAGAAGGGAGCCCCTTCGGA
TGGAAAGACATCAAGGAATGGTACGAGATGCTGATGGGACATTGCACCTATTTTCCAGAAGA
GCTGAGAAGCGTCAAGTACGCTTATAACGCAGATCTGTACAACGCCCTGAATGACCTGAACA
ACCTGGTCATCACCAGGGATGAAAACGAGAAACTGGAATACTATGAGAAGTTCCAGATCATC
GAAAACGTGTTTAAGCAGAAGAAAAAGCCTACACTGAAACAGATTGCTAAGGAGATCCTGGT
CAACGAAGAGGACATCAAGGGCTACCGGGTGACAAGCACTGGAAAACCAGAGTTCACCAATC
TGAAAGTGTATCACGATATTAAGGACATCACAGCACGGAAAGAAATCATTGAGAACGCCGAA
CTGCTGGATCAGATTGCTAAGATCCTGACTATCTACCAGAGCTCCGAGGACATCCAGGAAGA
GCTGACTAACCTGAACAGCGAGCTGACCCAGGAAGAGATCGAACAGATTAGTAATCTGAAGG
GGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGA
CCTGAGTCAGCAGAAAGAGATCCCAACCACACTGGTGGACGATTTCATTCTGTCACCCGTGG
TCAAGCGGAGCTTCATCCAGAGCATCAAAGTGATCAACGCCATCATCAAGAAGTACGGCCTG
CCCAATGATATCATTATCGAGCTGGCTAGGGAGAAGAACAGCAAGGACGCACAGAAGATGAT
CAATGAGATGCAGAAACGAAACCGGCAGACCAATGAACGCATTGAAGAGATTATCCGAACTA
CCGGGAAAGAGAACGCAAAGTACCTGATTGAAAAAATCAAGCTGCACGATATGCAGGAGGGA
AAGTGTCTGTATTCTCTGGAGGCCATCCCCCTGGAGGACCTGCTGAACAATCCATTCAACTA
CGAGGTCGATCATATTATCCCCAGAAGCGTGTCCTTCGACAATTCCTTTAACAACAAGGTGC
TGGTCAAGCAGGAAGAGAACTCTAAAAAGGGCAATAGGACTCCTTTCCAGTACCTGTCTAGT
TCAGATTCCAAGATCTCTTACGAAACCTTTAAAAAGCACATTCTGAATCTGGCCAAAGGAAA
GGGCCGCATCAGCAAGACCAAAAAGGAGTACCTGCTGGAAGAGCGGGACATCAACAGATTCT
CCGTCCAGAAGGATTTTATTAACCGGAATCTGGTGGACACAAGATACGCTACTCGCGGCCTG
ATGAATCTGCTGCGATCCTATTTCCGGGTGAACAATCTGGATGTGAAAGTCAAGTCCATCAA
CGGCGGGTTCACATCTTTTCTGAGGCGCAAATGGAAGTTTAAAAAGGAGCGCAACAAAGGGT
ACAAGCACCATGCCGAAGATGCTCTGATTATCGCAAATGCCGACTTCATCTTTAAGGAGTGG
AAAAAGCTGGACAAAGCCAAGAAAGTGATGGAGAACCAGATGTTCGAAGAGAAGCAGGCCGA
ATCTATGCCCGAAATCGAGACAGAACAGGAGTACAAGGAGATTTTCATCACTCCTCACCAGA
TCAAGCATATCAAGGATTTCAAGGACTACAAGTACTCTCACCGGGTGGATAAAAAGCCCAAC
AGAGAGCTGATCAATGACACCCTGTATAGTACAAGAAAAGACGATAAGGGGAATACCCTGAT
TGTGAACAATCTGAACGGACTGTACGACAAAGATAATGACAAGCTGAAAAAGCTGATCAACA
AAAGTCCCGAGAAGCTGCTGATGTACCACCATGATCCTCAGACATATCAGAAACTGAAGCTG
ATTATGGAGCAGTACGGCGACGAGAAGAACCCACTGTATAAGTACTATGAAGAGACTGGGAA
CTACCTGACCAAGTATAGCAAAAAGGATAATGGCCCCGTGATCAAGAAGATCAAGTACTATG
GGAACAAGCTGAATGCCCATCTGGACATCACAGACGATTACCCTAACAGTCGCAACAAGGTG
GTCAAGCTGTCACTGAAGCCATACAGATTCGATGTCTATCTGGACAACGGCGTGTATAAATT
TGTGACTGTCAAGAATCTGGATGTCATCAAAAAGGAGAACTACTATGAAGTGAATAGCAAGT
GCTACGAAGAGGCTAAAAAGCTGAAAAAGATTAGCAACCAGGCAGAGTTCATCGCCTCCTTT
TACAACAACGACCTGATTAAGATCAATGGCGAACTGTATAGGGTCATCGGGGTGAACAATGA
TCTGCTGAACCGCATTGAAGTGAATATGATTGACATCACTTACCGAGAGTATCTGGAAAACA
TGAATGATAAGCGCCCCCCTCGAATTATCAAAACAATTGCCTCTAAGACTCAGAGTATCAAA
AAGTACTCAACCGACATTCTGGGAAACCTGTATGAGGTGAAGAGCAAAAAGCACCCTCAGAT
TATCAAAAAGGGCGGATCCCCCAAGAAGAAGAGGAAAGTCTCGAGCTAGCAATAAAGGATCG
TTTATTTTCATTGGAAGCGTGTGTTGGTTTTTTGATCAGGCGCGTCCAAGCTTGCATGCTGG
GGAGAGATCTGCGGCCGCCTAGCAATAAAGGATCGTTTATTTTCATTGGAAGCGTGTGTTGG
TTTTTTGATCAGGCGCGAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGCGGCC
TCAGTGAGCGAGCGAGCGCGCAGCTGCCTGCAGG

### REFERENCES

Ambati et al., Invest Ophthalmol Vis Sci 41(5):1181-1185 (2000a)
Ambati et al., Invest Ophthalmol Vis Sci 41(5):1186-1191 (2000b)
Amrani et al., Genome Biol. 19(1):214 (2018)
Bae et al., Bioinformatics 30(10):1473-1475 (2014)
Berson et al., N Engl J Med. 323(19):1302-1307 (1990)
Briner et al., Mol Cell 56(2):333-339 (2014)
Burstein et al., Nature. 542(7640):237-241 (2017)
Casini et al., Nat Biotechnol. 36(3):265-271 (2018)
Chen et al., Nature. 550(7676):407-410 (2017)
Cideciyan et al., PNAS. 115 (36):E8547-E8556 (2018)
Cong et al., Science. 339(6121):819-23 (2013)
Daiger et al., Arch Ophthalmol. 125(2):151-8 (2007)
Heigwer et al., Nat Methods 11(2):122-3 (2014)
Hsu et al., Nat Biotechnol 31(9):827-832 (2013)
Jiang et al., Nat Biotechnol 31(3):233-239 (2013)
Jinek et al., Science 337(6096):816-821 (2012)
Kim et al., Nat Commun. 8:14500 (2017)
Kleinstiver et al., Nat Biotechnol 33(12):1293-1298 (2015)
Kleinstiver et al., Nature 529(7587):490-495 (2016)
Lee et al., Nat Commun. 9(1):3048 (2018)
Li et al., The CRISPR Journal 01:01 (2018)
Mali et al., Science 339(6121):823-826 (2013)
Nishimasu et al., Cell 156(5):935-949 (2014)
Nishimasu et al., Cell 162(5):1113-1126 (2015)
Nishimasu et al., Science. 361(6408):1259-1262 (2018)
Pellissier et al., Mol Ther Methods Clin Dev. 1:14009 (2014)
Ran et al., Cell 154(6):1380-1389 (2013)
Ran et al., Nature. 520(7546):186-91 (2015)
Strecker et al., Nat Commun. January 22; 10(1):212 (2019)
Teng et al., Cell Discov. 4:63 (2018)
Wang et al., Plant Biotechnol J. pbi.13053 (2018)
Xiao et al. Bioinformatics 30(8):1180-1182 (2014)
Yadav et al., Human Molecular Genetics 23(8):2132-2144 (2014)
Yan et al., Science 363(6422):88-91 (2019)
Zetsche et al., Nat Biotechnol 33(2):139-142 (2015)
Zetsche et al., Nat Biotechnol. 35(1):31-34 (2017)

What is claimed is:

1. A guide RNA (“gRNA”) molecule comprising a targeting domain that binds a target sequence of a RHO gene.

2. The gRNA molecule of claim 1, wherein the targeting domain is complementary to a target domain of a RHO gene.

3. The gRNA molecule of claim 1, wherein said targeting domain is configured to provide a cleavage event selected from a double strand break and a single strand break, within 10 nucleotides of a RHO target position.

4. The gRNA molecule of claim 1, wherein the RHO target position is in the 5′ region of the RHO gene.

5. The gRNA molecule of claim 4, wherein the 5′ region of the RHO gene is selected from the group consisting of the 5′ untranslated (“UTR”) region, exon 1, the exon 1/intron 1 border, exon 2, and the exon 2/intron 1 border of the RHO gene.

6. The gRNA molecule of claim 1, wherein said targeting domain comprises a sequence that is the same as, or differs by no more than 3 nucleotides from, a targeting domain sequence from any of Tables 1-3 and 18.

7. The gRNA molecule of claim 1, wherein said targeting domain is selected from those in Tables 1-3 and 18.

8. The gRNA molecule of any of claims 1-7, wherein said gRNA is a modular gRNA molecule or a chimeric gRNA molecule.

9. The gRNA molecule of any of claims 1-8, comprising from 5′ to 3′:
a targeting domain;
a first complementarity domain;
a linking domain;
a second complementarity domain;
a proximal domain; and
a tail domain.

10. A nucleic acid that comprises: (a) sequence that encodes a gRNA molecule comprising a targeting domain that is complementary to a target domain in the RHO gene.

11. The nucleic acid of claim 10, wherein said gRNA molecule is a gRNA molecule of any of claims 1-9.

12. The nucleic acid of claim 11, wherein said targeting domain is configured to provide a cleavage event selected from a double strand break and a single strand break, within 10 nucleotides of a RHO target position.

13. The nucleic acid of claim 11, wherein said targeting domain comprises a sequence that is the same as, or differs by no more than 3 nucleotides from, a targeting domain sequence from any of Tables 1-3 and 18.

14. The nucleic acid of claim 11, wherein said targeting domain is selected from those in Tables 1-3 and 18.

15. The nucleic acid of any of claims 10-14, wherein said gRNA is a modular gRNA molecule or a chimeric molecule.

16. The nucleic acid of any of claims 10-15, wherein said nucleic acid comprises a promoter operably linked to the sequence that encodes said gRNA molecule of (a).

17. The nucleic acid of claim 16, wherein the promoter operably linked to the sequence that encodes said gRNA molecule of (a) is a U6 promoter.

18. The nucleic acid of any of claims 10-17, further comprising: (b) sequence that encodes an RNA-guided nuclease molecule.

19. The nucleic acid of claim 18, wherein said RNA-guided nuclease molecule forms a double strand break in a target nucleic acid.

20. The nucleic acid of claim 18, wherein said RNA-guided nuclease molecule forms a single strand break in a target nucleic acid.

21. The nucleic acid of claim 20, wherein said single strand break is formed in the strand of the target nucleic acid to which the targeting domain of said gRNA molecule is complementary.

22. The nucleic acid of claim 21, wherein said single strand break is formed in the strand of the target nucleic acid other than the strand to which to which the targeting domain of said gRNA is complementary.

23. The nucleic acid of claim 18, wherein said RNA-guided nuclease molecule is a Cas9 molecule.

24. The nucleic acid of claim 23, wherein said Cas9 molecule comprises a nickase molecule.

25. The nucleic acid of claim 18, wherein said RNA-guided nuclease molecule is a Cpfl molecule.

26. The nucleic acid of any of claims 18-25, wherein said nucleic acid comprises a promoter operably linked to the sequence that encodes the RNA-guided nuclease molecule of (b).

27. The nucleic acid of claim 26, wherein said promoter operably linked to the sequence that encodes the RNA-guided nuclease molecule of (b) comprises a promoter selected from the group consisting of RHO, CMV, EFS, GRK1, CRX, NRL, and RCVRN promoter.

28. The nucleic acid of any of claims 10-27, further comprising: (c) a RHO cDNA molecule.

29. The nucleic acid of claim 24, wherein said RHO cDNA molecule is not codon modified to be resistant to hybridization with the gRNA molecule.

30. The nucleic acid of claim 28, wherein said RHO cDNA molecule comprises a nucleotide sequence comprising exon 1, exon 2, exon 3, exon 4, and exon 5 of the RHO gene.

31. The nucleic acid of claim 28, wherein said RHO cDNA molecule comprises a nucleotide sequence comprising exon 1, intron 1, exon 2, exon 3, exon 4, and exon 5 of the RHO gene.

32. The nucleic acid of claim 28, wherein intron 1 comprises one or more truncations at a 5′ end of intron 1, a 3′ end of intron 1, or both.

33. The nucleic acid of any of claims 28-32, wherein said nucleic acid comprises a 3′ UTR nucleotide sequence downstream of the RHO cDNA molecule.

34. The nucleic acid of claim 33, wherein said 3′ UTR nucleotide sequence comprises a RHO gene 3′ UTR nucleotide sequence.

35. The nucleic acid of claim 33, wherein said 3′ UTR nucleotide sequence comprises an α-globin 3′ UTR nucleotide sequence.

36. The nucleic acid of claim 33, wherein said 3′ UTR nucleotide sequence comprises a β-globin 3′ UTR nucleotide sequence.

37. The nucleic acid of any of claims 33-36, wherein said 3′ UTR nucleotide sequence comprises one or more truncations at a 5′ end of said 3′ UTR nucleotide sequence, a 3′ end of said 3′ UTR nucleotide sequence, or both.

38. The nucleic acid of any of claims 28-37, wherein said nucleic acid comprises a promoter operably linked to the RHO cDNA molecule (c).

39. The nucleic acid of claim 38, wherein said promoter operably linked to the RHO cDNA molecule (c) is a rod-specific promoter.

40. The nucleic acid of claim 39, wherein said rod-specific promoter is a human RHO promoter.

41. The nucleic acid of claim 40, wherein said human RHO promoter comprises an endogenous RHO promoter.

42. The nucleic acid of claim 41, further comprising: (d) sequence that encodes a second gRNA molecule having a targeting domain that is complementary to a second target domain of the RHO gene.

43. The nucleic acid of claim 42, wherein said targeting domain of said second gRNA is configured to provide a cleavage event selected from a double strand break and a single strand break, within 10 nucleotides of a RHO target position.

44. The nucleic acid of any of claim 42 or 43, wherein said second gRNA molecule is a modular gRNA molecule or chimeric gRNA molecule.

45. The nucleic acid of any of claims 42-44, wherein said second gRNA molecule is a chimeric gRNA molecule.

46. The nucleic acid of any of claims 42-45, wherein said second gRNA molecule comprises from 5′ to 3′:
a targeting domain;
a first complementarity domain;
a linking domain;
a second complementarity domain;
a proximal domain; and
a tail domain.

47. The nucleic acid of any of claims 42-46, further comprising a third gRNA molecule.

48. The nucleic acid of claim 47, further comprising a fourth gRNA molecule.

49. The nucleic acid of any of claims 18-27, wherein each of (a) and (b) is present on the same nucleic acid molecule.

50. The nucleic acid of claim 49, wherein said nucleic acid molecule is an AAV vector.

51. The nucleic acid of any of claims 18-27, wherein: (a) is present on a first nucleic acid molecule; and (b) is present on a second nucleic acid molecule.

52. The nucleic acid of claim 51, wherein said first and second nucleic acid molecules are AAV vectors.

53. The nucleic acid of any of claims 18-27, further comprising (c) a RHO cDNA molecule of any of claims 28-41.

54. The nucleic acid of claim 53, wherein each of (a) and (c) is present on the same nucleic acid molecule.

55. The nucleic acid of claim 54, wherein said nucleic acid molecule is an AAV vector.

56. The nucleic acid of claim 53, wherein: (a) is present on a first nucleic acid molecule; and (c) is present on a second nucleic acid molecule.

57. The nucleic acid of claim 56, wherein said first and second nucleic acid molecules are AAV vectors.

58. The nucleic acid of any of claims 10-17, further comprising:
(b) sequence that encodes an RNA-guided nuclease molecule of any of claims 18-27; and
(c) RHO cDNA molecule of any of claims 28-41.

59. The nucleic acid of claim 58, wherein each of (a), (b), and (c) are present on the same nucleic acid molecule.

60. The nucleic acid of claim 59, wherein said nucleic acid molecule is an AAV vector.

61. The nucleic acid of claim 58, wherein:
one of (a), (b), and (c) are present on a first nucleic acid molecule; and
and a second and third of (a), (b), and (c) is present on a second nucleic acid molecule.

62. The nucleic acid of claim 61, wherein said first and second nucleic acid molecules are AAV vectors.

63. The nucleic acid of claim 58, wherein: (a) is present on a first nucleic acid molecule; and (b) and (c) are present on a second nucleic acid molecule.

64. The nucleic acid of claim 63, wherein said first and second nucleic acid molecules are AAV vectors.

65. The nucleic acid of claim 58, wherein: (b) is present on a first nucleic acid molecule; and (a) and (c) are present on a second nucleic acid molecule.

66. The nucleic acid of claim 65, wherein said first and second nucleic acid molecules are AAV vectors.

67. The nucleic acid of claim 58, wherein: (c) is present on a first nucleic acid molecule; and (b) and (a) are present on a second nucleic acid molecule.

68. The nucleic acid of claim 67, wherein said first and second nucleic acid molecules are AAV vectors.

69. The nucleic acid of any of claims 51, 56, 61, 63, 65, and 67, wherein said first nucleic acid molecule is other than an AAV vector and said second nucleic acid molecule is an AAV vector.

70. A composition comprising a gRNA molecule of any of claims 1-17.

71. The composition of claim 70, further comprising (b) a Cas9 molecule of any of claims 18-27.

72. The composition of claim 71, further comprising (c) a RHO cDNA molecule of any of claims 28-41.

73. The composition of claim 72, further comprising a second gRNA molecule.

74. The composition of claim 73, further comprising a third gRNA molecule.

75. The composition of claim 75, further comprising a fourth gRNA molecule.

76. A method of altering a cell comprising contacting said cell with:
(a) a gRNA of any of claims 1-17;
(b) an RNA-guided nuclease molecule of any of claims 18-27;
(c) a RHO cDNA molecule of any of claims 28-41; and
optionally, (d) a second gRNA molecule of any of claims 42-46.

77. The method of claim 76, further comprising a third gRNA molecule.

78. The method of claim 77, further comprising a fourth gRNA molecule.

79. The method of claim 76, comprising contacting said cell with (a), (b), (c) and optionally (d).

80. The method of any of claims 76-79, wherein said cell is from a subject suffering from adRP.

81. The method of any of claims 76-80, wherein said cell is from a subject having a mutation in the RHO gene.

82. The method of any of claims 76-81, wherein said cell is a retinal cell.

83. The method of claim 82, wherein the retinal cell is a rod photoreceptor.

84. The method of any of claims 76-83, wherein said cell is an embryonic stem cell, an induced pluripotent stem cell, a hematopoietic stem cell, a neuronal stem cell or a mesenchymal stem cell.

85. The method of any of claims 76-83, wherein said contacting is performed ex vivo.

86. The method of claim 84, wherein said contacted cell is returned to said subject's body.

87. The method of any of claims 76-83, wherein said contacting is performed in vivo.

88. The method of any of claims 80-87, comprising acquiring knowledge of the presence of the mutation in the RHO gene in said cell.

89. The method of claim 88, comprising acquiring knowledge of the presence of the presence of the mutation in the RHO gene in said cell by sequencing a portion of the RHO gene.

90. The method of any of claims 76-89, comprising altering a RHO target position to knock-out function of the RHO gene.

91. The method of any of claims 76-90, wherein contacting comprises contacting said cell with a nucleic acid that encodes at least one of (a), (b), (c) and optionally (d).

92. The method of any of claims 76-90, wherein contacting comprises delivering to said cell said RNA-guided nuclease molecule of (b) and a nucleic acid which encodes (a) and (c) and optionally (d).

93. The method of any of claims 76-90, wherein contacting comprises delivering to said cell said RNA-guided nuclease molecule of (b), said gRNA molecule of (a), and said RHO cDNA molecule of (c).

94. The method of any of claims 76-90, wherein contacting comprises delivering to said cell said gRNA molecule of (a), said RHO cDNA molecule of (c) and a nucleic acid that encodes the RNA-guided nuclease molecule of (b).

95. A method of contacting a subject (or a cell from said subject) with:
(a) a gRNA of any of claims 1-17;
(b) an RNA-guided nuclease molecule of any of claims 18-27;
(c) a RHO cDNA molecule of any of claims 28-41; and
optionally, (d) a second gRNA of any of claims 42-46.

96. The method of claim 95, further comprising a third gRNA molecule.

97. The method of claim 96, further comprising a fourth gRNA molecule.

98. The method of claim 97, further comprising contacting said subject with (a), (b), (c) and optionally (d).

99. The method of claims any one of claims 95-98, wherein said subject is suffering from adRP.

100. The method of any of claims 95-99, wherein said subject has a mutation in the RHO gene.

101. The method of any of claims 95-100, comprising acquiring knowledge of the presence of the mutation in the RHO gene in said subject.

102. The method of claim 101, comprising acquiring knowledge of the presence of the mutation in the RHO gene in said subject by sequencing a portion of the RHO gene.

103. The method of claims 95-102, comprising altering a RHO target position to knock-out function of the RHO gene.

104. The method of any of claims 95-103, wherein a cell of said subject is contacted ex vivo with (a), (b), (c) and optionally (d).

105. The method of claim 104, wherein said cell is returned to the subject's body.

106. The method of any of claims 95-105, wherein treatment comprises introducing a cell into said subject's body, wherein said cell subject is contacted ex vivo with (a), (b), (c), and optionally (d).

107. The method of any of claims 95-106, wherein said contacting is performed in vivo.

108. The method of claim 107, wherein said contacting comprises intravenous delivery.

109. The method of any of claims 95-108, wherein contacting comprises contacting said subject with a nucleic acid that encodes at least one of (a), (b), and (c), and optionally (d).

110. The method of any of claims 95-108, wherein contacting comprises contacting said subject with a nucleic acid of any of claims 10-69.

111. The method of any of claims 95-108, wherein contacting comprises delivering to said subject said RNA-guided nuclease molecule of (b) and a nucleic acid which encodes and (a) and (c), and optionally (d).

112. The method of any of claims 95-108, wherein contacting comprises delivering to said subject said RNA-guided nuclease molecule of (b), said gRNA of (a) and said RHO cDNA molecule of (c), and optionally said second gRNA of (d).

113. The method of any of claims 95-108, wherein contacting comprises delivering to said subject said gRNA of (a), said RHO cDNA molecule of (c) and a nucleic acid that encodes the RNA-guided nuclease molecule of (b).

114. A reaction mixture comprising a gRNA, a nucleic acid, or a composition described herein, and a cell from a subject having adRP, or a subject having a mutation in the RHO gene.

115. A kit comprising, (a) gRNA molecule of any of claims 1-17, or nucleic acid that encodes said gRNA, and one or more of the following:
(b) a Cas9 molecule of any of claims 18-27;
(c) a RHO cDNA molecule of any of claims 28-41;
optionally, (d) a second gRNA molecule of any of claims 42-46; and
(e) nucleic acid that encodes one or more of (b) and (c).

116. The kit of claim 115, comprising nucleic acid that encodes one or more of (a), (b) (c) and (d).

117. The kit of claim 116, further comprising a third gRNA molecule targeting a RHO target position of the RHO gene.

118. The kit of claim 117, further comprising a fourth gRNA molecule targeting a RHO target position of the RHO gene.


SEQUENCE LISTING (1002 sequences)

SEQ ID NO: 1 - DNA, 6706 bp, Artificial Sequence
  RHO genomic
agagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccaccaagacctactgatctggagtcccacgttccccaaggccagcgggatgtgtgcccctcctcctcccaactcatctttcaggaacacgaggattcttgctttctggaaaagtgtcccagcttagggataagtgtctagcacagaatggggcacacagtaggtgcttaataaatgctggatggatgcaggaaggaatggaggaatgaatgggaagggagaacatatctatcctctcagaccctcgcagcagcagcaactcatacttggctaatgatatggagcagttgtttttccctccctgggcctcactttcttctcctataaaatggaaatcccagatccctggtcctgccgacacgcagctactgagaagaccaaaagaggtgtgtgtgtgtctatgtgtgtgtttcagcactttgtaaatagcaagaagctgtacagattctagttaatgttgtgaataacatcaattaatgtaactagttaattactatgattatcacctcctgatagtgaacattttgagattgggcattcagatgatggggtttcacccaaccttggggcaggtttttaaaaattagctaggcatcaaggccagaccagggctgggggttgggctgtaggcagggacagtcacaggaatgcagaatgcagtcatcagacctgaaaaaacaacactgggggagggggacggtgaaggccaagttcccaatgagggtgagattgggcctggggtctcacccctagtgtggggccccaggtcccgtgcctccccttcccaatgtggcctatggagagacaggcctttctctcagcctctggaagccacctgctcttttgctctagcacctgggtcccagcatctagagcatggagcctctagaagccatgctcacccgcccacatttaattaacagctgagtccctgatgtcatccttatctcgaagagcttagaaacaaagagtgggaaattccactgggcctaccttccttggggatgttcatgggccccagtttccagtttcccttgccagacaagcccatcttcagcagttgctagtccattctccattctggagaatctgctccaaaaagctggccacatctctgaggtgtcagaattaagctgcctcagtaactgctcccccttctccatataagcaaagccagaagctctagctttacccagctctgcctggagactaaggcaaattgggccattaaaagctcagctcctatgttggtattaacggtggtgggttttgttgctttcacactctatccacaggatagattgaaactgccagcttccacctgatccctgaccctgggatggctggattgagcaatgagcagagccaagcagcacagagtcccctggggctagaggtggaggaggcagtcctgggaatgggaaaaacccca

SEQ ID NO: 2 - DNA, 1047 bp, Artificial Sequence
  Wild-type RHO cDNA
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 3 - skipped

SEQ ID NO: 4 - DNA, 2828 bp, Artificial Sequence
  RHO cDNA with intron 1
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 5 - DNA, 2253 bp, Artificial Sequence
  RHO cDNA with intron 2
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 6 - DNA, 1163 bp, Artificial Sequence
  RHO cDNA with intron 3
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 7 - DNA, 1882 bp, Artificial Sequence
  RHO cDNA with intron 4
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaa

SEQ ID NO: 8 - DNA, 1670 bp, Artificial Sequence
  Vector 2
ccacgtcagaatcaaaccctcaccttaacctcattagcgttgggcataatcaccaggccaagcgccttaaactacgagaggccccatcccacccgccctgccttagccctgccacgtgtgccaaacgctgttagacccaacaccacccaggccaggtagggggctggagcccaggtgggcatttgagtcaccaacccccaggcagtctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccaccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaagctggagcctcggtggccatgcttcttgccccttgggcctccccccagcccctcctccccttcctgcacccgtacccccgtggtctttgaataaagtctgagtgggcggca

SEQ ID NO: 9 - DNA, 4498 bp, Artificial Sequence
  Vector 1: 550 bp minimal RHO promoter driving wt Cas9 with SV40 polyA signal
  misc_feature (1)..(141): ITR
  misc_feature (172)..(796): MinRHO promoter
  misc_feature (797)..(960): SV40 SA/SD
  misc_feature (964)..(976): Kozak start
  misc_feature (979)..(1008): NLS
  misc_feature (1009)..(4167): Sa Cas9
  misc_feature (4174)..(4194): SV40 NLS
  misc_feature (4201)..(4260): Minimal polyA
  misc_feature (4358)..(4498): ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaagcggccgcggttcctcagatctgaattctcatgttacaggcagggagacgggcacaaaacacaaataaaaagcttccatgctgtcagaagcactatgcaaaaagcaagatgctgaggtcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaatctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgcggccgccaccatgggaccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcggatcccccaagaagaagaggaaagtctcgagctagcaataaaggatcgtttattttcattggaagcgtgtgttggttttttgatcaggcgcgtccaagcttgcatgctggggagagatctgcggccgcctagcaataaaggatcgtttattttcattggaagcgtgtgttggttttttgatcaggcgcgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 10 - DNA, 4170 bp, Artificial Sequence
  Cas9 Vector 2: 250 bp minimal RHO promoter driving Cas9 w/ alpha globin UTR
  misc_feature (1)..(141): ITR
  misc_feature (179)..(427): MinRHO promoter (250bp)
  misc_feature (428)..(590): SV40 intron
  misc_feature (607)..(636): NLS
  misc_feature (637)..(3795): Sa Cas9
  misc_feature (3802)..(3822): SV40 NLS
  misc_feature (3832)..(3942): HBA1 UTR
  misc_feature (4030)..(4170): ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaagcggccgcggttcctcagatctgaattcggtacctgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttactccgccaccatgggaccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcggatcccccaagaagaagaggaaagtctcgagctaggctggagcctcggtggccatgcttcttgccccttgggcctccccccagcccctcctccccttcctgcacccgtacccccgtggtctttgaataaagtctgagtgggcggcacatgctggggagagatctgcggccgcctagcaataaaggatcgtttattttcattggaagcgtgtgttggttttttgatcaggcgcgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - DNA, 2691 bp, Artificial Sequence
  Exemplary replacement vector: 250 bp minimal RHO promoter driving codon-optimized RHO cDNA; U6 promoter driving gRNA targeting RHO
  misc_feature (1)..(139): ITR
  misc_feature (174)..(437): U6 promoter
  misc_feature (439)..(460): RHO-7 gRNA targeting domain
  misc_feature (461)..(536): Sa gRNA scaffold
  misc_feature (563)..(826): U6 promoter
  misc_feature (828)..(849): RHO-7 gRNA targeting domain
  misc_feature (850)..(925): Sa gRNA scaffold
  misc_feature (947)..(1195): minRHO promoter (250bp)
  misc_feature (1196)..(1359): SV40 intron
  misc_feature (1369)..(2412): Codon Optimized RHO cDNA
  misc_feature (2416)..(2526): HBA1 UTR
  misc_feature (2553)..(2691): ITR
tgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggttcctcagatctgaattcggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcccacacccggctcataccgccgttatagtactctggaaacagaatctactataacaaggcaaaatgccgtgtttatctcgtcaacttgttggcgagattttttcgacttagttcgatcgaaggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcccacacccggctcataccgccgttatagtactctggaaacagaatctactataacaaggcaaaatgccgtgtttatctcgtcaacttgttggcgagattttttggtaccgctagcgctgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttactccgccaccatgaatggcacagagggccctaacttctacgtgccctttagcaatgccacaggcgtcgtgcggagcccttttgagtaccctcagtactatctggccgagccttggcagtttagcatgctggccgcctacatgttcctgctgatcgtgctgggcttccccatcaactttctgaccctgtacgtgaccgtgcagcacaagaagctgcggacccctctgaactacatcctgctgaatctggccgtggccgacctgtttatggtgctcggcggctttaccagcacactgtacacaagcctgcacggctacttcgtgtttggccccaccggctgcaatctggaaggcttttttgccacactcggcggcgaaattgctctgtggtcactggtggtgctggccatcgagagatacgtggtcgtgtgcaagcccatgagcaacttcagattcggcgagaaccacgccatcatgggcgtcgcctttacatgggttatggccctggcttgtgcagctcctcctcttgccggctggtccagatatattcctgagggcctgcagtgcagctgcggcatcgattactacaccctgaagcctgaagtgaacaacgagagcttcgtgatctacatgtttgtggtgcacttcacgatccccatgatcatcatattcttttgctacggccagctggtgttcaccgtgaaagaagccgctgctcagcagcaagagagcgccacaacacagaaagccgagaaagaagtgacccggatggtcattatcatggttatcgcctttctgatctgttgggtgccctacgccagcgtggccttctacatctttacccaccaaggcagcaacttcggccccatctttatgacaatccccgccttctttgccaagagcgccgccatctacaaccccgtgatctatatcatgatgaacaagcagttccgcaactgcatgctgaccaccatctgctgcggaaagaaccctctgggagatgatgaggccagcgccaccgtgtctaagaccgaaacatctcaggtggcccctgcatgagctggagcctcggtggccatgcttcttgccccttgggcctccccccagcccctcctccccttcctgcacccgtacccccgtggtctttgaataaagtctgagtgggcggcacatgctggggagagatctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgca

SEQ ID NO: 12 - DNA, 76 bp, Artificial Sequence
  gRNA scaffold sequence
gttatagtactctggaaacagaatctactataacaaggcaaaatgccgtgtttatctcgtcaacttgttggcgaga

SEQ ID NO: 13 - DNA, 1047 bp, Artificial Sequence
  Codon optimized RHO-encoding sequence 1 (Codon 1)
atgaacggcaccgagggccccaacttctacgtccccttcagcaacgccaccggcgtcgtccgcagccccttcgagtacccccagtactacctggccgagccctggcagttcagcatgctggccgcctacatgttcctgctgatcgtcctgggcttccccatcaacttcctgaccctgtacgtcaccgtccagcacaagaagctgcgcacccccctgaactacatcctgctgaacctggccgtcgccgacctgttcatggtcctgggcggcttcaccagcaccctgtacaccagcctgcacggctacttcgtcttcggccccaccggctgcaacctggagggcttcttcgccaccctgggcggcgagatcgccctgtggagcctggtcgtcctggccatcgagcgctacgtcgtcgtctgcaagcccatgagcaacttccgcttcggcgagaaccacgccatcatgggcgtcgccttcacctgggtcatggccctggcctgcgccgccccccccctggccggctggagccgctacatccccgagggcctgcagtgcagctgcggcatcgactactacaccctgaagcccgaggtcaacaacgagagcttcgtcatctacatgttcgtcgtccacttcaccatccccatgatcatcatcttcttctgctacggccagctggtcttcaccgtcaaggaggccgccgcccagcagcaggagagcgccaccacccagaaggccgagaaggaggtcacccgcatggtcatcatcatggtcatcgccttcctgatctgctgggtcccctacgccagcgtcgccttctacatcttcacccaccagggcagcaacttcggccccatcttcatgaccatccccgccttcttcgccaagagcgccgccatctacaaccccgtcatctacatcatgatgaacaagcagttccgcaactgcatgctgaccaccatctgctgcggcaagaaccccctgggcgacgacgaggccagcgccaccgtcagcaagaccgagaccagccaggtcgcccccgcctaa

SEQ ID NO: 14 - DNA, 1047 bp, Artificial Sequence
  Codon optimized RHO-encoding sequence 2 (Codon 2)
atgaacggcaccgagggccccaacttctacgtgcccttctccaacgccaccggcgtggtgcgctcccccttcgagtacccccagtactacctggccgagccctggcagttctccatgctggccgcctacatgttcctgctgatcgtgctgggcttccccatcaacttcctgaccctgtacgtgaccgtgcagcacaagaagctgcgcacccccctgaactacatcctgctgaacctggccgtggccgacctgttcatggtgctgggcggcttcacctccaccctgtacacctccctgcacggctacttcgtgttcggccccaccggctgcaacctggagggcttcttcgccaccctgggcggcgagatcgccctgtggtccctggtggtgctggccatcgagcgctacgtggtggtgtgcaagcccatgtccaacttccgcttcggcgagaaccacgccatcatgggcgtggccttcacctgggtgatggccctggcctgcgccgccccccccctggccggctggtcccgctacatccccgagggcctgcagtgctcctgcggcatcgactactacaccctgaagcccgaggtgaacaacgagtccttcgtgatctacatgttcgtggtgcacttcaccatccccatgatcatcatcttcttctgctacggccagctggtgttcaccgtgaaggaggccgccgcccagcagcaggagtccgccaccacccagaaggccgagaaggaggtgacccgcatggtgatcatcatggtgatcgccttcctgatctgctgggtgccctacgcctccgtggccttctacatcttcacccaccagggctccaacttcggccccatcttcatgaccatccccgccttcttcgccaagtccgccgccatctacaaccccgtgatctacatcatgatgaacaagcagttccgcaactgcatgctgaccaccatctgctgcggcaagaaccccctgggcgacgacgaggcctccgccaccgtgtccaagaccgagacctcccaggtggcccccgcctaa

SEQ ID NO: 15 - DNA, 1047 bp, Artificial Sequence
  Codon Optimized RHO-encoding sequence 3 (Codon 3)
atgaacggcaccgagggccccaacttctacgtccccttcagcaacgccaccggcgtcgtccgcagccccttcgagtacccccagtactacctggccgagccctggcagttctctatgctggccgcctacatgttcctgctgatcgtcctgggcttccctatcaacttcctcaccctctacgtcaccgtccagcacaagaagctccgcacccctctcaactacatcctccttaaccttgccgtcgccgaccttttcatggtccttggcggcttcacctctactctttacacttctttgcacgggtacttcgtgttcggtcctactggttgcaacttggagggtttcttcgccactttgggtggtgagatcgccttgtggtcgttggtggtgttagctatcgagcgatacgtggtggtgtgcaagcctatgtcgaacttccggttcggtgagaatcatgctatcatgggagtggcttttacttgggtgatggctttagcttgcgctgctcctccgttagctggatggtcgcgttatatcccggagggattacagtgctcatgcggaatcgactattatactctaaagccggaagttaataatgaatcatttgttatttatatgtttgttgttcattttacaattccgatgattattatttttttttgttatggacagctagtttttacagttaaggaagcagcagcacagcaacaagaatcagcaacaacacaaaaggcagaaaaagaagttacaaggatggttattattatggtaattgcatttctaatatgttgggtaccgtatgcatccgtagcattttatatatttacacatcaagggtccaattttgggccaatatttatgacgataccagcgttttttgcgaaatccgcggcgatatataatccagtaatatatataatgatgaataaacaatttagaaattgtatgctaacgacgatatgttgtgggaaaaatccactaggggatgatgaagcgagtgcgacggtaagtaaaacggaaacgagtcaagtagcgccagcgtaa

SEQ ID NO: 16 - DNA, 1047 bp, Artificial Sequence
  Codon Optimized RHO-encoding sequence 4 (Codon 4)
atgaacggcaccgagggtcccaatttctacgtcccattttccaacgccacgggggtggtacgcagccctttcgaatatccgcagtactatctggctgagccctggcagttttctatgctcgcagcgtacatgttcttgctaatcgttctgggatttccaattaatttcctcacattgtatgtcaccgtgcagcacaagaagctacggacgcctctgaactacatcctcttgaatctagccgtcgctgacctgtttatggttctcggcggtttcacatcgaccttgtatacgtcactacatgggtactttgtcttcggaccgacaggctgcaacctggaaggttttttcgcaaccctcgggggagagattgcgttgtggtccctagtggtactggccatcgaaaggtatgttgtcgtgtgtaagcccatgagcaattttcgcttcggcgagaaccacgctattatgggtgtagcatttacgtgggttatggcgctcgcctgcgctgcaccacctttggcggggtggtctcggtacatcccggaaggactacagtgttcgtgcggcattgattattacacactgaagcccgaggtcaataacgaatcattcgtgatctatatgtttgtagttcatttcaccattccaatgatcattatctttttctgttacggtcagctcgtctttacggtgaaggaggccgctgcacagcagcaggaatccgcgacaacccagaaggccgagaaggaagtaacgaggatggttattatcatggtcattgctttcttgatctgctgggtgccttatgcaagcgtagcgttttacattttcacacaccaggggtctaattttggaccgatcttcatgaccattcccgcctttttcgctaagtcggcagcgatctataacccagttatttacatcatgatgaataagcagtttcgcaactgtatgctaacgacaatttgctgtggcaagaatcctctgggtgacgatgaggcctcagctaccgtctccaagacggaaacaagccaggtggcaccggcgtaa

SEQ ID NO: 17 - DNA, 1047 bp, Artificial Sequence
  Codon Optimized RHO-encoding sequence 5 (Codon 5)
atgaatgggactgaaggacctaatttctatgtgccatttagcaatgctactggcgttgtcagaagccccttcgaatatccacaatactatctggccgaaccttggcagttcagcatgctcgctgcctatatgtttctgctgattgtgctgggctttcccataaatttcctcaccctgtatgttactgttcaacacaaaaagctgcggacgcctctgaactacatactgctgaacctggccgtcgccgacctgtttatggtcctgggaggctttacaagcactctgtatacaagcctgcacggctacttcgtgttcggccccacaggctgcaacctcgaaggcttctttgccaccctcggaggagagattgccctgtggagcctggtggtgctggccatcgaaaggtatgtggtggtgtgtaaacccatgtccaattttcggttcggcgagaaccacgctattatgggagtggctttcacttgggtgatggccctggcctgcgccgccccaccactggccgggtggagccggtacatcccagaggggctgcaatgtagctgcggaatcgactattataccctgaaaccagaggtgaacaacgagagctttgtgatttatatgtttgtggtgcattttacaattcctatgattatcattttcttctgttacgggcaactggtgtttaccgtgaaggaagccgccgctcaacagcaggagagcgccacaacccaaaaggccgagaaggaggtgaccagaatggtgattattatggtgatcgcttttctgatttgctgggtgccatacgctagcgtcgctttctatattttcactcaccaggggagcaacttcggccccattttcatgacaatccctgccttttttgctaaaagcgccgccatctataacccagtgatctacatcatgatgaacaaacagtttaggaactgtatgctcacaacaatctgctgtggaaagaaccccctcggcgatgacgaagccagcgccaccgtcagcaagacagaaacaagccaggtggcccctgcctaa

SEQ ID NO: 18 - DNA, 1047 bp, Artificial Sequence
  Codon Optimized RHO-encoding sequence 6 (Codon 6)
atgaatggcacagagggccctaacttctacgtgccctttagcaatgccacaggcgtcgtgcggagcccttttgagtaccctcagtactatctggccgagccttggcagtttagcatgctggccgcctacatgttcctgctgatcgtgctgggcttccccatcaactttctgaccctgtacgtgaccgtgcagcacaagaagctgcggacccctctgaactacatcctgctgaatctggccgtggccgacctgtttatggtgctcggcggctttaccagcacactgtacacaagcctgcacggctacttcgtgtttggccccaccggctgcaatctggaaggcttttttgccacactcggcggcgaaattgctctgtggtcactggtggtgctggccatcgagagatacgtggtcgtgtgcaagcccatgagcaacttcagattcggcgagaaccacgccatcatgggcgtcgcctttacatgggttatggccctggcttgtgcagctcctcctcttgccggctggtccagatatattcctgagggcctgcagtgcagctgcggcatcgattactacaccctgaagcctgaagtgaacaacgagagcttcgtgatctacatgtttgtggtgcacttcacgatccccatgatcatcatattcttttgctacggccagctggtgttcaccgtgaaagaagccgctgctcagcagcaagagagcgccacaacacagaaagccgagaaagaagtgacccggatggtcattatcatggttatcgcctttctgatctgttgggtgccctacgccagcgtggccttctacatctttacccaccaaggcagcaacttcggccccatctttatgacaatccccgccttctttgccaagagcgccgccatctacaaccccgtgatctatatcatgatgaacaagcagttccgcaactgcatgctgaccaccatctgctgcggaaagaaccctctgggagatgatgaggccagcgccaccgtgtctaagaccgaaacatctcaggtggcccctgcatga

SEQ ID NO: 19-37 - skipped (19 entries)

SEQ ID NO: 38 - DNA, 111 bp, Artificial Sequence
  HBA1 3′UTR
gctggagcctcggtggccatgcttcttgccccttgggcctccccccagcccctcctccccttcctgcacccgtacccccgtggtctttgaataaagtctgagtgggcggca

SEQ ID NO: 39 - DNA, 101 bp, Artificial Sequence
  short HBA1 3′UTR
gctggagcctcggtggccatgcttcttgccccttgggcctccccccagcccctcctccccttcctgcacccgtacccccgtggtctttgaataaagtctga

SEQ ID NO: 40 - DNA, 304 bp, Artificial Sequence
  TH 3′UTR
gtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctcta

SEQ ID NO: 41 - DNA, 1406 bp, Artificial Sequence
  COL1A1 3′UTR
actccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 42 - DNA, 681 bp, Artificial Sequence
  ALOX15 3′UTR
gcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaa

SEQ ID NO: 43 - DNA, 516 bp, Artificial Sequence
  Human rhodopsin promoter
ccacgtcagaatcaaaccctcaccttaacctcattagcgttgggcataatcaccaggccaagcgccttaaactacgagaggccccatcccacccgccctgccttagccctgccacgtgtgccaaacgctgttagacccaacaccacccaggccaggtagggggctggagcccaggtgggcatttgagtcaccaacccccaggcagtctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccaccaccatgg

SEQ ID NO: 44 - DNA, 249 bp, Artificial Sequence
  Minimal Human rhodopsin promoter
gtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 45 - DNA, 617 bp, Artificial Sequence
  CMV promoter
cattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgc

SEQ ID NO: 46 - DNA, 252 bp, Artificial Sequence
  EFS promoter
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgg

SEQ ID NO: 47 - DNA, 292 bp, Artificial Sequence
  Human GRK1 (rhodopsin kinase) promoter
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttcgcctggtgctgtgtcagccccggtctcccaggggcttcccagtggtccccaggaaccctcgacagggcccggtctctctcgtccagcaagggcagggacgggccacaggccaagggc

SEQ ID NO: 48 - DNA, 113 bp, Artificial Sequence
  Human CRX (cone rod homeobox transcription factor) promoter
gcctgtagccttaatctctcctagcagggggtttgggggagggaggaggagaaagaaagggccccttatggctgagacacaatgacccagccacaaggagggattaccgggcg

SEQ ID NO: 49 - DNA, 281 bp, Artificial Sequence
  Human NRL (neural retina leucine zipper transcription factor enhance upstream of the human TK terminal promoter)
aggtaggaagtggcctttaactccatagaccctatttaaacagcttcggacaggtttaaacatctccttggataattcctagtatccctgttcccactcctactcagggatgatagctctaagaggtgttaggggattaggctgaaaatgtaggtcacccctcagccatctgggaactagaatgagtgagagaggagagaggggcagagacacacacattcgcatattaaggtgacgcgtgtggcctcgaacaccgagcgaccctgcagcgacccgcttaa

SEQ ID NO: 50 - DNA, 235 bp, Artificial Sequence
  Human RCVRN (recoverin) promoter
attttaatctcactagggttctgggagcacccccccccaccgctcccgccctccacaaagctcctgggcccctcctcccttcaaggattgcgaagagctggtcgcaaatcctcctaagccaccagcatctcggtcttcagctcacaccagccttgagcccagcctgcggccaggggaccacgcacgtcccacccacccagcgactccccagccgctgcccactcttcctcactca

SEQ ID NO: 51 - protein, 22 aa, Artificial Sequence
  3X Flag tag
DYKDHDGDYKDHDIDYKDDDDK

SEQ ID NO: 52 - protein, 8 aa, Artificial Sequence
  Flag tag (single)
DYKDDDDK

SEQ ID NO: 53 - protein, 9 aa, Artificial Sequence
  HA tag
YPYDVPDYA

SEQ ID NO: 54 - protein, 10 aa, Artificial Sequence
  Myc tag
EQKLISEEDL

SEQ ID NO: 55 - protein, 6 aa, Artificial Sequence
  HIS tag
HHHHHH

SEQ ID NO: 56 - DNA, 60 bp, Artificial Sequence
  Mini polyA
tagcaataaaggatcgtttattttcattggaagcgtgtgttggttttttgatcaggcgcg

SEQ ID NO: 57 - DNA, 228 bp, Artificial Sequence
  bGH polyA
gctgcaggatgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggaca

SEQ ID NO: 58 - DNA, 134 bp, Artificial Sequence
  SV40 polyA
atgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 59 - DNA, 143 bp, Artificial Sequence
  AAV1 Left ITR
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaa

SEQ ID NO: 60 - DNA, 145 bp, Artificial Sequence
  AAV2 Left ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 61 - DNA, 142 bp, Artificial Sequence
  AAV3B Left ITR
tggccactccctctatgcgcactcgctcgctcggtggggcctggcgaccaaaggtcgccagacggacgtgctttgcacgtccggccccaccgagcgagcgagtgcgcatagagggagtggccaactccatcactagaggtat

SEQ ID NO: 62 - DNA, 146 bp, Artificial Sequence
  AAV4 Left ITR
ttggccactccctctatgcgcgctcgctcactcactcggccctggagaccaaaggtctccagactgccggcctctggccggcagggccgagtgagtgagcgagcgcgcatagagggagtggccaactccatcatctaggtttgccc

SEQ ID NO: 63 - DNA, 155 bp, Artificial Sequence
  AAV5 Left ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaa

SEQ ID NO: 64 - DNA, 143 bp, Artificial Sequence
  AAV6 Left ITR
atacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 65 - DNA, 145 bp, Artificial Sequence
  AAV7 Left ITR
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccg

SEQ ID NO: 66 - DNA, 145 bp, Artificial Sequence
  AAV8 Left ITR
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacacc

SEQ ID NO: 67 - DNA, 145 bp, Artificial Sequence
  AAV9 Left ITR
cagagagggagtggccaactccatcactaggggtaatcgcgaagcgcctcccacgctgccgcgtcagcgctgacgtagattacgtcataggggagtggtcctgtattagctgtcacgtgagtgcttttgcgacattttgcgacac

SEQ ID NO: 68 - DNA, 143 bp, Artificial Sequence
  AAV1 Right ITR
ttacccctagtgatggagttgcccactccctctctgcgcgctcgctcgctcggtggggccggcagagcagagctctgccgtctgcggacctttggtccgcaggccccaccgagcgagcgagcgcgcagagagggagtgggcaa

SEQ ID NO: 69 - DNA, 145 bp, Artificial Sequence
  AAV2 Right ITR
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 70 - DNA, 142 bp, Artificial Sequence
  AAV3B Right ITR
atacctctagtgatggagttggccactccctctatgcgcactcgctcgctcggtggggccggacgtgcaaagcacgtccgtctggcgacctttggtcgccaggccccaccgagcgagcgagtgcgcatagagggagtggcca

SEQ ID NO: 71 - DNA, 146 bp, Artificial Sequence
  AAV4 Right ITR
gggcaaacctagatgatggagttggccactccctctatgcgcgctcgctcactcactcggccctgccggccagaggccggcagtctggagacctttggtctccagggccgagtgagtgagcgagcgcgcatagagggagtggccaa

SEQ ID NO: 72 - DNA, 155 bp, Artificial Sequence
  AAV5 Right ITR
ttgcttgagagtgtggcactctcccccctgtcgcgttcgctcgctcgctggctcgtttgggggggcgacggccagagggccgtcgtctggcagctctttgagctgccacccccccaaacgagccagcgagcgagcgaacgcgacaggggggagag

SEQ ID NO: 73 - DNA, 143 bp, Artificial Sequence
  AAV6 Right ITR
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggtat

SEQ ID NO: 74 - DNA, 145 bp, Artificial Sequence
  AAV7 Right ITR
cggtacccctagtgatggagttggccactccctctatgcgcgctcgctcgctcggtggggccggcagagcagagctctgccgtctgcggacctttggtccgcaggccccaccgagcgagcgagcgcgcatagagggagtggccaa

SEQ ID NO: 75 - DNA, 145 bp, Artificial Sequence
  AAV8 Right ITR
ggtgtcgcaaaatgccgcaaaagcactcacgtgacagctaatacaggaccactcccctatgacgtaatttacgtcagcgctgacgcggcagcgtgggaggcgcttcgcgctacccctagtgatggagttggccactccctctctg

SEQ ID NO: 76 - DNA, 145 bp, Artificial Sequence
  AAV9 Right ITR
gtgtcgcaaaatgtcgcaaaagcactcacgtgacagctaatacaggaccactcccctatgacgtaatctacgtcagcgctgacgcggcagcgtgggaggcgcttcgcgattacccctagtgatggagttggccactccctctctg

SEQ ID NO: 77 - DNA, 19 bp, Artificial Sequence
  Spacer
cagatctgaattcggtacc

SEQ ID NO: 78 - DNA, 264 bp, Artificial Sequence
  U6 Promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 79 - DNA, 82 bp, Artificial Sequence
  gRNA scaffold domain
gttttagtactctggaaacagaatctactaaaacaaggcaaaatgccgtgtttatctcgtcaacttgttggcgagatttttt

SEQ ID NO: 80 - DNA, 50 bp, Artificial Sequence
  Spacer
ggtaccgctagcgcttaagtcgcgatgtacgggccagatatacgcgttga

SEQ ID NO: 81 - DNA, 30 bp, Artificial Sequence
  N-ter NLS nucleotide sequence
ccgaagaaaaagcgcaaggtcgaagcgtcc

SEQ ID NO: 82 - protein, 7 aa, Artificial Sequence
  N-ter NLS amino acid sequence
PKKKRKV

SEQ ID NO: 83 - DNA, 21 bp, Artificial Sequence
  C-ter NLS sequence
cccaagaagaagaggaaagtc

SEQ ID NO: 84 - protein, 7 aa, Artificial Sequence
  C-ter NLS amino acid sequence
PKKKRKV

SEQ ID NO: 85 - DNA, 86 bp, Artificial Sequence
  Spacer
tccaagcttcgcaggaaagaacatgtgagcaaaaggccagcaaaaggcgttaactctagatttaaatgcatgctggggagagatct

SEQ ID NO: 86 - DNA, 66 bp, Artificial Sequence
  3X FLAG nucleotide sequence
gactacaaagaccatgacggtgattataaagatcatgacatcgattacaaggatgacgatgacaag

SEQ ID NO: 87 - DNA, 20 bp, Artificial Sequence
  Spacer
cgacttagttcgatcgaagg

SEQ ID NO: 88 - DNA, 106 bp, Artificial Sequence
  Unimolecular S. aureus gRNA
  misc_feature (1)..(16): a, c, t, g, unknown or other
  misc_feature (1)..(24): targeting domain (between 16 and 24 bases in length)
  misc_feature (17)..(24): a, c, t, g, unknown, absent or other
  misc_feature (25)..(38): crRNA
  misc_feature (39)..(42): GAAA linker
  misc_feature (43)..(106): tracrRNA
nnnnnnnnnnnnnnnnnnnnnnnngttttagtactctggaaacagaatctactaaaacaaggcaaaatgccgtgtttatctcgtcaacttgttggcgagatttttt

SEQ ID NO: 89 - RNA, 106 nt, Artificial Sequence
  Unimolecular S. aureus gRNA
  misc_feature (1)..(16): a, c, g, u, unknown or other
  misc_feature (1)..(24): targeting domain (between 16 and 24 bases in length)
  misc_feature (17)..(24): a, c, g, u, unknown, absent or other
  misc_feature (25)..(38): crRNA
  misc_feature (39)..(42): GAAA linker
  misc_feature (43)..(106): tracrRNA
nnnnnnnnnnnnnnnnnnnnnnnnguuuuaguacucuggaaacagaaucuacuaaaacaaggcaaaaugccguguuuaucucgucaacuuguuggcgagauuuuuu

SEQ ID NO: 90 - DNA, 106 bp, Artificial Sequence
  Unimolecular S. aureus gRNA
  misc_feature (1)..(16): a, c, t, g, unknown or other
  misc_feature (1)..(24): targeting domain (between 16 and 24 bases in length)
  misc_feature (17)..(24): a, c, t, g, unknown, absent or other
  misc_feature (25)..(38): crRNA
  misc_feature (39)..(42): GAAA linker
  misc_feature (43)..(106): tracrRNA
nnnnnnnnnnnnnnnnnnnnnnnngttatagtactctggaaacagaatctactataacaaggcaaaatgccgtgtttatctcgtcaacttgttggcgagatttttt

SEQ ID NO: 91 - RNA, 106 nt, Artificial Sequence
  Unimolecular S. aureus gRNA
  misc_feature (1)..(16): a, c, u, g, unknown or other
  misc_feature (1)..(24): targeting domain (between 16 and 24 bases in length)
  misc_feature (17)..(24): a, c, u, g, unknown, absent or other
  misc_feature (25)..(38): crRNA
  misc_feature (39)..(42): GAAA linker
  misc_feature (43)..(106): tracrRNA
nnnnnnnnnnnnnnnnnnnnnnnnguuauaguacucuggaaacagaaucuacuauaacaaggcaaaaugccguguuuaucucgucaacuuguuggcgagauuuuuu

SEQ ID NO: 92 - DNA, 139 bp, Artificial Sequence
  AAV Left ITR
tgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 93 - DNA, 139 bp, Artificial Sequence
  AAV Right ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgca

SEQ ID NO: 94 - DNA, 164 bp, Artificial Sequence
  SV40 intron
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttac

SEQ ID NO: 95-99 - skipped (5 entries)

SEQ ID NO: 100 - RNA, 22 nt, Artificial Sequence
  RHO-1 targeting domain
gucagccacaagggccacagcc

SEQ ID NO: 101 - RNA, 22 nt, Artificial Sequence
  RHO-2 targeting domain
ccgaagacgaaguauccaugca

SEQ ID NO: 102 - RNA, 22 nt, Artificial Sequence
  RHO-3 targeting domain
aguauccaugcagagaggugua

SEQ ID NO: 103 - RNA, 22 nt, Artificial Sequence
  RHO-4 targeting domain
cuagguugagcaggauguaguu

SEQ ID NO: 104 - RNA, 22 nt, Artificial Sequence
  RHO-5 targeting domain
cauggcucagccagguaguacu

SEQ ID NO: 105 - RNA, 22 nt, Artificial Sequence
  RHO-6 targeting domain
acgggugugguacgcagccccu

SEQ ID NO: 106 - RNA, 22 nt, Artificial Sequence
  RHO-7 targeting domain
cccacacccggcucauaccgcc

SEQ ID NO: 107 - RNA, 22 nt, Artificial Sequence
  RHO-8 targeting domain
cccugggcgguaugagccgggu

SEQ ID NO: 108 - RNA, 22 nt, Artificial Sequence
  RHO-9 targeting domain
ccaucaugggcguugccuucac

SEQ ID NO: 109 - RNA, 22 nt, Artificial Sequence
  RHO-10 targeting domain
gugccauuaccuggaccagccg

SEQ ID NO: 110 - RNA, 22 nt, Artificial Sequence
  RHO-11 targeting domain
uuaccuggaccagccggcgagu

SEQ ID NO: 111 - RNA, 22 nt, Artificial Sequence
  RHO-12 targeting domain
gcauucuugggugggagcagcc

SEQ ID NO: 112 - RNA, 22 nt, Artificial Sequence
  RHO-13 targeting domain
gcucagccacucagggcuccag

SEQ ID NO: 113 - RNA, 22 nt, Artificial Sequence
  RHO-14 targeting domain
ugacccguggcugcucccaccc

SEQ ID NO: 114 - RNA, 22 nt, Artificial Sequence
  RHO-15 targeting domain
agcucaggccuucgcagcauuc

SEQ ID NO: 115 - RNA, 22 nt, Artificial Sequence
  RHO-16 targeting domain
uagcagaagaaugcauccuaau

SEQ ID NO: 116 - RNA, 22 nt, Artificial Sequence
  RHO-17 targeting domain
acacgcugaggagagcugggca

SEQ ID NO: 117 - RNA, 22 nt, Artificial Sequence
  RHO-18 targeting domain
gcaaauaacuucccccauuccc

SEQ ID NO: 118 - RNA, 22 nt, Artificial Sequence
  RHO-19 targeting domain
agacccaggcugggcacugagg

SEQ ID NO: 119 - RNA, 22 nt, Artificial Sequence
  RHO-20 targeting domain
cuaggucuccuggcugugaucc

SEQ ID NO: 120 - RNA, 22 nt, Artificial Sequence
  RHO-21 targeting domain
ccagaaggugggugugccacuu

SEQ ID NO: 121 - RNA, 22 nt, Artificial Sequence
  RHO-22 targeting domain
aacaaggaacucugccccacau

SEQ ID NO: 122 - RNA, 22 nt, Artificial Sequence
  RHO-23 targeting domain
caggauugaacugggaacccgg

SEQ ID NO: 123 - RNA, 22 nt, Artificial Sequence
  RHO-24 targeting domain
gggcgucacacagggacggggg

SEQ ID NO: 124 - RNA, 22 nt, Artificial Sequence
  RHO-25 targeting domain
cugugauccaggaauaucucug

SEQ ID NO: 125 - RNA, 22 nt, Artificial Sequence
  RHO-26 targeting domain
uugcauuuaacaggaaaacaga

SEQ ID NO: 126 - RNA, 22 nt, Artificial Sequence
  RHO-27 targeting domain
ggagugcacccuccuuaggcag

SEQ ID NO: 127 - RNA, 22 nt, Artificial Sequence
  RHO-28 targeting domain
caucuguccugcucaccacccc

SEQ ID NO: 128 - RNA, 22 nt, Artificial Sequence
  RHO-29 targeting domain
gaggggaggcagaggaugccag

SEQ ID NO: 129 - RNA, 22 nt, Artificial Sequence
  RHO-30 targeting domain
cucagggaaucucuggccauug

SEQ ID NO: 130 - RNA, 22 nt, Artificial Sequence
  RHO-31 targeting domain
ugcacuccccccuagacaggga

SEQ ID NO: 131 - RNA, 22 nt, Artificial Sequence
  RHO-32 targeting domain
ugcuguuugugcagggcuggca

SEQ ID NO: 132 - RNA, 22 nt, Artificial Sequence
  RHO-33 targeting domain
acugggacauuccuaacaguga

SEQ ID NO: 133 - RNA, 22 nt, Artificial Sequence
  RHO-34 targeting domain
aucagggggucaggauugaacu

SEQ ID NO: 134 - RNA, 22 nt, Artificial Sequence
  RHO-35 targeting domain
cuccucucugggggcccaagcu

SEQ ID NO: 135 - RNA, 22 nt, Artificial Sequence
  RHO-36 targeting domain
cugcaucucagcagagauauuc

SEQ ID NO: 136 - RNA, 22 nt, Artificial Sequence
  RHO-37 targeting domain
uguuucccuuggagcagcugug

SEQ ID NO: 137 - RNA, 22 nt, Artificial Sequence
  RHO-38 targeting domain
gcgcucugggcccauaagggac

SEQ ID NO: 138 - RNA, 22 nt, Artificial Sequence
  RHO-39 targeting domain
aggauugaacugggaacccggu

SEQ ID NO: 139 - RNA, 22 nt, Artificial Sequence
  RHO-40 targeting domain
ccuaggagaggcccccacaugu

SEQ ID NO: 140 - RNA, 22 nt, Artificial Sequence
  RHO-41 targeting domain
aucacucaguucuggccagaag

SEQ ID NO: 141 - RNA, 22 nt, Artificial Sequence
  RHO-42 targeting domain
agagcugggcaaagaaauucca

SEQ ID NO: 142 - RNA, 22 nt, Artificial Sequence
  RHO-43 targeting domain
ccaccccaugaaguuccauagg

SEQ ID NO: 143 - RNA, 22 nt, Artificial Sequence
  RHO-44 targeting domain
ccacccugagcuugggccccca

SEQ ID NO: 144 - RNA, 22 nt, Artificial Sequence
  RHO-45 targeting domain
cagaggaagaagaaggaaauga

SEQ ID NO: 145 - RNA, 22 nt, Artificial Sequence
  RHO-46 targeting domain
aaacagcagcccggcuaucacc

SEQ ID NO: 146 - RNA, 22 nt, Artificial Sequence
  RHO-47 targeting domain
ggauugaacugggaacccggua

SEQ ID NO: 147 - RNA, 22 nt, Artificial Sequence
  RHO-48 targeting domain
uguguguguguguguuuagcag

SEQ ID NO: 148 - RNA, 22 nt, Artificial Sequence
  RHO-49 targeting domain
ucacacagggacgggugcagag

SEQ ID NO: 149 - RNA, 22 nt, Artificial Sequence
  RHO-50 targeting domain
guguguguguguguguguuuag

SEQ ID NO: 150 - RNA, 22 nt, Artificial Sequence
  RHO-51 targeting domain
ugagcuugggcccccagagagg

SEQ ID NO: 151 - RNA, 22 nt, Artificial Sequence
  RHO-52 targeting domain
aauaucucugcugagaugcagg

SEQ ID NO: 152 - RNA, 22 nt, Artificial Sequence
  RHO-53 targeting domain
ggagaggggaagagacucauuu

SEQ ID NO: 153 - RNA, 22 nt, Artificial Sequence
  RHO-54 targeting domain
agaacugagugaucugugauua

SEQ ID NO: 154 - RNA, 22 nt, Artificial Sequence
  RHO-55 targeting domain
ccacucucccuauggaacuuca

SEQ ID NO: 155 - RNA, 22 nt, Artificial Sequence
  RHO-56 targeting domain
auaagggacacgaaucagauca

SEQ ID NO: 156 - RNA, 22 nt, Artificial Sequence
  RHO-57 targeting domain
uggauuuuccauucuccaguca

SEQ ID NO: 157 - RNA, 22 nt, Artificial Sequence
  RHO-58 targeting domain
gugcaggagcccgggagcaugg

SEQ ID NO: 158 - RNA, 22 nt, Artificial Sequence
  RHO-59 targeting domain
ggguggugagcaggacagaugu

SEQ ID NO: 159 - RNA, 22 nt, Artificial Sequence
  RHO-60 targeting domain
cagcucucccucagugcccagc

SEQ ID NO: 160 - RNA, 22 nt, Artificial Sequence
  RHO-61 targeting domain
ccugcuggggcgucacacaggg

SEQ ID NO: 161 - RNA, 22 nt, Artificial Sequence
  RHO-62 targeting domain
cacacacacacaaaacucccua

SEQ ID NO: 162 - RNA, 22 nt, Artificial Sequence
  RHO-63 targeting domain
acuuacgggugguuguucucug

SEQ ID NO: 163 - RNA, 22 nt, Artificial Sequence
  RHO-64 targeting domain
cacagggaagacccaaugacug

SEQ ID NO: 164 - RNA, 22 nt, Artificial Sequence
  RHO-65 targeting domain
agcacagaccccacugccuaag

SEQ ID NO: 165 - RNA, 22 nt, Artificial Sequence
  RHO-66 targeting domain
accugaggacaggggcugagag

SEQ ID NO: 166 - RNA, 22 nt, Artificial Sequence
  RHO-67 targeting domain
caacaauggccagagauucccu

SEQ ID NO: 167 - RNA, 22 nt, Artificial Sequence
  RHO-68 targeting domain
ugcugccucggucccauucuca

SEQ ID NO: 168 - RNA, 22 nt, Artificial Sequence
  RHO-69 targeting domain
ugcugccuggccacaucccuaa

SEQ ID NO: 169 - RNA, 22 nt, Artificial Sequence
  RHO-70 targeting domain
gccacucucccuauggaacuuc

SEQ ID NO: 170 - RNA, 22 nt, Artificial Sequence
  RHO-71 targeting domain
gagggaggaaggacugccaauu

SEQ ID NO: 171 - RNA, 22 nt, Artificial Sequence
  RHO-72 targeting domain
gaggguagcuaggaaggcaacc

SEQ ID NO: 172 - RNA, 22 nt, Artificial Sequence
  RHO-73 targeting domain
ggaaggcaaccaggagugggag

SEQ ID NO: 173 - RNA, 22 nt, Artificial Sequence
  RHO-74 targeting domain
gcugagaugcaggaggagacgc

SEQ ID NO: 174 - RNA, 22 nt, Artificial Sequence
  RHO-75 targeting domain
aggcuggaggggcaccugagga

SEQ ID NO: 175 - RNA, 22 nt, Artificial Sequence
  RHO-76 targeting domain
aggaaggcaaccaggaguggga

SEQ ID NO: 176 - RNA, 22 nt, Artificial Sequence
  RHO-77 targeting domain
ccgggagcauggaggggucugg

SEQ ID NO: 177 - RNA, 22 nt, Artificial Sequence
  RHO-78 targeting domain
ggauaacagaucccacuuaaca

SEQ ID NO: 178 - RNA, 22 nt, Artificial Sequence
  RHO-79 targeting domain
aggcagaggaugccagagggga

SEQ ID NO: 179 - RNA, 22 nt, Artificial Sequence
  RHO-80 targeting domain
gggcccaagcucagggugggaa

SEQ ID NO: 180 - RNA, 22 nt, Artificial Sequence
  RHO-81 targeting domain
uaacuauauggccacucucccu

SEQ ID NO: 181 - RNA, 22 nt, Artificial Sequence
  RHO-82 targeting domain
ucccacuuaacagagaggaaaa

SEQ ID NO: 182 - RNA, 22 nt, Artificial Sequence
  RHO-83 targeting domain
gaaugcagaggugguggaaacc

SEQ ID NO: 183 - RNA, 22 nt, Artificial Sequence
  RHO-84 targeting domain
gggagacagggcaaggcuggca

SEQ ID NO: 184 - RNA, 22 nt, Artificial Sequence
  RHO-85 targeting domain
caccaccccaugaaguuccaua

SEQ ID NO: 185 - RNA, 22 nt, Artificial Sequence
  RHO-86 targeting domain
gccauauaguuaaucaaccaaa

SEQ ID NO: 186 - RNA, 22 nt, Artificial Sequence
  RHO-87 targeting domain
guagcuaggaaggcaaccagga

SEQ ID NO: 187 - RNA, 22 nt, Artificial Sequence
  RHO-88 targeting domain
cacauugcuucauggcuccuag

SEQ ID NO: 188 - RNA, 22 nt, Artificial Sequence
  RHO-89 targeting domain
cugagcuugggcccccagagag

SEQ ID NO: 189 - RNA, 22 nt, Artificial Sequence
  RHO-90 targeting domain
accgagcccauugcccagcaca

SEQ ID NO: 190 - RNA, 22 nt, Artificial Sequence
  RHO-91 targeting domain
cucaaagaagucaagcgcccug

SEQ ID NO: 191 - RNA, 22 nt, Artificial Sequence
  RHO-92 targeting domain
gcuacccucucccugucuaggg

SEQ ID NO: 192 - RNA, 22 nt, Artificial Sequence
  RHO-93 targeting domain
acccugagcuugggcccccaga

SEQ ID NO: 193 - RNA, 22 nt, Artificial Sequence
  RHO-94 targeting domain
ggcagagggaccacacgcugag

SEQ ID NO: 194 - RNA, 22 nt, Artificial Sequence
  RHO-95 targeting domain
ucugacucagcacagcugcucc

SEQ ID NO: 195 - RNA, 22 nt, Artificial Sequence
  RHO-96 targeting domain
cucucagccaccaccgccaagc

SEQ ID NO: 196 - RNA, 22 nt, Artificial Sequence
  RHO-97 targeting domain
agggauguggccaggcagcaac

SEQ ID NO: 197 - RNA, 22 nt, Artificial Sequence
  RHO-98 targeting domain
caccugaggacaggggcugaga

SEQ ID NO: 198 - RNA, 22 nt, Artificial Sequence
  RHO-99 targeting domain
gcccaugauggcaugguucucc

SEQ ID NO: 199 - RNA, 22 nt, Artificial Sequence
  RHO-100 targeting domain
gaaggggcagagggaccacacg

SEQ ID NO: 200 - RNA, 22 nt, Artificial Sequence
  RHO-101 targeting domain
agcacccucuacaccucucugc

SEQ ID NO: 201 - RNA, 22 nt, Artificial Sequence
  RHO-102 targeting domain
cuuuggauaacauugacaggac

SEQ ID NO: 202 - RNA, 22 nt, Artificial Sequence
  RHO-103 targeting domain
ggugaagccaccuaggaccaug

SEQ ID NO: 203 - RNA, 22 nt, Artificial Sequence
  RHO-104 targeting domain
uaacauugacaggacaggagaa

SEQ ID NO: 204 - RNA, 22 nt, Artificial Sequence
  RHO-105 targeting domain
gggagaggggaagagacucauu

SEQ ID NO: 205 - RNA, 22 nt, Artificial Sequence
  RHO-106 targeting domain
gcugugcugagucagacccagg

SEQ ID NO: 206 - RNA, 22 nt, Artificial Sequence
  RHO-107 targeting domain
uugaggaggccuuggggaagga

SEQ ID NO: 207 - RNA, 22 nt, Artificial Sequence
  RHO-108 targeting domain
gcccgggagcauggaggggucu

SEQ ID NO: 208 - RNA, 22 nt, Artificial Sequence
  RHO-109 targeting domain
guaaacugggacugacccugca

SEQ ID NO: 209 - RNA, 22 nt, Artificial Sequence
  RHO-110 targeting domain
auaacauugacaggacaggaga

SEQ ID NO: 210 - RNA, 22 nt, Artificial Sequence
  RHO-111 targeting domain
ggcagggaggcuggaggggcac

SEQ ID NO: 211 - RNA, 22 nt, Artificial Sequence
  RHO-112 targeting domain
gcaaacauggcccgagauagau

SEQ ID NO: 212 - RNA, 22 nt, Artificial Sequence
  RHO-113 targeting domain
ggaccgagcccauugcccagca

SEQ ID NO: 213 - RNA, 22 nt, Artificial Sequence
  RHO-114 targeting domain
gcucuacgucaccguccagcac

SEQ ID NO: 214 - RNA, 22 nt, Artificial Sequence
  RHO-115 targeting domain
agcacagcugcuccaagggaaa

SEQ ID NO: 215 - RNA, 22 nt, Artificial Sequence
  RHO-116 targeting domain
cuaaagcaaaaaggaacugcuu

SEQ ID NO: 216 - RNA, 22 nt, Artificial Sequence
  RHO-117 targeting domain
gagaggaaaacugaggcaggga

SEQ ID NO: 217 - RNA, 22 nt, Artificial Sequence
  RHO-118 targeting domain
cauugcaaagcugggugacggg

SEQ ID NO: 218 - RNA, 22 nt, Artificial Sequence
  RHO-119 targeting domain
uugccacccugggcgguaugag

SEQ ID NO: 219 - RNA, 22 nt, Artificial Sequence
  RHO-120 targeting domain
agcuaggaaggcaaccaggagu

SEQ ID NO: 220 - RNA, 22 nt, Artificial Sequence
  RHO-121 targeting domain
ucucugggggcccaagcucagg

SEQ ID NO: 221 - RNA, 22 nt, Artificial Sequence
  RHO-122 targeting domain
agcacagggaagacccaaugac

SEQ ID NO: 222 - RNA, 22 nt, Artificial Sequence
  RHO-123 targeting domain
guugacugaauauaugagggcu

SEQ ID NO: 223 - RNA, 22 nt, Artificial Sequence
  RHO-124 targeting domain
uuguaaacugggacugacccug

SEQ ID NO: 224 - RNA, 22 nt, Artificial Sequence
  RHO-125 targeting domain
cacacccaccuucuggccagaa

SEQ ID NO: 225 - RNA, 22 nt, Artificial Sequence
  RHO-126 targeting domain
ccagaggaagaagaaggaaaug

SEQ ID NO: 226 - RNA, 22 nt, Artificial Sequence
  RHO-127 targeting domain
gagauauuccuggaucacagcc

SEQ ID NO: 227 - RNA, 22 nt, Artificial Sequence
  RHO-128 targeting domain
aggggcagagggaccacacgcu

SEQ ID NO: 228 - RNA, 22 nt, Artificial Sequence
  RHO-129 targeting domain
aacuauauggccacucucccua

SEQ ID NO: 229 - RNA, 22 nt, Artificial Sequence
  RHO-130 targeting domain
gcugcuugcgguucucaacacc

SEQ ID NO: 230 - RNA, 22 nt, Artificial Sequence
  RHO-131 targeting domain
caccaugaaugguguuuguuga

SEQ ID NO: 231 - RNA, 22 nt, Artificial Sequence
  RHO-132 targeting domain
gcagccauugcaaagcugggug

SEQ ID NO: 232 - RNA, 22 nt, Artificial Sequence
  RHO-133 targeting domain
ugacucagcacagcugcuccaa

SEQ ID NO: 233 - RNA, 22 nt, Artificial Sequence
  RHO-134 targeting domain
cugggaggagggggaaggggca

SEQ ID NO: 234 - RNA, 22 nt, Artificial Sequence
  RHO-135 targeting domain
gauaacauugacaggacaggag

SEQ ID NO: 235 - RNA, 22 nt, Artificial Sequence
  RHO-136 targeting domain
ccaaacugggacauuccuaaca

SEQ ID NO: 236 - RNA, 22 nt, Artificial Sequence
  RHO-137 targeting domain
aggaaaacagauggggugcugc

SEQ ID NO: 237 - RNA, 22 nt, Artificial Sequence
  RHO-138 targeting domain
cggacaugugggggccucuccu

SEQ ID NO: 238 - RNA, 22 nt, Artificial Sequence
  RHO-139 targeting domain
gcaaagaaauuccagggaaugg

SEQ ID NO: 239 - RNA, 22 nt, Artificial Sequence
  RHO-140 targeting domain
ccaggagacuuggaacgcggca

SEQ ID NO: 240 - RNA, 22 nt, Artificial Sequence
  RHO-141 targeting domain
ugguccuuggugguccuggcca

SEQ ID NO: 241 - RNA, 22 nt, Artificial Sequence
  RHO-142 targeting domain
aauggaaaauccacuucccacc

SEQ ID NO: 242 - RNA, 22 nt, Artificial Sequence
  RHO-143 targeting domain
gcccgaagacgaaguauccaug

SEQ ID NO: 243 - RNA, 22 nt, Artificial Sequence
  RHO-144 targeting domain
gugcuggacggugacguagagc

SEQ ID NO: 244 - RNA, 22 nt, Artificial Sequence
  RHO-145 targeting domain
agaaacauguaggcggccagca

SEQ ID NO: 245 - RNA, 22 nt, Artificial Sequence
  RHO-146 targeting domain
ccgcucgauggccaggaccacc

SEQ ID NO: 246 - RNA, 22 nt, Artificial Sequence
  RHO-147 targeting domain
ucagcacagaccccacugccua

SEQ ID NO: 247 - RNA, 22 nt, Artificial Sequence
  RHO-148 targeting domain
gaauaucucugcugagaugcag

SEQ ID NO: 248 - RNA, 22 nt, Artificial Sequence
  RHO-149 targeting domain
gaguacccacaguacuaccugg

SEQ ID NO: 249 - RNA, 22 nt, Artificial Sequence
  RHO-150 targeting domain
caaccaggagugggagagggau

SEQ ID NO: 250 - RNA, 22 nt, Artificial Sequence
  RHO-151 targeting domain
uugagaaccgcaagcagccgcu

SEQ ID NO: 251 - RNA, 22 nt, Artificial Sequence
  RHO-152 targeting domain
gcaagccagaccccuccucucu

SEQ ID NO: 252 - RNA, 22 nt, Artificial Sequence
  RHO-153 targeting domain
gagagcugggcaaagaaauucc

SEQ ID NO: 253 - RNA, 22 nt, Artificial Sequence
  RHO-154 targeting domain
cgaggcagcagccuggacaugg

SEQ ID NO: 254 - RNA, 22 nt, Artificial Sequence
  RHO-155 targeting domain
aggaauaucucugcugagaugc

SEQ ID NO: 255 - RNA, 22 nt, Artificial Sequence
  RHO-156 targeting domain
uucccgagaagggagagggagg

SEQ ID NO: 256 - RNA, 22 nt, Artificial Sequence
  RHO-157 targeting domain
uccuuccucccucucccuucuc

SEQ ID NO: 257 - RNA, 22 nt, Artificial Sequence
  RHO-158 targeting domain
uguuuugcccagaggaagaaga

SEQ ID NO: 258 - RNA, 22 nt, Artificial Sequence
  RHO-159 targeting domain
ccggcugguccagguaauggca

SEQ ID NO: 259 - RNA, 22 nt, Artificial Sequence
  RHO-160 targeting domain
cagcacagggaagacccaauga

SEQ ID NO: 260 - RNA, 22 nt, Artificial Sequence
  RHO-161 targeting domain
accaggagugggagagggauuu

SEQ ID NO: 261 - RNA, 22 nt, Artificial Sequence
  RHO-162 targeting domain
gcuggugaagccaccuaggacc

SEQ ID NO: 262 - RNA, 22 nt, Artificial Sequence
  RHO-163 targeting domain
ggcgguaugagccggguguggg

SEQ ID NO: 263 - RNA, 22 nt, Artificial Sequence
  RHO-164 targeting domain
cagccauugcaaagcuggguga

SEQ ID NO: 264 - RNA, 22 nt, Artificial Sequence
  RHO-165 targeting domain
acauugacaggacaggagaagg

SEQ ID NO: 265 - RNA, 22 nt, Artificial Sequence
  RHO-166 targeting domain
ugggucuucccugugcugggca

SEQ ID NO: 266 - RNA, 22 nt, Artificial Sequence
  RHO-167 targeting domain
guacguggugguguguaagccc

SEQ ID NO: 267 - RNA, 22 nt, Artificial Sequence
  RHO-168 targeting domain
agcaaauaacuucccccauucc

SEQ ID NO: 268 - RNA, 22 nt, Artificial Sequence
  RHO-169 targeting domain
ggauuugaggaggccuugggga

SEQ ID NO: 269 - RNA, 22 nt, Artificial Sequence
  RHO-170 targeting domain
cccugagcuugggcccccagag

SEQ ID NO: 270 - RNA, 22 nt, Artificial Sequence
  RHO-171 targeting domain
cagagauucccugagaauggga

SEQ ID NO: 271 - RNA, 22 nt, Artificial Sequence
  RHO-172 targeting domain
gaguuggaagcccgcaucuauc

SEQ ID NO: 272 - RNA, 22 nt, Artificial Sequence
  RHO-173 targeting domain
aguccuuccucccucucccuuc

SEQ ID NO: 273 - RNA, 22 nt, Artificial Sequence
  RHO-174 targeting domain
guuauuucauuucccgagaagg

SEQ ID NO: 274 - RNA, 22 nt, Artificial Sequence
  RHO-175 targeting domain
auuucauuucccgagaagggag

SEQ ID NO: 275 - RNA, 22 nt, Artificial Sequence
  RHO-176 targeting domain
gacguagagcgugaggaaguug

SEQ ID NO: 276 - RNA, 22 nt, Artificial Sequence
  RHO-177 targeting domain
cauuucccgagaagggagaggg

SEQ ID NO: 277 - RNA, 22 nt, Artificial Sequence
  RHO-178 targeting domain
guagagcgugaggaaguugaug

SEQ ID NO: 278 - RNA, 22 nt, Artificial Sequence
  RHO-179 targeting domain
caggccuucgcagcauucuugg

SEQ ID NO: 279 - RNA, 22 nt, Artificial Sequence
  RHO-180 targeting domain
agguaguacuguggguacucga

SEQ ID NO: 280 - RNA, 22 nt, Artificial Sequence
  RHO-181 targeting domain
aaacauguaggcggccagcaug

SEQ ID NO: 281 - RNA, 22 nt, Artificial Sequence
  RHO-182 targeting domain
uuucauuucccgagaagggaga

SEQ ID NO: 282 - RNA, 22 nt, Artificial Sequence
  RHO-183 targeting domain
gggaagacccaaugacuggaga

SEQ ID NO: 283 - RNA, 22 nt, Artificial Sequence
  RHO-184 targeting domain
aaaacugaggcagggagagggg

SEQ ID NO: 284 - RNA, 22 nt, Artificial Sequence
  RHO-185 targeting domain
ugagucagacccaggcugggca

SEQ ID NO: 285 - RNA, 22 nt, Artificial Sequence
  RHO-186 targeting domain
gggauuugaggaggccuugggg

SEQ ID NO: 286 - RNA, 22 nt, Artificial Sequence
  RHO-187 targeting domain
ucugggggcccaagcucagggu

SEQ ID NO: 287 - RNA, 22 nt, Artificial Sequence
  RHO-188 targeting domain
cgggcccacaggaugcaauuug

SEQ ID NO: 288 - RNA, 22 nt, Artificial Sequence
  RHO-189 targeting domain
acguagagcgugaggaaguuga

SEQ ID NO: 289 - RNA, 22 nt, Artificial Sequence
  RHO-190 targeting domain
gaccgaggcagcagccuggaca

SEQ ID NO: 290 - RNA, 22 nt, Artificial Sequence
  RHO-191 targeting domain
caggcugggcacugagggagag

SEQ ID NO: 291 - RNA, 22 nt, Artificial Sequence
  RHO-192 targeting domain
uauuucauuucccgagaaggga

SEQ ID NO: 292 - RNA, 22 nt, Artificial Sequence
  RHO-193 targeting domain
gucccgggcuuggcgguggugg

SEQ ID NO: 293 - RNA, 22 nt, Artificial Sequence
  RHO-194 targeting domain
cugcugccucggucccauucuc

SEQ ID NO: 294 - RNA, 22 nt, Artificial Sequence
  RHO-195 targeting domain
agcgucuccuccugcaucucag

SEQ ID NO: 295 - RNA, 22 nt, Artificial Sequence
  RHO-196 targeting domain
ucagacccaggcugggcacuga

SEQ ID NO: 296 - RNA, 22 nt, Artificial Sequence
  RHO-197 targeting domain
agcuacccucucccugucuagg

SEQ ID NO: 297 - RNA, 22 nt, Artificial Sequence
  RHO-198 targeting domain
cagagaggaaaacugaggcagg

SEQ ID NO: 298 - RNA, 22 nt, Artificial Sequence
  RHO-199 targeting domain
ggagagggauuugaggaggccu

SEQ ID NO: 299 - RNA, 22 nt, Artificial Sequence
  RHO-200 targeting domain
guccuuccucccucucccuucu

SEQ ID NO: 300 - RNA, 22 nt, Artificial Sequence
  RHO-201 targeting domain
agagagcuuggugcugggagga

SEQ ID NO: 301 - RNA, 22 nt, Artificial Sequence
  RHO-202 targeting domain
ccuucucgggaaaugaaauaac

SEQ ID NO: 302 - RNA, 22 nt, Artificial Sequence
  RHO-203 targeting domain
gcgguucucaacaccaggagac

SEQ ID NO: 303 - RNA, 22 nt, Artificial Sequence
  RHO-204 targeting domain
cucugggggcccaagcucaggg

SEQ ID NO: 304 - RNA, 22 nt, Artificial Sequence
  RHO-205 targeting domain
ugugcaggagcccgggagcaug

SEQ ID NO: 305 - RNA, 22 nt, Artificial Sequence
  RHO-206 targeting domain
cagagagguguagagggugcug

SEQ ID NO: 306 - RNA, 22 nt, Artificial Sequence
  RHO-207 targeting domain
cuccccgaagcggaaguugcuc

SEQ ID NO: 307 - RNA, 22 nt, Artificial Sequence
  RHO-208 targeting domain
gcuagaagcagccauugcaaag

SEQ ID NO: 308 - RNA, 22 nt, Artificial Sequence
  RHO-209 targeting domain
caaacaccauucauggugauag

SEQ ID NO: 309 - RNA, 22 nt, Artificial Sequence
  RHO-210 targeting domain
ucauuucccgagaagggagagg

SEQ ID NO: 310 - RNA, 22 nt, Artificial Sequence
  RHO-211 targeting domain
ucaccaccccaugaaguuccau

SEQ ID NO: 311 - RNA, 22 nt, Artificial Sequence
  RHO-212 targeting domain
gggagugcacccuccuuaggca

SEQ ID NO: 312 - RNA, 22 nt, Artificial Sequence
  RHO-213 targeting domain
aauggccagagauucccugaga

SEQ ID NO: 313 - RNA, 22 nt, Artificial Sequence
  RHO-214 targeting domain
agaaugggaccgaggcagcagc

SEQ ID NO: 314 - RNA, 22 nt, Artificial Sequence
  RHO-215 targeting domain
ggcaagccagaccccuccucuc

SEQ ID NO: 315 - RNA, 22 nt, Artificial Sequence
  RHO-216 targeting domain
cccgggcuuggcggugguggcu

SEQ ID NO: 316 - RNA, 22 nt, Artificial Sequence
  RHO-217 targeting domain
agcccgggagcauggagggguc

SEQ ID NO: 317 - RNA, 22 nt, Artificial Sequence
  RHO-218 targeting domain
ccggguuauuucauuucccgag

SEQ ID NO: 318 - RNA, 22 nt, Artificial Sequence
  RHO-219 targeting domain
gguguuuguugacugaauauau

SEQ ID NO: 319 - RNA, 22 nt, Artificial Sequence
  RHO-220 targeting domain
ccgucccugugugacgccccag

SEQ ID NO: 320 - RNA, 22 nt, Artificial Sequence
  RHO-221 targeting domain
ggacaggggcugagaggggagg

SEQ ID NO: 321 - RNA, 22 nt, Artificial Sequence
  RHO-222 targeting domain
agagggugcuggugaagccacc

SEQ ID NO: 322 - RNA, 22 nt, Artificial Sequence
  RHO-223 targeting domain
auugcauccugugggcccgaag

SEQ ID NO: 323 - RNA, 22 nt, Artificial Sequence
  RHO-224 targeting domain
cggguuauuucauuucccgaga

SEQ ID NO: 324 - RNA, 22 nt, Artificial Sequence
  RHO-225 targeting domain
ggaaaugaaauaacccggacau

SEQ ID NO: 325 - RNA, 22 nt, Artificial Sequence
  RHO-226 targeting domain
cugacucagcacagcugcucca

SEQ ID NO: 326 - RNA, 22 nt, Artificial Sequence
  RHO-227 targeting domain
ggcaccugaggacaggggcuga

SEQ ID NO: 327 - RNA, 22 nt, Artificial Sequence
  RHO-228 targeting domain
ggagagcugggcaaagaaauuc

SEQ ID NO: 328 - RNA, 22 nt, Artificial Sequence
  RHO-229 targeting domain
gggcgguaugagccgggugugg

SEQ ID NO: 329 - RNA, 22 nt, Artificial Sequence
  RHO-230 targeting domain
ccucccucucccuucucgggaa

SEQ ID NO: 330 - RNA, 22 nt, Artificial Sequence
  RHO-231 targeting domain
uccagguaauggcacugagcag

SEQ ID NO: 331 - RNA, 22 nt, Artificial Sequence
  RHO-232 targeting domain
gugggggccucuccuaggagcc

SEQ ID NO: 332 - RNA, 22 nt, Artificial Sequence
  RHO-233 targeting domain
gauggcaugguucuccccgaag

SEQ ID NO: 333 - RNA, 22 nt, Artificial Sequence
  RHO-234 targeting domain
cgucgcauuggagaagggcacg

SEQ ID NO: 334 - RNA, 22 nt, Artificial Sequence
  RHO-235 targeting domain
uggguggggugugcaggagccc

SEQ ID NO: 335 - RNA, 22 nt, Artificial Sequence
  RHO-236 targeting domain
cuggacggugacguagagcgug

SEQ ID NO: 336 - RNA, 22 nt, Artificial Sequence
  RHO-237 targeting domain
gaggaaaacugaggcagggaga

SEQ ID NO: 337 - RNA, 22 nt, Artificial Sequence
  RHO-238 targeting domain
cugaacacugccuugaucuuau

SEQ ID NO: 338 - RNA, 22 nt, Artificial Sequence
  RHO-239 targeting domain
cauuaccuggaccagccggcga

SEQ ID NO: 339 - RNA, 22 nt, Artificial Sequence
  RHO-240 targeting domain
ggagagagcuuggugcugggag

SEQ ID NO: 340 - RNA, 22 nt, Artificial Sequence
  RHO-241 targeting domain
agaauaaugucuugcauuuaac

SEQ ID NO: 341 - RNA, 22 nt, Artificial Sequence
  RHO-242 targeting domain
cuaggaaggcaaccaggagugg

SEQ ID NO: 342 - RNA, 22 nt, Artificial Sequence
  RHO-243 targeting domain
ucucccagaccccuccaugcuc

SEQ ID NO: 343 - RNA, 22 nt, Artificial Sequence
  RHO-244 targeting domain
acaggggcugagaggggaggca

SEQ ID NO: 344 - RNA, 22 nt, Artificial Sequence
  RHO-245 targeting domain
ggggcagagggaccacacgcug

SEQ ID NO: 345 - RNA, 22 nt, Artificial Sequence
  RHO-246 targeting domain
aggggaggcagaggaugccaga

SEQ ID NO: 346 - RNA, 22 nt, Artificial Sequence
  RHO-247 targeting domain
ugguccagguaauggcacugag

SEQ ID NO: 347 - RNA, 22 nt, Artificial Sequence
  RHO-248 targeting domain
ccggacaugugggggccucucc

SEQ ID NO: 348 - RNA, 22 nt, Artificial Sequence
  RHO-249 targeting domain
gcaggccagcgccaugacccag

SEQ ID NO: 349 - RNA, 22 nt, Artificial Sequence
  RHO-250 targeting domain
cuagcuacccucucccugucua

SEQ ID NO: 350 - RNA, 22 nt, Artificial Sequence
  RHO-251 targeting domain
gcuuuggauaacauugacagga

SEQ ID NO: 351 - RNA, 22 nt, Artificial Sequence
  RHO-252 targeting domain
gccauugcaaagcugggugacg

SEQ ID NO: 352 - RNA, 22 nt, Artificial Sequence
  RHO-253 targeting domain
ccuaggucuccuggcugugauc

SEQ ID NO: 353 - RNA, 22 nt, Artificial Sequence
  RHO-254 targeting domain
aacagagaggaaaacugaggca

SEQ ID NO: 354 - RNA, 22 nt, Artificial Sequence
  RHO-255 targeting domain
auuaccuggaccagccggcgag

SEQ ID NO: 355 - RNA, 22 nt, Artificial Sequence
  RHO-256 targeting domain
gaggggcaccugaggacagggg

SEQ ID NO: 356 - RNA, 22 nt, Artificial Sequence
  RHO-257 targeting domain
ggguuauuucauuucccgagaa

SEQ ID NO: 357 - RNA, 22 nt, Artificial Sequence
  RHO-258 targeting domain
agggugcacuccccccuagaca

SEQ ID NO: 358 - RNA, 22 nt, Artificial Sequence
  RHO-259 targeting domain
ccaggagugggagagggauuug

SEQ ID NO: 359 - RNA, 22 nt, Artificial Sequence
  RHO-260 targeting domain
agaggggaggcagaggaugcca

SEQ ID NO: 360 - RNA, 22 nt, Artificial Sequence
  RHO-261 targeting domain
ccgccugcugacugccuugcag

SEQ ID NO: 361 - RNA, 22 nt, Artificial Sequence
  RHO-262 targeting domain
ggcuuggugcugcaaacauggc

SEQ ID NO: 362 - RNA, 22 nt, Artificial Sequence
  RHO-263 targeting domain
cagguaauggcacugagcagaa

SEQ ID NO: 363 - RNA, 22 nt, Artificial Sequence
  RHO-264 targeting domain
uuggaacgcggcagggaggcug

SEQ ID NO: 364 - RNA, 22 nt, Artificial Sequence
  RHO-265 targeting domain
uguccggguuauuucauuuccc

SEQ ID NO: 365 - RNA, 22 nt, Artificial Sequence
  RHO-266 targeting domain
cagguaguacuguggguacucg

SEQ ID NO: 366 - RNA, 22 nt, Artificial Sequence
  RHO-267 targeting domain
auaacagaucccacuuaacaga

SEQ ID NO: 367 - RNA, 22 nt, Artificial Sequence
  RHO-268 targeting domain
agggacgggugcagaguugagu

SEQ ID NO: 368 - RNA, 22 nt, Artificial Sequence
  RHO-269 targeting domain
gaaggagagagcuuggugcugg

SEQ ID NO: 369 - RNA, 22 nt, Artificial Sequence
  RHO-270 targeting domain
ggucagccacggcuagguugag

SEQ ID NO: 370 - RNA, 22 nt, Artificial Sequence
  RHO-271 targeting domain
auuucacagcaagaaaacugag

SEQ ID NO: 371 - RNA, 22 nt, Artificial Sequence
  RHO-272 targeting domain
ucaaagaagucaagcgcccugc

SEQ ID NO: 372 - RNA, 22 nt, Artificial Sequence
  RHO-273 targeting domain
gcugcucccacccaagaaugcu

SEQ ID NO: 373 - RNA, 22 nt, Artificial Sequence
  RHO-274 targeting domain
gcaacaaacacccaacaauggc

SEQ ID NO: 374 - RNA, 22 nt, Artificial Sequence
  RHO-275 targeting domain
aaauccacuucccacccugagc

SEQ ID NO: 375 - RNA, 22 nt, Artificial Sequence
  RHO-276 targeting domain
cagggaggcuggaggggcaccu

SEQ ID NO: 376 - RNA, 22 nt, Artificial Sequence
  RHO-277 targeting domain
gggcaagccagaccccuccucu

SEQ ID NO: 377 - RNA, 22 nt, Artificial Sequence
  RHO-278 targeting domain
caggaaaacagauggggugcug

SEQ ID NO: 378 - RNA, 22 nt, Artificial Sequence
  RHO-279 targeting domain
uuggagaagggcacguagaagu

SEQ ID NO: 379 - RNA, 22 nt, Artificial Sequence
  RHO-280 targeting domain
agagcuuggugcugggaggagg

SEQ ID NO: 380 - RNA, 22 nt, Artificial Sequence
  RHO-281 targeting domain
uagcuaggaaggcaaccaggag

SEQ ID NO: 381 - RNA, 22 nt, Artificial Sequence
  RHO-282 targeting domain
ggcuagguugagcaggauguag

SEQ ID NO: 382 - RNA, 22 nt, Artificial Sequence
  RHO-283 targeting domain
cucaccaccccaugaaguucca

SEQ ID NO: 383 - RNA, 22 nt, Artificial Sequence
  RHO-284 targeting domain
aagcaaugugcaauguuuugcc

SEQ ID NO: 384 - RNA, 22 nt, Artificial Sequence
  RHO-285 targeting domain
ggaagacccaaugacuggagaa

SEQ ID NO: 385 - RNA, 22 nt, Artificial Sequence
  RHO-286 targeting domain
uggccaggaccaccaaggacca

SEQ ID NO: 386 - RNA, 22 nt, Artificial Sequence
  RHO-287 targeting domain
aaauauugucccuuucacuguu

SEQ ID NO: 387 - RNA, 22 nt, Artificial Sequence
  RHO-288 targeting domain
caugagcaacuuccgcuucggg

SEQ ID NO: 388 - RNA, 22 nt, Artificial Sequence
  RHO-289 targeting domain
agagauauuccuggaucacagc

SEQ ID NO: 389 - RNA, 22 nt, Artificial Sequence
  RHO-290 targeting domain
cauggaggggucugggagaguc

SEQ ID NO: 390 - RNA, 22 nt, Artificial Sequence
  RHO-291 targeting domain
auguuuugcccagaggaagaag

SEQ ID NO: 391 - RNA, 22 nt, Artificial Sequence
  RHO-292 targeting domain
guggguggggugugcaggagcc

SEQ ID NO: 392 - RNA, 22 nt, Artificial Sequence
  RHO-293 targeting domain
ccagguaauggcacugagcaga

SEQ ID NO: 393 - RNA, 22 nt, Artificial Sequence
  RHO-294 targeting domain
cccaacaauggccagagauucc

SEQ ID NO: 394 - RNA, 22 nt, Artificial Sequence
  RHO-295 targeting domain
gcaccugaggacaggggcugag

SEQ ID NO: 395 - RNA, 22 nt, Artificial Sequence
  RHO-296 targeting domain
gucagacccaggcugggcacug

SEQ ID NO: 396 - RNA, 22 nt, Artificial Sequence
  RHO-297 targeting domain
ggggcaccugaggacaggggcu

SEQ ID NO: 397 - RNA, 22 nt, Artificial Sequence
  RHO-298 targeting domain
agaggaaaacugaggcagggag

SEQ ID NO: 398 - RNA, 22 nt, Artificial Sequence
  RHO-299 targeting domain
agggauaacagaucccacuuaa

SEQ ID NO: 399 - RNA, 22 nt, Artificial Sequence
  RHO-300 targeting domain
cuuggugcugggaggaggggga

SEQ ID NO: 400 - RNA, 22 nt, Artificial Sequence
  RHO-301 targeting domain
agaggguagcuaggaaggcaac

SEQ ID NO: 401 - RNA, 22 nt, Artificial Sequence
  RHO-302 targeting domain
uugcacauugcuucauggcucc

SEQ ID NO: 402 - RNA, 22 nt, Artificial Sequence
  RHO-303 targeting domain
gaccgagcccauugcccagcac

SEQ ID NO: 403 - RNA, 22 nt, Artificial Sequence
  RHO-304 targeting domain
ugaacacugccuugaucuuauu

SEQ ID NO: 404 - RNA, 22 nt, Artificial Sequence
  RHO-305 targeting domain
ggugcacuccccccuagacagg

SEQ ID NO: 405 - RNA, 22 nt, Artificial Sequence
  RHO-306 targeting domain
gcuuggugcugggaggaggggg

SEQ ID NO: 406 - RNA, 22 nt, Artificial Sequence
  RHO-307 targeting domain
ggauacuucgucuucgggccca

SEQ ID NO: 407 - RNA, 22 nt, Artificial Sequence
  RHO-308 targeting domain
agucagacccaggcugggcacu

SEQ ID NO: 408 - RNA, 22 nt, Artificial Sequence
  RHO-309 targeting domain
agcaccaagccucuguuucccu

SEQ ID NO: 409 - RNA, 22 nt, Artificial Sequence
  RHO-310 targeting domain
ugggcaaagaaauuccagggaa

SEQ ID NO: 410 - RNA, 22 nt, Artificial Sequence
  RHO-311 targeting domain
agagggauuugaggaggccuug

SEQ ID NO: 411 - RNA, 22 nt, Artificial Sequence
  RHO-312 targeting domain
gcaauguuuugcccagaggaag

SEQ ID NO: 412 - RNA, 22 nt, Artificial Sequence
  RHO-313 targeting domain
cauguccggguuauuucauuuc

SEQ ID NO: 413 - RNA, 22 nt, Artificial Sequence
  RHO-314 targeting domain
aagcccaugagcaacuuccgcu

SEQ ID NO: 414 - RNA, 22 nt, Artificial Sequence
  RHO-315 targeting domain
ucccacccugagcuugggcccc

SEQ ID NO: 415 - RNA, 22 nt, Artificial Sequence
  RHO-316 targeting domain
gagagagcuuggugcugggagg

SEQ ID NO: 416 - RNA, 22 nt, Artificial Sequence
  RHO-317 targeting domain
cuacgugcccuucuccaaugcg

SEQ ID NO: 417 - RNA, 22 nt, Artificial Sequence
  RHO-318 targeting domain
cuugcauuuaacaggaaaacag

SEQ ID NO: 418 - RNA, 22 nt, Artificial Sequence
  RHO-319 targeting domain
gaaaugaaauaacccggacaug

SEQ ID NO: 419 - RNA, 22 nt, Artificial Sequence
  RHO-320 targeting domain
cgaaggccugagcucagccacu

SEQ ID NO: 420 - RNA, 22 nt, Artificial Sequence
  RHO-321 targeting domain
ggagggugcacuccccccuaga

SEQ ID NO: 421 - RNA, 22 nt, Artificial Sequence
  RHO-322 targeting domain
cagcaccaagccucuguuuccc

SEQ ID NO: 422 - RNA, 22 nt, Artificial Sequence
  RHO-323 targeting domain
gggcaaagaaauuccagggaau

SEQ ID NO: 423 - RNA, 22 nt, Artificial Sequence
  RHO-324 targeting domain
cuucggggagaaccaugccauc

SEQ ID NO: 424 - RNA, 22 nt, Artificial Sequence
  RHO-325 targeting domain
ugggaggagggggaaggggcag

SEQ ID NO: 425 - RNA, 22 nt, Artificial Sequence
  RHO-326 targeting domain
ccuagacagggagaggguagcu

SEQ ID NO: 426 - RNA, 22 nt, Artificial Sequence
  RHO-327 targeting domain
uaacagagaggaaaacugaggc

SEQ ID NO: 427 - RNA, 22 nt, Artificial Sequence
  RHO-328 targeting domain
ucucagccaccaccgccaagcc

SEQ ID NO: 428 - RNA, 22 nt, Artificial Sequence
  RHO-329 targeting domain
gucagcacagaccccacugccu

SEQ ID NO: 429 - RNA, 22 nt, Artificial Sequence
  RHO-330 targeting domain
aggaaaacugaggcagggagag

SEQ ID NO: 430 - RNA, 22 nt, Artificial Sequence
  RHO-331 targeting domain
agccauugcaaagcugggugac

SEQ ID NO: 431 - RNA, 22 nt, Artificial Sequence
  RHO-332 targeting domain
aaaugaaauaacccggacaugu

SEQ ID NO: 432 - RNA, 22 nt, Artificial Sequence
  RHO-333 targeting domain
uagcuacccucucccugucuag

SEQ ID NO: 433 - RNA, 22 nt, Artificial Sequence
  RHO-334 targeting domain
uguggguggggugugcaggagc

SEQ ID NO: 434 - RNA, 22 nt, Artificial Sequence
  RHO-335 targeting domain
uggggaaggagagagcuuggug

SEQ ID NO: 435 - RNA, 22 nt, Artificial Sequence
  RHO-336 targeting domain
gacuuggaacgcggcagggagg

SEQ ID NO: 436 - RNA, 22 nt, Artificial Sequence
  RHO-337 targeting domain
aaggagagagcuuggugcuggg

SEQ ID NO: 437 - RNA, 22 nt, Artificial Sequence
  RHO-338 targeting domain
gggaaggagagagcuuggugcu

SEQ ID NO: 438 - RNA, 22 nt, Artificial Sequence
  RHO-339 targeting domain
auuugaggaggccuuggggaag

SEQ ID NO: 439 - RNA, 22 nt, Artificial Sequence
  RHO-340 targeting domain
auccagcuggagcccugagugg

SEQ ID NO: 440 - RNA, 22 nt, Artificial Sequence
  RHO-341 targeting domain
gagagcuuggugcugggaggag

SEQ ID NO: 441 - RNA, 22 nt, Artificial Sequence
  RHO-342 targeting domain
uccuagcuacccucucccuguc

SEQ ID NO: 442 - RNA, 22 nt, Artificial Sequence
  RHO-343 targeting domain
ccgaggcagcagccuggacaug

SEQ ID NO: 443 - RNA, 22 nt, Artificial Sequence
  RHO-344 targeting domain
ggggaaggagagagcuuggugc

SEQ ID NO: 444 - RNA, 22 nt, Artificial Sequence
  RHO-345 targeting domain
ugcugggaggagggggaagggg

SEQ ID NO: 445 - RNA, 22 nt, Artificial Sequence
  RHO-346 targeting domain
cuucuugugcuggacggugacg

SEQ ID NO: 446 - RNA, 22 nt, Artificial Sequence
  RHO-347 targeting domain
uaccacacccgucgcauuggag

SEQ ID NO: 447 - RNA, 22 nt, Artificial Sequence
  RHO-348 targeting domain
agcagccuggacaugggggaga

SEQ ID NO: 448 - RNA, 22 nt, Artificial Sequence
  RHO-349 targeting domain
agccagguaguacuguggguac

SEQ ID NO: 449 - RNA, 22 nt, Artificial Sequence
  RHO-350 targeting domain
ggcugcuugcgguucucaacac

SEQ ID NO: 450 - RNA, 22 nt, Artificial Sequence
  RHO-351 targeting domain
ggaccgaggcagcagccuggac

SEQ ID NO: 451 - RNA, 22 nt, Artificial Sequence
  RHO-352 targeting domain
cugggcaaagaaauuccaggga

SEQ ID NO: 452 - RNA, 22 nt, Artificial Sequence
  RHO-353 targeting domain
ugagaggggaggcagaggaugc

SEQ ID NO: 453 - RNA, 22 nt, Artificial Sequence
  RHO-354 targeting domain
gagggugcacuccccccuagac

SEQ ID NO: 454 - RNA, 22 nt, Artificial Sequence
  RHO-355 targeting domain
cgguucucaacaccaggagacu

SEQ ID NO: 455 - RNA, 22 nt, Artificial Sequence
  RHO-356 targeting domain
ugugcaauguuuugcccagagg

SEQ ID NO: 456 - RNA, 22 nt, Artificial Sequence
  RHO-357 targeting domain
gggggagacagggcaaggcugg

SEQ ID NO: 457 - RNA, 22 nt, Artificial Sequence
  RHO-358 targeting domain
gccggguguggguggggugugc

SEQ ID NO: 458 - RNA, 22 nt, Artificial Sequence
  RHO-359 targeting domain
cugcguaccacacccgucgcau

SEQ ID NO: 459 - RNA, 22 nt, Artificial Sequence
  RHO-360 targeting domain
cacccaagaaugcugcgaaggc

SEQ ID NO: 460 - RNA, 22 nt, Artificial Sequence
  RHO-361 targeting domain
ccuagcuacccucucccugucu

SEQ ID NO: 461 - RNA, 22 nt, Artificial Sequence
  RHO-362 targeting domain
caccaggagacuuggaacgcgg

SEQ ID NO: 462 - RNA, 22 nt, Artificial Sequence
  RHO-363 targeting domain
uuggauaacauugacaggacag

SEQ ID NO: 463 - RNA, 22 nt, Artificial Sequence
  RHO-364 targeting domain
uucgggcccacaggaugcaauu

SEQ ID NO: 464 - RNA, 22 nt, Artificial Sequence
  RHO-365 targeting domain
gaaguauccaugcagagaggug

SEQ ID NO: 465 - RNA, 22 nt, Artificial Sequence
  RHO-366 targeting domain
ggugugcaggagcccgggagca

SEQ ID NO: 466 - RNA, 22 nt, Artificial Sequence
  RHO-367 targeting domain
ggagcagccacgggucagccac

SEQ ID NO: 467 - RNA, 22 nt, Artificial Sequence
  RHO-368 targeting domain
agcgcccugcuggggcgucaca

SEQ ID NO: 468 - RNA, 22 nt, Artificial Sequence
  RHO-369 targeting domain
gagcccgggagcauggaggggu

SEQ ID NO: 469 - RNA, 22 nt, Artificial Sequence
  RHO-370 targeting domain
agggccacagccaugaauggca

SEQ ID NO: 470 - RNA, 22 nt, Artificial Sequence
  RHO-371 targeting domain
gcaaugugcaauguuuugccca

SEQ ID NO: 471 - RNA, 22 nt, Artificial Sequence
  RHO-372 targeting domain
gaagaggucagccacggcuagg

SEQ ID NO: 472 - RNA, 22 nt, Artificial Sequence
  RHO-373 targeting domain
ggccuucgcagcauucuugggu

SEQ ID NO: 473 - RNA, 22 nt, Artificial Sequence
  RHO-374 targeting domain
uuaacagagaggaaaacugagg

SEQ ID NO: 474 - RNA, 22 nt, Artificial Sequence
  RHO-375 targeting domain
ugauggcaugguucuccccgaa

SEQ ID NO: 475 - RNA, 22 nt, Artificial Sequence
  RHO-376 targeting domain
accgaggcagcagccuggacau

SEQ ID NO: 476 - RNA, 22 nt, Artificial Sequence
  RHO-377 targeting domain
agggaccacacgcugaggagag

SEQ ID NO: 477 - RNA, 22 nt, Artificial Sequence
  RHO-378 targeting domain
uggaacgcggcagggaggcugg

SEQ ID NO: 478 - RNA, 22 nt, Artificial Sequence
  RHO-379 targeting domain
ugcacauugcuucauggcuccu

SEQ ID NO: 479 - RNA, 22 nt, Artificial Sequence
  RHO-380 targeting domain
gcguuccaagucuccugguguu

SEQ ID NO: 480 - RNA, 22 nt, Artificial Sequence
  RHO-381 targeting domain
gggugugcaggagcccgggagc

SEQ ID NO: 481 - RNA, 22 nt, Artificial Sequence
  RHO-382 targeting domain
ggcaaagaaauuccagggaaug

SEQ ID NO: 482 - RNA, 22 nt, Artificial Sequence
  RHO-383 targeting domain
ggcuggaggggcaccugaggac

SEQ ID NO: 483 - RNA, 22 nt, Artificial Sequence
  RHO-384 targeting domain
gcgcccugcuggggcgucacac

SEQ ID NO: 484 - RNA, 22 nt, Artificial Sequence
  RHO-385 targeting domain
gcguaccacacccgucgcauug

SEQ ID NO: 485 - RNA, 22 nt, Artificial Sequence
  RHO-386 targeting domain
accaggagacuuggaacgcggc

SEQ ID NO: 486 - RNA, 22 nt, Artificial Sequence
  RHO-387 targeting domain
gcugcugccucggucccauucu

SEQ ID NO: 487 - RNA, 22 nt, Artificial Sequence
  RHO-388 targeting domain
gaagcccuccaaauugcauccu

SEQ ID NO: 488 - RNA, 22 nt, Artificial Sequence
  RHO-389 targeting domain
cguagagcgugaggaaguugau

SEQ ID NO: 489 - RNA, 22 nt, Artificial Sequence
  RHO-390 targeting domain
cugaagcaguuccuuuuugcuu

SEQ ID NO: 490 - RNA, 22 nt, Artificial Sequence
  RHO-391 targeting domain
gcuggacggugacguagagcgu

SEQ ID NO: 491 - RNA, 22 nt, Artificial Sequence
  RHO-392 targeting domain
ugagggcuuuggauaacauuga

SEQ ID NO: 492 - RNA, 22 nt, Artificial Sequence
  RHO-393 targeting domain
agccggguguggguggggugug

SEQ ID NO: 493 - RNA, 22 nt, Artificial Sequence
  RHO-394 targeting domain
cucaguuuuccucucuguuaag

SEQ ID NO: 494 - RNA, 22 nt, Artificial Sequence
  RHO-395 targeting domain
caagacauuauucuaaagcaaa

SEQ ID NO: 495 - RNA, 22 nt, Artificial Sequence
  RHO-396 targeting domain
uggaacuucaugggguggugag

SEQ ID NO: 496 - RNA, 22 nt, Artificial Sequence
  RHO-397 targeting domain
gagagggauuugaggaggccuu

SEQ ID NO: 497 - RNA, 22 nt, Artificial Sequence
  RHO-398 targeting domain
cuucgggcccacaggaugcaau

SEQ ID NO: 498 - RNA, 22 nt, Artificial Sequence
  RHO-399 targeting domain
acuuggaacgcggcagggaggc

SEQ ID NO: 499 - RNA, 22 nt, Artificial Sequence
  RHO-400 targeting domain
auggccagagauucccugagaa

SEQ ID NO: 500 - RNA, 22 nt, Artificial Sequence
  RHO-401 targeting domain
ccucaguuuuccucucuguuaa

SEQ ID NO: 501 - RNA, 22 nt, Artificial Sequence
  RHO-402 targeting domain
uaacagaucccacuuaacagag

SEQ ID NO: 502 - RNA, 22 nt, Artificial Sequence
  RHO-403 targeting domain
gggagagggauuugaggaggcc

SEQ ID NO: 503-599 - skipped (97 entries)

SEQ ID NO: 600 - DNA, 22 bp, Artificial Sequence
  RHO-1 targeting domain
gtcagccacaagggccacagcc

SEQ ID NO: 601 - DNA, 22 bp, Artificial Sequence
  RHO-2 targeting domain
ccgaagacgaagtatccatgca

SEQ ID NO: 602 - DNA, 22 bp, Artificial Sequence
  RHO-3 targeting domain
agtatccatgcagagaggtgta

SEQ ID NO: 603 - DNA, 22 bp, Artificial Sequence
  RHO-4 targeting domain
ctaggttgagcaggatgtagtt

SEQ ID NO: 604 - DNA, 22 bp, Artificial Sequence
  RHO-5 targeting domain
catggctcagccaggtagtact

SEQ ID NO: 605 - DNA, 22 bp, Artificial Sequence
  RHO-6 targeting domain
acgggtgtggtacgcagcccct

SEQ ID NO: 606 - DNA, 22 bp, Artificial Sequence
  RHO-7 targeting domain
cccacacccggctcataccgcc

SEQ ID NO: 607 - DNA, 22 bp, Artificial Sequence
  RHO-8 targeting domain
ccctgggcggtatgagccgggt

SEQ ID NO: 608 - DNA, 22 bp, Artificial Sequence
  RHO-9 targeting domain
ccatcatgggcgttgccttcac

SEQ ID NO: 609 - DNA, 22 bp, Artificial Sequence
  RHO-10 targeting domain
gtgccattacctggaccagccg

SEQ ID NO: 610 - DNA, 22 bp, Artificial Sequence
  RHO-11 targeting domain
ttacctggaccagccggcgagt

SEQ ID NO: 611 - DNA, 22 bp, Artificial Sequence
  RHO-12 targeting domain
gcattcttgggtgggagcagcc

SEQ ID NO: 612 - DNA, 22 bp, Artificial Sequence
  RHO-13 targeting domain
gctcagccactcagggctccag

SEQ ID NO: 613 - DNA, 22 bp, Artificial Sequence
  RHO-14 targeting domain
tgacccgtggctgctcccaccc

SEQ ID NO: 614 - DNA, 22 bp, Artificial Sequence
  RHO-15 targeting domain
agctcaggccttcgcagcattc

SEQ ID NO: 615 - DNA, 22 bp, Artificial Sequence
  RHO-16 targeting domain
tagcagaagaatgcatcctaat

SEQ ID NO: 616 - DNA, 22 bp, Artificial Sequence
  RHO-17 targeting domain
acacgctgaggagagctgggca

SEQ ID NO: 617 - DNA, 22 bp, Artificial Sequence
  RHO-18 targeting domain
gcaaataacttcccccattccc

SEQ ID NO: 618 - DNA, 22 bp, Artificial Sequence
  RHO-19 targeting domain
agacccaggctgggcactgagg

SEQ ID NO: 619 - DNA, 22 bp, Artificial Sequence
  RHO-20 targeting domain
ctaggtctcctggctgtgatcc

SEQ ID NO: 620 - DNA, 22 bp, Artificial Sequence
  RHO-21 targeting domain
ccagaaggtgggtgtgccactt

SEQ ID NO: 621 - DNA, 22 bp, Artificial Sequence
  RHO-22 targeting domain
aacaaggaactctgccccacat

SEQ ID NO: 622 - DNA, 22 bp, Artificial Sequence
  RHO-23 targeting domain
caggattgaactgggaacccgg

SEQ ID NO: 623 - DNA, 22 bp, Artificial Sequence
  RHO-24 targeting domain
gggcgtcacacagggacgggtg

SEQ ID NO: 624 - DNA, 22 bp, Artificial Sequence
  RHO-25 targeting domain
ctgtgatccaggaatatctctg

SEQ ID NO: 625 - DNA, 22 bp, Artificial Sequence
  RHO-26 targeting domain
ttgcatttaacaggaaaacaga

SEQ ID NO: 626 - DNA, 22 bp, Artificial Sequence
  RHO-27 targeting domain
ggagtgcaccctccttaggcag

SEQ ID NO: 627 - DNA, 22 bp, Artificial Sequence
  RHO-28 targeting domain
catctgtcctgctcaccacccc

SEQ ID NO: 628 - DNA, 22 bp, Artificial Sequence
  RHO-29 targeting domain
gaggggaggcagaggatgccag

SEQ ID NO: 629 - DNA, 22 bp, Artificial Sequence
  RHO-30 targeting domain
ctcagggaatctctggccattg

SEQ ID NO: 630 - DNA, 22 bp, Artificial Sequence
  RHO-31 targeting domain
tgcactcccccctagacaggga

SEQ ID NO: 631 - DNA, 22 bp, Artificial Sequence
  RHO-32 targeting domain
tgctgtttgtgcagggctggca

SEQ ID NO: 632 - DNA, 22 bp, Artificial Sequence
  RHO-33 targeting domain
actgggacattcctaacagtga

SEQ ID NO: 633 - DNA, 22 bp, Artificial Sequence
  RHO-34 targeting domain
atcagggggtcaggattgaact

SEQ ID NO: 634 - DNA, 22 bp, Artificial Sequence
  RHO-35 targeting domain
ctcctctctgggggcccaagct

SEQ ID NO: 635 - DNA, 22 bp, Artificial Sequence
  RHO-36 targeting domain
ctgcatctcagcagagatattc

SEQ ID NO: 636 - DNA, 22 bp, Artificial Sequence
  RHO-37 targeting domain
tgtttcccttggagcagctgtg

SEQ ID NO: 637 - DNA, 22 bp, Artificial Sequence
  RHO-38 targeting domain
gcgctctgggcccataagggac

SEQ ID NO: 638 - DNA, 22 bp, Artificial Sequence
  RHO-39 targeting domain
aggattgaactgggaacccggt

SEQ ID NO: 639 - DNA, 22 bp, Artificial Sequence
  RHO-40 targeting domain
cctaggagaggcccccacatgt

SEQ ID NO: 640 - DNA, 22 bp, Artificial Sequence
  RHO-41 targeting domain
atcactcagttctggccagaag

SEQ ID NO: 641 - DNA, 22 bp, Artificial Sequence
  RHO-42 targeting domain
agagctgggcaaagaaattcca

SEQ ID NO: 642 - DNA, 22 bp, Artificial Sequence
  RHO-43 targeting domain
ccaccccatgaagttccatagg

SEQ ID NO: 643 - DNA, 22 bp, Artificial Sequence
  RHO-44 targeting domain
ccaccctgagcttgggccccca

SEQ ID NO: 644 - DNA, 22 bp, Artificial Sequence
  RHO-45 targeting domain
cagaggaagaagaaggaaatga

SEQ ID NO: 645 - DNA, 22 bp, Artificial Sequence
  RHO-46 targeting domain
aaacagcagcccggctatcacc

SEQ ID NO: 646 - DNA, 22 bp, Artificial Sequence
  RHO-47 targeting domain
ggattgaactgggaacccggta

SEQ ID NO: 647 - DNA, 22 bp, Artificial Sequence
  RHO-48 targeting domain
tgtgtgtgtgtgtgtttagcag

SEQ ID NO: 648 - DNA, 22 bp, Artificial Sequence
  RHO-49 targeting domain
tcacacagggacgggtgcagag

SEQ ID NO: 649 - DNA, 22 bp, Artificial Sequence
  RHO-50 targeting domain
gtgtgtgtgtgtgtgtgtttag

SEQ ID NO: 650 - DNA, 22 bp, Artificial Sequence
  RHO-51 targeting domain
tgagcttgggcccccagagagg

SEQ ID NO: 651 - DNA, 22 bp, Artificial Sequence
  RHO-52 targeting domain
aatatctctgctgagatgcagg

SEQ ID NO: 652 - DNA, 22 bp, Artificial Sequence
  RHO-53 targeting domain
ggagaggggaagagactcattt

SEQ ID NO: 653 - DNA, 22 bp, Artificial Sequence
  RHO-54 targeting domain
agaactgagtgatctgtgatta

SEQ ID NO: 654 - DNA, 22 bp, Artificial Sequence
  RHO-55 targeting domain
ccactctccctatggaacttca

SEQ ID NO: 655 - DNA, 22 bp, Artificial Sequence
  RHO-56 targeting domain
ataagggacacgaatcagatca

SEQ ID NO: 656 - DNA, 22 bp, Artificial Sequence
  RHO-57 targeting domain
tggattttccattctccagtca

SEQ ID NO: 657 - DNA, 22 bp, Artificial Sequence
  RHO-58 targeting domain
gtgcaggagcccgggagcatgg

SEQ ID NO: 658 - DNA, 22 bp, Artificial Sequence
  RHO-59 targeting domain
gggtggtgagcaggacagatgt

SEQ ID NO: 659 - DNA, 22 bp, Artificial Sequence
  RHO-60 targeting domain
cagctctccctcagtgcccagc

SEQ ID NO: 660 - DNA, 22 bp, Artificial Sequence
  RHO-61 targeting domain
cctgctggggcgtcacacaggg

SEQ ID NO: 661 - DNA, 22 bp, Artificial Sequence
  RHO-62 targeting domain
cacacacacacaaaactcccta

SEQ ID NO: 662 - DNA, 22 bp, Artificial Sequence
  RHO-63 targeting domain
acttacgggtggttgttctctg

SEQ ID NO: 663 - DNA, 22 bp, Artificial Sequence
  RHO-64 targeting domain
cacagggaagacccaatgactg

SEQ ID NO: 664 - DNA, 22 bp, Artificial Sequence
  RHO-65 targeting domain
agcacagaccccactgcctaag

SEQ ID NO: 665 - DNA, 22 bp, Artificial Sequence
  RHO-66 targeting domain
acctgaggacaggggctgagag

SEQ ID NO: 666 - DNA, 22 bp, Artificial Sequence
  RHO-67 targeting domain
caacaatggccagagattccct

SEQ ID NO: 667 - DNA, 22 bp, Artificial Sequence
  RHO-68 targeting domain
tgctgcctcggtcccattctca

SEQ ID NO: 668 - DNA, 22 bp, Artificial Sequence
  RHO-69 targeting domain
tgctgcctggccacatccctaa

SEQ ID NO: 669 - DNA, 22 bp, Artificial Sequence
  RHO-70 targeting domain
gccactctccctatggaacttc

SEQ ID NO: 670 - DNA, 22 bp, Artificial Sequence
  RHO-71 targeting domain
gagggaggaaggactgccaatt

SEQ ID NO: 671 - DNA, 22 bp, Artificial Sequence
  RHO-72 targeting domain
gagggtagctaggaaggcaacc

SEQ ID NO: 672 - DNA, 22 bp, Artificial Sequence
  RHO-73 targeting domain
ggaaggcaaccaggagtgggag

SEQ ID NO: 673 - DNA, 22 bp, Artificial Sequence
  RHO-74 targeting domain
gctgagatgcaggaggagacgc

SEQ ID NO: 674 - DNA, 22 bp, Artificial Sequence
  RHO-75 targeting domain
aggctggaggggcacctgagga

SEQ ID NO: 675 - DNA, 22 bp, Artificial Sequence
  RHO-76 targeting domain
aggaaggcaaccaggagtggga

SEQ ID NO: 676 - DNA, 22 bp, Artificial Sequence
  RHO-77 targeting domain
ccgggagcatggaggggtctgg

SEQ ID NO: 677 - DNA, 22 bp, Artificial Sequence
  RHO-78 targeting domain
ggataacagatcccacttaaca

SEQ ID NO: 678 - DNA, 22 bp, Artificial Sequence
  RHO-79 targeting domain
aggcagaggatgccagagggga

SEQ ID NO: 679 - DNA, 22 bp, Artificial Sequence
  RHO-80 targeting domain
gggcccaagctcagggtgggaa

SEQ ID NO: 680 - DNA, 22 bp, Artificial Sequence
  RHO-81 targeting domain
taactatatggccactctccct

SEQ ID NO: 681 - DNA, 22 bp, Artificial Sequence
  RHO-82 targeting domain
tcccacttaacagagaggaaaa

SEQ ID NO: 682 - DNA, 22 bp, Artificial Sequence
  RHO-83 targeting domain
gaatgcagaggtggtggaaacc

SEQ ID NO: 683 - DNA, 22 bp, Artificial Sequence
  RHO-84 targeting domain
gggagacagggcaaggctggca

SEQ ID NO: 684 - DNA, 22 bp, Artificial Sequence
  RHO-85 targeting domain
caccaccccatgaagttccata

SEQ ID NO: 685 - DNA, 22 bp, Artificial Sequence
  RHO-86 targeting domain
gccatatagttaatcaaccaaa

SEQ ID NO: 686 - DNA, 22 bp, Artificial Sequence
  RHO-87 targeting domain
gtagctaggaaggcaaccagga

SEQ ID NO: 687 - DNA, 22 bp, Artificial Sequence
  RHO-88 targeting domain
cacattgcttcatggctcctag

SEQ ID NO: 688 - DNA, 22 bp, Artificial Sequence
  RHO-89 targeting domain
ctgagcttgggcccccagagag

SEQ ID NO: 689 - DNA, 22 bp, Artificial Sequence
  RHO-90 targeting domain
accgagcccattgcccagcaca

SEQ ID NO: 690 - DNA, 22 bp, Artificial Sequence
  RHO-91 targeting domain
ctcaaagaagtcaagcgccctg

SEQ ID NO: 691 - DNA, 22 bp, Artificial Sequence
  RHO-92 targeting domain
gctaccctctccctgtctaggg

SEQ ID NO: 692 - DNA, 22 bp, Artificial Sequence
  RHO-93 targeting domain
accctgagcttgggcccccaga

SEQ ID NO: 693 - DNA, 22 bp, Artificial Sequence
  RHO-94 targeting domain
ggcagagggaccacacgctgag

SEQ ID NO: 694 - DNA, 22 bp, Artificial Sequence
  RHO-95 targeting domain
tctgactcagcacagctgctcc

SEQ ID NO: 695 - DNA, 22 bp, Artificial Sequence
  RHO-96 targeting domain
ctctcagccaccaccgccaagc

SEQ ID NO: 696 - DNA, 22 bp, Artificial Sequence
  RHO-97 targeting domain
agggatgtggccaggcagcaac

SEQ ID NO: 697 - DNA, 22 bp, Artificial Sequence
  RHO-98 targeting domain
cacctgaggacaggggctgaga

SEQ ID NO: 698 - DNA, 22 bp, Artificial Sequence
  RHO-99 targeting domain
gcccatgatggcatggttctcc

SEQ ID NO: 699 - DNA, 22 bp, Artificial Sequence
  RHO-100 targeting domain
gaaggggcagagggaccacacg

SEQ ID NO: 700 - DNA, 22 bp, Artificial Sequence
  RHO-101 targeting domain
agcaccctctacacctctctgc

SEQ ID NO: 701 - DNA, 22 bp, Artificial Sequence
  RHO-102 targeting domain
ctttggataacattgacaggac

SEQ ID NO: 702 - DNA, 22 bp, Artificial Sequence
  RHO-103 targeting domain
ggtgaagccacctaggaccatg

SEQ ID NO: 703 - DNA, 22 bp, Artificial Sequence
  RHO-104 targeting domain
taacattgacaggacaggagaa

SEQ ID NO: 704 - DNA, 22 bp, Artificial Sequence
  RHO-105 targeting domain
gggagaggggaagagactcatt

SEQ ID NO: 705 - DNA, 22 bp, Artificial Sequence
  RHO-106 targeting domain
gctgtgctgagtcagacccagg

SEQ ID NO: 706 - DNA, 22 bp, Artificial Sequence
  RHO-107 targeting domain
ttgaggaggccttggggaagga

SEQ ID NO: 707 - DNA, 22 bp, Artificial Sequence
  RHO-108 targeting domain
gcccgggagcatggaggggtct

SEQ ID NO: 708 - DNA, 22 bp, Artificial Sequence
  RHO-109 targeting domain
gtaaactgggactgaccctgca

SEQ ID NO: 709 - DNA, 22 bp, Artificial Sequence
  RHO-110 targeting domain
ataacattgacaggacaggaga

SEQ ID NO: 710 - DNA, 22 bp, Artificial Sequence
  RHO-111 targeting domain
ggcagggaggctggaggggcac

SEQ ID NO: 711 - DNA, 22 bp, Artificial Sequence
  RHO-112 targeting domain
gcaaacatggcccgagatagat

SEQ ID NO: 712 - DNA, 22 bp, Artificial Sequence
  RHO-113 targeting domain
ggaccgagcccattgcccagca

SEQ ID NO: 713 - DNA, 22 bp, Artificial Sequence
  RHO-114 targeting domain
gctctacgtcaccgtccagcac

SEQ ID NO: 714 - DNA, 22 bp, Artificial Sequence
  RHO-115 targeting domain
agcacagctgctccaagggaaa

SEQ ID NO: 715 - DNA, 22 bp, Artificial Sequence
  RHO-116 targeting domain
ctaaagcaaaaaggaactgctt

SEQ ID NO: 716 - DNA, 22 bp, Artificial Sequence
  RHO-117 targeting domain
gagaggaaaactgaggcaggga

SEQ ID NO: 717 - DNA, 22 bp, Artificial Sequence
  RHO-118 targeting domain
cattgcaaagctgggtgacggg

SEQ ID NO: 718 - DNA, 22 bp, Artificial Sequence
  RHO-119 targeting domain
ttgccaccctgggcggtatgag

SEQ ID NO: 719 - DNA, 22 bp, Artificial Sequence
  RHO-120 targeting domain
agctaggaaggcaaccaggagt

SEQ ID NO: 720 - DNA, 22 bp, Artificial Sequence
  RHO-121 targeting domain
tctctgggggcccaagctcagg

SEQ ID NO: 721 - DNA, 22 bp, Artificial Sequence
  RHO-122 targeting domain
agcacagggaagacccaatgac

SEQ ID NO: 722 - DNA, 22 bp, Artificial Sequence
  RHO-123 targeting domain
gttgactgaatatatgagggct

SEQ ID NO: 723 - DNA, 22 bp, Artificial Sequence
  RHO-124 targeting domain
ttgtaaactgggactgaccctg

SEQ ID NO: 724 - DNA, 22 bp, Artificial Sequence
  RHO-125 targeting domain
cacacccaccttctggccagaa

SEQ ID NO: 725 - DNA, 22 bp, Artificial Sequence
  RHO-126 targeting domain
ccagaggaagaagaaggaaatg

SEQ ID NO: 726 - DNA, 22 bp, Artificial Sequence
  RHO-127 targeting domain
gagatattcctggatcacagcc

SEQ ID NO: 727 - DNA, 22 bp, Artificial Sequence
  RHO-128 targeting domain
aggggcagagggaccacacgct

SEQ ID NO: 728 - DNA, 22 bp, Artificial Sequence
  RHO-129 targeting domain
aactatatggccactctcccta

SEQ ID NO: 729 - DNA, 22 bp, Artificial Sequence
  RHO-130 targeting domain
gctgcttgcggttctcaacacc

SEQ ID NO: 730 - DNA, 22 bp, Artificial Sequence
  RHO-131 targeting domain
caccatgaatggtgtttgttga

SEQ ID NO: 731 - DNA, 22 bp, Artificial Sequence
  RHO-132 targeting domain
gcagccattgcaaagctgggtg

SEQ ID NO: 732 - DNA, 22 bp, Artificial Sequence
  RHO-133 targeting domain
tgactcagcacagctgctccaa

SEQ ID NO: 733 - DNA, 22 bp, Artificial Sequence
  RHO-134 targeting domain
ctgggaggagggggaaggggca

SEQ ID NO: 734 - DNA, 22 bp, Artificial Sequence
  RHO-135 targeting domain
gataacattgacaggacaggag

SEQ ID NO: 735 - DNA, 22 bp, Artificial Sequence
  RHO-136 targeting domain
ccaaactgggacattcctaaca

SEQ ID NO: 736 - DNA, 22 bp, Artificial Sequence
  RHO-137 targeting domain
aggaaaacagatggggtgctgc

SEQ ID NO: 737 - DNA, 22 bp, Artificial Sequence
  RHO-138 targeting domain
cggacatgtgggggcctctcct

SEQ ID NO: 738 - DNA, 22 bp, Artificial Sequence
  RHO-139 targeting domain
gcaaagaaattccagggaatgg

SEQ ID NO: 739 - DNA, 22 bp, Artificial Sequence
  RHO-140 targeting domain
ccaggagacttggaacgcggca

SEQ ID NO: 740 - DNA, 22 bp, Artificial Sequence
  RHO-141 targeting domain
tggtccttggtggtcctggcca

SEQ ID NO: 741 - DNA, 22 bp, Artificial Sequence
  RHO-142 targeting domain
aatggaaaatccacttcccacc

SEQ ID NO: 742 - DNA, 22 bp, Artificial Sequence
  RHO-143 targeting domain
gcccgaagacgaagtatccatg

SEQ ID NO: 743 - DNA, 22 bp, Artificial Sequence
  RHO-144 targeting domain
gtgctggacggtgacgtagagc

SEQ ID NO: 744 - DNA, 22 bp, Artificial Sequence
  RHO-145 targeting domain
agaaacatgtaggcggccagca

SEQ ID NO: 745 - DNA, 22 bp, Artificial Sequence
  RHO-146 targeting domain
ccgctcgatggccaggaccacc

SEQ ID NO: 746 - DNA, 22 bp, Artificial Sequence
  RHO-147 targeting domain
tcagcacagaccccactgccta

SEQ ID NO: 747 - DNA, 22 bp, Artificial Sequence
  RHO-148 targeting domain
gaatatctctgctgagatgcag

SEQ ID NO: 748 - DNA, 22 bp, Artificial Sequence
  RHO-149 targeting domain
gagtacccacagtactacctgg

SEQ ID NO: 749 - DNA, 22 bp, Artificial Sequence
  RHO-150 targeting domain
caaccaggagtgggagagggat

SEQ ID NO: 750 - DNA, 22 bp, Artificial Sequence
  RHO-151 targeting domain
ttgagaaccgcaagcagccgct

SEQ ID NO: 751 - DNA, 22 bp, Artificial Sequence
  RHO-152 targeting domain
gcaagccagacccctcctctct

SEQ ID NO: 752 - DNA, 22 bp, Artificial Sequence
  RHO-153 targeting domain
gagagctgggcaaagaaattcc

SEQ ID NO: 753 - DNA, 22 bp, Artificial Sequence
  RHO-154 targeting domain
cgaggcagcagcctggacatgg

SEQ ID NO: 754 - DNA, 22 bp, Artificial Sequence
  RHO-155 targeting domain
aggaatatctctgctgagatgc

SEQ ID NO: 755 - DNA, 22 bp, Artificial Sequence
  RHO-156 targeting domain
ttcccgagaagggagagggagg

SEQ ID NO: 756 - DNA, 22 bp, Artificial Sequence
  RHO-157 targeting domain
tccttcctccctctcccttctc

SEQ ID NO: 757 - DNA, 22 bp, Artificial Sequence
  RHO-158 targeting domain
tgttttgcccagaggaagaaga

SEQ ID NO: 758 - DNA, 22 bp, Artificial Sequence
  RHO-159 targeting domain
ccggctggtccaggtaatggca

SEQ ID NO: 759 - DNA, 22 bp, Artificial Sequence
  RHO-160 targeting domain
cagcacagggaagacccaatga

SEQ ID NO: 760 - DNA, 22 bp, Artificial Sequence
  RHO-161 targeting domain
accaggagtgggagagggattt

SEQ ID NO: 761 - DNA, 22 bp, Artificial Sequence
  RHO-162 targeting domain
gctggtgaagccacctaggacc

SEQ ID NO: 762 - DNA, 22 bp, Artificial Sequence
  RHO-163 targeting domain
ggcggtatgagccgggtgtggg

SEQ ID NO: 763 - DNA, 22 bp, Artificial Sequence
  RHO-164 targeting domain
cagccattgcaaagctgggtga

SEQ ID NO: 764 - DNA, 22 bp, Artificial Sequence
  RHO-165 targeting domain
acattgacaggacaggagaagg

SEQ ID NO: 765 - DNA, 22 bp, Artificial Sequence
  RHO-166 targeting domain
tgggtcttccctgtgctgggca

SEQ ID NO: 766 - DNA, 22 bp, Artificial Sequence
  RHO-167 targeting domain
gtacgtggtggtgtgtaagccc

SEQ ID NO: 767 - DNA, 22 bp, Artificial Sequence
  RHO-168 targeting domain
agcaaataacttcccccattcc

SEQ ID NO: 768 - DNA, 22 bp, Artificial Sequence
  RHO-169 targeting domain
ggatttgaggaggccttgggga

SEQ ID NO: 769 - DNA, 22 bp, Artificial Sequence
  RHO-170 targeting domain
ccctgagcttgggcccccagag

SEQ ID NO: 770 - DNA, 22 bp, Artificial Sequence
  RHO-171 targeting domain
cagagattccctgagaatggga

SEQ ID NO: 771 - DNA, 22 bp, Artificial Sequence
  RHO-172 targeting domain
gagttggaagcccgcatctatc

SEQ ID NO: 772 - DNA, 22 bp, Artificial Sequence
  RHO-173 targeting domain
agtccttcctccctctcccttc

SEQ ID NO: 773 - DNA, 22 bp, Artificial Sequence
  RHO-174 targeting domain
gttatttcatttcccgagaagg

SEQ ID NO: 774 - DNA, 22 bp, Artificial Sequence
  RHO-175 targeting domain
atttcatttcccgagaagggag

SEQ ID NO: 775 - DNA, 22 bp, Artificial Sequence
  RHO-176 targeting domain
gacgtagagcgtgaggaagttg

SEQ ID NO: 776 - DNA, 22 bp, Artificial Sequence
  RHO-177 targeting domain
catttcccgagaagggagaggg

SEQ ID NO: 777 - DNA, 22 bp, Artificial Sequence
  RHO-178 targeting domain
gtagagcgtgaggaagttgatg

SEQ ID NO: 778 - DNA, 22 bp, Artificial Sequence
  RHO-179 targeting domain
caggccttcgcagcattcttgg

SEQ ID NO: 779 - DNA, 22 bp, Artificial Sequence
  RHO-180 targeting domain
aggtagtactgtgggtactcga

SEQ ID NO: 780 - DNA, 22 bp, Artificial Sequence
  RHO-181 targeting domain
aaacatgtaggcggccagcatg

SEQ ID NO: 781 - DNA, 22 bp, Artificial Sequence
  RHO-182 targeting domain
tttcatttcccgagaagggaga

SEQ ID NO: 782 - DNA, 22 bp, Artificial Sequence
  RHO-183 targeting domain
gggaagacccaatgactggaga

SEQ ID NO: 783 - DNA, 22 bp, Artificial Sequence
  RHO-184 targeting domain
aaaactgaggcagggagagggg

SEQ ID NO: 784 - DNA, 22 bp, Artificial Sequence
  RHO-185 targeting domain
tgagtcagacccaggctgggca

SEQ ID NO: 785 - DNA, 22 bp, Artificial Sequence
  RHO-186 targeting domain
gggatttgaggaggccttgggg

SEQ ID NO: 786 - DNA, 22 bp, Artificial Sequence
  RHO-187 targeting domain
tctgggggcccaagctcagggt

SEQ ID NO: 787 - DNA, 22 bp, Artificial Sequence
  RHO-188 targeting domain
cgggcccacaggatgcaatttg

SEQ ID NO: 788 - DNA, 22 bp, Artificial Sequence
  RHO-189 targeting domain
acgtagagcgtgaggaagttga

SEQ ID NO: 789 - DNA, 22 bp, Artificial Sequence
  RHO-190 targeting domain
gaccgaggcagcagcctggaca

SEQ ID NO: 790 - DNA, 22 bp, Artificial Sequence
  RHO-191 targeting domain
caggctgggcactgagggagag

SEQ ID NO: 791 - DNA, 22 bp, Artificial Sequence
  RHO-192 targeting domain
tatttcatttcccgagaaggga

SEQ ID NO: 792 - DNA, 22 bp, Artificial Sequence
  RHO-193 targeting domain
gtcccgggcttggcggtggtgg

SEQ ID NO: 793 - DNA, 22 bp, Artificial Sequence
  RHO-194 targeting domain
ctgctgcctcggtcccattctc

SEQ ID NO: 794 - DNA, 22 bp, Artificial Sequence
  RHO-195 targeting domain
agcgtctcctcctgcatctcag

SEQ ID NO: 795 - DNA, 22 bp, Artificial Sequence
  RHO-196 targeting domain
tcagacccaggctgggcactga

SEQ ID NO: 796 - DNA, 22 bp, Artificial Sequence
  RHO-197 targeting domain
agctaccctctccctgtctagg

SEQ ID NO: 797 - DNA, 22 bp, Artificial Sequence
  RHO-198 targeting domain
cagagaggaaaactgaggcagg

SEQ ID NO: 798 - DNA, 22 bp, Artificial Sequence
  RHO-199 targeting domain
ggagagggatttgaggaggcct

SEQ ID NO: 799 - DNA, 22 bp, Artificial Sequence
  RHO-200 targeting domain
gtccttcctccctctcccttct

SEQ ID NO: 800 - DNA, 22 bp, Artificial Sequence
  RHO-201 targeting domain
agagagcttggtgctgggagga

SEQ ID NO: 801 - DNA, 22 bp, Artificial Sequence
  RHO-202 targeting domain
ccttctcgggaaatgaaataac

SEQ ID NO: 802 - DNA, 22 bp, Artificial Sequence
  RHO-203 targeting domain
gcggttctcaacaccaggagac

SEQ ID NO: 803 - DNA, 22 bp, Artificial Sequence
  RHO-204 targeting domain
ctctgggggcccaagctcaggg

SEQ ID NO: 804 - DNA, 22 bp, Artificial Sequence
  RHO-205 targeting domain
tgtgcaggagcccgggagcatg

SEQ ID NO: 805 - DNA, 22 bp, Artificial Sequence
  RHO-206 targeting domain
cagagaggtgtagagggtgctg

SEQ ID NO: 806 - DNA, 22 bp, Artificial Sequence
  RHO-207 targeting domain
ctccccgaagcggaagttgctc

SEQ ID NO: 807 - DNA, 22 bp, Artificial Sequence
  RHO-208 targeting domain
gctagaagcagccattgcaaag

SEQ ID NO: 808 - DNA, 22 bp, Artificial Sequence
  RHO-209 targeting domain
caaacaccattcatggtgatag

SEQ ID NO: 809 - DNA, 22 bp, Artificial Sequence
  RHO-210 targeting domain
tcatttcccgagaagggagagg

SEQ ID NO: 810 - DNA, 22 bp, Artificial Sequence
  RHO-211 targeting domain
tcaccaccccatgaagttccat

SEQ ID NO: 811 - DNA, 22 bp, Artificial Sequence
  RHO-212 targeting domain
gggagtgcaccctccttaggca

SEQ ID NO: 812 - DNA, 22 bp, Artificial Sequence
  RHO-213 targeting domain
aatggccagagattccctgaga

SEQ ID NO: 813 - DNA, 22 bp, Artificial Sequence
  RHO-214 targeting domain
agaatgggaccgaggcagcagc

SEQ ID NO: 814 - DNA, 22 bp, Artificial Sequence
  RHO-215 targeting domain
ggcaagccagacccctcctctc

SEQ ID NO: 815 - DNA, 22 bp, Artificial Sequence
  RHO-216 targeting domain
cccgggcttggcggtggtggct

SEQ ID NO: 816 - DNA, 22 bp, Artificial Sequence
  RHO-217 targeting domain
agcccgggagcatggaggggtc

SEQ ID NO: 817 - DNA, 22 bp, Artificial Sequence
  RHO-218 targeting domain
ccgggttatttcatttcccgag

SEQ ID NO: 818 - DNA, 22 bp, Artificial Sequence
  RHO-219 targeting domain
ggtgtttgttgactgaatatat

SEQ ID NO: 819 - DNA, 22 bp, Artificial Sequence
  RHO-220 targeting domain
ccgtccctgtgtgacgccccag

SEQ ID NO: 820 - DNA, 22 bp, Artificial Sequence
  RHO-221 targeting domain
ggacaggggctgagaggggagg

SEQ ID NO: 821 - DNA, 22 bp, Artificial Sequence
  RHO-222 targeting domain
agagggtgctggtgaagccacc

SEQ ID NO: 822 - DNA, 22 bp, Artificial Sequence
  RHO-223 targeting domain
attgcatcctgtgggcccgaag

SEQ ID NO: 823 - DNA, 22 bp, Artificial Sequence
  RHO-224 targeting domain
cgggttatttcatttcccgaga

SEQ ID NO: 824 - DNA, 22 bp, Artificial Sequence
  RHO-225 targeting domain
ggaaatgaaataacccggacat

SEQ ID NO: 825 - DNA, 22 bp, Artificial Sequence
  RHO-226 targeting domain
ctgactcagcacagctgctcca

SEQ ID NO: 826 - DNA, 22 bp, Artificial Sequence
  RHO-227 targeting domain
ggcacctgaggacaggggctga

SEQ ID NO: 827 - DNA, 22 bp, Artificial Sequence
  RHO-228 targeting domain
ggagagctgggcaaagaaattc

SEQ ID NO: 828 - DNA, 22 bp, Artificial Sequence
  RHO-229 targeting domain
gggcggtatgagccgggtgtgg

SEQ ID NO: 829 - DNA, 22 bp, Artificial Sequence
  RHO-230 targeting domain
cctccctctcccttctcgggaa

SEQ ID NO: 830 - DNA, 22 bp, Artificial Sequence
  RHO-231 targeting domain
tccaggtaatggcactgagcag

SEQ ID NO: 831 - DNA, 22 bp, Artificial Sequence
  RHO-232 targeting domain
gtgggggcctctcctaggagcc

SEQ ID NO: 832 - DNA, 22 bp, Artificial Sequence
  RHO-233 targeting domain
gatggcatggttctccccgaag

SEQ ID NO: 833 - DNA, 22 bp, Artificial Sequence
  RHO-234 targeting domain
cgtcgcattggagaagggcacg

SEQ ID NO: 834 - DNA, 22 bp, Artificial Sequence
  RHO-235 targeting domain
tgggtggggtgtgcaggagccc

SEQ ID NO: 835 - DNA, 22 bp, Artificial Sequence
  RHO-236 targeting domain
ctggacggtgacgtagagcgtg

SEQ ID NO: 836 - DNA, 22 bp, Artificial Sequence
  RHO-237 targeting domain
gaggaaaactgaggcagggaga

SEQ ID NO: 837 - DNA, 22 bp, Artificial Sequence
  RHO-238 targeting domain
ctgaacactgccttgatcttat

SEQ ID NO: 838 - DNA, 22 bp, Artificial Sequence
  RHO-239 targeting domain
cattacctggaccagccggcga

SEQ ID NO: 839 - DNA, 22 bp, Artificial Sequence
  RHO-240 targeting domain
ggagagagcttggtgctgggag

SEQ ID NO: 840 - DNA, 22 bp, Artificial Sequence
  RHO-241 targeting domain
agaataatgtcttgcatttaac

SEQ ID NO: 841 - DNA, 22 bp, Artificial Sequence
  RHO-242 targeting domain
ctaggaaggcaaccaggagtgg

SEQ ID NO: 842 - DNA, 22 bp, Artificial Sequence
  RHO-243 targeting domain
tctcccagacccctccatgctc

SEQ ID NO: 843 - DNA, 22 bp, Artificial Sequence
  RHO-244 targeting domain
acaggggctgagaggggaggca

SEQ ID NO: 844 - DNA, 22 bp, Artificial Sequence
  RHO-245 targeting domain
ggggcagagggaccacacgctg

SEQ ID NO: 845 - DNA, 22 bp, Artificial Sequence
  RHO-246 targeting domain
aggggaggcagaggatgccaga

SEQ ID NO: 846 - DNA, 22 bp, Artificial Sequence
  RHO-247 targeting domain
tggtccaggtaatggcactgag

SEQ ID NO: 847 - DNA, 22 bp, Artificial Sequence
  RHO-248 targeting domain
ccggacatgtgggggcctctcc

SEQ ID NO: 848 - DNA, 22 bp, Artificial Sequence
  RHO-249 targeting domain
gcaggccagcgccatgacccag

SEQ ID NO: 849 - DNA, 22 bp, Artificial Sequence
  RHO-250 targeting domain
ctagctaccctctccctgtcta

SEQ ID NO: 850 - DNA, 22 bp, Artificial Sequence
  RHO-251 targeting domain
gctttggataacattgacagga

SEQ ID NO: 851 - DNA, 22 bp, Artificial Sequence
  RHO-252 targeting domain
gccattgcaaagctgggtgacg

SEQ ID NO: 852 - DNA, 22 bp, Artificial Sequence
  RHO-253 targeting domain
cctaggtctcctggctgtgatc

SEQ ID NO: 853 - DNA, 22 bp, Artificial Sequence
  RHO-254 targeting domain
aacagagaggaaaactgaggca

SEQ ID NO: 854 - DNA, 22 bp, Artificial Sequence
  RHO-255 targeting domain
attacctggaccagccggcgag

SEQ ID NO: 855 - DNA, 22 bp, Artificial Sequence
  RHO-256 targeting domain
gaggggcacctgaggacagggg

SEQ ID NO: 856 - DNA, 22 bp, Artificial Sequence
  RHO-257 targeting domain
gggttatttcatttcccgagaa

SEQ ID NO: 857 - DNA, 22 bp, Artificial Sequence
  RHO-258 targeting domain
agggtgcactcccccctagaca

SEQ ID NO: 858 - DNA, 22 bp, Artificial Sequence
  RHO-259 targeting domain
ccaggagtgggagagggatttg

SEQ ID NO: 859 - DNA, 22 bp, Artificial Sequence
  RHO-260 targeting domain
agaggggaggcagaggatgcca

SEQ ID NO: 860 - DNA, 22 bp, Artificial Sequence
  RHO-261 targeting domain
ccgcctgctgactgccttgcag

SEQ ID NO: 861 - DNA, 22 bp, Artificial Sequence
  RHO-262 targeting domain
ggcttggtgctgcaaacatggc

SEQ ID NO: 862 - DNA, 22 bp, Artificial Sequence
  RHO-263 targeting domain
caggtaatggcactgagcagaa

SEQ ID NO: 863 - DNA, 22 bp, Artificial Sequence
  RHO-264 targeting domain
ttggaacgcggcagggaggctg

SEQ ID NO: 864 - DNA, 22 bp, Artificial Sequence
  RHO-265 targeting domain
tgtccgggttatttcatttccc

SEQ ID NO: 865 - DNA, 22 bp, Artificial Sequence
  RHO-266 targeting domain
caggtagtactgtgggtactcg

SEQ ID NO: 866 - DNA, 22 bp, Artificial Sequence
  RHO-267 targeting domain
ataacagatcccacttaacaga

SEQ ID NO: 867 - DNA, 22 bp, Artificial Sequence
  RHO-268 targeting domain
agggacgggtgcagagttgagt

SEQ ID NO: 868 - DNA, 22 bp, Artificial Sequence
  RHO-269 targeting domain
gaaggagagagcttggtgctgg

SEQ ID NO: 869 - DNA, 22 bp, Artificial Sequence
  RHO-270 targeting domain
ggtcagccacggctaggttgag

SEQ ID NO: 870 - DNA, 22 bp, Artificial Sequence
  RHO-271 targeting domain
atttcacagcaagaaaactgag

SEQ ID NO: 871 - DNA, 22 bp, Artificial Sequence
  RHO-272 targeting domain
tcaaagaagtcaagcgccctgc

SEQ ID NO: 872 - DNA, 22 bp, Artificial Sequence
  RHO-273 targeting domain
gctgctcccacccaagaatgct

SEQ ID NO: 873 - DNA, 22 bp, Artificial Sequence
  RHO-274 targeting domain
gcaacaaacacccaacaatggc

SEQ ID NO: 874 - DNA, 22 bp, Artificial Sequence
  RHO-275 targeting domain
aaatccacttcccaccctgagc

SEQ ID NO: 875 - DNA, 22 bp, Artificial Sequence
  RHO-276 targeting domain
cagggaggctggaggggcacct

SEQ ID NO: 876 - DNA, 22 bp, Artificial Sequence
  RHO-277 targeting domain
gggcaagccagacccctcctct

SEQ ID NO: 877 - DNA, 22 bp, Artificial Sequence
  RHO-278 targeting domain
caggaaaacagatggggtgctg

SEQ ID NO: 878 - DNA, 22 bp, Artificial Sequence
  RHO-279 targeting domain
ttggagaagggcacgtagaagt

SEQ ID NO: 879 - DNA, 22 bp, Artificial Sequence
  RHO-280 targeting domain
agagcttggtgctgggaggagg

SEQ ID NO: 880 - DNA, 22 bp, Artificial Sequence
  RHO-281 targeting domain
tagctaggaaggcaaccaggag

SEQ ID NO: 881 - DNA, 22 bp, Artificial Sequence
  RHO-282 targeting domain
ggctaggttgagcaggatgtag

SEQ ID NO: 882 - DNA, 22 bp, Artificial Sequence
  RHO-283 targeting domain
ctcaccaccccatgaagttcca

SEQ ID NO: 883 - DNA, 22 bp, Artificial Sequence
  RHO-284 targeting domain
aagcaatgtgcaatgttttgcc

SEQ ID NO: 884 - DNA, 22 bp, Artificial Sequence
  RHO-285 targeting domain
ggaagacccaatgactggagaa

SEQ ID NO: 885 - DNA, 22 bp, Artificial Sequence
  RHO-286 targeting domain
tggccaggaccaccaaggacca

SEQ ID NO: 886 - DNA, 22 bp, Artificial Sequence
  RHO-287 targeting domain
aaatattgtccctttcactgtt

SEQ ID NO: 887 - DNA, 22 bp, Artificial Sequence
  RHO-288 targeting domain
catgagcaacttccgcttcggg

SEQ ID NO: 888 - DNA, 22 bp, Artificial Sequence
  RHO-289 targeting domain
agagatattcctggatcacagc

SEQ ID NO: 889 - DNA, 22 bp, Artificial Sequence
  RHO-290 targeting domain
catggaggggtctgggagagtc

SEQ ID NO: 890 - DNA, 22 bp, Artificial Sequence
  RHO-291 targeting domain
atgttttgcccagaggaagaag

SEQ ID NO: 891 - DNA, 22 bp, Artificial Sequence
  RHO-292 targeting domain
gtgggtggggtgtgcaggagcc

SEQ ID NO: 892 - DNA, 22 bp, Artificial Sequence
  RHO-293 targeting domain
ccaggtaatggcactgagcaga

SEQ ID NO: 893 - DNA, 22 bp, Artificial Sequence
  RHO-294 targeting domain
cccaacaatggccagagattcc

SEQ ID NO: 894 - DNA, 22 bp, Artificial Sequence
  RHO-295 targeting domain
gcacctgaggacaggggctgag

SEQ ID NO: 895 - DNA, 22 bp, Artificial Sequence
  RHO-296 targeting domain
gtcagacccaggctgggcactg

SEQ ID NO: 896 - DNA, 22 bp, Artificial Sequence
  RHO-297 targeting domain
ggggcacctgaggacaggggct

SEQ ID NO: 897 - DNA, 22 bp, Artificial Sequence
  RHO-298 targeting domain
agaggaaaactgaggcagggag

SEQ ID NO: 898 - DNA, 22 bp, Artificial Sequence
  RHO-299 targeting domain
agggataacagatcccacttaa

SEQ ID NO: 899 - DNA, 22 bp, Artificial Sequence
  RHO-300 targeting domain
cttggtgctgggaggaggggga

SEQ ID NO: 900 - DNA, 22 bp, Artificial Sequence
  RHO-301 targeting domain
agagggtagctaggaaggcaac

SEQ ID NO: 901 - DNA, 22 bp, Artificial Sequence
  RHO-302 targeting domain
ttgcacattgcttcatggctcc

SEQ ID NO: 902 - DNA, 22 bp, Artificial Sequence
  RHO-303 targeting domain
gaccgagcccattgcccagcac

SEQ ID NO: 903 - DNA, 22 bp, Artificial Sequence
  RHO-304 targeting domain
tgaacactgccttgatcttatt

SEQ ID NO: 904 - DNA, 22 bp, Artificial Sequence
  RHO-305 targeting domain
ggtgcactcccccctagacagg

SEQ ID NO: 905 - DNA, 22 bp, Artificial Sequence
  RHO-306 targeting domain
gcttggtgctgggaggaggggg

SEQ ID NO: 906 - DNA, 22 bp, Artificial Sequence
  RHO-307 targeting domain
ggatacttcgtcttcgggccca

SEQ ID NO: 907 - DNA, 22 bp, Artificial Sequence
  RHO-308 targeting domain
agtcagacccaggctgggcact

SEQ ID NO: 908 - DNA, 22 bp, Artificial Sequence
  RHO-309 targeting domain
agcaccaagcctctgtttccct

SEQ ID NO: 909 - DNA, 22 bp, Artificial Sequence
  RHO-310 targeting domain
tgggcaaagaaattccagggaa

SEQ ID NO: 910 - DNA, 22 bp, Artificial Sequence
  RHO-311 targeting domain
agagggatttgaggaggccttg

SEQ ID NO: 911 - DNA, 22 bp, Artificial Sequence
  RHO-312 targeting domain
gcaatgttttgcccagaggaag

SEQ ID NO: 912 - DNA, 22 bp, Artificial Sequence
  RHO-313 targeting domain
catgtccgggttatttcatttc

SEQ ID NO: 913 - DNA, 22 bp, Artificial Sequence
  RHO-314 targeting domain
aagcccatgagcaacttccgct

SEQ ID NO: 914 - DNA, 22 bp, Artificial Sequence
  RHO-315 targeting domain
tcccaccctgagcttgggcccc

SEQ ID NO: 915 - DNA, 22 bp, Artificial Sequence
  RHO-316 targeting domain
gagagagcttggtgctgggagg

SEQ ID NO: 916 - DNA, 22 bp, Artificial Sequence
  RHO-317 targeting domain
ctacgtgcccttctccaatgcg

SEQ ID NO: 917 - DNA, 22 bp, Artificial Sequence
  RHO-318 targeting domain
cttgcatttaacaggaaaacag

SEQ ID NO: 918 - DNA, 22 bp, Artificial Sequence
  RHO-319 targeting domain
gaaatgaaataacccggacatg

SEQ ID NO: 919 - DNA, 22 bp, Artificial Sequence
  RHO-320 targeting domain
cgaaggcctgagctcagccact

SEQ ID NO: 920 - DNA, 22 bp, Artificial Sequence
  RHO-321 targeting domain
ggagggtgcactcccccctaga

SEQ ID NO: 921 - DNA, 22 bp, Artificial Sequence
  RHO-322 targeting domain
cagcaccaagcctctgtttccc

SEQ ID NO: 922 - DNA, 22 bp, Artificial Sequence
  RHO-323 targeting domain
gggcaaagaaattccagggaat

SEQ ID NO: 923 - DNA, 22 bp, Artificial Sequence
  RHO-324 targeting domain
cttcggggagaaccatgccatc

SEQ ID NO: 924 - DNA, 22 bp, Artificial Sequence
  RHO-325 targeting domain
tgggaggagggggaaggggcag

SEQ ID NO: 925 - DNA, 22 bp, Artificial Sequence
  RHO-326 targeting domain
cctagacagggagagggtagct

SEQ ID NO: 926 - DNA, 22 bp, Artificial Sequence
  RHO-327 targeting domain
taacagagaggaaaactgaggc

SEQ ID NO: 927 - DNA, 22 bp, Artificial Sequence
  RHO-328 targeting domain
tctcagccaccaccgccaagcc

SEQ ID NO: 928 - DNA, 22 bp, Artificial Sequence
  RHO-329 targeting domain
gtcagcacagaccccactgcct

SEQ ID NO: 929 - DNA, 22 bp, Artificial Sequence
  RHO-330 targeting domain
aggaaaactgaggcagggagag

SEQ ID NO: 930 - DNA, 22 bp, Artificial Sequence
  RHO-331 targeting domain
agccattgcaaagctgggtgac

SEQ ID NO: 931 - DNA, 22 bp, Artificial Sequence
  RHO-332 targeting domain
aaatgaaataacccggacatgt

SEQ ID NO: 932 - DNA, 22 bp, Artificial Sequence
  RHO-333 targeting domain
tagctaccctctccctgtctag

SEQ ID NO: 933 - DNA, 22 bp, Artificial Sequence
  RHO-334 targeting domain
tgtgggtggggtgtgcaggagc

SEQ ID NO: 934 - DNA, 22 bp, Artificial Sequence
  RHO-335 targeting domain
tggggaaggagagagcttggtg

SEQ ID NO: 935 - DNA, 22 bp, Artificial Sequence
  RHO-336 targeting domain
gacttggaacgcggcagggagg

SEQ ID NO: 936 - DNA, 22 bp, Artificial Sequence
  RHO-337 targeting domain
aaggagagagcttggtgctggg

SEQ ID NO: 937 - DNA, 22 bp, Artificial Sequence
  RHO-338 targeting domain
gggaaggagagagcttggtgct

SEQ ID NO: 938 - DNA, 22 bp, Artificial Sequence
  RHO-339 targeting domain
atttgaggaggccttggggaag

SEQ ID NO: 939 - DNA, 22 bp, Artificial Sequence
  RHO-340 targeting domain
atccagctggagccctgagtgg

SEQ ID NO: 940 - DNA, 22 bp, Artificial Sequence
  RHO-341 targeting domain
gagagcttggtgctgggaggag

SEQ ID NO: 941 - DNA, 22 bp, Artificial Sequence
  RHO-342 targeting domain
tcctagctaccctctccctgtc

SEQ ID NO: 942 - DNA, 22 bp, Artificial Sequence
  RHO-343 targeting domain
ccgaggcagcagcctggacatg

SEQ ID NO: 943 - DNA, 22 bp, Artificial Sequence
  RHO-344 targeting domain
ggggaaggagagagcttggtgc

SEQ ID NO: 944 - DNA, 22 bp, Artificial Sequence
  RHO-345 targeting domain
tgctgggaggagggggaagggg

SEQ ID NO: 945 - DNA, 22 bp, Artificial Sequence
  RHO-346 targeting domain
cttcttgtgctggacggtgacg

SEQ ID NO: 946 - DNA, 22 bp, Artificial Sequence
  RHO-347 targeting domain
taccacacccgtcgcattggag

SEQ ID NO: 947 - DNA, 22 bp, Artificial Sequence
  RHO-348 targeting domain
agcagcctggacatgggggaga

SEQ ID NO: 948 - DNA, 22 bp, Artificial Sequence
  RHO-349 targeting domain
agccaggtagtactgtgggtac

SEQ ID NO: 949 - DNA, 22 bp, Artificial Sequence
  RHO-350 targeting domain
ggctgcttgcggttctcaacac

SEQ ID NO: 950 - DNA, 22 bp, Artificial Sequence
  RHO-351 targeting domain
ggaccgaggcagcagcctggac

SEQ ID NO: 951 - DNA, 22 bp, Artificial Sequence
  RHO-352 targeting domain
ctgggcaaagaaattccaggga

SEQ ID NO: 952 - DNA, 22 bp, Artificial Sequence
  RHO-353 targeting domain
tgagaggggaggcagaggatgc

SEQ ID NO: 953 - DNA, 22 bp, Artificial Sequence
  RHO-354 targeting domain
gagggtgcactcccccctagac

SEQ ID NO: 954 - DNA, 22 bp, Artificial Sequence
  RHO-355 targeting domain
cggttctcaacaccaggagact

SEQ ID NO: 955 - DNA, 22 bp, Artificial Sequence
  RHO-356 targeting domain
tgtgcaatgttttgcccagagg

SEQ ID NO: 956 - DNA, 22 bp, Artificial Sequence
  RHO-357 targeting domain
gggggagacagggcaaggctgg

SEQ ID NO: 957 - DNA, 22 bp, Artificial Sequence
  RHO-358 targeting domain
gccgggtgtgggtggggtgtgc

SEQ ID NO: 958 - DNA, 22 bp, Artificial Sequence
  RHO-359 targeting domain
ctgcgtaccacacccgtcgcat

SEQ ID NO: 959 - DNA, 22 bp, Artificial Sequence
  RHO-360 targeting domain
cacccaagaatgctgcgaaggc

SEQ ID NO: 960 - DNA, 22 bp, Artificial Sequence
  RHO-361 targeting domain
cctagctaccctctccctgtct

SEQ ID NO: 961 - DNA, 22 bp, Artificial Sequence
  RHO-362 targeting domain
caccaggagacttggaacgcgg

SEQ ID NO: 962 - DNA, 22 bp, Artificial Sequence
  RHO-363 targeting domain
ttggataacattgacaggacag

SEQ ID NO: 963 - DNA, 22 bp, Artificial Sequence
  RHO-364 targeting domain
ttcgggcccacaggatgcaatt

SEQ ID NO: 964 - DNA, 22 bp, Artificial Sequence
  RHO-365 targeting domain
gaagtatccatgcagagaggtg

SEQ ID NO: 965 - DNA, 22 bp, Artificial Sequence
  RHO-366 targeting domain
ggtgtgcaggagcccgggagca

SEQ ID NO: 966 - DNA, 22 bp, Artificial Sequence
  RHO-367 targeting domain
ggagcagccacgggtcagccac

SEQ ID NO: 967 - DNA, 22 bp, Artificial Sequence
  RHO-368 targeting domain
agcgccctgctggggcgtcaca

SEQ ID NO: 968 - DNA, 22 bp, Artificial Sequence
  RHO-369 targeting domain
gagcccgggagcatggaggggt

SEQ ID NO: 969 - DNA, 22 bp, Artificial Sequence
  RHO-370 targeting domain
agggccacagccatgaatggca

SEQ ID NO: 970 - DNA, 22 bp, Artificial Sequence
  RHO-371 targeting domain
gcaatgtgcaatgttttgccca

SEQ ID NO: 971 - DNA, 22 bp, Artificial Sequence
  RHO-372 targeting domain
gaagaggtcagccacggctagg

SEQ ID NO: 972 - DNA, 22 bp, Artificial Sequence
  RHO-373 targeting domain
ggccttcgcagcattcttgggt

SEQ ID NO: 973 - DNA, 22 bp, Artificial Sequence
  RHO-374 targeting domain
ttaacagagaggaaaactgagg

SEQ ID NO: 974 - DNA, 22 bp, Artificial Sequence
  RHO-375 targeting domain
tgatggcatggttctccccgaa

SEQ ID NO: 975 - DNA, 22 bp, Artificial Sequence
  RHO-376 targeting domain
accgaggcagcagcctggacat

SEQ ID NO: 976 - DNA, 22 bp, Artificial Sequence
  RHO-377 targeting domain
agggaccacacgctgaggagag

SEQ ID NO: 977 - DNA, 22 bp, Artificial Sequence
  RHO-378 targeting domain
tggaacgcggcagggaggctgg

SEQ ID NO: 978 - DNA, 22 bp, Artificial Sequence
  RHO-379 targeting domain
tgcacattgcttcatggctcct

SEQ ID NO: 979 - DNA, 22 bp, Artificial Sequence
  RHO-380 targeting domain
gcgttccaagtctcctggtgtt

SEQ ID NO: 980 - DNA, 22 bp, Artificial Sequence
  RHO-381 targeting domain
gggtgtgcaggagcccgggagc

SEQ ID NO: 981 - DNA, 22 bp, Artificial Sequence
  RHO-382 targeting domain
ggcaaagaaattccagggaatg

SEQ ID NO: 982 - DNA, 22 bp, Artificial Sequence
  RHO-383 targeting domain
ggctggaggggcacctgaggac

SEQ ID NO: 983 - DNA, 22 bp, Artificial Sequence
  RHO-384 targeting domain
gcgccctgctggggcgtcacac

SEQ ID NO: 984 - DNA, 22 bp, Artificial Sequence
  RHO-385 targeting domain
gcgtaccacacccgtcgcattg

SEQ ID NO: 985 - DNA, 22 bp, Artificial Sequence
  RHO-386 targeting domain
accaggagacttggaacgcggc

SEQ ID NO: 986 - DNA, 22 bp, Artificial Sequence
  RHO-387 targeting domain
gctgctgcctcggtcccattct

SEQ ID NO: 987 - DNA, 22 bp, Artificial Sequence
  RHO-388 targeting domain
gaagccctccaaattgcatcct

SEQ ID NO: 988 - DNA, 22 bp, Artificial Sequence
  RHO-389 targeting domain
cgtagagcgtgaggaagttgat

SEQ ID NO: 989 - DNA, 22 bp, Artificial Sequence
  RHO-390 targeting domain
ctgaagcagttcctttttgctt

SEQ ID NO: 990 - DNA, 22 bp, Artificial Sequence
  RHO-391 targeting domain
gctggacggtgacgtagagcgt

SEQ ID NO: 991 - DNA, 22 bp, Artificial Sequence
  RHO-392 targeting domain
tgagggctttggataacattga

SEQ ID NO: 992 - DNA, 22 bp, Artificial Sequence
  RHO-393 targeting domain
agccgggtgtgggtggggtgtg

SEQ ID NO: 993 - DNA, 22 bp, Artificial Sequence
  RHO-394 targeting domain
ctcagttttcctctctgttaag

SEQ ID NO: 994 - DNA, 22 bp, Artificial Sequence
  RHO-395 targeting domain
caagacattattctaaagcaaa

SEQ ID NO: 995 - DNA, 22 bp, Artificial Sequence
  RHO-396 targeting domain
tggaacttcatggggtggtgag

SEQ ID NO: 996 - DNA, 22 bp, Artificial Sequence
  RHO-397 targeting domain
gagagggatttgaggaggcctt

SEQ ID NO: 997 - DNA, 22 bp, Artificial Sequence
  RHO-398 targeting domain
cttcgggcccacaggatgcaat

SEQ ID NO: 998 - DNA, 22 bp, Artificial Sequence
  RHO-399 targeting domain
acttggaacgcggcagggaggc

SEQ ID NO: 999 - DNA, 22 bp, Artificial Sequence
  RHO-400 targeting domain
atggccagagattccctgagaa

SEQ ID NO: 1000 - DNA, 22 bp, Artificial Sequence
  RHO-401 targeting domain
cctcagttttcctctctgttaa

SEQ ID NO: 1001 - DNA, 22 bp, Artificial Sequence
  RHO-402 targeting domain
taacagatcccacttaacagag

SEQ ID NO: 1002 - DNA, 22 bp, Artificial Sequence
  RHO-403 targeting domain
gggagagggatttgaggaggcc